(19) United States
(12) Patent Application Publication
(10) Pub. No.: US 2007/0082337 A1
(43) Pub. Date: Apr. 12, 2007

(54) Methods of identifying putative gene products by interspecies sequence comparison and biomolecular sequences uncovered thereby

(75) Inventors: Rotem Sorek, Tel-Aviv (IL); Sarah Pollock, Tel-Aviv (IL); Alex Diber, Rishon-LeZion (IL); Zurit Levine, Herzlia (IL); Sergey Nemzer, RaAnana (IL); Guy Kol, Givat Shmuel (IL); Assaf Wool, Kiryat-Ono (IL); Ami Haviv, Hod-HaSharon (IL); Yuval Cohen, Petach-Tikva (IL); Yossi Cohen, Woking (GB); Ronen Shemesh, ModiIn (IL); Kinneret Savitsky, Tel-Aviv (IL)

Correspondence Address: Martin D. Moynihan;PRTSI, Inc., P.O. Box 16446, Arlington, VA, 22215, US

(73) Assignee: Compugen Ltd., Tel Aviv (IL)

(21) Appl. No.: 11/043,591

(22) Filed: Jan. 27, 2005

Related U.S. Application Data
(60) Provisional application No. 60/579,202, filed on Jun. 15, 2004.
(60) Provisional application No. 60/539,128, filed on Jan. 27, 2004.

(51) Int. Cl. C12Q 1/68; G06F 19/00

(52) U.S. Cl. 435/6; 702/20

(57) ABSTRACT

A method of identifying alternatively spliced exons is provided. The method comprising, scoring each of a plurality of exon sequences derived from genes of a species according to at least one sequence parameter, wherein exon sequences of the plurality of exon sequences scoring above a predetermined threshold represent alternatively spliced exons, thereby identifying the alternatively spliced exons.

### RELATIONSHIP TO EXISTING APPLICATIONS

The present application claims priority from U.S. Provisional Patent Application No. 60/579,202, filed Jun. 15, 2004, and from U.S. Provisional Patent Application No. 60/539,128 filed Jan. 27, 2004, the contents of which are hereby incorporated by reference.

### FIELD OF THE INVENTION

The present invention relates to methods of identifying putative gene products by interspecies sequence comparison and, more particularly, to biomolecular sequences uncovered using these methodologies.

### BACKGROUND OF THE INVENTION

Alternative splicing of eukaryotic pre-mRNAs is a mechanism for generating many transcript isoforms from a single gene. It is known to play important regulatory functions. A classic example is the Drosophila sex-determination pathway, in which alternative splicing acts as a sex-specific genetic switch that forms the basis of a regulatory hierarchy [Boggs et al. (1987). Cell 50:739-747; Baker (1989) Nature 340:521-524; Lopez (1999) Annu. Rev. Genet. 32:279-305]. Another intriguing example was found in the inner ear of the chicken, where differential distribution of splice variants for the calcium-activated potassium channel gene slo may form a tonotopic gradient and attune sensory hair cells to the detection of different sound frequencies [Black (1998) Neuron 20:165-168; Ramanathan et al. (1999) Science 283:215-217; Graveley (2001) Trends Genet. 17:100-107]. Alternative splicing is also implicated in human diseases. For example, the neurodegenerative disease FTDP-17 has been associated with mutations that affect the alternative splicing of tau pre-mRNAs [Goedert et al. (2000) Ann. NY Acad. Sci. 920:74-83; Jiang et al. (2000) Mol. Cell. Biol. 20:4036-4048].

Initial sequencing and analysis of the human genome has placed further attention on the role of alternative splicing. The surprising finding that the genome contains about 30,000 protein-coding genes, significantly less than previously estimated, led to the proposal that alternative splicing contributes greatly to functional diversity [Ewing and Green (2000) Nat. Genet. 25:232-234; Lander et al. (2001) Nature 409:860-921; Venter et al. (2001) Science 291:1304-1351].

Expressed sequence tags (ESTs) provide a primary resource for analyzing gene products and predicting alternative splicing events. More than 5 million human ESTs are available to date, which provide a comprehensive sample of the transcriptome. In recent years, numerous studies attempted to computationally assess the extent of alternative splicing in the human genome. With the availability of a nearly complete sequence of the human genome, aligning ESTs to the genome has become a common strategy.

A number of methods based on this strategy have been developed, to enable large-scale analysis of alternative splicing [Brett (2000) FEBS Lett. 47:83-86; Kan (2002) Genome Res. 12:1837-1845; Kan (2001) Genome Res. 11:875-888; Lander (2001) Nature 409:860-921; Mironov (1999) Genome Res. 9:1288-1293; Modrek (2001) Nucleci Acids Res. 29:2850-2859; Hide (2001) Genome Res. 11:1848-1853]. Some of these are summarized infra.

Mironov et al. have developed an algorithm for predicting exon-intron structure of genomic DNA fragments using EST data. This algorithm (Procrustes-EST) is based on the previously published spliced alignment algorithm [Gelfand et al. (1996) Proc. Natl. Acad. Sci. USA. 93:9061-9066], which explores all possible exon assemblies in polynomial time and finds the multiexon structure with the best fit to a related protein. When applied to known human genes and TIGR EST assemblies, the software found a large number of alternatively spliced genes (˜35%). Most of the alternative splicing events occurred in 5′-untranslated regions. In many cases the use of this software allowed for linking and merging multiple existing assemblies into single contigs [Mironov (1999) Genome Reseach 9:1288-1293].

Kan et al. have developed a software tool, Transcript Assembly Program (TAP), that infers the predominant gene structure and reports alternative splicing events using genomic EST alignments [Kan (2001) Genome Research 11:889-900. The gene structure is assembled from individual splice junction pairs using connectivity information encoded in the ESTs. A method called PASS (Polyadenylation Site Scan) is used to infer poly-A sites from 3′ EST clusters. The gene boundaries are identified using the poly-A site predictions. Reconstructing about one thousand known transcripts, TAP scored a sensitivity of 60% and a specificity of 92% at the exon level. The gene boundary identification process was found to be accurate 78% of the time. TAP also reports alternative splicing patterns in EST alignments. An analysis of alternative splicing in 1124 genomic regions suggested that more than half of human genes undergo alternative splicing. Furthermore, the evolutionary conservation of alternative splicing between human and mouse was analyzed using an EST-based approach.

Modrek et al. have performed a genome-wide analysis of alternative splicing based on human EST data. Tens of thousands of splices and thousands of alternative splices were identified in thousands of human genes. These were mapped onto the human genome sequence to verify that the putative splice junctions detected in the expressed sequences map onto genomic exon intron junctions that match the known splice site consensus [Modrek (2001) Nucleic Acids Research, 29:2850-2859].

As mentioned, the above-described approaches use EST data or full-length cDNA sequences to detect alternative splicing. However, expressed sequences present a problematic source of information, as they are merely a sample of the transcriptome. Thus, the detection of a splice variant is possible only if it is expressed above a certain expression level, or if there is EST library prepared from the tissue type in which the variant is expressed. In addition, ESTs are very noisy and contain numerous erroneous sequences [Sorek (2003) Nucleic Acids. Res. 31: 1067-1074]. For example, many wrongly termed splice events represent incompletely spliced heteronuclear RNA (hnRNA) or oligo(dT)-primed genomic DNA contaminants of cDNA library constructions. Furthermore, the splicing apparatus is known to make errors, resulting in aberrant transcripts that are degraded by the mRNA surveillance system and amount to little that is functionality important [Maquat and Charmichael (2001) Cell 104:173-176; Modrek and Lee (2001) Nat. Genet. 30:13-19]. Consequently the mere presence of a transcript isoform in the ESTs cannot establish a functional role for it. Thus, the use of expressed sequence data allows only very general estimates regarding the number of genes that have splice variants (currently running between 35% and 75%), but does not allow specific estimation regarding the actual number of exons that can be alternatively spliced.

### SUMMARY OF THE INVENTION

The background art fails to teach or suggest a method for large-scale prediction of alternative splicing events, which is devoid of the previously described limitations.

According to one aspect of the present invention there is provided a method of identifying alternatively spliced exons, the method comprising, scoring each of a plurality of exon sequences derived from genes of a species according to at least one sequence parameter, wherein exon sequences of the plurality of exon sequences scoring above a predetermined threshold represent alternatively spliced exons, thereby identifying the alternatively spliced exons.

According to another aspect of the present invention there is provided a system for generating a database of alternatively spliced exons, the system comprising a processing unit, the processing unit executing a software application configured for: (a) scoring each of a plurality of exon sequences derived from genes of a species according to at least one sequence parameter, wherein exon sequences of the plurality of exon sequences scoring above a predetermined threshold represent alternatively spliced exons, to thereby identify the alternatively spliced exons; and (b) storing the identified alternatively spliced exons to thereby generate the database of alternatively spliced exons.

According to yet another aspect of the present invention there is provided a computer readable storage medium comprising data stored in a retrievable manner, the data including sequence information as set forth in the files “transcripts. fasta” and “proteins.fasta” of enclosed CD-ROM1 and in the files “transcripts” and “proteins” of enclosed CD-ROM2 and sequence annotations as set forth in the file “AnnotationForPatent.txt” of enclosed, CD-ROM1.

According to still another aspect of the present invention there is provided a method of predicting expression products of a gene of interest, the method comprising: (a) scoring exon sequences of the gene of interest according to at least one sequence parameter and identifying exon sequences scoring above a predetermined threshold as alternatively spliced exons of the gene of interest; and (b) analyzing chromosomal location of each of the alternatively spliced exons with respect to coding sequence of the gene of interest to thereby predict expression products of the gene of interest.

According to an additional aspect of the present invention there is provided a method of predicting expression products of a gene of interest in a given species, the method comprising (a) providing a contig of exon sequences of the gene of interest of a first species; (b) identifying exon sequences of an orthologue of the gene of interest of the first species which align to a genome of the first species (c) assembling the exon sequences of the orthologue of the gene of interest in the contig, thereby generating a hybrid contig; (d) identifying in the hybrid contig, exon sequences of the orthologue of the gene of interest, which do not align with the exon sequences of the gene of interest of the first species, thereby uncovering non-overlapping exon sequences of the gene of interest; and (e) analyzing chromosomal location of non-overlapping exon sequences of the gene of interest with respect to the chromosomal location of the gene of interest to thereby predict expression products of the gene of interest in a given species.

According to further features in preferred embodiments of the invention described below, at least a portion of the exon sequences are alternatively spliced sequences.

According to still further features in the described preferred embodiments the alternatively spliced sequences are identified by scoring exon sequences of the gene of interest according to at least one sequence parameter, wherein exon sequences scoring above a predetermined threshold represent the alternatively spliced exons of the gene of interest.

According to still further features in the described preferred embodiments the at least one sequence parameter is selected from the group consisting of: (i) exon length; (ii) division by 3; (iii) conservation level between the plurality of exon sequences of genes of a species and corresponding exon sequences of genes of ortholohgous species; (iv) length of conserve intron sequences upstream of each of the plurality of exon sequences; (v) length of conserved intron sequences downstream of each of the plurality of exon sequences; (vi) conservation level of the intron sequences upstream of each of the plurality of exon sequences; and (vii) conservation level of the intron sequences downstream of each of the plurality of exon sequences;

According to still further features in the described preferred embodiments the exon length does not exceed 1000 bp.

According to still further features in the described preferred embodiments the conservation level is at least 95%.

According to still further features in the described preferred embodiments the length of conserved intron sequences upstream of each of the plurality of exon sequences is at least 12.

According to still further features in the described preferred embodiments the length of conserved intron sequences downstream of each of the plurality of exon sequences is at least 15.

According to still further features in the described preferred embodiments the conservation level off the intron sequences upstream of each of the plurality of exon sequences is at least 85%.

According to still further features in the described preferred embodiments the conservation level of the intron sequences downstream of each of the plurality of exon sequences is at least 60%.

According to yet an additional aspect of the present invention there is provided an isolated polynucleotide comprising a nucleic acid sequence being at least 70% identical to a nucleic acid sequence of the sequences set forth in file “transcripts.fasta” of CD-ROM1 or in the file “transcripts” of CD-ROM2.

According to still further features in the described preferred embodiments the nucleic acid sequence is set forth in the file “transcripts.fasta” of enclosed CD-ROM1 or in the file “transcripts” of enclosed CD-ROM 2.

According to still an additional aspect of the present invention there is provided an isolated polynucleotide comprising a nucleic acid sequence encoding a polypeptide having an amino acid sequence at least 70% homologous to a sequence set forth in the file “proteins.fasta” of enclosed CD-ROM1 or in the file “proteins” of enclosed CD-ROM2.

According to a further aspect of the present invention there is provided an isolated polypeptide having an amino sequence at least 80% homologous to a sequence set forth in the file proteins fasta” of enclosed CD-ROM1 or in the file “proteins” of enclosed CD-ROM2.

According to yet a further aspect of the present invention there is provided use of a polynucleotide or polypeptide set forth in the file “transcripts.fasta” of CD-ROM1 or in the file “transcripts” of CD-ROM2 or in the file “proteins.fasta” of enclosed CD-ROM1 or in the file “proteins” of enclosed CD-ROM2 for the diagnosis and/or treatment of the diseases listed in Example 8.

In addition, a brief description of exemplary, non-limiting embodiments of the present invention related to the proteins listed in Table 3 is given below, with regard to the amino acid sequences of the splice variants as compared to the wild type sequences. As is further described hereinbelow, the present invention encompasses both nucleic acid and amino acid sequences, as well as homologs, analogs and derivatives thereof. The present invention also encompasses the exemplary protein (amino acid) sequences as described below.

The below description is given as follows. Each sequence is described with regard to the name of the splice variant as given in the included file. For example, for the first sequence below, the name of the splice variant is “ANGPT1_Skippingexon_{—}5_#PEP_NUM_{—}117”, which is a variant of the wild type protein “ANGPT1”. The splice variant sequence for this variant is described with reference to the wild type amino acid sequence: the amino acid sequence of the splice variant ANGPT1_Skippingexon_{—}5_#PEP_NUM_{—}117 is comprised of a first amino acid sequence that is at least about 90% homologous to amino acids 1-269 of the amino acid sequence of the wild type protein ANGPT1; and a second amino acid sequence that is at least about 70%, optionally at least about 80%, preferably at least about 85%, more preferably at least about 90% and most preferably at least homologous to a polypeptide having the sequence GVLQYGCQWGRLDCNTTS (SEQ ID NO: 205), which corresponds to the unique “tail” sequence. Therefore, the splice variant has a first portion having at least about 90% homology to the specified part of the wild type amino acid sequence, and a second portion with the described homology to the unique tail sequence.

The phrase “contiguous and in a sequential order” indicates that these two portions are part of the same polypeptide (are contiguous) and are in the order given (in a sequential order), as described above with regard to the example.

Also as described above, the term “tail” refers to a portion at the C-terminus of the splice variant protein. An “edge portion” occurs at the junction of two exons that are now contiguous in the splice variant, but were not contiguous in the corresponding wild type protein. A “bridging polypeptide” is a unique sequence (of the splice variant). Located between two amino acid sequences that correspond to portions of the wild type protein. Any of the tail, the edge portion or the bridging polypeptide may be at least about 70%, optionally at least about 80%, preferably at least about 85%, more preferably at least about 90%, and most preferably at least about 95% homologous to the sequences given below. A “bridging amino acid” is an amino acid in the splice variant that is located between two amino acid sequences that correspond to portions of the wild type protein.

Optionally and preferably the edge portion, the bridging polypeptide or the tail may optionally be used as a peptide therapeutic, and/or in an assay (such as a diagnostic assay for example), and/or or as partial or complete antibody epitope that is capable of being specifically bound by and/or elicited by an antibody, preferably a monoclonal antibody and/or a fragment of an antibody. For example, a splice variant may be differentially expressed as compared to the wild type protein with regard to

Optionally, although the percent homology of the portion(s) of a splice variant that correspond to a wild type sequence is preferably at least about 90%, optionally the percent homology is at least about 70%, also optionally at least about 80%, preferably at least about 85%, and most preferably at least about 95% homologous to the corresponding part of the wild type sequence.

It should also be noted that although the edge portions are described as being 22 amino acids in length (11 on either side of the join that is present in the splice variant between two portions of the wild type protein), or 23 amino acids in length if a bridge amino acid is present, the length of an edge portion can also optionally be any number of amino acids from about 10 to about 50, or any number within this range, optionally from about 15 to about 30, preferably from about 20 to about 25 amino acids.

The exemplary embodiments of the present invention are given below with regard to the described sequences.

An isolated ANGPT1_Skippingexon_{—}5_#PEP_NUM_{—}117 polypeptide, comprising a first amino acid sequence being at least about 90% homologous to amino acids 1-269 of ANGPT1, and a second amino acid sequence being at least about 70%, optionally at least about 80%, preferably at least about 85% more preferably at least about 90% and most preferably at least about 95% homologous to a polypeptide having the sequence GVLQYGCQWGRLDCNTTS (SEQ ID NO: 205), Wherein said first and said second amino acid sequences are contiguous and in a sequential order.

An isolated polypeptide corresponding to a tail of ANGPT1_Skippingexon_{—}5_#PEP_NUM_{—}117, comprising a polypeptide having the sequence GVLQYGCQWGRLDCNTTS (SEQ ID NO: 205).

An isolated ANGPT1_Skippingexon_{—}6_#PEP_NUM_{—}118 polypeptide, comprising a first amino acid sequence being at least about 90% homologous to amino acids 1-312 of ANGPT1, and a second amino acid sequence being at least about 90% homologous to amino acids 347-498 of ANGPT1 wherein said first and said second amino acid sequences are contiguous and in a sequential order.

An isolated polypeptide of an edge portion of ANGPT1_Skippingexon_{—}6_#PEP_NUM_{—}118, comprising a first amino acid sequence being at least about 90% homologous to amino acids 302-312, of ANGPT1, and a second amino acid sequence being at least about 90% homologous to amino acids 347-357 of ANGPT1, wherein said first and said second amino acid sequences are contiguous and in a sequential order.

An isolated ANGPT1_Skippingexon_{—}8_#PEP_NUM_{—}119 polypeptide, comprising a first amino acid sequence being at least about 90% homologous to amino acids 1-401 of ANGPT1, and a second amino acid sequence being at least about 70%, optionally at least about 80%, preferably at least about 85%, more preferably at least about 90% and most preferably at least about 95% homologous to a polypeptide having the sequence MW, wherein said first and said second amino acid sequences are contiguous and in a sequential order.

An isolated polypeptide corresponding to a tail of ANGPT1₁₃ Skippingexon_{—}8_#PEP_NUM_{—}119, comprising a polypeptide having the sequence MW.

An isolated APBB1_Skippingexon_{—}10_#PEP_NUM_{—}159 polypeptide, comprising a first amino acid sequence being at least about 90% homologous to amino acids 1-501 of APBB1, and a second amino acid sequence being at least about about 70%, optionally at least about 80%, preferably at least about 85%, more preferably at least about 90% and most preferably at least about 95% homologous to a polypeptide having the sequence WNSQRLRMSWSRSSKSITWGMYLLLNLLG (SEQ ID NO: 206), wherein said first and said second amino acid sequences are contiguous and in a sequential order.

An isolated polypeptide corresponding to a tail of APBB1_Skippingexon_{—}10_#PEP_NUM_{—}159, comprising a polypeptide having the sequence WNSQRLRMSWSRSSKSITWGMYLLLNLLG (SEQ ID NO: 206).

An isolated APBB1_Skippingexon_{—}12_#PEP_NUM_{—}160 polypeptide comprising a first amino acid sequence being at least about 90% homologous to amino acids 1-557 of APBB1, and a second amino acid sequence being at least about 70%, optionally at least about 80%, preferably at least about 85%, more preferably at least about 90% and most preferably at least about 95% homologous to a polypeptide having the sequence DRGSAGRVSGAFPLLPGRGQRCPHVCIHHGCRPSLLLLPHVLVRAQCCQPLR GCAGCVHASLPEVSGCPFPGLHLLPPSTPC (SEQ ID NO: 207), wherein said first and said second amino acid sequences are contiguous and in a sequential order.

An isolated polypeptide corresponding to a tail of APBB1_Skippingexon_{—}12_#PEP_NUM_{—}160, comprising a polypeptide having the sequence DRGSAGRVSGAFPLLPGRGQRCPHVCIHHGCRPSLLLLPHVLVRAQCCQPLR GCAGCVHASLPEVSGCPFPGLHLLPPSTPC (SEQ ID NO: 207).

An isolated APBB1_Skippingexon_{—}3_#PEP_NUM_{—}156 polypeptide, comprising a first amino acid sequence being 16 at least about 90% homologous to amino acids 1-240 of APBB1, and a second amino acid sequence being at least about 70%, optionally at least about 80%, preferably at least about 85%, more preferably at least about 90% and most preferably at least about 95% homologous to a polypeptide having the sequence AHLDRFCSWRRL (SEQ ID NO: 208), wherein said first and said second amino acid sequences are contiguous and in a sequential order.

An isolated polypeptide corresponding to a tail of APBB1_Skippingexon_{—}3_#PEP_NUM_{—}156, comprising polypeptide having the sequence AHLDRFCSWRRL (SEQ ID NO: 208).

An isolated APBB1_Skippingexon_{—}7_#PEP_NUM_{—}157 polypeptide, comprising a first amino acid sequence being at least about 90% homologous to amino acids 1-368 of APBB1, and a second amino acid sequence being at least about 90% homologous to amino acids 414-710 of APBB1, wherein said first and said second amino acid sequences are contiguous and in a sequential order.

An isolated polypeptide of an edge portion of APBB1_Skippingexon_{—}7_#PEP_NUM_{—}157, comprising a first amino acid sequence being at least about 90% homologous to amino acids 358-368 of APBB1, and a second amino acid sequence being at least about 90% homologous to amino acids 414-424 of APBB1, wherein said first and said second amino acid sequences are contiguous and in a sequential order.

An isolated APBB1_Skippingexon_{—}9_#PEP_NUM_{—}158 polypeptide, comprising a first amino acid sequence being at least about 90% homologous to amino acids 1-462 of APBB1, and a second amino acid sequence being at least about 90% homologous to amino acids 502-710 of APBB1, wherein said first and said second amino acid sequences are contiguous and in a sequential order.

An isolated polypeptide of an edge portion of APBB1_Skippingexon_{—}9_#PEP_NUM_{—}158, comprising a first amino acid sequence being at least about 90% homologous to amino acids 452-462 of APBB1, and a second amino acid sequence being at least about 90% homologous to amino acids 502-512 of APBB1, wherein said first and said second amino acid sequences are contiguous and in a sequential order.

An isolated CUL5_Skippingexon_{—}2_#PEP_NUM_{—}137 polypeptide, comprising a first amino acid sequence being at least about 90% homologous to amino acids 1-8 of CUL5, and a second amino acid sequence being at least about 70%, optionally at least about 80%, preferably at least about 85%, more preferably at least about 90% and most preferably at least about 95% homologous to a polypeptide having the sequence GCACSLSLG (SEQ ID NO: 209), wherein said first and said second amino acid sequences are contiguous and in a sequential order.

An isolated polypeptide corresponding to a tail of CUL5_Skippingexon_{—}2_#PEP_NUM_{—}137, comprising a polypeptide having the sequence GCACSLSLG (SEQ ID NO: 209).

An isolated CUL5_Skippingexon_{—}2_#PEP_NUM_{—}138 polypeptide, consisting essentially of an amino acid sequence being at least about 90% homologous to amino acids 119-780 of CUL5.

An isolated CUL5_Skippingexon_{—}8_#PEP_NUM_{—}139 polypeptide, comprising a first amino acid sequence being at least 90% homologous to amino acids 1-260 of CUL5, and a second amino acid sequence being at least 70% optionally at least 80%, preferably at least 85%, more preferably at least 90% and most preferably at least 95% homologous to a polypeptide having the sequence NYI, wherein said first and said second amino acid sequences are contiguous and in a sequential order.

An isolated polypeptide corresponding to a tail of CUL5_Skippingexon_{—}8_#PEP_NUM_{—}139, comprising a polypeptide having the sequence NYI.

An isolated ECE1_Skippingexon_{—}2_#PEP_NUM_{—}129 polypeptide, comprising a first amino acid sequence being at least about 90% homologous to amino acids 1-17 of ECE1, and a second amino acid sequence being at least about 90% homologous to amino acids 47-770 of ECE1, wherein said first and said second amino acid sequences are contiguous and in a sequential order.

An isolated polypeptide of an edge portion of ECE1_Skippingexon_{—}2_#PEP_NUM_{—}129, comprising a first amino acid sequence being at least about 90% homologous to amino acids 7-17 of ECE1, and a second amino acid sequence being at least about 90% homologous to amino acids 47-57 of ECE1, wherein said first and said second amino acid sequences are contiguous and in a sequential order.

An isolated ECE2_Skippingexon_{—}12_#PEP_NUM_{—}132 polypeptide comprising a first ammo acid sequence being at least 90% homologous to amino acids 1-458 of ECE2 and a second amino acid sequence being at least 90% homologous to amino acids 492-765 of ECE2 or a portion thereof wherein said first and said second amino acid sequences are contiguous and in a sequential order.

An isolated polypeptide of an edge portion of ECE2_Skippingexon_{—}12_#PEP_NUM_{—}132, comprising a first amino acid sequence being at least 90% homologous to amino acids 448-458 of ECE2 or a portion thereof, and a second amino acid sequence being at least 90% homologous to amino acids 492-502 of ECE2 or a portion thereof, wherein said first and said second amino acid sequences are contiguous and in a sequential order.

An isolated ECE2_Skippingexon_{—}13_#PEP_NUM_{—}133 polypeptide, comprising a first amino acid sequence being at least 90% homologous to amino acids 1-491 of ECE2, and a second amino acid sequence being at least 90% homologous to amino acids 518-765 of ECE2 or a portion thereof, wherein said first and said second amino acid sequences are contiguous and in a sequential order.

An isolated polypeptide of an edge portion of ECE2_Skippingexon_{—}13_#PEP_NUM_{—}133, comprising a first amino acid sequence being at least 90% homologous to amino acids 481-491 of ECE2 or a portion thereof, and a second amino acid sequence being at least 90% homologous to amino acids 518-528 of ECE2 or a portion thereof, wherein said first and said second amino acid sequences are contiguous and in a sequential order.

An isolated ECE2_Skippingexon_{—}15_#PEP_NUM_{—}134 polypeptide, comprising a first amino acid sequence being at least 90% homologous to amino acids 1-552 of ECE2, and a second amino acid sequence being at least 90% homologous to amino acids 590-765 of ECE2 or a portion thereof, wherein said first and said second amino acid sequences are contiguous and in a sequential order.

An isolated polypeptide of an edge portion of ECE2_Skippingexon_{—}15_#PEP_NUM_{—}134, comprising a first amino acid sequence being at least 90% homologous to amino acids 542-552 of ECE2 or a portion thereof, and a second amino acid sequence being at least 90% homologous to amino acids 590-600 of ECE2 or a portion thereof, wherein said first and said second amino acid sequences are contiguous and in a sequential order.

An isolated ECE2_Skippingexon_{—}2_#PEP_{—}130 polypeptide, comprising a first amino acid sequence being at least about 90% homologous to amino acids 1-13 of ECE2, and a second amino acid sequence being at least about 90% homologous to amino acids 43-765 of ECE2, wherein said first and said second amino acid sequences are contiguous and in a sequential order.

An isolated polypeptide of an edge portion of ECE2_Skippingexon_{—}2_#PEP_NUM_{—}130, comprising a first amino acid sequence being at least about 90% homologous to amino acids 3-13 of ECE2, and a second amino acid sequence being at least about 90% to amino acids 43-53 of ECE2, wherein said first and said second amino acid sequences are contiguous and in a sequential order.

An isolated ECE2_Skippingexon_{—}8_#PEP_NUM_{—}131 polypeptide, comprising a first amino acid sequence being at least about 90% homologous to amino acids 1-272 of ECE2, and a second amino acid sequence being at least about 90% homologous to amino acids 336-765 of ECE2, wherein said first and said second amino acid sequences are contiguous and in a sequential order.

An isolated polypeptide of an edge portion of ECE2_Skippingexon_{—}8_#PEP_NUM_{—}131, comprising a first amino acid sequence being at least about 90% homologous to amino acids 262-272 of ECE2, and a second amino acid sequence being at least about 90% homologous to amino acids 336-346 of ECE2, wherein said first and said second amino acid sequences are contiguous and in a sequential order.

An isolated EDNRB_Skippingexon_{—}4_#PEP_NUM_{—}128 polypeptide, comprising a first amino acid, sequence being at least about 90% homologous to amino acids 1-198 of EDNRB, and a second amino acid sequence being at least about 70%, optionally at least about 80%, preferably at least about 85%, more preferably at least about 90% and most preferably at least about 95% homologous to a polypeptide having the sequence SFTRQQKIGGYSVSISACHWPSLHFFIH (SEQ ID NO: 210), wherein said first and said second amino acid sequences are contiguous and in a sequential order.

An isolated polypeptide corresponding to a tail of EDNRB_Skippingexon_{—}4_#PEP_NUM_{—}128, comprising a polypeptide having the sequence SFTRQQKIGGYSVSISACHWPSLHFFIH (SEQ ID NO: 210).

An isolated EFNA1_Skipping_exon_{—}3_#PEP_NUM_{—}42 polypeptide, comprising a first amino acid sequence being at least about 90% homologous to amino acids 1-130 of EFNA1, and a second amino acid sequence being at least about 90% homologous to amino acids 153-205 of EFNA1, wherein said first and said second amino acid sequences are contiguous and in a sequential order.

An isolated polypeptide, of an edge portion of EFNA1_Skipping_exon_{—}3_#PEP_NUM 42, comprising a first amino acid sequence being at least 90% homologous to amino acids 120-130 of EFNA1, and a second amino acid sequence being at least about 90% homologous to amino acids 153-163 of EFNA1, wherein said first and said second amino acid sequences are contiguous and in a sequential order.

An isolated EFNA3_Skippingexon_{—}3_#PEP_NUM_{—}43 polypeptide, comprising a first amino acid sequence being at least about 90% homologous to amino acids 1-148 of EFNA3, and a second amino acid sequence being at least about 90% homologous to amino acids 171-238 of EFNA3, wherein said first and said second amino acid sequences are contiguous and in a sequential order.

An isolated polypeptide of an edge portion of EFNA3_Skippingexon_{—}3_#PEP_NUM 43, comprising a firsts amino acid sequence being at least about 90% homologous to ammo acids 138-148 of EFNA3, and a second amino acid sequence being at least about 90% homologous to amino acids 171-181 of EFNA3, wherein said first and said second amino acid sequences are contiguous and in a sequential order.

An isolated EFNA3_Skippingexon_{—}4_#PEP_NUM_{—}44 polypeptide, comprising a first amino acid sequence being at least about 90% homologous to amino acids 1-169 of EFNA3, a bridging amino acid K and a second amino acid sequence being at least about 90% homologous to amino acids 197-238 of EFNA3, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of EFNA3_Skippingexon_{—}4_#PEP_NUM_{—}44, comprising a first amino acid sequence being at least about 90% homologous to amino acids 159-169 of EFNA3, a bridging amino acid K and a second amino acid sequence being at least about 90% homologous to amino acids 197-207 of EFNA3, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated EFNA5_Skipping_exon_{—}3_#PEP_NUM_{—}45 polypeptide, comprising a first ammo acid sequence being at least about 90% homologous to amino acids 1-139 of EFNA5, a bridging amino acid Y and a second amino acid sequence being at least 90% homologous to amino acids 163-228 of EFNA5, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of EFNA5_Skipping_exon_{—}3_#PEP_NUM_{—}45, comprising a first amino acid sequence being at least about 90% homologous to amino acids 129-139 of EFNA5, a bridging amino acid Y and a second amino acid sequence being at least about 90% homologous to amino acids 163-173 of EFNA5, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated EFNA5_Skipping_exon_{—}4_#PEP_NUM_{—}46 polypeptide, comprising a first amino acid sequence being at least about 90% homologous to amino acids 1-162 of EFNA5, and a second amino acid sequence being at least about 90% homologous to amino acids 189-228 of EFNA5, wherein said first and said second amino acid sequences are contiguous and in a sequential order.

An isolated polypeptide of an edge portion of EFNA5_Skipping_exon_{—}4_#PEP_NUM_{—}46, comprising a first amino acid sequence being at least about 90% homologous to amino acids 152-162 of EFNA5, and at second amino acid sequence being at least about 90% homologous to amino acids 189-199 of EFNA5, wherein said first and said second amino acid sequences are contiguous and in a sequential order.

An isolated EFNB2_Skipping_exon_{—}2_#PEP_NUM_{—}47 polypeptide, comprising a first amino acid sequence being at least about 90% homologous to amino acids 1-40 of EFNB2, and a second amino acid sequence being at least about 70%, optionally at least about 80%, preferably at least about 85%, more preferably at least 90% and most preferably at least about 95% homologous to a polypeptide having the sequence NYIKWVFGGPG (SEQ ID NO: 211), wherein said first and said second amino acid sequences are contiguous and in a sequential order.

An isolated polypeptide corresponding to a tail of EFNB2_Skipping_exon_{—}2_#PEP_NUM 47, comprising a polypeptide having the sequence NYIKWVFGGPG (SEQ ID NO: 211).

An isolated EFNB2_Skipping_exon_{—}3_#PEP_NUM_{—}48 polypeptide, comprising a first amino acid sequence being at least about 90% homologous to amino acids 1-135 of EFNB2, a bridging amino acid Y and a second amino acid sequence being at least about 90% homologous to amino acids 169-333 of EFNB2, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of EFNB2_Skipping_exon_{—}3_#PEP_NUM_{—}48, comprising a first amino acid sequence being at least about 90% homologous to amino acids 125-135 of EFNB2, a bridging amino acid Y and a second amino acid sequence being at least about 90% homologous to amino acids 169-179 of EFNB2, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated EFNB2_Skipping_exon_{—}4_#PEP_NUM_{—}49 polypeptide, comprising a first amino acid sequence being at least about 90% homologous to amino acids 1-166 of EFNB2, and a second amino acid sequence being at least about 90% homologous to amino acids 205-333 of EFNB2, wherein said first and said second amino acid sequences are contiguous hand in a sequential order.

An isolated polypeptide of an edge portion of EFNB2_Slipping_exon_{—}4_#PEP_NUM 49, comprising a first amino acid sequence being at least about 90% homologous to amino acids 156-166 of EFNB2, and a second amino acid sequence being at least about 90% homologous to amino acids 205-215 of EFNB2, wherein said first and said second amino acid sequences are contiguous and in a sequential order.

An isolated EPHA4_Skipping_exon_{—}12_#PEP_NUM_{—}53 polypeptide, consisting essentially of an amino acid sequence being at least about 90% homologous to amino acids 1-691 of EPHA4.

An isolated EPHA4_Skipping_exon_{—}2_#PEP_NUM_{—}50 polypeptide, comprising a first amino acid sequence being at least about 90% homologous to amino acids 1-31 of EPHA4, and a second amino acid sequence being at least about 70%, optionally at least about 80% preferably at least about 85%, more preferably at least about 90% and most preferably at least about 95% homologous to a polypeptide having the sequence GGSEYHG (SEQ ID NO: 212), wherein said first and said second amino acid sequences are contiguous and in a sequential order.

An isolated polypeptide corresponding to a tail of EPHA4_Skipping_exon_{—}2_#PEP_NUM_{—}50, comprising a polypeptide having the sequence GGSEYHG (SEQ ID NO: 212).

An isolated EPH4_Skipping_exon_{—}3_#PEP_NUM_{—}51 polypeptide comprising a first amino acid sequence being at least about 90% homologous to amino acids 1-53 of EPHA4, and a second amino acid sequence being at least about 70%, optionally at least about 80%, preferably at least about 85%, more preferably at least about 90% and most preferably at least about 95% homologous to a polypeptide having the sequence LAKLDITRLSPRMPPVPSAHPTATLSGKEPPRAPVTEAFSELTTMLPLCPAPVH HLLP (SEQ ID NO: 213), wherein said first and said second amino acid sequences are contiguous and in a sequential order.

An isolated polypeptide corresponding to a tall of EPHA4_Skipping_exon_{—}3_#PEP_NUM 51, comprising a polypeptide having the sequence LAKLDITRLSPRMPPVPSAHPTATLSGKEPPRAPVTEAFSELTTMLPLCPAPVH HLLP (SEQ ID NO: 213).

An isolated EPHA4_Skipping_exon_{—}4_#PEP_NUM_{—}52 polypeptide, comprising a first amino acid sequence being at least about 90% homologous to amino acids 1-274 of EPHA4, a bridging amino acid G and a second amino acid sequence being at least about 90% homologous to amino acids 328-986 of EPHA4, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of EPHA4_Skipping_exon_{—}4_#PEP_NUM_{—}52, comprising a first amino acid sequence being at least about 90% homologous to amino acids 264-274 of EPHA4, a bridging amino acid G and a second amino acid sequence being at least about 90% homologous to amino acids 328-338 of EPHA4, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated EPHA5_Skipping_exon_{—}10_#PEP_NUM_{—}57 polypeptide, consisting essentially of an amino acid sequence being at least about 90% homologous to amino acids 1-618 of EPHA5-followed by C.

An isolated EPHA5_Skipping_exon_{—}14_#PEP_NUM_{—}58 polypeptide, comprising a first amino acid sequence being at least about 90% homologous to amino acids 1-766 of EPHA5, and a second amino acid sequence being at least about 90% homologous to amino acids 837-1037 of EPHA5, wherein said first and said second amino acid sequences are contiguous and in a sequential order.

An isolated polypeptide of an edge portion of EPHA5_Skipping_exon_{—}14_#PEP_NUM_{—}58, comprising a first amino acid sequence being at least about 90% homologous to amino acids 756-766 of EPHA5, and a second amino acid sequence being at least about 90% homologous to amino acids 837-847 of EPHA5, wherein said first and said second amino acid sequences are contiguous and in a sequential order.

An isolated EPHA5_Skipping_exon_{—}16_#PEP_NUM_{—}59 polypeptide, comprising amino acid sequence being at least about 90% homologous to amino acids 1-886 of EPHA5, and a second amino acid sequence being at least about 70%, optionally at least about 80% preferably at least about 85%, more preferably at least about 90% and most preferably at least about 95% homologous to a polypeptide having the sequence SI, wherein said first and said second amino acid sequences are contiguous and in a sequential order.

An isolated polypeptide corresponding to a tail of EPHA5_Skipping_exon_{—}16_#PEP_NUM_{—}59, comprising a polypeptide having the sequence SI.

An isolated EPHA5_Skipping_exon_{—}4_#PEP_NUM_{—}54 polypeptide, comprising a first amino acid sequence being at least about 90% homologous to amino acids 1-303 of EPHA5, a bridging amino acid G and a second amino acid sequence being at least about 90-% homologous to amino acids 357-1037 of EPHA5, wherein said first amino acid sequence is contiguous to said bridging amino acid, and said second amino acid sequence is contiguous to said bridging amino acid, and wherein said first amino acid sequence, said bridging amino acid and said second amino acid sequence are in a sequential order.

An isolated polypeptide of an edge portion of EPHA5_Skipping_exon_{—}4_#PEP_NUM_{—}54, comprising a first amino acid sequence being at least about 90% homologous to amino acids 293-303 of EPHA5, a bridging amino acid G and a second amino acid sequence being at least about 90% homologous to amino acids 357-367 of EPHA5, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated EPHA5_Skipping_exon_{—}5_#PEP_NUM_{—}55 polypeptide, comprising a first amino acid sequence being at least 90% homologous to amino acids 1-355 of EPHA5, bridged by T and a second amino acid sequence being at least 90% homologous to amino acids 469-1037 of EPHA5, wherein said first amino acid is contiguous to said bridging amino acid and said second amino acid sequence, is contiguous to said bridging amino acid, and wherein said first amino acid, said bridging amino acid and said second amino acid sequence are in a sequential order.

An isolated polypeptide of an edge portion of EPHA5_Skipping_exon_{—}5_#PEP_NUM_{—}55, comprising a first amino acid sequence being at least 90% homologous to amino acids 345-355 of EPHA5, bridged by T and a second amino acid sequence being at least 90% homologous to amino acids 469-479 of EPHA5, wherein said first amino acid is contiguous to said bridging amino acid and said second amino acid sequence is contiguous to said bridging amino acid, and wherein said first amino acid, said bridging amino acid and said second amino acid sequence are in a sequential order.

An isolated polypeptide of an edge portion of EPHA5_Skipping_exon_{—}5_#PEP_NUM_{—}55, comprising a first amino acid sequence being at least about 90% homologous to amino acids 345-355 of EPHA4, a bridging amino acid T and a second amino acid sequence being at least about 90% homologous to amino acids 469-479 of EPHA5, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated EPHA5_Skipping_exon_{—}8_#PEP_NUM_{—}56 polypeptide, comprising a first amino acid sequence being at least about 90% homologous to amino acids 1-565 of EPHA5, and a second amino acid sequence being at least about 70%, optionally at least about 80%, preferably at least about 85%, more preferably at least about 90% and most preferably at least about 95% homologous to a polypeptide having the sequence IVAVGGLLPCALLPIQA (SEQ ID NO: 214), wherein said first and said second amino acid sequences are contiguous and in a sequential order.

An isolated polypeptide corresponding to a tail of EPHA5_Skipping_exon_{—}8_#PEP_NUM_{—}56, comprising a polypeptide having the sequence IVAVGGLLPCALLPIQA (SEQ ID NO: 214).

An isolated EPHA5_Skippingexon_{—}17_#PEP_NUM_{—}60 polypeptide, comprising a first amino acid sequence being at least about 90% homologous to amino acids 1-951 of EPHA5, and a second amino acid sequence being at least about 90% homologous to amino acids 1004-1037 of EPHA5, wherein said first and said second amino acid sequences are contiguous and in a sequential order.

An isolated polypeptide of an edge portion of EPHA5_Skippingexon_{—}17_#PEP_NUM_{—}60, comprising a first amino acid sequence being at least about 90% homologous to amino acids 941-951 of EPHA5, and a second amino acid sequence being at least about 90% homologous to amino acids 1004-1014 of EPHA5, wherein said first and said second amino acid sequences are contiguous and in a sequential order.

An isolated EPHA7_Skippingexon_{—}10_#PEP_NUM_{—}61 polypeptide, consisting essentially of an amino acid sequence being at least about 90% homologous to amino acids 1-599 of EPHA7.

An isolated EPHA7_Skippingexon_{—}15_#PEP_NUM_{—}62 polypeptide, comprising a first amino acid sequence being at least about 90% homologous to amino acids 1-844 of EPHA7, and a second amino acid sequence being at least about 70%, optionally at least about 80%, preferably at least about 85%, more preferably at least about 90% and most preferably at least about 95% homologous to a polypeptide having the sequence ANKPSSGSKHS (SEQ ID NO: 215), wherein said first and said second amino acid sequences are contiguous and in a sequential order.

An isolated polypeptide corresponding to a tail of EPHA7_Skippingexon_{—}15_#PEP_NUM_{—}62, comprising a polypeptide having the sequence ANKPSSGSKHS (SEQ ID NO: 215).

An isolated EPHB1_Skippingexon_{—}10_#PEP_NUM_{—}65 polypeptide, comprising a first namo acid sequence being at least about 90% homologous to amino acids 1-586 of EPHB1, and a second amino acid sequence being at least about 90% homologous to amino acids 628-984 of EPHB1, wherein said first and said second amino acid sequences are contiguous and in a sequential order.

An isolated polypeptide of an edge portion of EPHB1_Skippingexon_{—}10_#PEP_NUM_{—}65, comprising a first amino acid sequence being at least about 90% homologous to amino acids 576-586 of EPHB1, and a second amino acid sequence being at least about 90% homologous to amino acids 628-638 of EPHB1, wherein said first and said second amino acid sequences are contiguous and in a sequential order.

An isolated EPHB1_Skippingexon_{—}6_#PEP_NUM_{—}63 polypeptide, comprising a first amino acid sequence being at least about 90% homologous to amino acids 1-432 of EPHB1, and a second amino acid sequence being at least about 70%, optionally at least about 80%, preferably at least about 85%, more preferably at least about 90% and most preferably at least about 95% homologous to a polypeptide having the sequence GTG, wherein said first and said second amino acid sequences are contiguous and in a sequential order.

An isolated polypeptide corresponding to a tail of EPHB1_Skippingexon_{—}6_#PEP_NUM_{—}63, comprising a polypeptide having the sequence GTG.

An isolated EPHB1_Skippingexon_{—}8_#PEP_NUM_{—}64 polypeptide, comprising a first amino acid sequence at least about 90% homologous to amino acids 1-528 of EPHB1, and a second amino acid sequence being at least about 70%, optionally at least about 80%, preferably at least about 85%, more preferably at least about 90% and most preferably at least about 95% homologous to a polypeptide having the sequence GNGLIAKRLCTAISSSITAQAEGSLEKCTRGV (SEQ ID NO: 216), wherein said first and said second amino acid sequences are contiguous and in a sequential order.

An isolated polypeptide corresponding to a tail of EPHB1_Skippingexon_{—}8_#PEP_NUM_{—}64, comprising polypeptide having the sequence GNGLIAKRLCTAISSSITAQAEGSLEKCTRGV (SEQ ID NO: 216).

An isolated ErbB2_Skippingexon_{—}6_#PEP_NUM_{—}76 polypeptide, comprising a first amino acid sequence being at least about 90% homologous to amino acid 1-214 of ErbB2 and a second amino acid sequence being at lest about 70%, optionally at least about 80%, preferably at least about 85%, more preferably at least about 90% and most preferably at least about 95% homologous to a polypeptide having the sequence RLPPLQPQWHL (SEQ ID NO: 217), wherein said first and said second amino acid sequences are contiguous and in a sequential order.

An isolated polypeptide corresponding to a tail of ErbB2_Skippingexon_{—}6_#PEP_NUM_{—}76, comprising a polypeptide having the sequence RLPPLQPQWHL (SEQ ID NO: 217).

An isolated ErbB3_Skippingexon_#PEP_NUM_{—}78 polypeptide, consisting essentially of an amino acid sequence being at least 90% homologous to amino acids 1-468 of ErbB3, followed by V.

An isolated ErbB3 Skippingexon_{—}18_#PEP_NUM_{—}79 polypeptide comprising a first amino acid sequence being at least about 90% homologous to amino acids 1-685 of ErbB3, and a second amino acid sequence being at least about 90% homologous to amino acids 726-1342 of ErbB3, wherein said first and said second amino acid sequences are contiguous and in sequential order.

An isolated polypeptide of an edge portion of ErbB3_Skippingexon_{—}18_#PEP_NUM_{—}79, comprising a first amino acid sequence being at least about 90% homologous to amino acids 675-685 of ErbB3, and a second amino acid sequence being at least about 90% homologous to amino acids 726-736 of ErbB3, wherein said first and said second amino acid sequences are contiguous and in a sequential order.

An isolated ErbB3_Skippingexon_{—}4_#PEP_NUM_{—}77 polypeptide, comprising a first amino acid sequence being at least about 90% homologous to amino acids 1-140 of ErbB3, a bridging amino acid G and a second amino acid sequence being at least about 90% homologous to amino acids 174-1342 of ErbB3, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of ErbB3_Skippingexon_{—}4_#PEP_NUM_{—}77, comprising a first amino acid sequence being at least about 90% homologous to amino acids 130-140 of ErbB3, a bridging amino acid G and a second amino acid sequence being at least about 90% homologous to amino acids 174-184 of ErbB3, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated ErbB4_Skippingexon_{—}14_#PEP_NUM 80 polypeptide, comprising a first amino acid sequence being at least about 90% homologous to amino acids 1-541 of ErbB4, and a second amino acid sequence being at least about 70%, optionally at least about 80%, preferably at least about 85%, more preferably at least about 90% and most preferably at least about 95% homologous to a polypeptide having the sequence VLTTVQSALILKMAQTVWKNVQMAYRGQTVSFSSMLIQIGSATHAIQTAPKG VTVPLVMTAFTHGRAIPLYHNMLELP (SEQ ID NO: 218), wherein said firsthand said second amino acid sequences are contiguous and in a sequential order.

An isolated polypeptide corresponding to a tail of ErbB4_Skippingexon_{—}14_#PEP_NUM_{—}80, comprising a polypeptide having the sequence VLTTVQSALILKMAQTVWKNVQMAYRGQTVSFSSMLIQIGSATHAIQTAPKG VTVPLVMTAFTHGRAIPLYHNMLELP (SEQ ID NO: 218).

An isolated “ErbB4_Skippingexon_{—}16_#PEP_NUM_{—}81 polypeptide, comprising a first amino acid sequence being at least about 90% homologous to amino acids 1-624 of ErbB4, and a second amino acid sequence being at least about 90% homologous to amino acids 650-1308 of ErbB4, wherein said first and said second amino acid sequences are contiguous and in a sequential order.

An isolated polypeptide of an edge portion of ErbB4_Skippingexon_{—}16_#PEP_NUM_{—}81, comprising a first amino acid sequence being at least about 90% homologous to amino acids 614-624 of ErbB4, and a second amino acid sequence being at least about 90% homologous to amino acids 650-660 of ErbB4, wherein said first and said second amino acid sequences are contiguous and in a sequential order.

An isolated FGF10_Skippingexon_{—}2_#PEP_NUM_{—}114 polypeptide, comprising a first amino acid sequence being at least about 90% homologous to amino acids 1-108 of FGF10, and a second amino acid sequence being at least about 70%, optionally at least about, 80%, preferably at least about 85%, more preferably at least about 90% and most preferably at least about 95% homologous to a polypeptide having the sequence KRI, wherein said first and said second amino acid sequences are contiguous and in a sequential order.

An isolated polypeptide corresponding to a tail of FGF10_Skippingexon_{—}2_#PEP_NUM_{—}114, comprising a polypeptide having the sequence KRI.

An isolated FGF11_Skipping_exon_{—}2_#PEP_NUM_{—}37 polypeptide, comprising a first amino acid sequence being at least about 90% homologous to amino acids 1-64 of FGF11, a bridging amino acid A and a second amino acid sequence being at least about 90% homologous to amino acids 101-225 of FGF11, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion, of FGF11_Skipping_exon_{—}2_#PEP_NUM_{—}37, comprising a first amino acid sequence being at least about 90% homologous to amino acids 54-64 of FGF11, a bridging amino acid A and a second amino acid sequence being at least about 90% homologous to amino acids 101-111 of FGF11, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated FGF12_Skipping_exon_{—}2_Short_isoform_#PEP_NUM_{—}39 polypeptide, comprising a first amino acid sequence being at least about 90% homologous to amino acids 1-4 of FGF12_Short_isoform, a bridging amino acid A and a second amino acid sequence being at least about 90% homologous to amino acids 43-181 of FGF12 Short isoform, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of FGF12_Skipping_exon_{—}2_Short_isoform_#PEP_NUM_{—}39, comprising a first amino acid sequence being at least about 90% homologous to amino acids 1-4 of FGF12_Short_isoform, a bridging amino acid A and a second amino acid sequence being at least about 90% homologous to amino acids 43-53 of FGF12_Short_isoform, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated FGF12_Skipping_exon_{—}2_long_isoform_#PEP_NUM_{—}38 polypeptide, comprising a first amino acid sequence being at least about 90% homologous to amino acids 1-66 of FGF12_Long_isoform, a bridging amino acid A and a second amino acid sequence being at least about 90% homologous to amino acids 105-243 of FGF12_Long_isoform, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of FGF12_Skipping_exon_{—}2_long_isoform_#PEP_NUM_{—}38, comprising a first amino acid sequence beings at least about 90. % homologous to amino acids 56-66 of FGF12_Long_isoform, a bridging amino acid A and a second amino acid sequence being at least about 90%, homologous to amino acids 105-115 of FGF12_Long_isoform, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated FGF13_Skipping_exon_{—}2_Long_isoform_#PEP_NUM_{—}40 polypeptide, comprising a first amino acid sequence being at least about 90% homologous to amino acids 1-62 of FGF13_Long_isoform, a bridging amino acid A and a second amino acid sequence being at least about 90% homologous to amino acids 101-245 of FGF13_Long_isoform, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of FGF13_Skipping_exon_{—}2_Long_isoform_#PEP_NUM_{—}40, comprising a first amino acid sequence being at least about 90% homologous to amino acids 52-62 of FGF12_Long_isoform, a bridging amino acid A and a second amino acid sequence being at least about 90% homologous to amino acids 101-115 of FGF13_Long_isoform, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated FGF13_Skipping_exon_{—}3_Long_isoform_#PEP_NUM_{—}41 polypeptide, comprising a first amino acid sequence being at least about 90% homologous to amino acids 1-99 of FGF13_Long_isoform, and a second amino acid sequence being at least about 70%, optionally at least about 80%, preferably at least about 85%, more preferably at least about 90% and most preferably at least about 95% homologous to a polypeptide having the sequence RTFHT, wherein said first and said second amino acid sequences are contiguous and in a sequential order.

An isolated polypeptide corresponding to a tail of FGF13_Skipping_exon_{—}3_Long_isoform_#PEP_NUM_{—}41, comprising a polypeptide having the sequence RTFHT.

An isolated. FGF13_Skipping_exon_{—}2_Short_isoform_#PEP_NUM_{—}40a polypeptide, comprising a first amino acid sequence being at least about 90% homologous to amino acids 1-9 of FGF13_Short_isoform, a bridging amino acid A and a second amino acid sequence being at least about 90% homologous to amino acids 48-192 of FGF13_Short_isoform, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of FGF13_Skipping_exon_{—}2_Short_isoform_#PEP_NUM_{—}40a, comprising a first amino acid sequence being at least about 90% homologous to amino acids 1-9 of FGF13_Short_isoform, a bridging amino acid A and a second amino acid sequence being at least about 90% homologous to amino acids 48-58 of FGF13_Short_isoform, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated FGF13_Skipping_exon_{—}3_Short isoform_#PEP_NUM_{—}41a polypeptide, comprising a first amino acid sequence being at least about 90% homologous to amino acids 1-46 of FGF13_Short_isoform, and a second amino acid sequence being at least about 70%, optionally at least about 80%, preferably at least about 85%, more preferably at least about 90% and most preferably at least about 95% homologous to a polypeptide having the sequence RTFHT (SEQ ID NO: 219), wherein said first and said second amino acid sequences are contiguous and in a sequential order.

An isolated polypeptide corresponding to a tail of FGF13_Skipping_exon_{—}3_Short_isoform_#PEP_NUM_{—}41a, comprising a polypeptide having the RTFHT (SEQ ID NO: 219).

An isolated FGF18_Skipping_exon_{—}2_#PEP_NUM_{—}115 polypeptide, comprising a first amino acid sequence being at least about 90% homologous to amino acids 1-12 of FGF18, and a second amino acid sequence being at least about 70%, optionally at least about 80%, preferably at least about 85%, more preferably at least about 90% and most preferably at least about 95% homologous to a polypeptide having the sequence WLPRRTWTSAASTWRTRRGLGTM (SEQ ID NO: 220), wherein said first and said second amino acid sequences are contiguous and in a sequential order.

An isolated polypeptide corresponding to a tail of FGF18_Skippingexon_{—}2_#PEP_NUM_{—}115, comprising a polypeptide having the sequence WLPRRTWTSAASTWRTRRGLGTM (SEQ ID NO: 220).

An isolated FGF18_Skippingexon_{—}4_#PEP_NUM_{—}116 polypeptide, comprising a first amino acid sequence being at least about 90% homologous to amino acids 1-84 of FGF18, and a second amino acid sequence being at least about 70%, optionally at least about 80%, preferably at least about 85%, more preferably at least about 90% and most preferably at least about 95% homologous to a polypeptide having the sequence RWHQQGVWVHREGSGEQLHGPDVG (SEQ ID NO: 221), wherein said first and said second amino acid sequences are contiguous and in a sequential order.

An isolated polypeptide corresponding to a tail of FGF18_Skippingexon_{—}4_#PEP_NUM_{—}116, comprising a polypeptide having the sequence RWHQQGVWVHREGSGEQLHGPDVG (SEQ ID NO: 221).

An isolated FGF9_Skippingexon_{—}2_#PEP_NUM_{—}113 polypeptide, comprising a first amino acid sequence being at least about 90% homologous to amino acids 1-93 of FGF9, and a second amino acid sequence being at least about 70%, optionally at least about 80%, preferably at least about 85%, more preferably at least about 90% and most preferably at least about 95% homologous to a polypeptide having the sequence KTNPRVCIQRTVRRKLV (SEQ ID NO: 222), wherein said first and said second amino acid sequences are contiguous and in a sequential order.

An isolated polypeptide corresponding to a tail of FGF9_Skippingexon_{—}2_#PEP_NUM_{—}113, comprising a polypeptide having the sequence KTNPRVCIQRTVRRKLV (SEQ ID NO: 222).

An isolated FSHR_Intron_{—}7 retention_#PEP_NUM_{—}28 polypeptide, consisting essentially of an amino acid sequence being at least about 90% homologous to amino acids 1-198 of FSHR.

An isolated FSHR_Skipping exon_{—}7_#PEP_NUM_{—}26 polypeptide, comprising a first amino acid sequence being at least about 90% homologous to amino acids 1-174 of FSHR, and a second amino acid sequence being at least about 90% homologous to amino acids 198-695 of FSHR, wherein said first and said second amino acid sequences are contiguous and in a sequential order.

An isolated polypeptide of an edge portion of FSHR_Skipping_exon_{—}7_#PEP_NUM_{—}26, comprising a first amino acid sequence being at least about 90% homologous to amino acids 164-174 of FSHR, and a second amino acid sequence being at least about 90% homologous to amino acids 198-208 of FSHR, wherein said first and said second amino acid sequences are contiguous and in a sequential order.

An isolated FSHR_Skipping_exon_{—}8_#PEP_NUM_{—}27 polypeptide, comprising a first amino acid sequence being at least about 90% homologous to amino acids 1-197 of FSHR, and a second amino acid sequence being at least about 90% homologous to amino acids 223-695 of FSHR, wherein said first and said second amino acid sequences are contiguous and in a sequential order.

An isolate polypeptide of an edge portion of FSHR_Skipping_exon_{—}8_#PEP_NUM_{—}27, comprising a first amino acid sequence being at least about 90% homologous to amino acids 187-197 of FSHR, and a second amino acid sequence being at least about 90% homologous to amino acids 223-233 of FSHR, wherein said first and said second amino acid sequences are contiguous and in a sequential order.

An isolated FSHR_with_Novel_exon_{—}8A_#PEP_NUM_{—}29 polypeptide comprising a first amino acid sequence being at least about 90% homologous to amino acids 1-223 of FSHR, an amino acid sequence being at least about 70%, optionally at least about 80%, preferably at least about 85%, more preferably at least about 90% and most preferably at least about 95% homologous to a bridging polypeptide having the sequence NRRTRTPTEPNVLLAKYPSGQGVLEEPESLSSSI (SEQ ID NO: 223), and a second amino acid sequence being at least about 90% homologous to amino acids 224-695 of FSHR, wherein said first amino acid sequence is contiguous to said bridging polypeptide and said second amino acid sequence is contiguous to said bridging polypeptide, and wherein said first amino acid, said bridging polypeptide and said second amino acid sequence are in a sequential order.

An isolated polypeptide of an edge portion of FSHR_with_Novel_exon_{—}8A_#PEP_NUM_{—}29, comprising an amino acid sequence of NRRTRTPTEPNVLLAKYPSGQGVLEEPESLSSSI (SEQ ID NO: 223).

An isolated GFRA1_Skippingexon_{—}4_#PEP_NUM_{—}107 polypeptide comprising a first amino acid sequence being at least about 90% homologous to amino acids 1-111 of GFRA1, and a second amino acid sequence being at least about 90% homologous to amino acids 140-465 of GFRA1, wherein said first and said second amino acid sequences are contiguous and in a sequential order.

An isolated polypeptide of an edge portion of GFRA1_Skipping_exon_{—}4_#PEP_NUM_{—}107, comprising a first amino acid sequence being at least about 90% homologous to amino acids 101-111 of GFRA1, and a second amino acid sequence being at least about 990% homologous to amino acids 140-150 of GFRA1, wherein said first and said second amino acid sequences are contiguous and in a sequential order.

An isolated GFRA2_Skippingexon_{—}3_#PEP_NUM_{—}108 polypeptide, consisting essentially of an amino acid sequence being at least about 90% homologous to amino acids 1-60 of GFRA2.

An isolated HSFLT_Skipping_exon_{—}19_#PEP_NUM_{—}8 polypeptide, comprising a first amino acid sequence being at least 90% homologous to amino acids 1-864 of HSFLT, and a second amino acid sequence being at least 90% homologous to amino acids 903-1338 of HSFLT or a portion thereof, wherein said first and said second amino acid sequences are contiguous and in a sequential order.

An isolated polypeptide of an edge portion of HSFLT_Skipping_exon_{—}19_#PEP_NUM_{—}8, comprising a first amino acid sequence being at least 90% homologous to amino acids 854-864 of HSFLT or a portion thereof, and a second amino acid sequence being at least 90% homologous to amino acids 903-913 of HSFLT or a portion thereof, wherein said first and said second amino acid sequences are contiguous and in a sequential order.

An isolated Heparanase2_Skippingexon_{—}10_#PEP_NUM_{—}146 polypeptide, comprising a first amino acid sequence being at least about 90% homologous to amino acids 1-440 of Heparanase2, and a second amino acid sequence being at least about 70%, optionally at least about 80%, preferably at least about 85%, more preferably at least about 90% and most preferably at least about 95% homologous to a polypeptide having the sequence PQLRSWVHYTFYHQLASIKKENQAGWDSQRQAGSPVPAAALWAGGPKVQV SATEWPALSDGGRRDPPRIEAPPPSGRPDIGHPSSHHGLLCGQECQCFGLPLPIS YPHTHGYQWACWAASTPPLQ (SEQ ID NO: 224), wherein said first and said second amino acid sequences are contiguous and in a sequential order.

An isolated polypeptide corresponding to a tail of Heparanase2_Skipping_exon_{—}10_#PEP_NUM_{—}146, comprising a polypeptide having the sequence PQLRSWVHYTFYHQLASIKKENQAGWDSQRQAGSPVPAAALWAGGPKVQV SATEWPALSDGGRRDPPRIEAPPPSGRPDIGHPSSHHGLLCGQECQCFGLPLPIS YPHTHGYQWACWAASTPPLQ (SEQ ID NO: 224).

An isolated Heparanase2_Skippingexon_{—}11_#PEP_NUM_{—}147 polypeptide, comprising a first amino acid sequence being at least about 90% homologous to amino acids 1-489 of Heparanase2, and a second amino acid sequence being at least about 90% homologous to amino acids 538-592 of Heparanase2, wherein said first and said second amino acid sequences are contiguous and in a sequential order.

An isolated polypeptide of an edge portion of Heparanase2_Skippingexon_{—}11_#PEP_NUM_{—}147, comprising a first amino acid sequence being at least about 90% homologous to amino acids 479-489 acid Heparanase2, and a second amino acid sequence being a at least about 90 homologous to amino acids 538-548 of Heparanase2, wherein said first and said second amino acid sequences are contiguous and in a sequential order.

An isolated Heparanase2_Skippingexon_{—}5_#PEP_{—}141 polypeptide, comprising a first amino acid sequence being at least about 90% homologous to amino acids 1-261 of Heparanase2, and a second amino acid sequence being at least about 90% homologous to amino acids 395-396 of Heparanase2, wherein said first and said second amino acid” sequences are contiguous and in a sequential order.

An isolated polypeptide of an edge portion of Heparanase2_Skippingexon_{—}5_#PEP_NUM_{—}141, comprising a first amino acid sequence being at least about 90% homologous to amino acids 251-261 of Heparanase2, and a second amino acid sequence being at least about 90% homologous to amino acids 395-396 of Heparanase2, wherein said first and said second amino acid sequences are contiguous and in a sequential order.

An isolated Heparanase2_Skippingexon_{—}6_#PEP_NUM_{—}142 polypeptide, comprising a first amino acid sequence being at least about 90% homologous to amino acids 1-3119 of Heparanase2, and a second amino acid sequence being at least about 90% homologous to amino acids 335-592 of Heparanase2, wherein said first and said second amino acid sequences are contiguous and in a sequential order.

An isolated polypeptide of an edge portion of Heparanase2_Skippingexon_{—}6_#PEP_NUM_{—}142, comprising a first amino acid sequence being at least about 90% homologous to amino acids 309-319 of Heparanase2, and a second amino acid sequence being at least about 90% homologous to amino acids 335-345 of Heparanase2, wherein said first and said second amino acid sequences are contiguous and in a sequential order.

An isolated Heparanase2_Skippingexon_{—}7_#PEP_NUM_{—}143 polypeptide, comprising a first amino acid sequence being at least about 90% homologous to amino acids 1-334 of Heparanase2, and a second amino acid sequence being at least about 70%, optionally at least about 80%, preferably at least about 85%, more preferably at least about 90% and most preferably at least about 95% homologous to a polypeptide having the sequence QWLIHTLQERRFGLKVW: (SEQ ID NO: 225), wherein said first and said second amino acid sequences are contiguous and in a sequential order.

An isolated polypeptide corresponding to a tail of Heparanase2_Skippingexon_{—}7_#PEP_NUM_{—}143, comprising a polypeptide having the sequence QWLIHTLQERRFGLKVW (SEQ ID NO: 225).

An isolated Heparanase2_Skippingexon_{—}8_#PEP_NUM_{—}144 polypeptide, comprising a first amino acid sequence being at least about 90% homologous to amino acids 1-366 of Heparanase2, and a second amino acid sequence being at least about 70%, optionally at least about 80%, preferably at least about 85%, more preferably at least about 90% and most preferably at least about 95% homolgous to a polypeptide having the sequence MVEHFIRIAGQSGH (SEQ ID NO: 226), wherein said first and said second amino acid sequences are contiguous and in a sequential order.

An isolated polypeptide corresponding to a tail of Heparanase2_Skippingexon_{—}8_#PEP_NUM_{—}144, comprising a polypeptide having the sequence MVEHFRIAGQSGH (SEQ ID NO: 226).

An isolated Heparanase2_Skippingexon_{—}9_#PEP_NUM_{—}145 polypeptide, comprising a first amino acid sequence being at least about 90% homologous to amino acids 1-401 of Heparanase2, and a second amino acid sequence being at least about 70%, optionally at least about 80%, preferably at least about 85%, more preferably at least about 90% and most preferably at least about 95% homologous to a polypeptide having the sequence TTGSLSSTSA (SEQ ID NO: 227), wherein said first and said second amino acid sequences are contiguous and in a sequential order.

An isolated polypeptide corresponding to a tail of Heparanase2_Skippingexon_{—}9_#PEP_NUM_{—}145, comprising a polypeptide having the sequence TTGSLSSTSA (SEQ ID NO: 227).

An isolated Heparanase_Skipping_exon_{—}10_#PEP_NUM_{—}140 polypeptide, comprising a first amino acid sequence being at least 90% homologous to amino acids 1-364 of Heparanase, and a second amino acid sequence being at least, 70%, optionally at least 80%, preferably at least 85%, more preferably at least 90% and most preferably at least 95% homologous to a polypeptide having the sequence IIGYLFCSRNWWAPRC, wherein said first and said second amino acid sequences are contiguous and in a sequential order.

An isolated polypeptide corresponding to a tail of Heparanase_Skipping_exon_{—}10_#PEP_NUM_{—}140, comprising a polypeptide having the sequence IIGYLFCSRNWWAPRC.

An isolated IGFBP4_Skippingexon_{—}3_#PEP_NUM_{—}111 polypeptide, comprising a first amino acid sequence being at least 90% homologous to amino acids 1-169 of IGFBP4, and a second amino acid sequence being at least 90% homologous to amino acids 215-258 of IGFBP4 or a portion thereof, wherein said first and said second amino acid sequences are contiguous and infra sequential order.

An isolated polypeptide of an edge portion of IGFBP4_Skippingexon_{—}3_#PEP_NUM_{—}111, comprising a first amino acid sequence being at least 90% homologous to amino acids 159-169 of IGFBP4 or a portion thereof, and a second amino acid sequence being at least 90% homologous to amino acids 215-225 of IGFBP4 or a portion thereof, wherein said first and said second amino acid sequences are contiguous and in a sequential order.

An isolated IL16_Long_Skippingexon_{—}18_#PEP_NUM_{—}110 polypeptide, comprising a first amino acid sequence being at least about 90% homologous to amino acids 1-1060 of IL16, and a second amino acid sequence being at least about 90% homologous to amino acids 1095-1244 of IL16, wherein said first and said second amino acid sequences are contiguous and in a sequential order.

An isolated polypeptide of an edge portion of IL16_Long_Skippingexon_{—}18_#PEP_NUM_{—}110, comprising a first amino acid sequence being at least about 90% homologous to amino acids 1050-1060 of IL16, and a second amino acid sequence being at least about 90% homologous to amino acids 1095-1105 of IL16, wherein said first and said second amino acid sequences are contiguous and in a sequential order.

An isolated IL16_Long_Skippingexon_{—}5_#PEP_NUM_{—}109 polypeptide, comprising a first amino acid sequence being at least about 90% homologous to amino acids 1-103 of IL16, and a second amino acid sequence being at least about 70%, optionally at least about 80, preferably at least about 85%, more preferably at least about 90% and most preferably at least about 95% homologous to a polypeptide having the sequence VLIPIAQEKLIFQ (SEQ ID NO: 228), wherein said first and said second amino acid sequences are contiguous and in a sequential order.

An isolated polypeptide corresponding to a tail of IL16_Long_Skippingexon_{—}5_#PEP_NUM_{—}109, comprising a polypeptide having the sequence VLIPIAQEKLIFQ (SEQ ID NO: 228).

An isolated IL18R_Skippingexon_{—}9_#PEP_NUM_{—}164 polypeptide, comprising a first amino acid sequence being at least about 90% homologous to amino acids 1-370 of IL18R, and a second amino acid sequence being at least about 90% homologous to amino acids 424-541 of IL18R, wherein said first and said second amino acid sequences are contiguous and in a sequential order.

An isolated polypeptide of an edge portion of IL18R_Skippingexon_{—}9_#PEP_NUM_{—}164, comprising a first amino acid sequence being at least about 90% homologous to amino acids 360-370 of IL18R, and % a second amino acid sequence being at least about 90% homologous to amino acids 424-434 of IL18R, wherein said first and said second amino acid sequences are contiguous and in a sequential order.

An isolated IL1RAPL1_Skippingexon_{—}4_#PEP_NUM_{—}170 polypeptide, comprising a first amino acid sequence being at least about 90% homologous to amino acids 1-122 of IL1RAPL1, and a second amino acid sequence being at least about 70%, optionally at least about 80%, preferably at least about 85%, more preferably at least about 90% and most preferably at least about 95% homologous to a polypeptide having the sequence AGQKHGGQVLYSKEILCL (SEQ ID NO: 229), wherein said first and said second amino acid sequences are contiguous and fin a sequential order.

An isolated polypeptide corresponding to a tail of IL1RAPL1_Skippingexon_{—}4_#PEP_NUM_{—}170, comprising a polypeptide having the sequence AGQKHGGQVLYSKEILCL (SEQ ID NO: 229).

An isolated IL1RAPL1_Skippingexon_{—}5_#PEP_NUM_{—}171 polypeptide, comprising a first amino acid sequence being at least about 90% homologous to amino acids 1-183 of IL1RAPL1, and a second amino acid sequence being at least about 90% homologous to amino acids 236-237 of IL1RAPL1, Wherein said first and said second amino acid sequences are contiguous and in a sequential order.

An isolated polypeptide of an edge portion of IL1RAPL1_Skippingexon_{—}5_#PEP_NUM_{—}171, comprising a first amino acid sequence being at least about 90% homologous to amino acids 173-183 of IL1RAPL1, and a second amino acid sequence being at least about 90% homologous to amino acids 236-246 of IL1RAPL1, wherein said first and said second amino acid sequences are contiguous and in a sequential order.

An isolated IL1RAPL1_Skippingexon_{—}6_#PEP_NUM_{—}172 polypeptide, comprising a first amino acid sequence being at least about 90% homologous to amino acids 1-234 of IL1RAPL1, and a second amino acid sequence being at least about 90% homologous to amino acids 260-696 of IL1RAPL1, wherein said first and said second amino acid sequences are contiguous and in a sequential order.

An isolated polypeptide of an edge portion of IL1RAPL1_Skippingexon_{—}6_#PEP_NUM_{—}172, comprising a first amino acid sequence being at least about 90% homologous to amino acids 224-234 of IL1RAPL1, and a second amino acid sequence being at least about 90% homologous to amino acids 260-270 of IL1RAPL1, wherein said first and said second amino acid sequences are contiguous and in a sequential order.

An isolated IL1RAPL1_Skippingexon_{—}7_#PEP_NUM_{—}173 polypeptide, comprising a first amino acid sequence being at least about 90% homologous to amino acids 1-259 of IL1RAPL1, and a second amino acid sequence being at least about 70%, optionally at least about 80%, preferably at least about 85%, more preferably at least about 90% and most preferably at least about 95% homologous to a polypeptide having the sequence EFLRSILGNRKFPSH (SEQ ID NO: 230), wherein said first and said second amino acid sequences are contiguous and in a sequential order.

An isolated polypeptide corresponding to a tail of IL1RAPL1_Skippingexon_{—}7_#PEP_NUM_{—}173, comprising a polypeptide having the sequence EFLRSILGNRKFPSH (SEQ ID NO: 230).

An isolated IL1RAPL1_Skippingexon_{—}8_#PEP_NUM_{—}174 polypeptide, comprising a first amino acid sequence being at least about 90% homologous to amino acids 1-304 of IL1RAPL1, and a second amino acid sequence being at least about 70%, optionally at least about 80%, preferably at least about 85%, more preferably at least about 90% and most preferably at least about 95% homologous to a polypeptide having the sequence ANVHSGTCCRPCCYSCCLYVW (SEQ ID NO: 231), wherein said first and said second amino acid sequences are contiguous and in a sequential order.

An isolated polypeptide corresponding to a tail of IL1RAPL1_Skippingexon_{—}8_#PEP_NUM_{—}174, comprising a polypeptide having the sequence ANVHSGTCCRPCCYSCCLYVW (SEQ ID NO: 231).

An isolated IL1RAPL12_Skippingexon_{—}4_#PEP_NUM_{—}175 polypeptide, comprising a first amino acid sequence at least about 90% homologous to amino acids 1-120 of IL1RAPL2, and a second amino acid sequence being at least about 70%, optionally at least about 80%, preferably at least about 85%, more preferably at least about 90% and most preferably at least about 95% homologous to a polypeptide having the sequence ASQKCGEA (SEQ ID NO: 232), wherein said first and said second amino acid sequences are contiguous and in a sequential order.

An isolated polypeptide corresponding to a tail of IL1RAPL2_Skippingexon_{—}4_#PEP_NUM_{—}175, comprising a polypeptide having the sequence ASQKCGEA (SEQ ID NO: 232).

An isolated IL1RAPL2_Skippingexon_{—}5_#PEP_NUM_{—}176 polypeptide, comprising a first amino acid sequence being at least about 90% homologous to amino acids 1-181 of IL1RAPL2, and a second amino acid sequence being at least about 70%, optionally at least about 80%, preferably at least about 85%, more preferably at least about 90% and most preferably at least about 95% homologous to a polypeptide having the sequence LYSQTSLPSHCSPWRISQVL (SEQ ID NO: 233), wherein said first and said second amino acid sequences are contiguous and in a sequential order.

An isolated polypeptide corresponding to a tail of IL1RAPL2_Skippingexon_{—}5_#PEP_NUM_{—}176, comprising a polypeptide having the sequence LYSQTSLPSHCSPWRISQVL (SEQ ID NO: 233).

An isolated IL1RAPL2_Skippingexon_{—}6_#PEP_NUM_{—}177 polypeptide, comprising a first amino acid sequence being at least about 90% homologous to amino acids 1-232 of IL1RAPL2, and a second amino acid sequence being at least about 90% homologous to amino acids 258-686 of IL1RAPL2, wherein said first and said second amino acid sequences are contiguous and in a sequential order.

An isolated, polypeptide of an edge portion of IL1RAPL2_Skippingexon_{—}6_#PEP_NUM_{—}177, comprising a first amino acid, sequence being at least about 90% homologous to amino acids 222-232 of IL1RAPL2, and a second amino acid sequence being least about 90% homologous to amino acids 258-268 of IL1RAPL2, wherein said first and said second amino acid sequences are contiguous and in a sequential order.

An isolated IL1RAPL2_Skippingexon_{—}7_#PEP_NUM_{—}178 polypeptide, comprising a first amino acid sequence being at least about 90% homologous to amino acids 1-258 of IL1RAPL2, and a second amino acid sequence being at least about 70%, optionally at least about 80% preferably at least about 85%, more preferably at least about 90% and most preferably at least about 95% homologous to a polypeptide having the sequence FSKSILEKKKLNWHSSLTQLWKLTWRIIPAMLKTEMDGNMPVFCCVKRI (SEQ ID NO: 234), wherein said first and said second amino acid sequences are contiguous and in a sequential order.

An isolated polypeptide corresponding to a tail of IL1RAPL2_Skippingexon_{—}7_#PEP_NUM_{—}178, comprising a polypeptide having the sequence FSKSILEKKKLNWHSSLTQLWKLTWRIIPAMLKTEMDGNMPVFCCVKRI (SEQ ID NO: 234).

An isolated IL1RAPL2_Skippingexon_{—}8_#PEP_NUM_{—}179 polypeptide, comprising a first amino acid sequence being at least about 90% homologous to amino acids 1-301 of IL1RAPL2, and a second amino acid sequence being at least about 70%, optionally at least about 80%, preferably at least about 85%, more preferably at least about 90% and most preferably at least about 95% homologous to a polypeptide having the sequence FNL, wherein said first and said second amino acid sequences are contiguous and in a sequential order.

An isolated polypeptide corresponding to a tail of IL1RAPL2_Skippingexon_{—}8_#PEP_NUM_{—}179, comprising a polypeptide having the sequence FNL.

An isolated IL1RAP_Skippingexon_{—}11_#PEP_NUM_{—}169 polypeptide comprising a first amino acid sequence being at least about 90% homologous to amino acids 1-400 of IL1RAP, a bridging amino acid V and a second amino acid sequence being at least about 90% homologous to amino acids 450-570 of IL1RAP, wherein said first amino acid sequence is contiguous to said bridging amino acid and said amino sequence is contiguous to said bridging amino acid, and wherein said first amino acid sequence, said bridging amino acid and said second amino acid sequence are in a sequential order.

An isolated polypeptide of an edge portion of IL1RAP_Skippingexon_{—}11_#PEP_NUM_{—}169, comprising a first amino acid sequence being at least about 90% homologous to amino acids 390-400 of IL1RAP, a bridging amino acid V and a second amino acid sequence being at least about 90% homologous to amino acids 450-460 of IL1RAP, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated ITAV_Skipping_exon_{—}11_#PEP_NUM_{—}14 polypeptide, comprising a first amino acid sequence being at least about 90% homologous to amino acids 1-301 of ITAV, and a second amino acid sequence being at least about 70%, optionally at least about 80%, preferably at least about 85%, more preferably. At least about 90% and most preferably at least about 95% homologous to a polypeptide having the sequence. LCRCVYWSTSLHGSWL (SEQ ID NO: 235), wherein said first and said second amino acid sequences are contiguous and in a sequential order.

An isolated polypeptide corresponding to a tail of ITAV_Skipping_exon_{—}11_#PEP_NUM_{—}14, comprising a polypeptide having the sequence LCRCVYWSTSLHGSWL (SEQ ID NO: 235).

An isolated ITAV_Skipping_exon_{—}20_#PEP_NUM_{—}15 polypeptide, comprising a first amino acid sequence being at least about 90% homologous to amino acids 1-641 of ITAV, and a second amino acid sequence being at least about 90% homologous to amino acids 1025-1026 of ITAV, wherein said first and said second amino acid sequences are contiguous and in a sequential order.

An isolated polypeptide of an edge portion of ITAV_Skipping_exon_{—}20_#PEP_NUM_{—}15, comprising a first amino acid sequence being at least about 90% homologous to amino acids 631-641 of ITAV, and a second amino acid sequence being at least about 90% homologous to amino acids 1025-1026 of ITAV, wherein said first and said second amino acid sequences are contiguous and in a sequential order.

An isolated —ITAV_Skipping_exon_{—}21_#PEP_NUM_{—}16 polypeptide, comprising a first amino acid sequence being of at least 90% homologous to amino acids 1-691 of ITAV, and a second amino acid sequence being at least 90% homologous to amino acids 723-1048 of ITAV or a portion thereof wherein said first and said second amino acid sequences are contiguous and in a sequential order.

An isolated polypeptide of an edge portion of ITAV_Skipping_exon_{—}21_#PEP_NUM_{—}16, comprising a first amino acid sequence being at least 90% homologous to amino acids 681-691 of ITAV or a portion thereof, and a second amino acid sequence being at least 90% homologous to amino acids 723-733 of ITAV or a portion thereof, wherein said first and said second amino acid sequences are contiguous and in a sequential order.

An isolated ITAV_Skipping_exon_{—}25_#PEP_NUM_{—}17 polypeptide, comprising a first amino acid sequence being at least about 90% homologous to amino acids 1-811 of ITAV, and a second amino acid sequence being at least about 90% homologous to amino acids 865-1048 of ITAV, wherein said first and said second amino acid sequences are contiguous and in a sequential order.

An isolated polypeptide of fan edge portion of ITAV_Skipping_exon_{—}25_#PEP_NUM_{—}17, comprising a first amino acid sequence being at least about 90% homologous to amino acids 801-811 of ITAV, and a second amino acid sequence being at least about 90% homologous to amino acids 865-875 of ITAV, wherein said first and said second amino acid sequences are contiguous and in a sequential order.

An isolated ITGA2B_Skippingexon_{—}3_#PEP_NUM_{—}135 polypeptide, comprising a first amino acid sequence being at least 90% homologous to amino acids 1-104 of ITGA2B, and a second amino acid sequence being at least 70%, optionally at least 80%, preferably at least 85%, more preferably at least 90% and most preferably at least 95% homologous to a polypeptide having the sequence LRPLAALERPRKD, wherein said first and said second amino acid sequences are contiguous and in a sequential order.

An isolated polypeptide corresponding to a tail of ITGA2B_Skippingexon_{—}3_#PEP_NUM_{—}135, comprising a polypeptide having a sequence LRPLAALERPRKD.

An isolated JAG1_Skippingexon_{—}10_#PEP_NUM_{—}96 polypeptide, comprising a first amino acid sequence being at least about 90% homologous to amino acids 1-412 of JAG1, and a second amino acid sequence being at least about 90% homologous to amino acids 451-1218 of JAG1, wherein said first and said second amino acid sequences are contiguous and in a sequential order.

An isolated polypeptide of an edge portion of JAG1_Skippingexon_{—}40_#PEP_NUM_{—}96, comprising a first amino acid sequence being at least about 90% homologous to amino acids 402-412 of JAG1, and a second amino acid sequence being at least about 90% homologous to amino acids 451-461 of JAG1, wherein said first and said second amino acid sequences are contiguous and in a sequential order.

An isolated JAG1_Skippingexon_{—}12_#PEP_NUM_{—}97 polypeptide, comprising a first amino acid sequence being at least about 90% homologous to amino acids 1-465 of JAG1, and a second amino acid sequence being at least about 90% homologous to amino acids 524-1218 of JAG1, wherein said first and said second amino acid sequences are contiguous and in a sequential order.

An isolated polypeptide of an edge portion of JAG1_Skippingexon_{—}12_#PEP_NUM_{—}97, comprising a first amino acid sequence being at least about 90% homologous to amino acids 455-465 of JAG1, and a second amino acid sequence being at least about 90% homologous to amino acids 524-534 of JAG1, wherein said first and said second amino acid sequences are contiguous and in a sequential order.

An isolated JAG1_Skippingexon_{—}18_#PEP_NUM_{—}98 polypeptide, comprising a first amino acid sequence being at least about 90% homologous to amino acids 1-742 of JAG1, a bridging amino acid D and a second amino acid sequence being at least about 90% homologous to amino acids 783-1218 of JAG1, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of JAG1_Skippingexon_{—}18_#PEP_NUM_{—}98, comprising a first amino acid sequence being at least about 90% homologous to amino acids 732-742 of JAG1, a bridging amino acid D and a second amino acid sequence being at least about 90% homologous to amino acids 783-793 of JAG1, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated JAG1_Skippingexon_{—}22_#PEP_NUM_{—}99 polypeptide, comprising a first amino acid sequence being at least about 90% homologous to amino acids 1-857 of JAG1, and a second amino acid sequence being at least about 70%, optionally at least about 80%, preferably at least about 85%, more preferably at least about 90% and most preferably at least about 95% homologous to a polypeptide having the sequence GLVPSILPAPQRAQRVPQRAELHPHPGRPVLRPPLHWCGRVSVFQSPAGEDK VHL (SEQ ID NO: 236), wherein said first and said second amino acid sequences are contiguous and in a sequential order.

An isolated polypeptide corresponding to a tail of JAG1_Skippingexon_{—}22_#PEP_NUM_{—}99, comprising a polypeptide having the sequence GLVPSILPAPQRAQRVPQRAELHPHPGRPVLRPPLHWCGRVSVFQSPAGEDK VHL (SEQ ID NO: 236).

An isolated KDR_Skipping_exon_{—}16_#PEP_NUM_{—}9 polypeptide, comprising a first amino acid sequence being at least about 90% homologous to amino acids 1-756 of KDR, and a second amino acid sequence being at least about 70%, optionally at least about 80%, preferably at least about 85%, more preferably at least about 90% and most preferably at least about 95% homologous to a polypeptide having the sequence QWRGTEDRLLVHRHGSR (SEQ ID NO: 237), wherein said first and said second amino acid sequences are contiguous and in a sequential order.

An isolated polypeptide corresponding to a tail of KDR_Skipping_exon_{—}16_#PEP_NUM_{—}9, comprising a polypeptide having the sequence QWRGTEDRLLVHRHGSR (SEQ ID NO: 237).

An isolated KDR_Skipping_exon_{—}17_#PEP_NUM_{—}10 polypeptide, comprising a first amino acid sequence being at least about 90% homologous to amino acids 1-791 of KDR, and a second amino acid sequence being at least about 70%, optionally at least about 80%, preferably at least about 85% more preferably at least about 90% and most preferably at least about 95% homologous to a polypeptide having the sequence VSLLAVVPLAK (SEQ ID NO: 238), wherein said first and said second amino acid sequences are contiguous and in a sequential order.

An isolated polypeptide corresponding to a tail of KDR_Skipping_exon_{—}17_#PEP_NUM_{—}10, comprising a polypeptide having the sequence VSLLAVVPLAK (SEQ ID NO: 238).

An isolated KDR_Skipping_exon_{—}27_#PEP_NUM_{—}11 polypeptide, comprising a first amino acid sequence being at least about 90% homologous to amino acids 1-1171 of KDR, and a second amino acid sequence being at least about 70%, optionally at least about 80%, preferably at least about 85%, more preferably at least about 90% and most preferably at least about 95% homologous to a polypeptide having the sequence SVSAEQ (SEQ ID NO: 239), wherein said first and said second amino acid sequences are contiguous and in a sequential order.

An isolated polypeptide corresponding to a tail of KDR_Skipping_exon_{—}27_#PEP_NUM_{—}11, comprising a polypeptide having the sequence SVSAEQ (SEQ ID NO: 239).

An isolated KDR_Skipping_exon_{—}28_#PEP_NUM_{—}12 polypeptide, comprising a first amino acid sequence being at least about 90% homologous to amino acids 1-1220 of KDR, and a second amino acid sequenced being at least about 70%, optionally at least about 80%, preferably at least about 85%, more preferably at least about 90% and most preferably at least about 95% homologous to a polypeptide having the sequence RTTRRTVVWFLPQKS (SEQ ID NO: 240), wherein said first and said second amino acid sequences are contiguous and in a sequential order.

An isolated polypeptide corresponding to a tail of KDR_Skipping_exon_{—}28_#PEP_NUM_{—}12, comprising a polypeptide having the sequence RTTRRTVVWFLPQKS (SEQ ID NO: 240).

An isolated KDR_Skipping_exon_{—}29_#PEP_NUM_{—}13 polypeptide, comprising a first amino acid of sequence being at least about 90% homologous td amino acids 1-1254 of KDR, and a second amino acid sequence being at least about 70%, optionally at least about 80%, preferably at least about 85%, more preferably at least about 90% and most preferably at least about 95% homologous to a polypeptide having the sequence WNGAQQKQGVCGI (SEQ ID NO: 241), wherein said first and said second amino acid sequences are contiguous and in a sequential order.

An isolated polypeptide corresponding to a tail of KDR_Skipping_exon_{—}29_#PEP_NUM_{—}13, comprising a polypeptide having the sequence WNGAQQKQGVCGI (SEQ ID NO: 241).

An isolated KITLG_Skippingexon_{—}8_#PEP_NUM_{—}73 polypeptide, comprising a first amino acid sequence being at least about 90% homologous to amino acids 1-238 of KITLG, and a second amino acid sequence being at least about 70%, optionally at least about 80%, preferably at least about 85%, more preferably at least about 90% and most preferably at least about 95% homologous to a polypeptide having the sequence YVARERERVSRSVIVACINTVTFVHWLVTVHVCFINEAALNKFIFCLE (SEQ ID NO: 242), wherein said first and said second amino acid sequences are contiguous and in a sequential order.

An isolated polypeptide corresponding to a tail of KITLG_Skippingexon_{—}8_#PEP_NUM_{—}73, comprising a polypeptide having the sequence YVARERERVSRSVIVACINTVTFVHWLVTVHVCFINEAALNKFIFCLE (SEQ ID NO: 242).

An isolated KIT_Skippingexon_{—}14_#PEP_NUM_{—}75 polypeptide, comprising a first amino acid sequence being at least about 90% homologous to amino acids 1-663 of KIT, and a second amino acid sequence being at least about 70%, optionally at least about 80%, preferably at least about 85%, more preferably at least about 90% and most preferably at least about 95% homologous to a polypeptide having the sequence AAIVLMSTWT (SEQ ID NO: 243), wherein said first and said second amino acid sequences are contiguous and in a sequential order.

An isolated polypeptide corresponding to a tail of KIT_Skippingexon_{—}14_#PEP_NUM_{—}75, comprising a polypeptide having the sequence AAIVLMSTWT (SEQ ID NO: 243).

An isolated KIT_Skippingexon_{—}8_#PEP_NUM_{—}74 polypeptide, comprising a first amino acid sequence being at least about 90% homologous to amino acids 1-410 of KIT, and a second amino acid sequence being at least about 70%, optionally at least about 80%, preferably at least about 85%, more preferably at least about 90% and most preferably at least about 95% homologous to a polypeptide having the sequence NALLLYCQWMCRH (SEQ ID NO: 244), wherein, said first and said second amino acid sequences are contiguous and in a sequential order.

An isolated polypeptide corresponding to a tail of KIT_Skippingexon_{—}8_#PEP_NUM_{—}4, comprising a polypeptide having the sequence NALLLYCQWMCRH (SEQ ID NO: 244).

An isolated LSHR_Intron_{—}5_retention_#PEP_NUM_{—}36 polypeptide, consisting essentially of an amino acid sequence being at least about 90% homologous to amino acids 1-153 of LSHR.

An isolated LSHR Skipping_exon_{—}10_#PEP_NUM_{—}35 polypeptide, comprising a first amino acid sequence being at least about 90% homologous to amino acids 1-289 of LSHR, and a second amino acid sequence being at least about 90% homologous to amino acids 317-699 of LSHR, wherein said first and said second amino acid sequences are contiguous and in a sequential order.

An isolated polypeptide of an edge portion of LSHR_Skipping_exon_{—}10_#PEP_NUM_{—}35, comprising a first amino acid sequence being at least about 90% homologous to amino acids 279-289 of LSHR, and a second amino acid sequence, being at least about 90% homologous to amino acids 317-327 of LSHR, wherein said first and said second amino acid sequences are contiguous and in a sequential order.

An isolated LSHR_Skipping_exon_{—}2_#PEP_NUM_{—}30 polypeptide, comprising a first amino acid sequence being at least about 90% homologous to amino acids 1-54 of LSHR, and a second amino acid sequence being at least about 90% homologous to amino acids 79-699 of LSHR, wherein said first and said second amino acid sequences are contiguous and in a sequential order.

An isolated polypeptide of an edge portion of LSHR_Skipping_exon_{—}2_#PEP_NUM_{—}30, comprising a first amino acid sequence being at least about 90% homologous to amino acids 44-54 of LSHR, and a second amino acid sequence being at least about 90% homologous to amino acids 79-89 of LSHR, wherein said first and said second amino acid sequences are contiguous and in a sequential order.

An isolated LSHR Skipping_exon_{—}3_#PEP_NUM_{—}31 polypeptide, comprising a first amino acid sequence being at least about 90% homologous to amino acids 1-78 of LSHR, and a second amino acid sequence being at least about 90% homologous to amino acids 101-699 of LSHR, wherein said first and said second amino acid sequences are contiguous and in a sequential order.

An isolated polypeptide of an edge portion of LSHR_Skipping_exon_{—}3_#PEP_NUM_{—}31, comprising a first amino acid sequence being at least about 90% homologous to amino acids 68-78 of LSHR, and a second amino acid sequence being at least about 90% homologous to amino acids 101-111 of LSHR, wherein said first and said second amino acid sequences are contiguous and in a sequential order.

An isolated LSHR_Skipping_exon_{—}5_#PEP_NUM_{—}32 polypeptide, comprising a first amino acid sequence being at least about 90% homologous to amino acids 1-128 of LSHR, and a second amino acid sequence being at least about 90% homologous to amino acids 151-699 of LSHR, wherein said first and said second amino acid sequences are contiguous and in a sequential order.

An isolated polypeptide of an edge portion of LSHR_Skipping_exon_{—}5_#PEP_NUM_{—}32, comprising a first amino acid sequence being at least about 90% homologous to amino acids 118-128 of LSHR, and a second amino acid sequence being at least about 90% homologous to amino acids 151-161 of LSHR, wherein said first and said second amino acid sequences are contiguous and in a sequential order.

An isolated LSHR_Skipping_exon_{—}6_#PEP_NUM_{—}33 polypeptide, comprising a first amino acid sequence being at least about 90% homologous to amino acids 1-152 of LSHR, and a second amino acid sequence being at least about 90% homologous to amino acids 179-699 of LSHR, wherein said first and said second amino acid sequences are contiguous and in a sequential order.

An isolated polypeptide of an edge portion of LSHR_Skipping_exon_{—}6_#PEP_NUM_{—}33, comprising a first amino acid sequence being at least about 90% homologous to amino acids 142-152 of LSHR, and a second amino acid sequence being at least about 90% homologous to amino acids 179-189 of LSHR, wherein said first and said second amino acid sequences are contiguous and in a sequential order.

An isolated LSHR_Skipping_exon_{—}7_#PEP_NUM_{—}34 polypeptide, comprising a first amino acid sequence being at least about 90% homologous to amino acids 1-179 of LSHR, and a second amino acid sequence being at least about 90% homologous to 6 amino acids 201-699 of LSHR, wherein said first and said second amino acid sequences are contiguous and in a sequential order.

An isolated polypeptide of an edge portion of LSHR_Skipping_exon_{—}7_#PEP_NUM_{—}34, comprising a first amino acid sequence being at least about 90% homologous to amino acids 169-179 of LSHR, and a second amino acid sequence being at least about 90% homologous to amino acids 201-211 of LSHR, wherein said first and said second amino acid sequences are contiguous and in a sequential order.

An isolated M17S2_Skippingexon_{—}14_#PEP_NUM_{—}189 polypeptide, consisting essentially of an amino acid sequence being at least about 90% homologous to amino acids 1-558 of M17S2, followed by M.

An isolated M17S2_Skippingexon_{—}15_#PEP_NUM_{—}190 polypeptide, comprising a first amino acid sequence being at least about 90% homologous to amino acids 1-583 of M17S2, and a second amino acid sequence being at least about 90% homologous to amino acids 621-966 of M17S2, wherein said first and said second amino acid sequences are contiguous and in a sequential order.

An isolated polypeptide of an edge portion of M17S2_Skippingexon_{—}15_#PEP_NUM_{—}190, comprising a first amino acid sequence being at least about 090% homologous to amino acids 573-583 of M17S2, and a second amino acid sequence being at least about 90% homologous to amino acids 621-631 of M17S2, wherein said first and said second amino acid sequences are contiguous and in a sequential order.

An isolated M17S2 Skippingexon_{—}20_#PEP_NUM_{—}191 polypeptide, comprising a first amino acid sequence being at least about 90% homologous to amino acids 1-873 of M17S2, and a second amino acid sequence being at least about 90% homologous to amino acids 963-964 of M17S2, wherein said first and said second amino acid sequences are contiguous and in a sequential order.

An isolated polypeptide of an edge portion of M17S2_Skippingexon_{—}20_#PEP_NUM_{—}191, comprising a first amino acid sequence being at least about 90% homologous to amino acids 863-873 of M17S2, and a second amino acid sequence being at least about 90% homologous to amino acids 963-964 of M17S2, wherein said first and said second amino acid sequences are contiguous and in a sequential order.

An isolated MET_Skipping_exon_{—}12_#PEP_NUM_{—}18 polypeptide, comprising a first amino acid sequence being at least about 90% homologous to amino acids 1-861 of MET, and a second amino acid sequence being at least about 90% homologous to amino acids 911-1390 of MET, wherein said first and said second amino acid sequences are continuous and in a sequential order.

An isolated polypeptide of an edge portion of MET_Skipping_exon_{—}12_#PEP_NUM_{—}18, comprising a first amino acid sequence being at least about 90% homologous to amino acids 851-861 of MET, and a second amino acid sequence being at least about 90% homologous to amino acids 911-921 of MET, wherein said first and said second amino acid sequences are contiguous and in a sequential order.

An isolated MET_Skipping_exon_{—}14_#PEP_NUM_{—}19 polypeptide, comprising a first amino acid sequence being at least about 90% homologous to amino acids 1-962 of MET, and a second amino acid sequence being at least about 90% homologous to amino acids 1010-1390 of MET, wherein said first and said second amino acid sequences are contiguous and in a sequential order.

An isolated polypeptide of an edge portion of MET_Skipping_exon_{—}14_#PEP_NUM_{—}19, comprising a first amino acid sequence being at least about 90% homologous to amino acids 952-962 of MET, and a second amino acid sequence being at least about 90% homologous to amino acids 1010-1020 of MET, wherein said first and said second amino acid sequences are contiguous and in a sequential order.

An isolated MET_Skipping_exon_{—}18_#PEP_NUM_{—}20 polypeptide, comprising a first amino acid sequence being at least about 90% homologous to amino acids 1-1174 of MET, and a second amino acid sequence being at least about 70%, optionally at least about 80%, preferably at least about 85%, more preferably at least about 90% and most preferably at least about 95% homologous to a polypeptide having the sequence AG, wherein said first and said second amino acid sequences are contiguous and in a sequential order.

An isolated polypeptide corresponding to a tail of MET_Skipping_exon_{—}18_#PEP_NUM_{—}20, comprising a polypeptide having the sequence AG.

An isolated MME_Skippingexon_{—}11_#PEP_NUM_{—}153 polypeptide, comprising a first amino acid sequence being at least about 90% homologous to amino acids 1-318 of MME, and a second amino acid sequence being at least about 70%, optionally at least about 80%, preferably at least about 85%, more preferably 4 at least about 90% and most preferably at least about 95% homologous to a polypeptide having the sequence RSSKFNVLEIHNGSCKQPQPNLQGVQKCFPQGPLWYNLRNSNLETLCKLCQW EYGKCCGEALCGSSICWRE (SEQ ID NO: 245), wherein said first and said second amino acid sequences are contiguous and in a sequential order.

An isolated polypeptide corresponding to a tail of MME_Skippingexon_{—}11_#PEP_NUM_{—}153, comprising a polypeptide having the sequence RSSKFNVLEIHNGSCKQPQPNLQGVQKCFPQGPLWYNLRNSNLETLCKLCQW EYGKCCGEALCGSSICWRE (SEQ ID NO: 245).

An isolated MME_Skippingexon_{—}12_#PEP_NUM_{—}154 polypeptide, comprising a first amino acid sequence being at least about 90% homologous to amino acids 1-364 of MME, and a second amino acid sequence being at least about 70%, optionally at least about 80%, preferably at least about 85%, more preferably at least about 90% and most preferably at least about 95% homologous to a polypeptide having the sequence PFMVQPQKQQLGDVVQTMSMGIWKMLWGGFMWKQHLLERVNMWSRI (SEQ ID NO: 246), wherein said first and said second amino acid sequences are contiguous and in a sequential order.

An isolated polypeptide corresponding to a tail of MME_Skippingexon_{—}12_#PEP_NUM_{—}154, comprising a polypeptide having the sequence PFMVQPQKQQLGDVVQTMSMGIWKMLWGGFMWKQHLLERVNMWSRI (SEQ ID NO: 246).

An isolated MME_Skipping_exon_{—}16_#PEP_NUM_{—}155 polypeptide, comprising a first amino acid sequence being at least about 90% homologous to amino acids 1-498 of MME, and a second amino acid sequence being at least about 70%, optionally at least about 80%, preferably at least about 85%, more preferably at least about 90% and most preferably at least about 95% homologous to a polypeptide having the sequence VDKWSSCSQCILLFRKKSDSLPSRHSAAPLL (SEQ ID NO: 247), wherein said first and said second amino acid sequences are contiguous and in a sequential order.

An isolated polypeptide corresponding to a tail of MME_Skippingexon_{—}16_#PEP_NUM_{—}155, comprising a polypeptide having the sequence VDKWSSCSQCILLFRKKSDSLPSRHSAAPLL (SEQ ID NO: 247).

An isolated MME_Skippingexon_{—}4_#PEP_NUM_{—}150 polypeptide, comprising a first amino acid sequence being at least bout % homologous to amino acids 1-64 of MME, and a second amino acid sequence being at least about 90% homologous to amino acids 119-749 of MME, wherein said first and said second amino acid sequences are contiguous and in a sequential order.

An isolated polypeptide of an edge portion of MME_Skippingexon_{—}4_#PEP_NUM_{—}150, comprising a first amino acid sequence being at least about 90% homologous to amino acids 54-64 of MME, and a second amino acid sequence being at least about 90% homologous to amino acids 119-129 of MME, wherein said first and said second amino acid sequences are contiguous and in a sequential order.

An isolated MME_Skippingexon_{—}7_#PEP_NUM_{—}151 polypeptide, consisting essentially of an amino acid sequence being at least about 90% homologous to amino acids 1-177 of MME, followed by D.

An isolated MME_Skippingexon_{—}9_#PEP_NUM_{—}152 polypeptide, comprising a first amino acid sequence being at least about 90% homologous to amino acids 1-239 of MME, and a second amino acid sequence being at least about 90% homologous to amino acids 285-749 of MME, wherein said first and said second amino acid sequences are contiguous and in a sequential order.

An isolated polypeptide of an edge portion of MME_Skippingexon_{—}9_#PEP_NUM_{—}152, comprising a first amino acid sequence being at least about 90% homologous to amino acids 229-239 of MME, and a second amino acid sequence being at least about 90% homologous to amino acids 285-295 of MME, wherein said first and said second amino acid sequences are contiguous and in a sequential order.

An isolated MPL_Skippingexon_{—}2_#PEP_NUM_{—}136 polypeptide, comprising a first amino acid sequence being at least about 90% homologous to ammo acids 1-26 of MPL, and a second amino acid sequence being at least about 70%, optionally at least about 80%, preferably about 85% more preferably at least about 90% and most preferably at least about 95% homologous to a polypeptide having the sequence GRSPVLAP (SEQ ID NO: 248), wherein said first and said second amino acid sequences are contiguous and in a sequential order.

An isolated polypeptide corresponding to a tail of MPL_Skippingexon_{—}2_#PEP_NUM_{—}136, comprising a polypeptide having the sequence GRSPVLAP (SEQ ID NO: 248).

An isolated NOTCH2_Skipping_exon_{—}12_#PEP_NUM_{—}101 polypeptide, comprising a first amino acid sequence being at least about 90% homologous to amino acids 1-638 of NOTCH2, and a second amino acid sequence being at least about 90% homologous to amino acids 676-2471 of NOTCH2, wherein said first and said second amino acid sequences are contiguous and in a sequential order.

An isolated polypeptide of an edge portion of NOTCH2_Skipping_exon_{—}12_#PEP_NUM_{—}101, comprising a first amino acid sequence being at least about 90% homologous to amino acids 628-638 of NOTCH2, and a second amino acid sequence being at least about 90% homologous to amino acids 676-686 of NOTCH2, wherein said first and said second amino acid sequences are contiguous and in a sequential order.

An isolated NOTCH2_Skippingexon_{—}9_#PEP_NUM_{—}100 polypeptide, comprising a first ammo acid sequence being at least about 90% homologous to amino acids 1-483 of NOTCH2, and a second amino acid sequence being at least about 90% homologous to amino acids 522-2471 of NOTCH2, wherein said first and said second amino acid sequences are contiguous and in a sequential order.

An isolated polypeptide of an edge portion of NOTCH2_Skippingexon_{—}9_#PEP_NUM_{—}100, comprising a first amino acid sequence being at least about 90% homologous to amino acids 473-483 of NOTCH2, and a second amino acid sequence being at least about 90% homologous to amino acids 522-532 of NOTCH2, wherein said first and said second amino acid sequences are contiguous and in a sequential order.

An isolated NOTCH3_Skippingexon_{—}2_#PEP_NUM_{—}102 polypeptide, comprising a first amino acid sequence being at least about 90% homologous to amino acids 1-39 of NOTCH3, and a second amino acid sequence being at least about 70%, optionally at least about 80%, preferably at least about 85%, more preferably at least about 90% and most preferably at least about 95% homologous to a polypeptide having the sequence GARLAGWVSGVSWRTPVTQAPVLAVVSARVQWWLAPPDSHAGAPVASEAL TAPCQIPASAALVPTVPAAQWGPMDASSAPAHLATRAAAAEATWMSAGWV SPAAMVAPASTHLAPSAASVQLATQGHYVRTPRCPVHPHHAVTGAPAGRVA TSLTTVPVFLGLRVRIVK (SEQ ID NO: 249), wherein said first and said second amino acid sequences are contiguous and in a sequential order.

An isolated polypeptide corresponding to a tail of NOTCH3_Skippingexon_{—}2_#PEP_NUM_{—}102, comprising a polypeptide having the sequence GARLAGWVSGVSWRTPVTQAPVLAVVSARVQWWLAPPDSHAGAPVASEAL TAPCQIPASAALVPTVPAAQWGPMDASSAPAHLATRAAAAEATWMSAGWV SPAAMVAPASTHLAPSAASVQLATQGHYVRTPRCPVHPHHAVTGAPAGRVA TSLTTVPVFLGLRVRIVK (SEQ ID NO: 249).

An isolated NOTCH4_Skipping_exon_{—}8_#PEP_NUM_{—}103 polypeptide, comprising a first amino acid sequence being at least about 90% homologous to amino acids 1-438 of NOTCH4, and a second amino acid sequence being at least about 90% homologous to amino acids 504-2003 of NOTCH4, wherein said first and said second amino acid sequences are contiguous and in a sequential order.

An isolated polypeptide of an edge portion of NOTCH4_Skipping exon_{—}8_#PEP_NUM_{—}103, comprising a first amino acid sequence being at least about 90% homologous to amino acids 428-438 of NOTCH4, and a second amino acid sequence being at least about 90% homologous to amino acids 504-514 of NOTCH4, wherein said first and said second amino acid sequences are contiguous and in a sequential order.

An isolated NRG1_HGR-ALPHA_skippingexon_{—}5_#PEP_NUM_{—}82 polypeptide, comprising a first amino acid sequence being at least about 90% homologous to amino acids 1-150 of NRG1-HRG-ALPHA, a bridging amino acid A and a second amino acid sequence being at least about 90% homologous to amino acids 169-640 of NRG1-HRG-ALPHA, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of NRG1_HGR-ALPHA_skippingexon_{—}5_#PEP_NUM_{—}82, comprising a first amino acid sequence being at least about 90% homologous to amino acids 140-150 of NRG1-HRG-ALPHA, a bridging amino acid A and a second amino acid sequence being at least about 90% homologous to amino acids 169-179 of NRG1-HRG-ALPHA, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated NRG1_HGR-ALPHA_skippingexon_{—}7_#PEP_NUM_{—}83 polypeptide, comprising a first amino acid sequence being at least about 90% homologous to amino acids 1-211 of NRG1-HRG-ALPHA, and a second amino acid sequence being at least about 70%, optionally at least about 80%, preferably at least about 85%, more preferably at least about 90% and most preferably at least about 95% homologous to a polypeptide having the sequence GGGAVPEESADHNRHLHRPPCGRHHVCGGLLQNQETAEKAA (SEQ ID NO: 250), wherein said first and said second amino acid sequences are contiguous and in a sequential order.

An isolated polypeptide corresponding to a tail of NRG1_HGR-ALPHA_skippingexon_{—}7_#PEP_NUM_{—}83, comprising a polypeptide having the sequence GGGAVPEESADHNRHLHRPPCGRHHVCGGLLQNQETAEKAA (SEQ ID NO: 250).

An isolated NRG1_HGR-BETA1_skippingexon_{—}5_#PEP_NUM_{—}84 polypeptide, comprising a first amino acid sequence being at least about 90% homologous to amino acids 1-150 of NRG1-HRG-BETA1, a bridging amino acid A and a second amino acid sequence being at least about 90% homologous to amino acids 169-645 of NRG1-HRG-BETA1, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of NRG1_HGR-BETA1_skippingexon_{—}5_#PEP_NUM_{—}84, comprising a first amino acid sequence being at least about 90% homologous to amino acids 140-150 of NRG1-HRG-BETA1, a bridging amino acid A and a second amino acid sequence being at least about 90% homologous to amino acids 169-179 of NRG1-HRG-BETA1, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated NRG1_HGR-BETA1_skippingexon_{—}7_#PEP_NUM_{—}85 polypeptide, comprising a first amino acid sequence being at least about 90% homologous to amino acids 1-211 of NRG1-HRG-BETA1 NRG1-HRG-BETA2 NRG1-HRG-BETA3, and a second amino acid sequence being at least about 70%, optionally at least about 80%, preferably at least about 85%, more preferably at least about 90% and most preferably at least about 95% homologous to a polypeptide having the sequence GGGAVPEESADHNRHLHRPPCGRHHVCGGLLQNQETAEKAA (SEQ ID NO: 251), wherein said first and said second amino acid sequences are contiguous and in a sequential order.

An isolated polypeptide corresponding to a tail of NRG1-HGR-BETA1_skippingexon_{—}7_#PEP_NUM_{—}85, comprising a polypeptide having the sequence GGGAVPEESADHNRHLHRPPCGRHHVCGGLLQNQETAEKAA (SEQ ID NO: 251).

An isolated NRG1_HGR-BETA1_skippingexon_{—}8_#PEP_NUM_{—}86 polypeptide, comprising a first amino acid sequence being at least about 90% homologous to amino acids 1-231 of NRG1-HRG-BETA1, and a second amino acid sequence being at least about 90% homologous to amino acids 240-645 of NRG1-HRG-BETA1, wherein said first and said second amino acid sequences are contiguous and in a sequential order.

An isolated polypeptide of an edge portion of NRG1_HGR-BETA1_skippingexon_{—}8_#PEP_NUM_{—}86, comprising a first amino acid sequence being at least about 90% homologous to amino acids 221-231 of NRG1-HRG-BETA1, and a second amino acid sequence being at least about 90% homologous to amino acids 240-250 of NRG1-HRG-BETA1, wherein said first and said second amino acid sequences are contiguous and in a sequential order.

An isolated NRG1_HGR-BETA1_skippingexon_{—}9_#PEP_NUM_{—}87, polypeptide, comprising a first amino acid sequence being at least about 90% homologous to amino acids 1-230 of NRG1-HRG-BETA1, and a second amino acid sequence being at least about 70%, optionally at least about 80%, preferably at least about 85%, more preferably at least about 90% and most preferably at least about 95% homologous to a polypeptide having the sequence RNSGKSCMTVFGRAFGLNETI (SEQ ID NO: 252), wherein said first and said second amino acid sequences are contiguous and in a sequential order.

An isolated polypeptide corresponding to a tail of NRG1_HGR-BETA1_skippingexon_{—}9_#PEP_NUM_{—}87, comprising a polypeptide having the sequence RNSGKSCMTVFGRAFGLNETI (SEQ ID NO: 252).

An isolated NRG1_HGR-BETA2_skippingexon_{—}5_#PEP_NUM_{—}88 polypeptide, comprising a first amino acid sequence being at least about 90% homologous to amino acids 1-150 of NRG1-HRG-BETA2, a bridging amino acid A and a second amino acid sequence being at least about 90% homologous to amino acids 169-636 of NRG1-HRG-BETA2, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of NRG1_HGR-BETA2_skippingexon_{—}5_#PEP_NUM_{—}88, comprising a first amino acid sequence being at least about 90% homologous to amino acids 140-150 of NRG1-HRG-BETA2, a bridging amino acid A and a second amino acid sequence being at least about 90% homologous to amino acids 169-179 of NRG1-HRG-BETA2, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated NRG1_HGR-BETA2_skippingexon_{—}8_#PEP_NUM_{—}89 polypeptide, comprising a first amino acid sequence being at least about 90% homologous to amino acids 1-230 of NRG1-HRG-BETA NRG1-HRG-BETA3, and a second amino acid sequence being at least about 70%, optionally at least about 80%, preferably at least about 85%, more preferably at least about 90% and most preferably at least about 95% homologous to a polypeptide having the sequence RNSGKSCMTVFIGRAFGLNETI (SEQ ID NO: 253), wherein said first and said second amino acid sequences are contiguous and in a sequential order.

An isolated polypeptide corresponding to a tail of NRG1_HGR-BETA2_skippingexon_{—}8_#PEP_NUM_{—}89, comprising a polypeptide having the sequence RNSGKSCMTVFGRAFGLNETI (SEQ ID NO: 253).

An isolated NRG1_HGR-BETA3_skippingexon_{—}5_#PEP_NUM_{—}90 polypeptide, comprising a first amino acid sequence being at least about 90% homologous to amino acids 1-150 of NRG1-HRG-BETA3, a bridging amino acid A and a second amino acid sequence being at least about 90% homologous to amino acids 169-241 of NRG1-HRG-BETA3, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of NRG1_HGR-BETA3_skippingexon_{—}5_#PEP_NUM_{—}90, comprising a first amino acid sequence being at least about 90% homologous to amino acids 140-150 of NRG1-HRG-BETA3, a bridging amino acid A and a second amino acid sequence being at least about 90% homologous to amino acids 169-179 of NRG1-HRG-BETA3, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated NRG1_HGR-GAMMA_skippingexon_{—}5_#PEP_NUM_{—}91 polypeptide, comprising a first amino acid sequence being at least about 90% homologous to amino-acids 1-150 of NRG1-HRG-GAMMA, a bridging amino acid, A and a second amino acid sequence being at least about 90% homologous to amino acids 169-211 of NRG1-HRG-GAMMA, wherein said first amino acid sequence is contiguous to said bridging no acid and said second amino acid sequence contiguous to said bridging amino acid and wherein said first amino acid sequence, said bridging amino acid and said second amino acid sequence are in a sequential order.

An isolated polypeptide of an edge portion of NRG1_HGR-GAMMA_skippingexon_{—}5_#PEP_NUM_{—}91, comprising a first amino acid sequence being at least about 90% homologous amino acids 140-150 of NRG1-HRG-GAMMA, a bridging amino acid A and a second amino acid sequence being at least about 90% homologous to amino acids 169-179 of NRG1-HRG-GAMMA, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated NRG1_HGR-GGF_skippingexon_{—}5_#PEP_NUM_{—}92 polypeptide, comprising a first amino acid sequence being at least about 90% homologous to amino acids 1-150 of NRG1-HRG-GGF, a bridging amino acid A and a second amino acid sequence being at least about 90% homologous to amino acids 169-241 of NRG1-HRG-GGF, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of NRG1_HGR-GGF_skippingexon_{—}5_#PEP_NUM_{—}92, comprising a first amino acid sequence being at least about 90% homologous to amino acids 140-150 of NRG1-HRG-GGF, a bridging amino acid A and a second amino acid sequence being at least about 90% homologous to amino acids 169-179 of NRG1-HRG-GGF, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated NRG1_NDF43_skippingexon_{—}12_#PEP_NUM_{—}95 polypeptide, comprising a first amino acid sequence being at least about 90% homologous to amino acids 1-423 of NRG1-NDF43, and a second amino acid sequence being at least about 70%, optionally at least about 80%, preferably at least about 8 more preferably at least about 90% and most preferably at least about 95% homologous to a polypeptide having the sequence YVSAMTTPARMSPVDFHTPSSPKSPPSEMSPPVSSMTVSMPSMAVSPFMEEER PLLLVTPPRLREKKFDHHPQQFSSFHHNPAHDSNSLPASPLRIVEDEEYETTQE YEPAQEPVK (SEQ ID NO: 254), wherein said first and said second amino acid sequences are contiguous and in a sequential order.

An isolated polypeptide corresponding to a tail of NRG1_NDF43_skippingexon_{—}12_#PEP_NUM_{—}95, comprising a polypeptide having the sequence YVSAMTTPARMSPVDFHTPSSPKSPPSEMSPPVSSMTVSMPSMAVSPFMEEER PLLLVTPPRLREKKFDHHPQQFSSFHHNPAHDSNSLPASPLRIVEDEEYETTQE YEPAQEPVK (SEQ ID NO: 254).

An isolated NRG1_NDF43_skippingexon_{—}5_#PEP_NUM_{—}93 polypeptide, comprising a first amino acid sequence being at least about 90% homologous to amino acids 1-150 of NRG1-NDF43, a bridging amino acid A and a second amino acid sequence being at least about 90% homologous to amino acids 169-462 of NRG1-NDF43, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of NRG1_NDF43_skippingexon_{—}5_#PEP_NUM_{—}93, comprising a first amino acid sequence being at least about 90% homologous to amino acids 140-150 of NRG1-NDF43, a bridging amino acid A and a second amino acid sequence being at least about 90% homologous to amino acids 169-179 of NRG1-NDF43, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated NRG1_NDF43_skippingexon_{—}7_#PEP_NUM_{—}94 polypeptide, comprising a first amino acid sequence being at least about 90% homologous to amino acids 1-211 of NRG1-NDF43, and a second amino acid sequence being at least about 70%, optionally at least about 80%, preferably at least about 85%, more preferably at least about 90% and most preferably at least about 95% homologous to a polypeptide having the sequence GGGAVPEESADHNRHLHRPPCGRHHVCGGLLQNQETAEKAA (SEQ ID NO: 255), wherein said first and said second amino acid sequences are contiguous and in a sequential order.

An isolated polypeptide corresponding to a tail of NRG1_NDF43_skippingexon_{—}7_#PEP_NUM_{—}94, comprising a polypeptide having the sequence. GGGAVPEESADHNRHLHRPPCGRHHVCGGLLQNQETAEKAA (SEQ ID NO: 255).

An isolated NRP1_Skippingexon_{—}5_#PEP_NUM_{—}112 polypeptide, comprising a first amino acid sequence being at least about 90% homologous to amino acids 1-219 of NRP1, and a second amino acid sequence being at least about 90% homologous to amino acids 272-923 of NRP1, wherein said first and said second amino acid sequences are contiguous and in a sequential order.

An isolated polypeptide of an edge portion of NRP1_Skippingexon_{—}5_#PEP_NUM_{—}112, comprising a first amino acid sequence being at least about 90% homologous to ammo acids 209-219 of NRP1, and a second amino acid sequence being at least about 90% homologous to amino acids 272-282 of NRP1, wherein said first and said second amino acid sequences are contiguous and in a sequential order.

An isolated NTRK2_skippingexon_{—}14_#PEP_NUM_{—}104 polypeptide, consisting essentially of an amino acid sequence being at least about 90% homologous to amino acids 1-240 of NTRK2.

An isolated NTRK3_Skippingexon_{—}16_#PEP_NUM_{—}106 polypeptide, comprising a first amino acid sequence being at least 90% homologous to amino acids 1-630 of NTRK3, and a second amino acid sequence being at least 70%, optionally at least 80%, preferably at least 85%, more preferably at least 90% and most preferably at least 95% homologous to a polypeptide having the sequence WEDTPCSPFAGCLLKASCTGSSLQRVMYGASG, wherein said first and said second amino acid sequences are contiguous and in a sequential order.

An isolated polypeptide corresponding to a tail of NTRK3_Skippingexon_{—}16_#PEP_NUM_{—}106, comprising a polypeptide having the sequence WEDTPCSFAGCLLKASCTGSSLQRVMYGASG.

An isolated NTRK3_Skippingexon_{—}5_#PEP_NUM_{—}105 polypeptide, comprising a first amino acid sequence being at least about 90 “% homologous to amino acids 1-131 of NTRK3, and a second amino acid sequence being at least about 90% homologous to amino acids 156-839 of NTRK3, wherein said first and said second amino acid sequences are contiguous and in a sequential order.

An isolated polypeptide of an edge portion of NTRK3_Skippingexon_{—}5_#PEP_NUM_{—}105, comprising a first amino acid sequence being at least about 90% homologous to amino acids 121-131 of NTRK3, and a second amino acid sequence being at least about 90% homologous to amino acids 156-166 of NTRK3, wherein said first and said second amino acid sequences are contiguous and in a sequential order.

An isolated PROS1_Skippingexon_{—}3_#PEP_NUM_{—}185 polypeptide comprising a first amino acid sequence being at least about 90% homologous to amino acids 1-78 of PROS1, and a second amino acid sequence being at least about 70%, optionally at least about 80%, preferably at least about 85%, more preferably at least about 90% and most preferably at least about 95% homologous to a polypeptide having the sequence FVFALFKLGYSLLHVSQLMLILT (SEQ ID NO: 256), wherein said first and said second amino acid sequences are contiguous and in a sequential order.

An isolated polypeptide corresponding to a tail of PROS1_Skippingexon_{—}3_#PEP_NUM_{—}185, comprising a polypeptide having the sequence FVFALFKLGYSLLHVSQLMLILT (SEQ ID NO: 256).

An isolated PTPRB_Skippingexon_{—}26_#PEP_NUM_{—}72 polypeptide, comprising a first amino acid sequence being at least about 90% homologous to amino acids 1-1738 of PTPRB, and a second amino acid sequence being at least about 70%, optionally at least about 80%, preferably at least about 85%, more preferably at least about 90% and most preferably at least about 95% homologous to a polypeptide having the sequence WQQLQKRIHCHSGTASWHQG (SEQ ID NO: 257), wherein said first and said second amino acid sequences are contiguous and in a sequential order.

An isolated polypeptide corresponding to a tail of PTPRB_Skippingexon_{—}26_#PEP_NUM_{—}72, comprising a polypeptide having the sequence WQQLQKRIHCHSGTASWHQG (SEQ ID NO: 257.)

An isolated PTPRZ1_Skippingexon_{—}11_#PEP_NUM_{—}67 polypeptide, comprising a first, amino acid sequence being at least about 90% homologous to amino acids 1-413 of PTPRZ1, and a second amino acid sequence being at least about 70%, optionally at least about 80% preferably at least about 85%, more preferably at least about 90% and most preferably at least about 95% homologous to a polypeptide having the sequence GGGRGKRH (SEQ ID NO: 258), wherein said first and said second amino acid sequences are contiguous and in a sequential order.

An isolated polypeptide corresponding to a tail of PTPRZ1_Skippingexon_{—}11_#PEP_NUM_{—}67, comprising a polypeptide having the sequence GGGRGKRH (SEQ ID NO: 258).

An isolated PTPRZ1_Skippingexon_{—}13_#PEP_NUM_{—}68 polypeptide, comprising a first amino acid sequence being at least about 90% homologous to amino acids 1-1613 of PTPRZ1, and a second amino acid sequence being at least about 70%, optionally at least about 80%, preferably at least about 85%, more preferably at least about 90% and most preferably at least about 95% homologous to a polypeptide having the sequence GNASRLHTFT (SEQ ID NO: 258), wherein said first and said second amino acid sequences are contiguous and in a sequential order.

An isolated polypeptide corresponding to a tail of PTPRZ1_Skippingexon_{—}13_#PEP_NUM_{—}68, comprising a polypeptide having the sequence GNASRLHTFT (SEQ ID NO: 259).

An isolated PTPRZ1_Skippingexon_{—}15_#PEP_NUM_{—}69 polypeptide, comprising a first amino acid sequence being at least about 90% homologous to amino acids 1-1693 of PTPRZ1, and a second amino acid sequence being at least about 70%, optionally at least about 80%, preferably at least about 85%, more preferably at least about 90% and most preferably at least about 95% homologous to a polypeptide having the sequence TEEVLPGLRYYDEQLQPPEQQAQESIHKYRCL (SEQ ID NO: 260), wherein said first and said second amino acid sequences are contiguous and in a sequential order.

An isolated polypeptide corresponding to a tail of PTPRZ1_Skippingexon_{—}15_#PEP_NUM_{—}69, comprising a polypeptide having the sequence TEEVLPGLRYYDEQLQPPEQQAQESIHKYRCL (SEQ ID NO: 260).

An isolated PTPRZ1_Skippingexon_{—}16_#PEP_NUM_{—}70 polypeptide, comprising a first amino acid sequence being at least about 90% homologous to amino acids 1-1721 of PTPRZ1, and a second amino acid sequence being at least about 90% homologous to amino acids 1729-2314 of PTPRZ1, wherein said first and said second amino acid sequences are contiguous and in a sequential order.

An isolated polypeptide of an edge portion of PTPRZ1_Skippingexon_{—}16_#PEP_NUM_{—}70, comprising a first amino acid sequence being at least about 90% homologous to amino acids 1711-1721 of PTPRZ1, and a second amino acid sequence being at least about 90% homologous to amino acids 1729-1739 of PTPRZ1, wherein said first and said second amino acid sequences are contiguous and in a sequential order.

An isolated PTPRZ1_Skippingexon_{—}22_#PEP_NUM_{—}71 polypeptide, comprising a first amino acid sequence being at least about 90% homologous to amino acids 1-1932 of PTPRZ1, and a second amino acid sequence being at least about 70%, optionally at least about 80%, preferably at least about 85%, more preferably at least about 90% and most preferably at least about 95% homologous to a polypeptide having the sequence RSNMSSFMIHWLRPYLVKKLRCWTVIFMPMLMHSSFLDQQAKQ (SEQ ID NO: 261), wherein said first and said second amino sequences are contiguous and in a sequential order.

An isolated polypeptide corresponding to a tail of PTPRZ1_Skippingexon_{—}22_#PEP_NUM_{—}71, comprising a polypeptide having the sequence RSNMSSFMIHWLRPYLVKKLRCWTVIFMPMLMHSSFLDQQAKQ (SEQ ID NO: 261).

An isolated PTPRZ1_Skippingexon_{—}7_#PEP_NUM_{—}66 polypeptide, comprising a first amino acid sequence being at least about 90% homologous to amino acids 1-206 of PTPRZ1, and a second amino acid sequence being at least about 70%, optionally at least about 80%, preferably at least about 85%, more preferably at least about 90% and most preferably at least about 95% homologous to a polypeptide having the sequence VGCFCEVLTCNNLVMSC (SEQ ID NO: 262), wherein said first and said second amino acid sequences are contiguous and in a sequential order.

An isolated polypeptide corresponding to a tail of PTPRZ1_Skippingexon_{—}7_#PEP_NUM_{—}66, comprising a polypeptide having the sequence VGCFCEVLTCNNLVMSC (SEQ ID NO: 262).

An isolated RSU1_Skippingexon_{—}6_#PEP_NUM_{—}163 polypeptide, comprising a first amino acid sequence being at least about 90% homologous to amino acids 1-134 of RSU1, and a second amino acid sequence being at least about 70%, optionally at least about 80%, preferably at least about 85%, more preferably at least about 90% and most preferably at least about 95% homologous to a polypeptide having the sequence QP, wherein said first and said second amino acid sequences are contiguous and in a sequential order.

An isolated polypeptide corresponding to a tail of RSU1_Skippingexon_{—}6_#PEP_NUM_{—}163, comprising a polypeptide having the sequence QP.

An isolated SCTR_Skippingexon_{—}10_#PEP_NUM_{—}162 polypeptide, comprising a first amino acid sequence being at least about 90% homologous to amino acids 1-307 of SCTR, and a second amino acid sequence being at least about 70%, optionally at least about 80%, preferably at least about 85%, more preferably at least about 90% and most preferably at least about 95% homologous to a polypeptide having the sequence APGQVHSPADPPLWHPLHRLRLLPRGRYGDPAVF (SEQ ID NO: 263), wherein said first and said second amino acid sequences are contiguous and in a sequential order.

An isolated polypeptide corresponding to a tail of SCTR_Skippingexon_{—}10_#PEP_NUM_{—}162, comprising a polypeptide having the sequence APGQVHSPADPPLWHPLHRLRLLPRGRYGDPAVF (SEQ ID NO: 263).

An isolated TGFB2_Skippingexon_{—}5_#PEP_NUM_{—}165 polypeptide, comprising a first amino acid sequence being at least about 90% homologous to amino acids 1-251 of TGFB2, and a second amino acid sequence being at least about 70%, optionally at least about 80%, preferably at least about 85%, more preferably at least about 90% and most preferably at least about 95% homologous to a polypeptide having the sequence EMCRIIAAYVHFTLISRGI (SEQ ID NO: 264), wherein said first and said second amino acid sequences are contiguous and in a sequential order.

An isolated polypeptide corresponding to a tail of TQFB2_Skippingexon_{—}5_#PEP_NUM_{—}165, comprising a polypeptide having the sequence EMCRIIAAYVHFTLISRGI (SEQ ID NO: 264).

An isolated THBS1_Skippingexon_{—}12_#PEP_NUM_{—}183 polypeptide, comprising a first amino acid sequence being at least about 90% homologous to amino acids 1-591 of THBS1, and a second amino acid sequence being at least about 90% homologous to amino acids 643-1170 of THBS1, wherein said first and said second amino acid sequences are contiguous and in a sequential order.

An isolated polypeptide of an edge portion of THBS1_Skippingexon_{—}12_#PEP_NUM_{—}183, comprising a first amino acid sequence being at least about 90% homologous to amino acids 581-591 of THBS1 and a second amino acid sequence being at least about 90% homologous to amino acids 643-653 of THBS1, wherein said first said second amino acid sequences are contiguous and in a sequential order.

An isolated THBS1_Skippingexon_{—}4_#PEP_NUM_{—}180 polypeptide, comprising a first amino acid sequence being at least about 90% homologous to amino acids 1-209 of THBS1, and a second amino acid sequence being at least about 70%, optionally at least about 80%, preferably at least about 85% more preferably at least about 90% and most preferably at least about 95% homologous to a polypeptide having the sequence LPVSSSPLTTTW (SEQ ID NO: 265), wherein said first and said second amino acid sequences are contiguous and in a sequential order.

An isolated polypeptide corresponding to a tail of THBS1_Skippingexon_{—}4_#PEP_NUM_{—}180, comprising a polypeptide having the sequence LPVSSSPLTTTW (SEQ ID NO: 265).

An isolated THBS1_Skippingexon_{—}7_#PEP_NUM_{—}181 polypeptide, comprising a first amino acid sequence being at least about 90% homologous to amino acids 1-342 of THBS1, and a second amino acid sequence being at least about 70%, optionally at least about 80%, preferably at least about 85%, more preferably at least about 90% and most preferably at least about 95% homologous to a polypeptide having the sequence PATLRTMAGLHGPSGPPVLRAVAMEFSSAAAPAIASTTDVRAPRSRHGPAIFR SVTRDLNRMVAGATGPRGHLVL (SEQ ID NO: 266), wherein said first and said second amino acid sequences are contiguous and in a sequential order.

An isolated polypeptide corresponding to a tail of THBS1_Skippingexon_{—}7_#PEP_NUM_{—}181, comprising a polypeptide having sequence PATLRTMAGLHGPSGPPVLRAVAMEFSSAAAPAIASTTDVRAPRSRHGPAIFR SVTRDLNRMVAGATGPRGHLVL (SEQ ID NO: 266).

An isolated THBS1_Skippingexon_{—}9_#PEP_NUM_{—}182 polypeptide, comprising a first amino acid sequence being at least about 90% homologous to amino acids 1-373 of THBS1, and a second amino acid sequence being at least about 90% homologous to amino acids 432-1170 of THBS1, wherein said first and said second amino acid sequences are contiguous and in a sequential order.

An isolated polypeptide of an edge portion of THBS1_Skippingexon_{—}9_#PEP_NUM_{—}182, comprising a first amino acid sequence being at least about 90% homologous to amino acids 363-373 of THBS1, and a second amino acid sequence being at least about 90% homologous to amino acids 432-442 of THBS1, wherein said first and said second amino acid sequences are contiguous and in a sequential order.

An isolated THBS4_Skippingexon_{—}15_#PEP_NUM_{—}184 polypeptide, consisting essentially of an amino acid sequence being at least about 90% homologous to amino acids 1-613 of THBS4.

An isolated TIAF1_Skippingexon_{—}11_#PEP_NUM_{—}166 polypeptide, comprising a first amino acid sequence being at least about 90% homologous to amino acids 1-679 of TIAF1, and a second amino acid sequence being at least about 90% homologous to amino acids 674-2054 of TIAF1, wherein said first and said second amino acid sequences are contiguous and in a sequential order.

An isolated polypeptide of an edge portion of TIAF1_Skippingexon_{—}11_#PEP_NUM_{—}166, comprising a first amino acid sequence being at least about 90% homologous to amino acids 669-679 of TIAF1, and a second amino acid sequence being at least about 90% homologous to amino acids 674-684 of TIAF1, wherein said first and said second amino acid sequences are contiguous and in a sequential order.

An isolated TIAF1_Skippingexon_{—}25_#PEP_NUM_{—}167 polypeptide, comprising a first amino acid sequence being at least about 90% homologous to amino acids 1-1290 of TIAF1, and a second amino acid sequence being at least about 90% homologous to amino acids 133-2054 of TIAF1, wherein said first and said second amino acid sequences are contiguous and in a sequential order.

An isolated polypeptide of an edge portion of TIAF1_Skippingexon_{—}25_#PEP_NUM_{—}167, comprising a first amino acid sequence being at least about 90% homologous to amino acids 1280-1290 of TIAF1, and a second amino acid sequence being at least about 90% homologous to amino acids 1331-1341 of TIAF1, wherein said first and said second amino acid sequences are contiguous and in a sequential order.

An isolated TIAF_{—}1_Skippingexon_{—}34_#PEP_NUM_{—}168 polypeptide, comprising a first amino acid sequence being at least about 90% homologous to amino acids 1-1691 of TIAF1, and a second amino acid sequence being at least about 90% homologous to amino acids 1730-2054 of TIAF1, wherein said first and said second amino acid sequences are contiguous and in a sequential order.

An isolated polypeptide of an edge portion of TIAF1_Skippingexon_{—}34_#PEP_NUM_{—}168, comprising a first amino acid sequence; being at least about 90% homologous to amino acids 1681-1691 of TIAF1, and a second amino acid sequence being at least about 90% homologous to amino acids 1730-1740 of TIAF1, wherein said first and said second amino acid sequences are contiguous and in a sequential order.

An isolated VEGFC_Skipping_exon_{—}4_#PEP_NUM_{—}7 polypeptide, comprising a first amino acid sequence being at least about 90% homologous to amino acids 1-184 of VEGFC, and a second amino acid sequence being at least about 70%, optionally at least about 80%, preferably at least about 85%, more preferably at least about 90% and most preferably at least about 95% homologous to a polypeptide having the sequence VSGSEQDLPHQLHVE (SEQ ID NO: 267), wherein said first and said second amino acid sequences are contiguous and in a sequential order.

An isolated polypeptide corresponding to a tail of VEGFC_Skipping_exon_{—}4_#PEP_NUM_{—}7, comprising a polypeptide having the sequence VSGSEQDLPHQLHVE (SEQ ID NO: 267).

An isolated VLDLR_Skipping_exon_{—}14_#PEP_{—NUM}_{—}4 polypeptide, comprising a first amino acid sequence being at least about 90% homologous to amino acids 1-654 of VLDLR, and a second amino acid sequence being at least about 70%, optionally at least about 80%, preferably at least about 85%, more preferably at least about 90% and most preferably at least about 95% homologous to a polypeptide having the sequence VKIGVKKTWRMEDVNTYACQHHRLMITLQNIPVPVPVGTM (SEQ ID NO: 268), wherein said first and said second amino acid sequences are contiguous and in a sequential order.

An isolated polypeptide corresponding to a tail of VLDLR_Skippingexon_{—}14_#PEP_NUM_{—}4, comprising a polypeptide having the sequence VKIGVKKTWRMEDVNTYACQHHRLMITLQNIPVPVPVGTM (SEQ ID NO: 268).

An isolated VLDLR_Skipping_exon_{—}15_#PEP_NUM_{—}5 polypeptide, comprising a first amino acid sequence being at least about 90% homologous to amino acids 1-702 of VLDLR, and a second amino acid sequence being at least about 90% homologous to amino acids 752-873 of VLDLR, wherein said first and said second amino acid sequences are contiguous and in a sequential order.

An isolated polypeptide of an edge portion of VLDLR_Skipping_exon_{—}15_#PEP_NUM_{—}5, comprising a first amino acid sequence being at least about 90% homologous to amino acids 692-702 of VLDLR, and a second amino acid sequence being at least about 90% homologous to amino acids 752-762 of VLDLR, wherein said first and said second amino acid sequences are contiguous and in a sequential order.

An isolated VLDLR_Skipping_exon_{—}8_#PEP_NUM_{—}1 polypeptide, comprising a first amino acid sequence being at least about 90% homologous to amino acids 1-356 of VLDLR, and a second amino acid sequence being at least about 90% homologous to amino acids 357-873 of VLDLR, wherein said first and said second amino acid sequences are contiguous and in a sequential order.

An isolated polypeptide of an edge portion of VLDLR_Skipping_exon_{—}8_#PEP_NUM_{—}1, comprising a first amino acid sequence being at least about 90% homologous to amino acids 346-356 of VLDLR, and a second amino acid sequence being at least about 90% homologous to amino acids 357-367 of VLDLR, wherein said first and said second amino acid sequences are contiguous and in a sequential order.

An isolated VLDLR_Skipping_exon_{—}9_#PEP_NUM_{—}2 polypeptide, comprising a first amino acid sequence being at least about 90% homologous to amino acids 1-395 of VLDLR, and a second amino acid sequence being at least about 90% homologous to amino acids 438-873 of VLDLR, wherein said first and said second amino acid sequences are contiguous and in a sequential order.

An isolated polypeptide of an edge portion of VLDLR_Skipping_exon_{—}9_#PEP_NUM_{—}2, comprising a first amino acid sequence being at least about 90% homologous to amino acids 385-395 of VLDLR, and a second amino acid sequence being at least about 90% homologous to amino acids 438-448 of VLDLR, wherein said first and said second amino acid sequences are contiguous and in a sequential order.

An isolated VLDLR_intron_{—}8_retention_#PEP_NUM_{—}6 polypeptide, comprising a first amino acid sequence being at least about 90% homologous to amino acids 1-395 of VLDLR, and a second amino acid sequence being at least about 70%, optionally at least about 80%, preferably at least about 85%, more preferably at least about 90% and most preferably at least about 95% homologous to a polypeptide having the sequence GESKKKTWTLQVMGKDSMYLVRYRSSKTNSDFPPRY (SEQ ID NO: 269), wherein said first and said second amino acid sequences are contiguous and in a sequential order.

An isolated polypeptide corresponding to a tail of VLDLR_intron_{—}8_retention_#PEP_NUM_{—}6, comprising a polypeptide having the sequence GESKKKTWTLQVMGKDSMYLVRYRSSKTNSDFPPRY (SEQ ID NO: 269).

An isolated VLDLR_skipping_exon_{—}12_#PEP_NUM_{—}3 polypeptide, comprising a first amino acid sequence being at least about 90% homologous to amino acids 1-568 of VLDLR, and a second amino acid sequence being at least about 70%, optionally at least about 80%, preferably at least about 85%, more preferably at least about 90% and most preferably at least about 95% homologous to a polypeptide having the sequence PYKKSPLLA (SEQ ID NO: 270), wherein said first and said second amino acid sequences are contiguous and in a sequential order.

An isolated polypeptide corresponding to a tail of VLDLR_skipping_exon_{—}12_#PEP_NUM_{—}3, comprising a polypeptide having the sequence PYKKSPLLA (SEQ ID NO: 270).

An isolated VWF_Skippingexon_{—}13#PEP_NUM_{—}187 polypeptide, comprising a first amino acid sequence being at least about 90% homologous to amino acids 1-477 of VWF, and a second amino acid sequence being at least about 70%, optionally at least about 80%, preferably at least about 85%, more preferably at least about 90% and most preferably at least about 95% homologous to a polypeptide having the sequence AGPRLCREDLRPVWELQWQPGRGLPYPLWAGGAPGGGLRERLEAARGLPGP AEAAQRSLRPQPAHEGSPRRRARS (SEQ ID NO: 271), wherein said first and said second amino acid sequences are contiguous and sequential order.

An isolated polypeptide corresponding to a tail of VWF_Skippingexon_{—}13_#PEP_NUM_{—}187, comprising a polypeptide having the sequence AGPRLCREDLRPVWELQWQPGRGLPYPLWAGGAPGGGLRERLEAARGLPGP AEAAQRSLRPQPAHEGSPRRRARS (SEQ ID NO: 271).

An isolated VWF_Skippingexon_{—}29_#PEP_NUM_{—}188 polypeptide, comprising a first amino acid sequence being at least about 90% homologous to amino acids 1-1684 of VWF, and a second amino acid sequence being at least about 90% homologous to amino acids 1724-2813 of VWF, wherein said first and said second amino acid sequences are contiguous and in a sequential order.

An isolated polypeptide, of an edge portion of VWF_Skippingexon_{—}29_#PEP_NUM_{—}188, comprising a first amino acid sequence being at least about 90% homologous to amino acids 1674-1684 of VWF and a second amino acid sequence being at least about 90% homologous to amino acids 1724-1734 of VWF, wherein said first and said second amino acid sequences are contiguous and in a sequential order.

An isolated VWF_Skippingexon_{—}8_#PEP_NUM_{—}186 polypeptide, comprising a first amino acid sequence being at least about 90% homologous to amino acids 1-291 of VWF, a bridging amino acid K and a second amino acid sequence being at least about 90% homologous to amino acids 334-2813 of VWF, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated polypeptide of an edge portion of VWF_Skippingexon_{—}8_#PEP_NUM_{—}186, comprising a first amino acid sequence being at least about 90% homologous to amino acids 281-291 of VWF, a bridging amino acid K and a second amino acid sequence being at least about 90% homologous to amino acids 334-344 of VWF, wherein said first amino acid sequence is contiguous to said bridging amino acid and said second amino acid sequence is contiguous to said bridging amino acid, and wherein said first amino acid sequence, said bridging amino acid and said second amino acid sequence are in a sequential order.

An isolated FGF12_Skipping_exon_{—}2_long_isoform #PEP_NUM 38 polypeptide, comprising a first amino acid sequence being at least about 70%, optionally at least about 80%, preferably at least about 85%, more preferably at least about 90% and most preferably at least about 95% homologous to a polypeptide having the sequence MAAAIASSLIRQKRQARESNSDRVSASKRRSSPSKDGRSLCERHVLGVFSKVR FCSGRKRPVRRRPA (SEQ ID NO: 272), and a second amino acid sequence being at least about 90% homologous to amino acids 43-181 of FGF12, wherein said first and second amino acid sequences are contiguous and in a sequential order.

The present invention successfully addresses the shortcomings of the presently known configurations by providing a method for large-scale prediction of alternative splicing events.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-e are graphs depicting the differences between alternative and constitutive exons as determined by analyzing human exon datasets (FIGS. 1a-c) and comparing human-mouse exon datasets (FIGS. 1d-e). For each of the curves, constitutive exons are denoted by squares, and alternative exons are denoted by diamond shapes. FIG. 1a—Length of conserved region in the last 100 nucleotides of an upstream intron flanking the exon. X axis, length of conserved region; Y axis, percent exons with upstream conserved region greater or equal to the value in X. Conservation was detected using local alignment with the mouse 100 counterpart intronic nucleotides. A minimum hit was 12 consecutive perfectly matching nucleotides. FIG. 1b—Length of conserved region in the first 100 nucleotides of a flanking intron downstream of the exon. Axes as in A. FIG. 1c shows human-mouse exon identity for percent exons. X axis, percent identity in the alignment of the human and the mouse exons; Y axis, percent exons with identity greater or equal to the value in X. FIG. 1d shows exon size distribution. X axis, exon size; Y axis, percent exons having size lesser or equal to the size in X. FIG. 1e shows human-mouse exon identity, for exons having a size that is a multiple of 3. X axis, percent identity in the alignment of the human and the mouse exons; Y axis, percent exons with identity greater or equal to the value in X.

FIG. 2a is a photograph depicting RT-PCR detection of a splice variant featuring skipping of exon 10 in Ephrine receptor B1 (GenBank Accession No. NM_{—}004441, SEQ ID Nos. 452, 453). Primers were taken from exon 9 (f, SEQ ID NO: 3) and 11 (r, SEQ ID NO: 4) of Ephrine receptor B1. Predicted size of full-length product was 324 bp, which was found in all samples but Placenta (lane 4). Skipping exon 10 variant (predicted size 201 bp) was detected in Testis (lane 11—Arrow) and slightly in Kidney (lane 12). A larger band was also found in Testis, and sequencing confimed it was a novel exon upstream of exon 10 (9A—Arrowhead, sequence of 3′ of exon 9a is set forth in SEQ ID NO: 201). All sequences were confirmed by sequencing. Tissue type cDNA, pools: 1—Cervix+HeLa; 2—Uterus; 3—Ovary; 4—Placenta; 5—Breast; 6—Colon; 7—Pancreas; 8—Liver+Spleen; 9—Brain; 10—Prostate; 11—Testis; 12—Kidney; 13—Thyroid; 14—Assorted Cell-lines. M denotes a 1 kb ladder marker; H denotes H₂O negative control.

FIG. 2b is a photograph depicting RT-PCR detection of a splice variant featuring skipping of exon 4 in VEGFC (GenBank Accession No. NM_{—}005429, SEQ ID Nos. 466, 467) Primers were taken from exon 3 (f, SEQ ID NO: 17) and 6 (r, SEQ ID NO: 18). Predicted size of full-length product was 351 bp, which was found in all samples. Skipping exon 4 variant (predicted sized 199 bp) was detected in all samples excluding Pancreas (lane 7) and a very weak expression in Breast and Colon (lanes 5 and 6). All sequences were confirmed by sequencing. A larger band was apparent in the testis and may represent a novel variant of VEGFC which sequence is yet to be determined. Tissue type cDNA pools: 1—Cervix+HeLa; 2—Uterus; 3—Ovary; 4—Placenta; 5—Breast; 6—Colon; 7—Pancreas; 8—Liver+Spleen; 9—Brain; 10—Prostate; 11—Testis; 12—Kidney; 13—Thyroid; 14—Assorted Cell-lines. M denotes a 1 kb ladder marker; H denotes H₂O negative control.

FIG. 2c is a photograph depicting RT-PCR detection of a splice variant featuring skipping of exon 4 in EphrinA5 (GenBank Accession No. NM_{—}001962, SEQ ID Nos. 450, 451) and a second splice variant featuring skipping of exon 11 in Heparanase 2 (GenBank Accession No. NM_{—}021828, SEQ ID Nos. 468, 469). Primers were taken from exon 1 (f, SEQ ID NO: 1) and 5 (r, SEQ ID NO: 2) for EFNA5 and exon 9 (f, SEQ ID NO: 19) and 12 (r, SEQ ID NO: 20) for HPA2. Predicted size of full length EFNA5 product was 287 bp, which was found in all samples (samples 1-8 not shown). Skipping exon 4 variant (predicted size 199 bp) was detected in all samples. Predicted size of full length HPA2 product (357 bp) was detected in all samples, excluding Breast and Pancreas (lanes 5 and 7). Skipping exon variant of HPA2 (199 bp) was found in Cervix (lane 1), Uterus (2), Prostate (10), Testis (11) and Kidney (1-2). In testis, two Novel exons were found and confirmed by sequencing (exons 11A and 11B, partial sequences are set forth in SEQ ID Nos: 203 and 204, respectively). All sequences were confirmed by sequencing.

FIG. 2d is a photograph depicting RT-PCR detection of a splice variant featuring skipping of exon 2 in FGF11 (GenBank Accession No. NM_{—}004112, SEQ ID Nos. 456, 457). Primers were taken from exon 1 (f, SEQ ID NO: 5) and 4 (r, SEQ ID NO: 6). Predicted full-length product was 344 bp, which was found in all samples. Skipping exon 2 variant (predicted size 233 bp) was detected in all samples excluding Uterus (lane 2), Placenta (lane 4), Colon (lane 6), Pancreas (lane 7), Brain (lane 9), Cell-lines (Lane 14) and very weakly in Breast and Liver and Spleen (lanes 5 and 8). All sequences were validated by sequencing. Tissue type cDNA pools: 1—Cervix+HeLa; 2—Uterus; 3—Ovary; 4—Placenta; 5—Breast; 6—Colon 7—Pancreas; 8—Liver+Spleen; 9—Brain; 10—Prostate; 11—Testis; 12—Kidney; 13—Thyroid; 14—Assorted Cell-lines. M denotes a 1\kb ladder marker; H denotes H₂O negative control.

FIG. 2e is a photograph depicting RT-PCR detection of a splice variant featuring skipping of exon 9 in NOTCH2 (GenBank Accession No. NM_{—}024408, SEQ ID Nos. 460, 461). Primers were taken from exon 8 (f, SEQ ID NO: 11) and 10 (r, SEQ ID NO: 12). Predicted full-length product was 352 bp, which was found only in Cervix and Breast. Skipping exon 9 variant (predicted size 169 bp) was detected in Testis (Lane 11—Marked by Arrow). Tissue type cDNA pools: 1—Cervix+HeLa; 2—Uterus; 3—Ovary; 4—Placenta; 5—Breast; 6—Colon; 7—Pancreas; 8—Liver+Spleen; 9—Brain; 10—Prostate; 11—Testis; 12—Kidney; 13—Thyroid; 14—Assorted Cell-lines. M denotes a 1 kb ladder marker; H denotes H₂O negative control.

FIG. 2f is a photograph depicting RT-PCR detection of a splice variant featuring skipping of exon 13, in PTPRZ1 (GenBank Accession No. NM_{—}002851, SEQ ID Nos. 464, 465). Primers were taken from the junction of exons 12-13 (f, SEQ ID NO: 15) and exons 14-15 junction (r, SEQ ID NO: 16). Predicted size of full-length product was 283 bp, which was found in Cervix (lane 1), Uterus (lane 2), Ovary (lane 3), Brain (lane 9), Prostate (lane 10) and Testis (lane 11). Exon 13 skipping (138 bp) was detected in Cervix (Lane 1), Ovary (lane 3), Brain (lane 9) and Testis (lane 11). All sequences were confirmed by sequencing. Tissue type cDNA pools: 1—Cervix+HeLa; 2—Uterus; 3—Ovary; 4—Placenta; 5—Breast; 6—Colon; 7—Pancreas; 8—Liver+Spleen; 9—Brain; 10—Prostate; 11—Testis; 12—Kidney; 13—Thyroid; 14—Assorted Cell-lines. M denotes 1 kb ladder marker; H denotes H₂O negative control.

FIG. 2g is a photograph depicting RT-PCR detection of splice variants featuring skipping of exons 13 and 14 in NTRK2 (GenBank Accession No. NM_{—}006180, SEQ; ID Nos. 462, 463). Primers were taken from exon 11-12 junction (f, SEQ ID NO: 13) and 15 (r, SEQ ID NO: 14). Predicted product of full-length product was 400 bp, which was found in all tissue samples excluding Placenta (lane 4), Breast (lane 5), Liver and Spleen (lane 8) and Cell-lines (lane 14). Exon 13 skipping (known—352 bp) was detected in all tissue samples excluding Placenta (lane 4), Liver and Spleen (lane 8) and Cell-lines (lane 14). Skipping both exons 13 and 14 (139 bp) was weakly found in Prostate (marked by an Arrow). All sequences were validated by sequencing. The sequence identity of the larger bands (e.g., 500 bp in lane 11) was not determined. Tissue type cDNA pools: 1—Cervix+HeLa; 2—Uterus; 3—Ovary; 4—Placenta; 5—Breast; 6—Colon; 7—Pancreas; 8—Liver+Spleen; 9—Brain; 10—Prostate; 11—Testis; 12—Kidney; 13—Thyroid; 14—Assorted Cell-lines. M denotes 1 kb ladder marker; H denotes H₂O negative control.

FIG. 2h is a photograph depicting RT-PCR detection of a splice variant featuring retention of intron 8 in Very Low Density Lipoprotein receptor (GenBank Accession No. NM_{—}003383 SEQ ID Nos. 457, 458). Primers were taken from exon 7-8 junction (f, SEQ D. NO: 7) and 10 (r, SEQ ID NO: 8). Predicted size of full-length product was 324 bp, which was found in all tissue samples excluding Brain (lane 9). Retention of intron 8 (predicted, size 427 bp) was detected in all tissue samples excluding Placenta (lane 4), Colon (lane 6), and Brain (lane 9). All sequences were confirmed by sequencing Tissue type cDNA pools: 1—Cervix+HeLa; 2—Uterus; 3—Ovary; 4—Placenta; 5—Breast; 6—Colon; 7—Pancreas; 8—Liver+Spleen; 9—Brain; 10—Prostate; 11—Tests; 12—Kidney; 13—Thyroid; 14—Assorted Cell-lines M denotes 1 kb ladder marker; H denotes H₂O negative control.

FIG. 2i is a photograph depicting RT-PCR detection of a first splice variant featuring skipping of exon 6 and a second splice variant featuring new exon 8a in FSH receptor (GenBank Accession No. NM_{—}000145, SEQ ID Nos. 459, 460). Primers were taken from exon 5 (f, SEQ ID NO: 9) and 10 (r, SEQ ID NO: 10). Predicted size of full-length product was 394 bp, which was found in Ovary, Testis and Thyroid (lanes 3, 11 and 13 respectively). Skipping exon 6 variant predicted size 316 bp—arrowhead) was detected in Ovary and Testis (lanes 3, 11). A larger band was also found in Ovary and Testis, and sequencing approved it was a novel exon upstream to exon 9 (was called 8a, SEQ ID NO: 202). All sequences were confirmed by sequencing Tissue type cDNA pools: 1—Cervix+HeLa, 2—Uterus; 3—Ovary; 4—Placenta; 5—Breast; 6—Colon; 7—Pancreas, 8—Liver+Spleen; 9—Brain; 10—Prostate; 11—Testis; 12—Kidney; 13—Thyroid; 14—Assorted Cell-lines. M denotes 1 kb ladder marker; H denotes H₂O negative control.

FIG. 2j is a photograph showing experimental validation for the existence of alternative splicing in selected predicted exons. RT-PCR for 15 exons (detailed in Table 8), for which no EST/cDNA indicating alternative splicing was found was conducted over 14 different tissue types and cell lines (see Methods). Detected splice variants were confirmed by sequencing. For nine of these exons a splice isoform was detected in at least one of the tissues tested. Only a single tissue is shown here for each of these nine exons. Lane 1, DNA size marker. Lane 2, exon 2 skipping in FGF11 in ovary, tissue (the 344 nt and 233 nt products are exon inclusion and skipping, respectively). Lane 3, exon 4 skipping in EFNA5 gene in ovary tissue (exon inclusion 287 nt; skipping 199 nt); Lane 4, exon 8 skipping in NCOA1 gene in placenta tissue (exon inclusion 377 nt; skipping 275 nt). Lane 5; exon 22 skipping in PAM gene in cervix tissue (exon inclusion 323 nt; skipping 215 nt). Additional upper band contains a novel exon in PAM. Lane 6, exon 9 skipping in GOLGA4 gene in uterus tissue (exon inclusion 288 nt; skipping 213 nt). Lane 7, exon 9 skipping of NPR2 gene in placenta tissue (282 nt inclusion; 207 nt; skipping). Lane 8, intron 8 retention in VLDLRV gene in ovary tissue (wild type 324 nt; intron retention 427 nt). Lane 9, alternative acceptor site in exon 12 of BAZ1A in ovary tissue (wild type 351 nt; alternative acceptor; variant 265 nt). The uppermost band represents a new exon in BAZ1A, inserted between; exons 12 and 13. Lane 10, alternative acceptor site in exon 7 of SMARCD1 in uterus tissue (wild type 353 nt; exon 7 extension 397 nt).

FIGS. 3a-z are schematic presentations of the proteins encoded by the selected splice variants compared to full length wild type proteins. A full description of the new variants is provided in Table 3, below. The protein domains are based on Swissprot annotation. FIG. 3a shows new alternatively spliced variants of VLDLR—Very low density Lipoprotein Receptor. The exon structure of the new variant is as follows: i. skipping exon 8 or 9; ii. extension of exon 8; iii. skipping exon 14; iv. skipping exon 15.

FIG. 3b shows a new alternatively spliced variant of VEGFC—Vascular endothelial growth factor C. The new variant skips exon 4.

FIG. 3c shows three new alternatively spliced variants of MET protooncogene, (HGF receptor). Exon structure of the new variants is as follows: i. extension of exon 12; ii. skipping of exon 4; iii skipping exon 18.

FIG. 3d shows four new alternatively spliced variants of ITGAV, integrin, alpha V (vitronectin receptor, alpha polypeptide). The exon structure of the new variants is as follows: i. skipping exon 11; ii. skipping exon 20; iii. skipping exon 21; iv. skipping exon 25.

FIG. 3e shows three new alternatively spliced variants of FSHR: follicle stimulating hormone receptor. The exon structure of the new variants is as follows: i. skipping exon 7; ii. skipping exon 8, iii. intron 7 retention.

FIG. 3f shows new alternatively spliced variants of LHCGR: luteinizing hormone/choriogonadotropin receptor. The exon structure of the new variants is as follows: i. skipping either exon 2, 3, 5, 6 or 7; ii. skipping exon 10; iii. intron 5 retention.

FIG. 3g shows a new alternatively spliced variant of Fibroblast growth factor—FGF11. The exon structure of the new variant new variant skips exon 2.

FIG. 3h shows two new alternatively spliced variants of Fibroblast growth factors—FGF12/13. The known FGF protein has two reported isoforms (isoform 1 and 2). The exon structure of the new splice variants is as follows: i. skipping exon 2 in both, isoform 1 and isoform 2; and ii. skipping exon 3 in both, isoform 1 and isoform 2.

FIG. 3i shows new alternatively spliced variants of Ephrin ligand A family proteins, EFNA 1, 3 and 5. The exon structure of the novel splice variants is as follows: i. skipping exon 3 in EFNA 13 and 5; ii. skipping exon 4 in EFNA 3 and 5; iii. skipping both exons 3 and 4 in EFNA 1, 3 and 5.

FIG. 3j shows three new alternatively spliced variants of Ephrin ligand B family (EFNB2). The exon structure of the new variants is as follows: i. skipping exon 2; ii. skipping exon 3; iii. skipping exon 4.

FIG. 3k shows four new alternatively spliced variants of Ephrin type A receptor 4 (EPHA4). The exon structure of the new variants is as follows: i. skipping exon 2; ii. skipping exon 3; iii. skipping exon 4; iv. skipping exon 12.

FIG. 3l shows seven new alternatively spliced variants of Ephrin type A receptor 5 (EPHA5). The exon structure of the new variants is as follows: i. skipping exon 4; ii. skipping exon 5; iii. skipping exon 8; iv. skipping exon 10; v. skipping exon 14; vi. skipping exon 17.

FIG. 3m shows two new alternatively spliced variants of Ephrin type A receptor 7 (EPHA7). The exon structure of the new variants is as follows: i. skipping exon 10; ii. skipping exon 15.

FIG. 3n shows three new alternatively spliced variants of Ephrin type B receptor 1 (EPHB1). The exon structure of the new variants is as follows: i. skipping exon 6; ii. skipping exon 8; iii. skipping exon 10.

FIG. 3o shows five new alternatively spliced variants of PTPRZ1—protein tyrosine phosphatase zeta 1. The exon structure of the new variants is as follows: i. skipping exon 7; ii. skipping exon 11, iii. skipping exon 13, iv. skipping exon 15; v. skipping exon 22.

FIG. 3p shows a new alternatively spliced variant of PTPRB1—protein tyrosine phosphatase beta 1. The new variant skips exon 26.

FIG. 3q shows new splice variants of ErbB2 and ErbB3 receptor tyrosine kinases. The exon structure of the new variants is as follows. i. new splice variant of ErbB2, skipping exon 6; ii. new splice variant of ErbB3 skipping exon 4; iii. new splice variant of ErbB3 skipping exon 15; iv. new splice variant of ErbB3, skipping exon 18.

FIG. 3r shows two new alternatively spliced variants of ErbB4 receptor tyrosine kinase. The exon structure of the new variants is as follows: i. skipping exon 14; ii. skipping exon 16.

FIG. 3s shows a new alternatively spliced variant of, Heparanase, skipping exon 10.

FIG. 3t shows seven new alternatively spliced variants of Heparanase 2. The exon structure of the new variants is as follows: i. skipping exon 5; ii. skipping exon 6; iii. skipping exon 7; iv. skipping exon 8; v. skipping exon 9; vi. skipping exon 10; vii. skipping exon 11.

FIG. 3u shows two new alternatively spliced variants of KIT oncogene (Tyrosine kinase receptor). The exon structure of the new variants is as follows: i. skipping exon 8; ii. skipping exon 14.

FIG. 3v shows a new alternatively spliced variant of KIT ligand, skipping exon 8.

FIG. 3w shows new alternatively spliced variants of JAG1. The exon structure of the new variants is as follows: i. skipping exon 10 or 18; ii. skipping exon 12; iii. skipping exon 22.

FIG. 3x shows new alternatively spliced variants of Notch homologs NTC2, NTC3 and NTC4. The exon structure new variants is as follows: i. is a new variant of NTC2, skipping exon 9 or 12; ii. is a new variant of NTC3, skipping exon 3; iii. is a new variant of NTC4, skipping exon 8.

FIG. 3y shows new alternatively spliced variants of BDNF/NT-3 growth factors receptors (NTRK2 and NTRK3). The exon structure of the new variants is as follows: i. is a new variant of NTRK2, skipping exon 14; ii. is a new variant of NTRK2, skipping exon 13 and 14; iii. is a new variant of NTRK3, skipping exon 5; iv. is a new variant of NTRK3, skipping exon 16.

FIG. 3z shows new alternatively spliced variants of GDNF receptor alpha (GFRA1) and Neurturin receptor alpha (GFRA2)-RET ligands. The exon structure of the new variants is as follows: i. is a new variant of GFRA1, skipping exon 4; ii. is a new variant of GFRA2, skipping exon 4.

FIGS. 4a-m are schematic presentations of the proteins encoded by the selected splice variants compared to full length wild type proteins. A full description of the new variants is provided in Table 3, below. The protein domains are based on Swissprot annotation.

FIG. 4a shows new alternatively spliced variants of Interleukin 16. The exon structure of the new variants is as follows: i. skipping exon 5; ii. skipping exon 18.

FIG. 4b shows new alternatively spliced variants of Insulin growth factor binding protein 4, IGFBP4, skipping exon 3.

FIG. 4c shows new alternatively spliced variants, of Angiopoietin 1. The exon structure of the new variants is as follows: i. skipping exon 5; ii. skipping exon 6; iii. skipping exon-8.

FIG. 4d shows new alternatively spliced variants of long and short isoforms of Neuropilin 1. The exon structure of the new variants is as follows: i. is a new variant of a long isoform, skipping exon 5; ii is a new variant of a short isoform, skipping exon 5.

FIG. 4e shows new alternatively spliced variant of Endothelin converting enzyme 1, skipping exon 2.

FIG. 4f shows new alternatively spliced variants of Endothelin converting enzyme 2. The exon structure of the new variants is as follows: i. skipping exon 8; ii. skipping exon 12, iii. skipping exon 13; iv. skipping exon 15.

FIG. 4g shows new alternatively spliced variants of Enkephalinase, Neutral endopeptidase (NME). The exon structure of the new variants is as follows: i. skipping exon 4; ii. skipping exon 7; iii. skipping exon 9; iv. skipping exon 11; v. skipping exon 12; vi. skipping exon 16.

FIG. 4h shows new alternatively spliced variants of APBB1—Alzheimer's disease amyloid A4 binding protein. The exon structure of the new variants is as follows: i. skipping exon 3; ii. skipping exon 7 or 9; iii. skipping exon 10; iv skipping exon 12.

FIG. 4i shows new alternatively spliced variant of Transforming growth factor beta 2 (TGFB2), skipping exon 5.

FIG. 4j shows new alternatively spliced variant of IL1 receptor accessory, protein (IL1RAP), skipping exon 11.

FIG. 4k shows new alternatively spliced variants of IL1 receptor accessory protein like family members IL1RAPL1 and IL1 RAPL2. The exon structure of the new variants is as follows: i. skipping exon 4; ii. skipping exon 5; iii. skipping exon 6; iv. skipping exon 7; v. skipping exon 8.

FIG. 4l shows new alternatively spliced variant of Vitamin K dependent protein S precursor (PROS1), skipping exon 3.

FIG. 4m shows new alternatively spliced variants of Ovarian carcinoma antigen CA125 (M17S2). The exon structure of the new variants is as follows: i. skipping exon 14; ii. skipping exon 15; iii. skipping exon 20.

FIG. 5a is a black box diagram illustrating a system designed and configured for generating a database of putative gene products and generated according to the teachings of the present invention.

FIG. 5b is a black box diagram illustrating a remote configuration of the system of FIG. 5a.

FIG. 6 shows the ROC curve of classification rules in the experiments according to the present invention.

### DESCRIPTION OF THE PREFERRED EMBODIMENTS

The present invention is of methods of identifying putative gene products by interspecies sequence comparison and biomolecular sequences identified thereby, which can be used in a variety of therapeutic and diagnostic applications.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Alternative splicing is a mechanism by which multiple expression products are generated from a single gene. It is estimated that between 35% to 60% of all human genes can putatively undergo alternative splicing. Currently, the only approach available for the detection of alternatively spliced products relies on the use of expressed sequence data, such as, Expressed Sequence Tags (ESTs) and cDNAs.

However, expressed sequences present a problematic source of information, as they present only a sample of the transcriptome. Thus, the detection of a splice variant is possible only if it is expressed above a certain expression level, or if there is an EST library prepared from the tissue type in which the variant is expressed. In addition, ESTs are very noisy and contain numerous sequence errors [Sorek (2003) Nucleic Acids Res. 31:1067-1074]. For example, many wrongly termed splice events, actually represent incompletely spliced heteronuclear RNA (hnRNA) or oligo(dT)-primed genomic DNA contaminants of cDNA library constructions. Furthermore, the splicing apparatus is known to make errors, resulting in aberrant transcripts that are degraded by the mRNA surveillance system and amount to little that is functionally important [Maquat and Charmichael (2001) Cell 104:173-176; Modrek and Lee (2001) Nat. Genet. 30:13-19]. Consequently the mere presence of a transcript isoform in the ESTs cannot establish a functional role for it.

Thus, the use of expressed sequence data allows only very genera estimates regarding the number of genes that have splice variants (currently running between 35% and 75%), but does not allow specific estimation regarding the actual number and identity of exons that can be alternatively spliced.

While reducing the present invention to practice, the present inventors uncovered a combination of sequence features unique to alternatively spliced exons, which allow distinction thereof from constitutively spliced ones. These findings allow to computationally identify alternatively spliced exons even when no expressed sequence data is available, to thereby predict yet unknown gene expression products.

Thus, according to one aspect of the present invention there is provided a method of identifying alternatively spliced exons.

As used herein “alternatively spliced exons” refer to exons, which are spliced into an expression product only under specific conditions such as specific tissue environment, stress conditions or development state.

The method according to this aspect of the present invention is effected by scoring each of a plurality of exon sequences derived from genes of a species (i.e., a eukaryotic organism such as human) according to at least one sequence parameter. Exon sequences of the plurality of exon sequences scoring above a predetermined threshold represent alternatively spliced exons, thereby identifying the alternatively spliced exons.

Typically, exon sequences are identified by screening genomic data for reliable exons which require canonical splice sites and elimination of possible genomic contamination events [Sorek (2003) Nucleic Acids Res. 31:1067-1074].

As mentioned hereinabove, the present inventors uncovered a number of sequence parameters, which can serve for the identification of alternatively spliced exon sequences. Preferred examples of such are summarized infra.

Exon length—Typically, conserved alternatively spliced exons are much shorter than constitutively spliced exons, probably since the spliceosome typically recognizes exons that are between 50 and 200 bp.

Division by three—Since, alternatively spliced exons are cassette exons, which may be incorporated in an expressed gene product or skipped, they should be divisible by three, such that the reading frame is maintained when they are skipped.

Conservation level between the exon sequences and corresponding exon sequences of ortholohgous species—Alternatively spliced exons are typically more conserved than constitutively spliced exons. This is probably since alternatively spliced exons contain sub-sequences that are important for inclusion/exclusion regulation [Exonic Splicing” Enhancers and Silencers, Cartegni (2002) Nat. Rev. Genet. 3:285-298]. This requirement imposes additional conservation constraint on the sequence of the exon.

Length of conserved intron sequences upstream of each of the exon sequences—Alternatively spliced exons exhibit high level of conservation in an intronic sequence of about 100 bases upstream of the exon. This is only sparsly so for constitutively spliced exons. This is probably since these sequences are involved regulation of inclusion/exclusion of the alternatively spliced exon. Alignment of intronic regions can be done using sim4 software. sim4 sources are available from http://globin.cse.psu.edu/globin/html/software.html. According to a presently known embodiment of the present invention the length of conserved intronic sequence is from about 12 to about 100 nucleotides.

Length of conserved intron sequences downstream of the exon sequences—Alternatively spliced exons exhibit high level of conservation in an intronic sequence of about 100 bases downstream of the exon. This is only sparsly so for constitutively spliced exons. This is probably since these sequences are involved in regulation of inclusion/exclusion of the alternatively spliced exon. Alignment of intronic regions can be done using sim4 software. sim4 sources are available from http://globin.cse.psu.edu/globin/html/software.html. According to a presently known embodiment of the present invention the length of conserved intronic sequence is from about 12 to about 100 nucleotides.

Conservation level of intron sequences upstream of each of the exon sequences—For alternatively spliced exons, the intronic sequences in the 100 bases upstream of the exon are frequently conserved between species. This correlation is less strongly shown by constitutively spliced exons [Sorek and Ast (2003) Genome Res. 13(7):1631-7]. This is probably since these sequences are involved in regulation of inclusion/exclusion of the alternatively spliced exon. Therefore, conservation level of intron sequences upstream of exon sequences can be used to distinguish alternative from constitutive exons. Alignment of intronic regions can be done using sim4 software, which may be obtained from http://globin.cse.psu.edu/globin/html/software.html. The measured length of the conserved sequence was generally found to be between 12 to 100 nucleotides.

Conservation level of intron sequences downstream of each of the exon sequences—For alternatively spliced exons, the intronic sequences in the 100 bases downstream of the exon are frequently conserved between species. This correlation is less strongly shown by constitutively spliced exons. This is probably since these sequences are involved in regulation of inclusion/exclusion of the alternatively spliced exon. Therefore, conservation level of intron sequences downstream of exon sequences can be used to distinguish alternative from constitutive exons. Alignment of intronic regions can be done using sim4 software, which are available from http://globin.cse.psu.edu/globin/html/software.html.

Each of the above-described parameters can be considered separately according to predetermined criteria however a combination with other parameters used, is preferred. In this case, each parameter is preferably also weighted according to its importance and a scoring system e.g., a scoring matrix, is preferably applied.

Such a scoring matrix can list the various exons across the X-axis of the matrix while each parameter can be listed on the Y-axis of the matrix. Parameters include both a predetermined range of values from which a single value is selected from each exon, and a weight. Each exon is scored at each parameter according to its value and the weight of the parameter.

Finally, the scores of each parameter of a specific exon sequence are summed and the results are analyzed.

Exons which exhibit a total score greater than a particular stringency threshold are grouped as alternatively spliced exons.

According to presently known preferred embodiments of this aspect of the present invention the best scored exons share at least about 95% identity with an ortholohgous exon; exon size is a multiple of 3; exon length of about 1000 bases; length of conserved intron sequences upstream of the exon sequence is at least about 12 bases; length of conserved intron sequences downstream of the exon sequence is at least about 15 bases; conservation level of the intron sequences upstream of the exon sequence is at least about 85%; conservation level of the intron sequences downstream of the exon sequence is at least about 60%.

As mentioned, the above-described methodology allows the prediction of yet unknown alternatively spliced exons, even in the absence of available expressed sequences. This allows the prediction of putative gene products of any known gene

Thus in order to predict expression products of a gene of interest, alternatively spliced exons thereof are identified as described above. Thereafter, chromosomal location of the identified exons is analyzed with respect to the coding sequence of the gene of interest, to thereby predict expression products of the gene of interest.

Chromosomal location of the newly uncovered sequences may be done as described by aligning the new sequence to the genome, as described for example by Modrek (2001) Nucleic Acids Research, 29:2850-2859. Genomic sequences, which are found to include these exons, are then manipulated to exclude them to thereby generate the new isoforms.

For example, when the newly identified alternative exon is predicted to be skipped, all transcripts that are known to include it are computationally or manually manipulated to delete the sequence of the exon therefrom, thus creating a new transcript that represents the exon-skipping splice variant.

Once putative transcripts are identified using the above methodology, corresponding protein products can be predicted using any translation software known in the art [e.g., ORF-finder (http://ww.nbi.nlm.rih.gov/gorf/gorf.html)].

According to another aspect of the present invention there is provided a method of predicting expression products of a gene of interest in a given species (any eukaryotic organism). The method according to this aspect of the present invention is effected by clustering expressed sequences of the given species to form a contig.

The term “contig” refers to a series of overlapping sequences with sufficient identity to create a longer contiguous sequence.

Expressed sequence clustering is effected using clustering methods which are well known in the art. Examples of clustering/assembly procedures with associated databases which are commercially available include, but are not limited to, UniGene (http://www.ncbi.nlm.nih.gov/UniGene), TIGR Gene Indices (http://www.tigr.org/tdb/tgi.shtml), STACKED (http://www.sanbi.ac.za/Dbases.html), trEST (ftp://ftp.isrec.isb_sib.ch/gub/databases/trest) and LEADS™ (http://www.cgen.com).

Following contig construction, exon sequences of orthologues of the gene of interest which display homology with the contig sequence are aligned to a genome of interest (i.e., genome of the given species). Orthologous exon sequences which alignment overlaps the chromosomal location of the given contig are added to the set of sequences in the contig. This larger set of sequences is then assembled to form a hybrid multi-species contig.

Expression products that are unique to the hybrid contig and do not appear in the original contig are identified. It will be appreciated that such unique expression products could not have been identified using prior art methods, which do not utilize expressed sequences from other species.

The above-described methodology is further described in Example 4 of the Examples section.

Once novel transcripts of the gene of interest the given species are identified, their corresponding protein products are predicted, as described above.

Biomolecular sequences uncovered as described herein can be experimentally validated using any method known in the art, such as northern blot, RT-PCR, western-blot and the like. For further details see Example 2 of the Examples section. Functional analysis of biomolecular sequences identified as described herein can be effected using biochemical, cell-biology and molecular methods which are well known in the art.

Biomolecular sequences (i.e., nucleic acid and polypeptide sequences) uncovered using the above-described methodology can be functionally annotated to discover their contribution to biological processes and physiological complexity. Numerous methods of automated gene annotation are known in the art (reviewed by Ashsurst and Collins (2003) Annu. Rev. Genomics Hum. Genet. (2003) 4:69-88. Such automatic annotation approaches are summarized in Example 5 of the Examples section below and are also the subject of U.S. Pat. Appl. No. 60/539,129.

Alternatively spliced exons and/or expression products derived therefrom (i.e., including the exons thus identified or skipping same) can be stored in a database, which can be generated by a suitable computing platform.

Although the present methodology can be effected using prior art systems modified for such purposes, in order to process large amounts of sequence data, the present methodologies are preferably effected using a dedicated computational system.

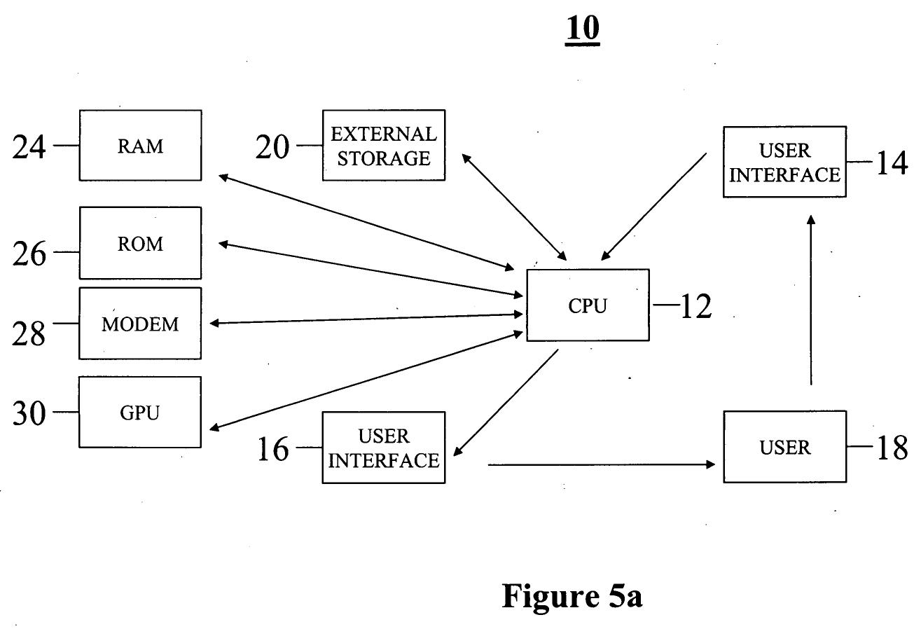

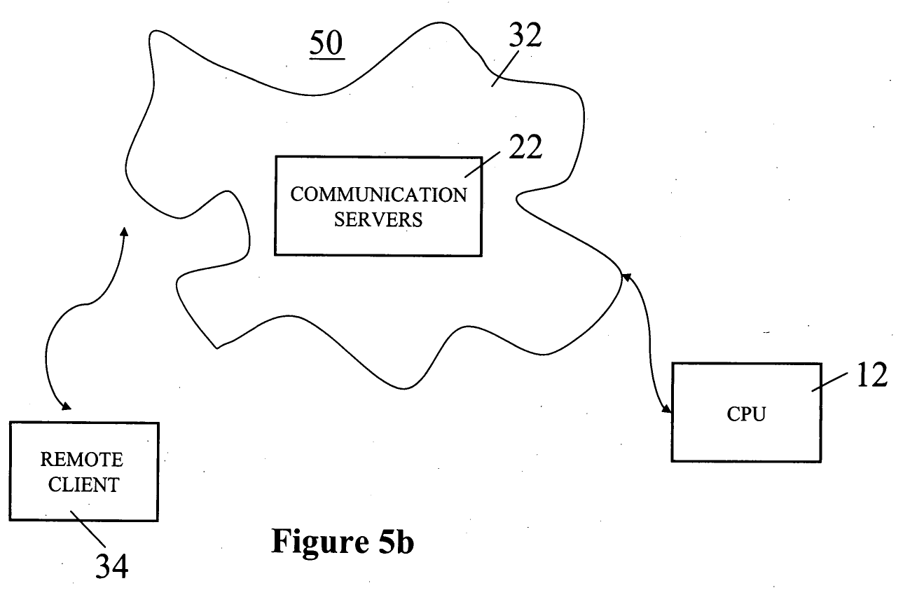

Thus, according to another aspect of the present invention and as illustrated in FIGS. 5a-b, there is provided a system for generating a database of alternatively spliced sequences.

System 10 includes at least one central processing unit (CPU) 12, which executes a software application designed and configured for identifying alternatively spliced sequences. System 10 may also include a user input interface 14 [e.g., a keyboard and/or a cursor control device (e.g., a joy stick)] for inputting database or database related information, and a user output interface 16 (e.g., a monitor) for providing database information to a user 18.

System 10 may also include random access memory 24, ROM memory 26, a modem 28 and a graphic processing unit (GPU) 30.

System 10 preferably stores sequence information of the alternatively spliced sequences identified thereby on an internal and/or external storage device 20 such as a magnetic, optico-magnetic or optical disk as a database of alternatively spliced sequences. Such a database further includes information pertaining to database generation (e.g., source library), parameters used for selecting polynucleotide sequences, putative uses of the stored sequences, and various other annotations (as described below) and references which relate to the stored sequences and respective expression products.

The hardware elements of system 10 may be tied together by a common bus or several interlinked buses for transporting data between the various elements. Examples of system 10 include but are not limited to, a personal computer, a work station, a mainframe and the like.

System 10 of the present invention may be used by a user to query the stored database of sequences, to retrieve nucleotide sequences stored, therein or to generate polynucleotide sequences from user inputted sequences.

The methods of the present invention can be effected by any software application executable by system 10. The software application can be stored in random access memory 24, or internal and/or external data storage device 20 of system 10.

The database generated and stored by system 10 can be accessed by an on-site user of system 10, or by a remote user communicating with system 10, through for example, a terminal or thin client.

The latter configuration is best exemplified by the client-server system 50 which is shown in FIG. 5b. System 50 is configured to perform similar functions to those performed by system 10. In system 50, communication between a remote client 34 (e.g., computer, PDA, cell phone etc) and CPU unit 12 of a local server or computer is typically effected via a communication network 32. Communication network 32 can be any private or public communication network including, but not limited to, a standard or cellular telephony network, a computer network such as the Internet or intranet, a satellite network or any combination thereof.

As illustrated in FIG. 5b, communication network 32 can include one or more communication servers 22 (one shown in FIG. 5b) which serve for communicating data pertaining to the sequence of interest between remote client 18 processing unit 12. Thus, a request for data or processed data is communicated from remote client 18 to processing unit 12 through communication network 32 and processing unit 12 sends back a reply which includes data or processed data to remote client 18. Such a system configuration is advantageous since it enables users of system 50 to store and share gathered information and to collectively analyze gathered information.

Such a remote configuration can be implemented over a local area network (LAN) or a wide area network (WAN) using standard communication protocols.

It will be appreciated that existing computer networks such as the Internet can provide the infrastructure and technology necessary for supporting data communication between any number of users 18 and processors 12.

By applying the algorithms described hereinabove and in the Examples section, which follows, the present inventors collected sequence information which is presented in the files “transcripts.fasta” and “proteins.fasta” of enclosed CD-ROM1 and in the files “transcripts” and “proteins” of enclosed CD-ROM2. Annotations of these sequences are provided in the file “AnnotationForPatent.txt” of enclosed CD-ROM 1.

Novel polynucleotide sequences uncovered using the above-described methodology can be used in various clinical applications (e.g., therapeutic and diagnostic) as is further described hereinbelow.

A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results form reverse transcription or messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Thus, the present invention encompasses nucleic acid sequences described hereinabove; fragments thereof, sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth in the file “transcripts.fasta” of enclosed CD-ROM1 and in the file “transcripts” of enclosed CD-ROM2], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

Thus, the present invention also encompasses polypeptides encoded by the polynucleotide sequences of the present invention. The present invention also encompasses homologues of these polypeptides such homologues can be at least 50%, at least 55%, at least 60%, at least 65%, at least 70%, at least 75%, at least 80%, at least 85%, at least 95% or more say 100% homologous to the amino acid sequences set forth in the file “proteins.fasta” of enclosed CD-ROM1 and in the file “proteins” of enclosed CD-ROM2, as can be determined using BlastP software of the National Center of Biotechnology Information (NCBI) using default parameters. Finally, the present invention also encompasses fragments of the above described polypeptides and polypeptides having mutations, such as deletions, insertions or substitutions of one or more amino acids, either naturally occurring or man induced, either randomly or in a targeted fashion.

As mentioned hereinabove, biomolecular sequences uncovered using the methodology of the present invention can be efficiently utilized as tissue or pathological markers and as putative drugs or drug targets for treating or preventing a disease, according to their annotations (see Examples 6 and 7 of the Examples section).

For example, it is conceivable that the biomolecular sequences of the present invention may be functionally altered, by the addition or deletion of exons as described above.

As used herein the phrase “functionally altered biomolecular sequences” refers to expressed sequences, which protein products exhibit gain of function or loss of function or modification of the original function. Specific examples of functionally altered gene products identified using the teachings of the present invention are provided in Table 3, below.

As used herein the phrase “gain of function” when made in reference to a gene product (e.g., product of alternative splicing, product of RNA editing), indicates increased functionality as compared to the wild type gene product. Such a gain of function may have a dominant effect on the wild-type gene product. An alternatively spliced variant of Max, a binding partner of the Myc oncogene, provides a typical example for a “gain of function” alteration. This variant is truncated at the COOH-terminus and while is still capable of binding to the CACGTG motif of c-Myc, it lacks the nuclear localization signal and the putative regulatory domain of Max. When tested in a myc-ras cotransformation assay in rat embryo fibroblasts, wild-type Max suppressed cellular transformation, whereas the above-described Max splice variant enhanced transformation [Makela T P, Koskinen P J, Vastrik I, Alitalo K., Science. 1992 Apr. 17; 256(5055):373-7]. Thus, it is envisaged that a protein product, which exhibits a gain of function contributing to disease onset or progression be down regulated to thereby treat the disease. Alternatively, when such a gain of function promotes positive biological processes such as enhanced wound-healing, it is highly desirable to up-regulate expression or activity of the protein product in the subject in need thereof. Methods of up-regulating or down-regulating expression or activity of gene products are summarized hereinbelow.

As used herein the phrase “loss of function” when made in reference to any gene product (mRNA or protein), indicates total or partial reduction in function as compared to the wild type gene product. Loss of function can also manifest itself through a dominant negative effect.

As used herein the phrase “dominant negative” refers to the dominant negative effect of a gene product (e.g., product of alternative splicing, product of RNA editing) on the activity of wild type protein. For example, a protein product of an altered splice variant may bind a wild type target protein without enzymatically activating it (e.g., receptor dimers), thus blocking and preventing the active enzymes from binding and activating the target protein. This mode of action provides a mechanism to the dominant negative action of soluble receptors on wild-type membrane anchored receptors. Such soluble receptors may compete with wild-type receptors on ligand-binding and as such may be used as antagonists. For example, two splice variants of guanylyl cyclase-B receptor were recently described (GC-B1, Tamura N and Garbers D L, J. Biol. Chem. (2003) 278(49):48880-9). One form has a 25 amino acid deletion in the kinase homology domain. This variant binds the ligand but fails to activate the cyclase. A second variant includes only a portion of the extracellular domain. This form fails to bind the ligand. Both variants. When co-expressed with the wild-type receptor both act as dominant negative isoforms by virtue of blocking formation of active GC-B1 homodimers.

A dominant negative effect may also be exerted by miss-localization of the altered variant or by multiple modes of action. For example, the splice variants of wild-type mytogen activated protein kinase 5a, ERK5b and mERK5c act as dominant negative inhibitors based on inhibition of mERK5a kinase activity and mERK5a-mediated MEF2C transactivation. The C-terminal tail, which contains a putative nuclear localization signal, is not required for activation and kinase activity but is responsible for the activation of nuclear transcription factor MEF2C due to nuclear targeting. In addition, the N-terminal domain spanning amino acids (aa) 1-77 is important for cytoplasmic targeting; the domain from aa 78 to 139 is required for association with the upstream kinase MEK5; and the domain from an 140-406 is necessary for oligomerization [Yan et al. J Biol Chem. (2001) 276(14):10870-8]. In the case of protein products which exhibit dominant negative effect, it may be highly desirable to up-regulate their expression when necessary. For example, in a malignant stage which is controlled by over-expression of a specific receptor tyrosine kinase it may be desirable to upregulate expression or activity of a dominant negative form thereof to thereby treat the disease. For example, the soluble isoform of ErbB-2 and/or ErbB-3 which were uncovered as described herein (further described in Table 3, below) may be exogenously upregulated so as to treat epithelial cancers. Alternatively, when a dominant negative form of a naturally occurring negative regulator of a biochemical proliferative pathway is expressed in cancer, it may be highly desirable to down-regulate expression or activity of this altered form to thereby treat the disease. In such a case this dominant negative isoform also serves as a valuable diagnostic tool which may be also used for monitoring disease progression with or without treatment.

The phrase “modification of the original function” may be exemplified by a changing a receptor function to a ligand function. For example, a soluble secreted receptor may exhibit change in functionality as compared to a membrane-anchored wild-type receptor by acting as a ligand, activating parallel signaling pathways by trans-signaling [e.g., the signaling reported for soluble IL-6R, Kallen Biochim Biophys Acta. (2002) Nov. 11; 1592(3):323-43], stabilizing ligand-receptor interactions or protecting the ligand or the wild-type receptor from degradation and/or prolonging their half-life. In this case the soluble receptor will function as an agonist.

Thus, the biomolecular sequences of the present invention can be used as drugs or drug targets for treating a disease in a subject either by upregulating or downregulating expression thereof in the subject (i.e., a mammal, preferably a human subject).

As used herein the term “treating” refers to alleviating or diminishing a symptom associated with the disease or the condition. Preferably, treating cures, e.g., substantially eliminates, and/or substantially, decreases, the symptoms associated with the diseases or conditions of the present invention.

Antibodies, oligonucleotides, polynucleotides, polypeptides (collectively termed herein “agents”) and methods of utilizing same for upregulating or downregulating activity or expression of biomolecular sequences in a subject are summarized infra.

Upregulating

An agent capable of upregulating expression of a specific protein product may be an exogenous polynucleotide sequence designed and constructed to express at least a functional portion thereof (e.g., a catalytic domain, a protein-protein interaction domain, etc.). Accordingly, the exogenous polynucleotide sequence may be a DNA or RNA sequence encoding the protein.

The exogenous polynucleotide may be cloned from any a normal origin which is a suitable to provide the desired protein product or compatible homologs thereof. Methods of molecular cloning are described in the Example section which follows.

To express an exogenous protein in mammalian cells, a polynucleotide same is preferably ligated into a nucleic acid construct suitable for mammalian cell expression. Such a nucleic acid construct includes a promoter sequence for directing transcription of the polynucleotide sequence in the cell in a constitutive or inducible manner. Any suitable promoter sequence can be used by the nucleic acid construct of the present invention. Preferably, the promoter utilized by the nucleic acid construct of the present invention is active ink the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733]0 and immunoglobulins; [Banerji et al. (1983) Cell 33729-740], neuron-specific promoters such as the neurofilament-promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

It will be appreciated that the nucleic acid construct can be administered to the subject employing any suitable mode of administration, described hereinbelow (i.e., in-vivo gene therapy). Alternatively, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Agents for upregulating endogenous expression of specific splice variants of a given gene include antisense oligonucleotides, which are directed at splice sites of interest, thereby altering the splicing pattern of the gene. This approach has been successfully used for shifting the balance of expression of the two isoforms of Bcl-x [Taylor (1999) Nat. Biotechnol. 17:1097-1100; and Mercatante (2001) J. Biol. Chem. 276:16411-16417]; IL-5R [Karras (2000) Mol. Pharmacol. 58:380-387]; and c-myc [Giles (1999) Antisense Acid Drug Dev. 9:213-220].

For example, interleukin 5 and its receptor play a critical role as regulators of hematopoiesis and as mediators in some inflammatory diseases such as allergy and asthma. Two alternatively spliced isoforms are generated from the IL-5R gene, which include (i.e., long form) or exclude (i.e., short form) exon 9. The long form encodes an intact membrane-bound receptor, while the shorter form encodes a secreted soluble non-functional receptor. Using 2′-O-MOE-oligonucleotides specific to regions of exon 9, Karras and co-workers (supra) were able to significantly decrease the expression of the wild type receptor and increase the expression of the shorter isoforms. Approaches which can be used to design and synthesize oligonucleotides according to the teachings of the present invention are described hereinbelow and by Sazani and Kole (2003) Progress in Molecular and Subcellular Biology 31:217-239.

Alternatively or additionally, upregulation may be effected by administering to the subject the polypeptide product per se or an active portion thereof, as described hereinabove. However, since the bioavailability of large polypeptides is relatively small due to high degradation rate and low penetration rate, administration of polypeptides is preferably confined to small peptide fragments (e.g., about 100 amino acids).

Polypeptide products can be biochemically synthesized such as by employing standard solid phase techniques. Such methods include exclusive solid phase synthesis, partial solid phase synthesis methods, fragment condensation classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the and further described by John Morrow Stewart and Janis Dillaha Young, Solid Phase Peptide Syntheses (2nd Ed.; Pierce Chemical Company, 1984).

Synthetic polypeptides can be purified by preparative high performance liquid chromatography [Creighton T. (1983) Proteins, structures and molecular principles. WH Freeman and Co. N.Y.]; and the composition of which can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514 Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant; Molecular Biology, Academic Press, NY, Section VIII, pp 421-463.

An agent capable of upregulating a biomolecular sequence of interest may also be any compound which is capable of increasing the transcription and/or translation of an endogenous DNA or mRNA encoding the desired protein product.

Downregulating

One example of an agent capable of downregulating the activity of a protein product is an antibody or antibody fragment capable of specifically binding to the specific protein product of the present invention and neutralizing its activity. Preferably, the antibody specifically binds at least one epitope of the protein product. As used herein, the term “epitope” refers to any antigenic determinant on an antigen to which the paratope of an antibody binds. For example, an antibody capable of specifically binding a truncated form of Follicular Stimulating Hormone Receptor (FSHR, SEQ ID NO: 46) may be used to downregulate this putative dysfunctional isoform of FSHR to thereby treat infertility problems associated therewith. Such an antibody is preferably directed at a bridging polypeptide (SEQ ID NO: 223) of SEQ ID NO: 46, to allow distinction of this isoform from the wild-type FSHR polypeptide.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme Papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and, the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, Which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci; USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide, linker. These single-chain antigen binding proteins (sFv) are, prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived form non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329′(1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Another agent capable of downregulating a biomolecular sequence of the present invention is a small interfering RNA (siRNA) molecule. RNA interference is a two-step process. The first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 119-21 bp duplexes (siRNA), each with 2-nucleotide 3′ overhangs [Hutvagner and Zamore Curr. Opin. Genetics and Development 12:225-232 (2002); and Bernstein Nature 409:363-366 (200.1)].

In the effector step, the siRNA duplexes bind to a nuclease complex to form the RNA-induced silencing complex (RISC). An ATP-dependent unwinding of the siRNA duplex is re for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001)]. Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2: 110-119 (2001), Sharp Genes. Dev. 15:485-90 (2001); Hutvagner and Zamore Curr. Opin. Genetics and Development 12:225-232 (2002)]. For more information on RNAi see the following reviews Tuschl Chem Biochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Another agent capable of downregulating a biomolecular sequence of the present invention is a DNAzyme molecule capable of specifically cleaving an mRNA transcript or DNA sequence of the biomolecular sequence. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

Downregulation of a biomolecular sequence can also be effected by using an antisense oligonucleotide capable of specifically hybridizing with an mRNA transcript of interest.

Design of antisense molecules must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model a target mRNAs (human lactate dehydrogenase A and B and rat gp130) in cell culture as evaluated by a kinetic PCR technique proved effective in almost all cases, including tests against three-different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16:1374-1375 (1998)].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patient [Geri Curr Opin Mol Ther 1:297-306 (1999)].

More recently, antisense-mediated suppression of human heparanase gene expression has been reported to inhibit pleural dissemination of human cancer cells in a mouse mode [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating a biomolecular sequence of interest is a ribozyme molecule capable of specifically cleaving an mRNA transcript encoding a specific protein product. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

An additional method of regulating the expression of a biomolecular sequence in cells is via triplex forming oligonuclotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 3754008. Modification of the oligonuclotides, such as the introduction of intercalators and backbone substitutions, and optimization of binding conditions (pH and cation concentration) have aided in overcoming, inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3'--A | G | G | T |
| duplex | 5'--A | G | C | T |
| duplex | 3'--T | C | G | A |

However, it has been shown that the A-AT and G-GC triplets have the greatest triple helical stability (Reither and Jeltsch, B M C Biochem, 2002, Sep. 12, Epub). The same authors have demonstrated that TFOs de signed according to the A-AT and G-GC rule do not form non-specific triplexes, indicating that the triplex formation is indeed sequence specific.

Triplex-forming oligonucleotides preferably are at least about 15, more preferably about 25, still more preferably about 30 or more nucleotides in length, up to about 50 or about 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 112:487-94). Detailed description of the design synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

Oligonucleotides designed for carrying out the methods of the present invention for any of the sequences provided herein (designed as described above) can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases.

The oligonucleotides of the present invention may comprise heterocylic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder. Such modifications can oftentimes facilitate oligonucleotide uptake and resistivity to intracellular conditions.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. ,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanine, 5-halo particularly 5-bromo, 5-trifluoromethyl and other 5-substituted uracils and cytosines, 7-methyl guanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclose in The Concise Encyclopedia Of Polymer Science and Engineering, pages 858-859, Kroschwitz, J. I., ed. John Wiley & Sons, 1990, those disclosed by Englisch et al., Angewandtet Chemie, International Edition, 1991, 30, 613, and those disclosed by Sanghvi, Y. S., Chapter 15, Antisense Research and Applications, pages. 289-302, Crooke, S. T. and Lebleu, B., ed., CRC Press, 1993. Such base snare particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 35-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are riot limited to lipid moieties such as a cholesterol moiety, cholic acid, thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene, glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

The above-described agents can be provided to the subject per se, or as part of a pharmaceutical composition where they are mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethyleneglycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal direct intraventricular, intravenous, intraperitoneal, intranasal, or intraocular injections. Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is upon the route of administration chosen.

For injection, the active ingredient of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize, starch, wheat starch, rice starch, potato starch, gelatin, gum tragacanth, methyl cellulose, hydroxypropylmethylcellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorofluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in in compatible pharmaceutical carrier may also be prepared, placed in an appropriate container, and labeled for treatment of an indicated condition.

Pharmaceutical compositions of the present invention may, if desired, be presented in a pack or dispenser device, such as FDA approved kit, with a contain one or more unit dosage forms containing the active ingredient. The pack may, for example, comprise metal or plastic foil, such as a blister pack. The pack or dispenser may also be accommodated by a notice associated with the container in a dispenser may also be accommodated by a notice associated with the container in a form prescribed by a governmental agency regulating the manufacture use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

It will be appreciated that treatment of a disease according to the present invention may be combined with other prior art treatment methods, also known as combination therapy.

As mentioned hereinabove, the splice variants of the present invention may also have diagnostic value. For example, the present inventors uncovered soluble extracellular isoforms of follicular stimulating hormone receptor (FSHR, GenBank Accession: FSHR_human) and lutheizing hormone receptor [LSHR_human, see Table 3 below), each of which can serve as a diagnostic marker for fertility and menopausal disorders.

Thus, the present invention envisages diagnosing in a subject predisposition to, or presence of a disease which depends on expression and/or activity of a biomolecular sequence of the present invention for its onset or progression or is associated with abnormal activity or expression of a biomolecular sequence of the present invention.

As used herein the term “diagnosing” refers to classifying a disease or a symptom, determining a severity of the disease, monitoring disease progression, forecasting an outcome of a disease and/or prospects of recovery.

Diagnosis of a disease according to the present invention can be effected by determining a level of a polynucleotide or a polypeptide of the present invention in a biological sample obtained from the subject, wherein the level determined can be correlated with predisposition to, or presence or absence of the disease.

As used herein, the term “level” refers to expression-levels of RNA and/or protein or to DNA copy number of a splice variant of the present invention. Typically the level of the splice variant in a biological sample obtained from the subject is different (i.e., increased or decreased) from the level of the same variant in a similar sample obtained from a healthy individual.

As used herein “a biological sample” refers to a sample or fluid isolated from a subject, including but not limited to, for example, plasma, serum, spinal fluid, lymph fluid, the external sections of the skin, respiratory, intestinal, and genitourinary tracts, tears, saliva, mil, blood cells, tumors, neuronal tissue, organs, and also samples of in vivo cell culture constituents.

Numerous well known tissue or fluid collection methods can be utilized to collect the biological sample from the subject in order to determine the level of DNA, RNA and/or polypeptide of the variant of interest in the subject.

Examples include, but are not limited to, fine needle biopsy needle biopsy, core needle biopsy and surgical biopsy (e.g., brain biopsy).

Regardless of the procedure employed, once a biopsy is obtained the level of the variant can be determined and a diagnosis can thus be made.

Determining the level of the same variant normal tissues of the same origin is preferably effected along-side to detect an elevated expression and/or amplification.

Typically, detection of a nucleic acid of interest in a biological sample is effected by hybridization-based assays using an oligonucleotide probe.

Hybridization based assays which allow the detection of a variant of interest (i.e., DNA or RNA) in a biological sample rely on the use of oligonucleotide which can be 10, 15, 20, or 30 to 100 nucleotides long preferably from 10 to 50, more preferably from 40 to 50 nucleotides.

Hybridization of short nucleic acids below 200 bp in length, e.g. 17-40 bp in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM; EDTA (pH 7.6), 0.5% SDS at 1-1.5° C. below the Tₘ; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 EDTA (pH 7.6), 0.5% SDS at 1-1.5° C. below the Tₘ, final wash solution of 6×SSC, and final wash at 22° C.; (ii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, FluorX (Amersham) and others [e.g., Kricka et al. (1992), Academic Press San Diego, Calif] can be attached to the oligonucleotides.

Traditional hybridization assays include PCR, RT-PCR, Real-time PCR, RNase protection, in-situ hybridization, primer extension, Southern blot, Northern Blot and dot blot analysis.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

It will be appreciated that antisense oligonucleotides may be employed to quantify expression of a “splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity [see Sazani and Kole (2003), supra].

Polymerase chain reaction (PCR)-based methods may be used to identify the presence of an mRNA off interest. For PCR-based methods a pair of oligonucleotides is used, which is specifically hybridizable with the polynucleotide sequences described hereinabove in an opposite orientation so as to direct exponential amplification of a portion thereof (including the herein above described sequence alteration) in a nucleic acid amplification reaction. Examples, of oligonucleotide pair of primers which can be used to detect variants of the present invention are listed in Table 2, below.

The polymerase chain reaction and other nucleic acid amplification reactions are well known in the art and require no further description herein. The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Hybridization to oligonucleotide arrays may be also used to determine expression of variants of the present invention. Such screening has been undertaken in the BRCA1 gene and in the protease gene of HIV-1 virus [see Hacia et al., (1996) Nat Genet 1996; 14(4):441-447; Shoemaker et al., (1996) Nat Genet 1996; 14(4):450-456; Kozal et al., (1996) Nat Med 1996; 2(7):753-759].

The nucleic acid sample which includes the candidate region to be analyzed is isolated, amplified and labeled with a reporter group. This reporter group can be a fluorescent group such as phycoerythrin. The labeled nucleic acid is then incubated with the probes immobilized on the chip using a fluidics station. For example, Manz et al. (1993) Adv in Chromatogr 1993; 33:1-66 describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for diseases both rapidly and easily.

The presence of the variant of interest may also be detected at the protein level. Numerous protein detection assays are known in the art, examples include, but are not limited to, chromatography, electrophoresis, immunodetection assays such as ELISA and western blot analysis, immunohistochemistry and the like, which may be effected using antibodies specific to the variants of the present invention.

Preferably used are antibodies, which specifically interact with the polypeptide variants of the present invention and not with wild type.

The diagnostic reagents described hereinabove can be included in diagnostic kits. For example a kit for diagnosing a fertility disorder in a subject can include the set of oligonucleotide primers set forth in SEQ ID NOs: 9 and 10 in a container and as second container with appropriate buffers and preservatives for executing a PCR reaction.

Diagnostics using the above-described methodology can be validated using other diagnostic methods which are well known in the art such as by imaging, molecular detection of known markers and the like.

Apart of clinical applications, the biomolecular sequences of the present invention can find other commercial uses such as in the food, agricultural electromechanical, optical and cosmetic, D industries [http://.physics.unc.edu/˜rsuper/XYZweb/XYZchipbiomotors.rs1.doc; http://www.bio.org/er/industrial.asp]. For example, newly uncovered gene products, which can disintegrate connective tissues, can be used as potent anti scarring agents for cosmetic purposes. For example, newly uncovered gene products, which can disintegrate connective tissues, can be used as potent anti scanning agents for cosmetic purposes. Non-limiting examples of such gene products include the matrix metalloproteinase family of proteins (MMP), which are a group of proteases having varying specificities for ECM components as substrates, non-limiting examples of which have the gene symbols “CLG” and “CGL4B” in, the attached files. These proteins are involved in ECM break-down as part of the wound healing process, for example for cell migration. The activity of these proteins is also modulated by specific tissue inhibitors of MMPs (TIMP) and other factors in the microenvironment in and around the wound area. Therefore, one possible optionally application for the present invention would be the selection of appropriate antisense oligonucleotides for either one or more MMPs and/or for factors related to TIMPs, in order to modulate wound healing activities (and/or as previously noted, for treatment of arthritis).

As another optional treatment, production of collagen may be optionally modulated through the use of appropriate antisense oligonucleotides. Collagen is an important connective tissue element, but is also involved in pathological conditions such as fibrosis and the formation of adhesions between tissues of different organs, a condition which may occur for example after surgery. Therefore, modulation of collagen production, for example to reduce collagen production, may optionally be performed according to the present invention.

Other applications include, but are not limited to, the making of gels, emulsions, foams and various specific products, including photographic films, tissue replacers and adhesives, food and animal feed, detergents, textiles, paper and pulp, and chemicals manufacturing (commodity and fine, e.g., bioplastics).

Research applications include, for example, differential cloning, detection of rearrangements in DNA sequences as disclosed in U.S. Pat. No. 5,994,320, drug discovery and the like.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques a thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, (Cold Spring Harbor Laboratory Press, New York (1998); methodologies as set forth in U.S. Pat. Nos. 4,666,828; 4,683,202; 4,801,531; 5,192,659 and 5,272,057; “Cell-Biology: A Laboratory Handbook”; Volumes I-III Cellis; J. E., ed. (1994); “Current Protocols in Immunology” Volumes I-III Coligan J. E., ed., (1994); Stites et al. (eds), “Basic & and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,8671,517; 3,879,262; 3,901,654; 3,935,074; 3,984,533, 3,996,345; 4,034,074; 4,098,876; 4,879,219; 5,011,771 and 5,281,521; “Oligonucleotide Synthesis” Gat, M. J., ed. (1984); “Nucleic Acid Hybridization” Hames; B. D., and Higgins S. J., eds. (1985); “Transcription and Translation” Hames, B. D., and Higgins S. J., Eds. (1984); “Animal Cell Culture”. Freshney, R. I., ed.; (1986); “Immobilized Cells and Enzymes” IRL Press, (1986); “A Practical Guide to Molecular Cloning” Perbal, B., (1984) and “Methods in Enzymology” Vol. 1-3177, Academic Press; “PCR Protocols: A Guide To Methods and Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporate by reference.

### Example 1

### Computational Identification of Alternative Splicing without Usage of Expressed Sequence Data and “Alternativeness Score”

Background

Alternative splicing is a mechanism by which multiple gene products are generated from a single gene. Currently, the only way for large-scale computational detection of alternative splicing is by Expressed Sequence Tags (ESTs) analysis, and microarray technology.

While reducing the present invention to practice, the present inventors designed a new approach for computational identification of splice variants without needing expressed sequence data. The present inventors have first uncovered that alternatively spliced exons have unique characteristics differentiating them from constitutively spliced ones. Using machine-learning techniques, a combination of these characteristics was found to identify alternatively spliced exons with very high probability.

Experimental Procedures

Compiling the training sets of conserved alternative and constitutive exons—Human and ESTs and cDNAs were obtained from NCBI GenBank version 131 (August 2002) (www.ncbi.nlm.nih.gov/dbEST) and aligned to the human genome build 30 (August 2002) (www.ncbi.nlm.nih.gov/genome/guide/human) using the LEADS clustering and assembly system as described in Sorek et al. (2002) Genome Res. 12:1060-1067. Briefly, the software cleans expressed sequences from repeats, vector contaminations and immunoglobulins. It then aligns expressed sequences to the genome taking alternative splicing into account, and clusters overlapping expressed sequences into “clusters” that represent genes or partial genes.

Alternatively spliced internal exons and constitutively spliced internal exons were identified using the same methods described in Sorek et al. (2002). In brief, these methods screen for reliable exons requiring canonical splice sites and discarding possible genomic contamination events. A constitutively spliced internal exon was defined as an internal exon supported by at least 4 sequences, for which no alternative splicing was observed. An alternatively spliced internal exon was defined as such if there was at least one sequence that contained both the internal exon and the 2 flanking exons (exon inclusion), and one sequence that contained the two flanking exons but skipped the middle one (exon skipping).

Mouse ESTs and cDNAs from GenBank version 131 were aligned to the human genome build 30 as follows. Mouse ESTs and cDNAs were cleaned from terminal vector sequences, and low complexity stretches and repeats in the expressed sequences were masked. Sequences with internal vector contamination were discarded. Sequences identified as immunoglobulins or T-cell receptors were discarded. In the next stage, expressed sequences were heuristically compared to the genome to find likely high-quality hits. They were then aligned to the genome using a spliced alignment model that allows long gaps. Single hits of mouse expressed sequences to the human genome shorter than 20 bases, or having less than 75% identity to the human genome, were discarded. Using these parameters, 1,341,274 mouse ESTs were mapped to the human genome, 511,381 of them having all their introns obeying the GT/AG or GC/AG rules.

To determine if the borders of a human intron (which define the borders of the flanking exons) were conserved in mouse, a mouse EST spanning the same intron-borders while aligned to the human genome was required (with alignment of at least 25 bp on each side of the exon-exon junction). In addition, this mouse EST was required to span an intron (i.e., open a long gap) at the same position along the EST while aligned to the mouse genome.

Alignment of intronic regions was done using sim4 (Florea (1998) Nat. Rev. Genet. 3:285-298]. An alignment was considered significant according to sim4 default parameters, i.e., at least one word of 10 consecutive identical nucleotides. Lengths of alignments and identity levels were parse from sim4 standard output. For per-position conservation calculation, the GCG GAP program was run of the 100 intronic nucleotides from each side of the exon, and the alignments were achieved.

Compilation of dataset of 110,932 human exons with mouse orthologues—Human and ESTs and cDNAs were obtained from NCBI GenBank version 136 (2003) (www.ncbi.nlm.nih.gov/dbEST) and were mapped to the human genome April 2003 assembly (www.ncbi.nlm.nih.gov/genome/guide/human) using the spliced alignment module of LEADS. For each expressed sequence, all mappings of internal exons on the human genome were retrieved. Only exons flanked by AG/GT or AG/GC splice sites were allowed 185,799 human exons mapped to the human genome were thus retrieved.

To find the mouse orthologue for each human exon, mouse expressed sequences from GenBank version 136 were first aligned to the human genome, as described above. Mouse sequences exactly spanning human exons were aligned to the mouse genome as well, and the corresponding sequence on the mouse genome was declared as the orthologous mouse exon, if AG/GT or AG/GC legal splice sates flanked it.

Human exons for which no spanning mouse expressed sequence was detected were aligned directly to the mouse genome using the LEADS “cluster” module. Hits spanning the full length of the exon, that were flanked by AG/GT or AG/GC legal splice sites, were declared as the orthologous mouse exons.

Altogether, these searches retrieved 110,932 pairs of exons in the human and mouse genomes. For each such exon, all classifying, parameters were calculated as follows. Conservation between exons was calculated from aligning the human exon to the mouse exon using the sim4 alignment program. Conservation in the flanking intronic sequences was calculated as described above (in the “Compiling the training sets.” section of the methods). Exon size and dividability by 3 were retrieved from the exon sequence itself. Score was calculated for each exon as described in the results section.

Results

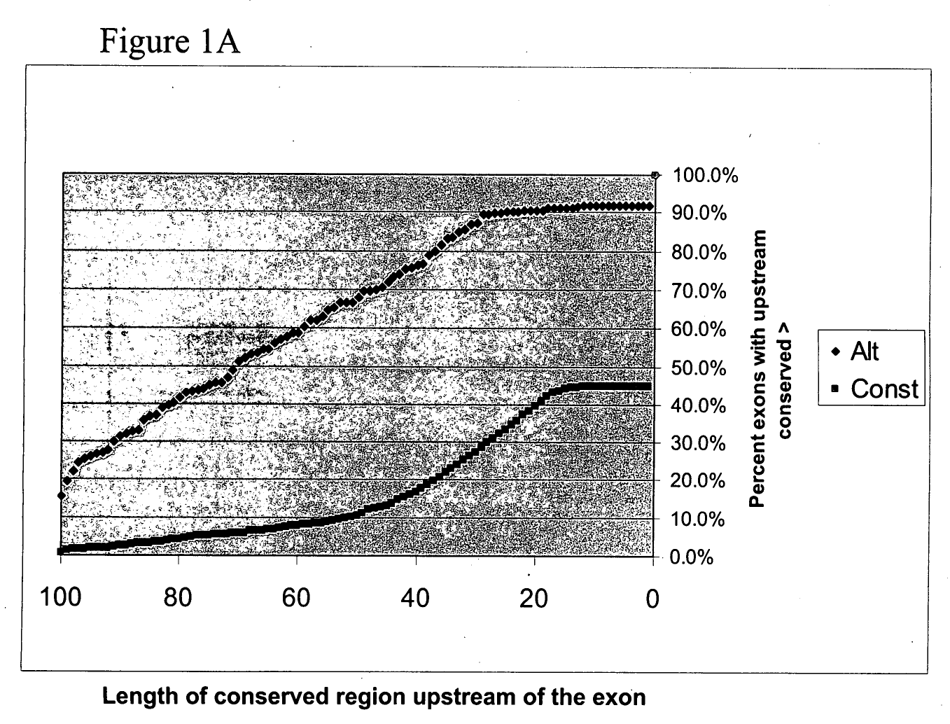

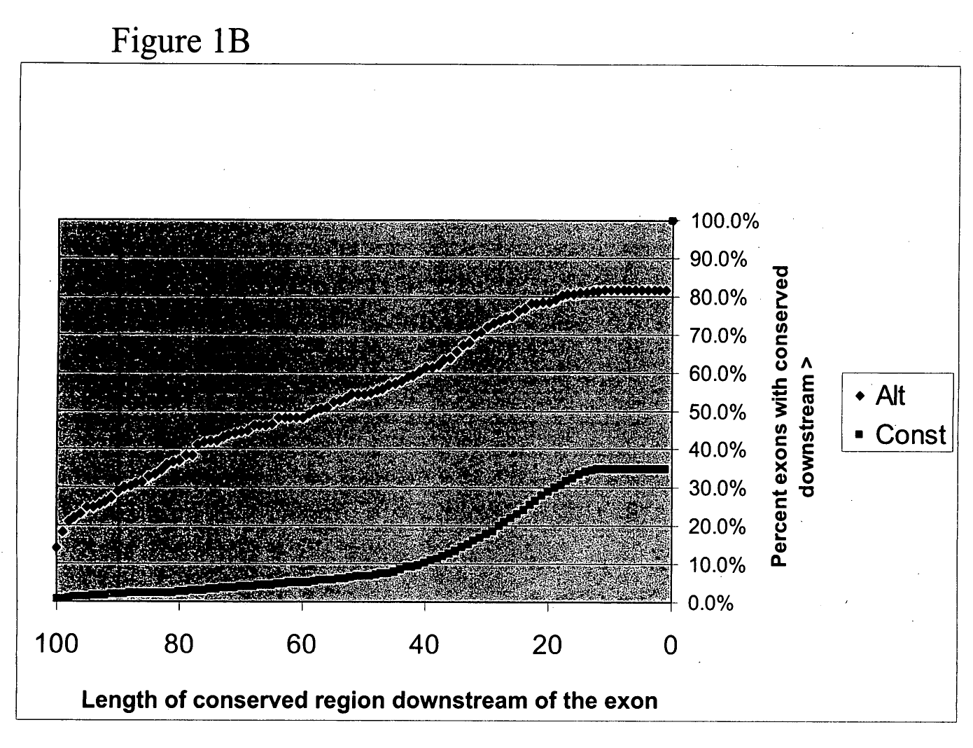

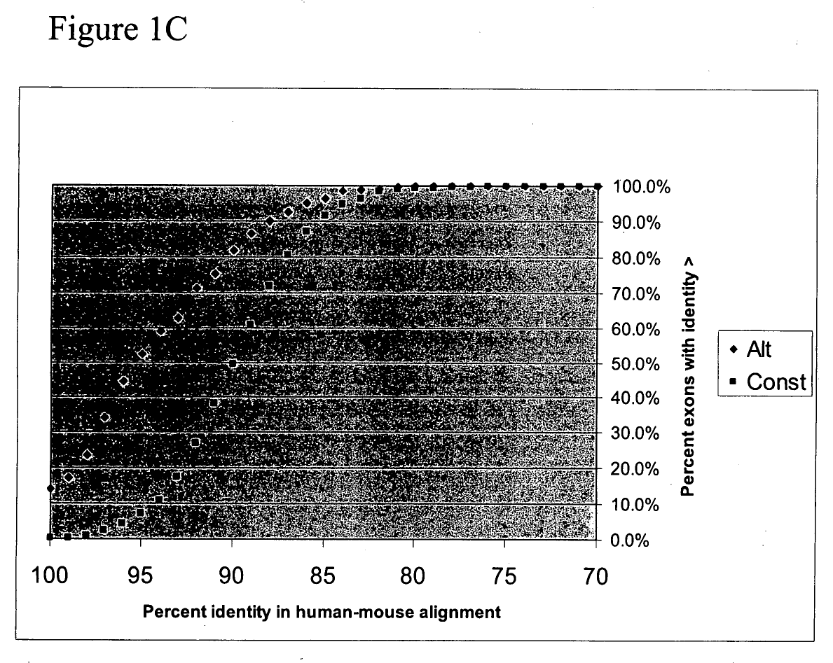

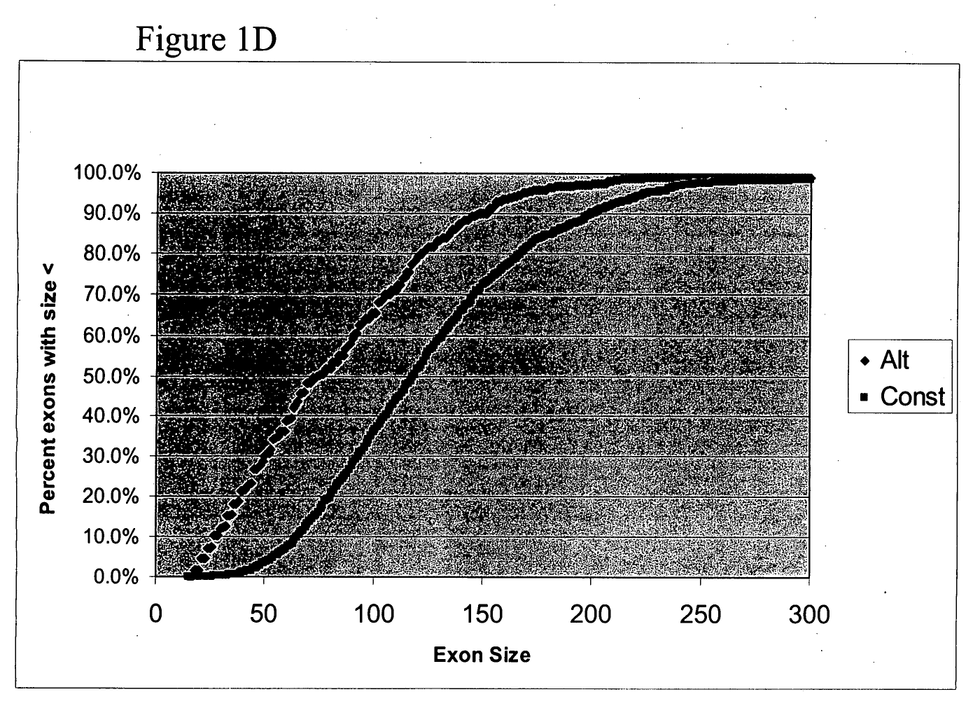

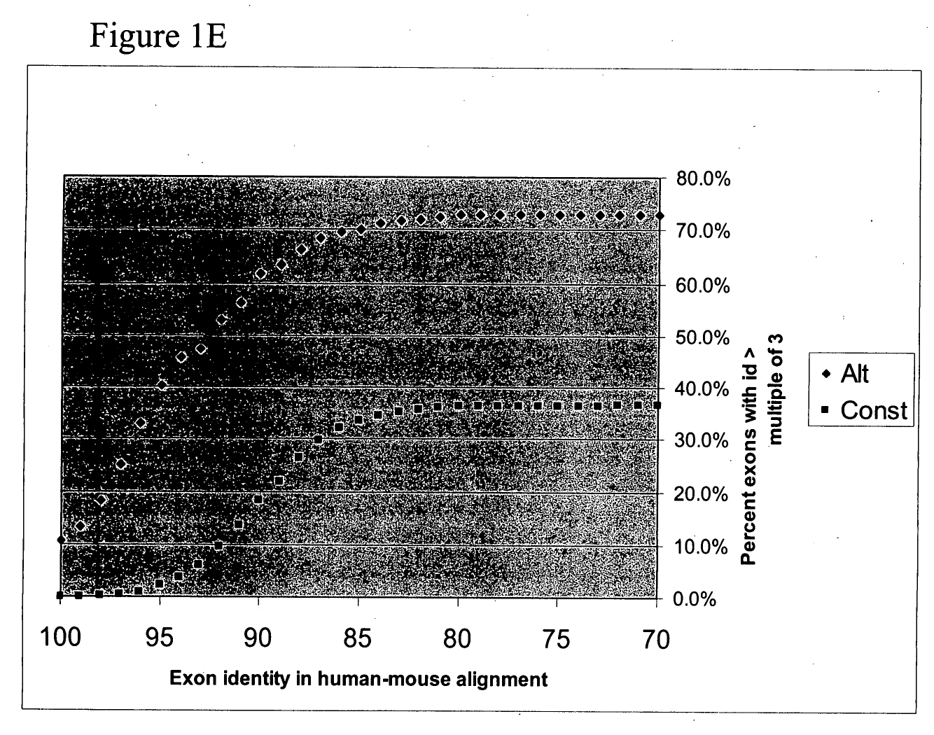

The present inventors have previously compiled sets of alternatively spliced (cassette) and constitutively spliced exons that are conserved between human and mouse [Sorek (2003) Genome Res. 13:1631-1637]. Interestingly, alternatively spliced exons were found to be frequently flanked by intronic sequences conserved between human and mouse, but constitutively spliced exons were not [Sorek (2003) supra and FIGS. 1a-b, as described below and in Table 1]. Such conserved intronic sequences are probably involved in the regulation of alternative splicing.

The training sets of exons used herein initially contained 243 alternative exons and 1966 constitutive exons. These sets were based on EST analyses of GenBank 131, where the constitutive exons were defined as such if there were at least 4 expressed sequences supporting them, and no EST skipping them, both in human and in mouse. For the present analysis constitutive exons for which an evidence for alternative splicing appeared in the newer version of GenBank, 136 were eliminated to provide a training set of 1753 constitutive exons.

Further features that distinct alternatively spliced exons from constitutively spliced exons were then sought. FIGS. 1a-e show structural differences between alternatively spliced exons and constitutively spliced exons. FIG. 1a shows high level of sequence conservation in the last 100 nucleotides of introns flanking alternative exons but not constitutive exons. A conserved sequence region refers to length of alignment between human and mouse DNA in that region. Similar conservation was seen in the first 100 nucleotides of downstream introns flanking alternative exons (FIG. 1b). Furthermore, alternatively spliced exons exhibited much higher level of human-mouse sequence conservation (i.e., 50% of exons showed more than 95% identity) than constitutively spliced exons (i.e., 50% of constitutively spliced exons showed 90% identity, see FIG. 1c). The size of alternative splices exons was found to be shorter than that of constitutive exons (FIG. 1d). Essentially, the average length of alternative exon (i.e., 50% of the exon data set) was about 75, while the average length of constitutive exons was almost twice as much. Finally, highly conserved exons which are divisible by 3 where much more frequent in the alternative exon dataset than in the constitutive exon dataset (FIG. 1e). Table l below, summarizes the major classifying features which were found.

TABLE 1
Features differentiating between alternatively spliced exons and
constitutively spliced exons

| | Alternatively | Constitutively | |
|---|---|---|---|
| | spliced exons | spliced exons | P value^{a} |
| Average size | 87 | 128 | p < 10⁻¹⁶ |
| Percent exons that are a multiple of 3 | 73% | 37% | p < 10⁻⁹ |
| | (177/243) | (642/1753) | |
| Average human-mouse exon conservation | 94% | 89% | p < 10⁻³⁶ |
| Percent exons with upstream intronic | 92% | 45% | p < 10⁻¹¹ |
| elements conserved in mouse^{b} | (223/243) | (788/1753) | |
| Percent exons with downstream intronic | 82% | 35% | p < 10⁻¹⁴ |
| elements conserved in mouse^{b} | (199/243) | (611/1753) | |
| Percent exons with both upstream and | 77% | 17% | p < 10⁻³⁷ |
| downstream intronic elements conserved in | (188/243) | (292/1753) | |
| mouse^{b} | | | |

^{a}P value was calculated using Fisher's exact test, except for the “average size” and “average human-mouse exon conservation”, for which p value was calculated using student's T test.
^{b}Conservation was detected in the 100 intronic nucleotides immediately upstream or downstream the exon using local alignment with the mouse 100 counterpart intronic nucleotides. A minimum hit was 12 consecutive perfectly matching nucleotides.

In short, conserved alternatively spliced exons are much shorter than constitutively spliced ones, their size tends to be a multiple of 3, and they share higher identity level with their mouse counterpart exon (FIGS. 1c-e). These differences probably stem from the unique function of the alternative exons: Since these exons are cassette exons that are sometimes inserted and sometimes skipped, they should be dividable by 3 such that the reading frame is kept when skipped. This constraint does not apply to constitutively spliced exons. The higher identity level between human and mouse could be explained by the fact that alternatively spliced exons frequently contain sequences that regulate their splicing [exonic splicing enhancers and silencers, reviewed by Cartegni (2002). Nat. Rev. Genet. 3:285-298]. These regulatory sequences add another level of conservation constraint on the exon sequence. The fact that alternatively spliced exons are smaller than constitutively spliced ones was previously reported [Thanaraj (2003) Prog. Mol. Subcell. Biol. 31:1-31] and may be attributed to the fact that the spliceosome sub-optimally recognizes smaller exons [Berget (1995) J. Biol. Chem. 270(6):2411-4].

The above-described sequence features can be used to identify alternatively spliced exons in the human and the mouse genomes. However, each feature by itself is not strong enough to classify an exon. Therefore a combination of features that would exclusively “define” alternative exons was determined by complete iteration on the above-described training sets of alternative and constitutive exons. The classifying parameters that were iterated over were the following: Exon length, dividable/not dividable by 3, percent identity when aligned to the mouse counterpart, length of conserved intronic sequence in the 100 bases immediately upstream the exon, identity level in the conserved upstream intronic sequence stretch, length of conserved intronic sequence in the 100 bases immediately downstream the exon, and identity level in the downstream conserved intronic sequence stretch. The output was a set of rules, from which a specific combination that would supply maximum specificity for identifying alternatively spliced exons was searched.

The best combination from this iteration was the following: At least 95% identity with the mouse exon counterpart; exon size is a multiple of 3; at least 15 conserved intronic nucleotides out of the first 100 nucleotides downstream the exon; and at least 12 conserved intronic nucleotides upstream the exon with at least 85% identity. 76 exons, or 31% of the training set of 243 alternatively spliced exons, exhibited this combination of features. However, none of the exons from the set of 1753 constitutively spliced exons matched these features.

The above combination of parameters can therefore be used to identify alternatively spliced exons with very high specificity and ˜30% sensitivity.

To test this 110,932 human exons were collected, for which a mouse counterpart could be identified (see methods). For each of these exons, all classifying parameters were calculated.

Out of the 110,932 human exons, 1,030, or ˜1%, were found to comply with the above-mentioned combination of parameters. To check if these exons are indeed alternatively spliced, human expressed sequences (ESTs or cDNAs) that skip the exons but contain the two exons flanking it were searched. For 518 (50%) of the candidate alternative exons there was such skipping evidence. For comparison, only 7% out of the entire set of 110,932 human exons had similar skipping EST evidence. This means that the combination of parameters, which were chosen indeed caused alternatively spliced exons to be retrieved.

The remaining 512 candidate alternative exons were manually examined using the UCSC genome browser (April 2003), and found that for 195 additional exons there was a human expressed sequence showing patterns of alternative splicing other than exon skipping (e.g., intron retention, alternative donor/acceptor, mutually exclusive exons). Thus, 707 (69%) of the candidate alternative exons identified by the above-described methodology were supported by independent evidence for alternative splicing deriving from dbEST and RefSeq.

But what about the remaining 317 (311%) of the candidate exons? These can still be alternatively spliced exons for which not enough ESTs exist, so that a skipping variant has not appeared in dbEST yet. Indeed, while on average there were 32 supporting expressed sequences per exon in the general set of 110,932 exons (median 10), the support for the 317 candidate alternatives was much smaller, averaging in 14 sequences (median 7).

The method of identifying cassette exons without using ESTs, as described herein, allows estimation of the absolute number of alternatively spliced exons in the human genome. The above-described results show that the combination of characteristics presented herein identifies 31% of the cassettes exons in the training set. This combination retrieved 1,030 (1%) out of the 110,932 exons tested. It can thus be concluded that 1%/0.31, or ˜3% of all human exons, are alternatively spliced in an exon skipping manner. Moreover, the exons in the initial training set of 243 cassette exons were all alternatively spliced in a pattern of exon skipping so that the present method would retrieve main sipped exons. Exon skipping is known to comprise only about 50% of all types of alternative splicing, with other types, such as alternative donor/acceptor, mutually exclusive exons, and intron retention comprise the remaining 50%. Therefore it is estimated that up to 2-3% (i.e., 6%) of all human exons, are alternatively spliced. As the human genome contains ˜210,000 exons [Lander (2001) Nature 409:860-921], 6% or ˜12,000 exons, are alternatively spliced.

Understanding this it is now possible to devise an “alternativeness score” that reports on the probability that a given exon is alternatively spliced. The characterizing features are characterized for a given exon (length of conserved introns upstream and downstream, exon length, conservation with mouse counterpart exon, and dividability by 3). Then, the fraction of alternative exons from the training set of 243 alternative exons (let X be this number) that answers to this combination of parameters is calculated (have intronic conservation greater or equal to its intronic conservation; have length lesser or equal to its length; has exon conservation greater or equal to its exon conservation; and divides/not divides by 3 as the tested exon). Similarly, the fraction of constitutive exons is calculated from the set of 1753 that answers to this combination of parameters (let Y be this number). Then the fraction of alternative exons is multiplied by 12,000 (the actual number of alternatives in the human genome), and the fraction of constitutive exons by 200,000 (the actual number of constitutive exons in the human genome). The sum of the resulting numbers is the actual number of exons that have this combination of parameters that are expected to be found in the human genome. The “alternativeness score” is the number of predicted alternative exons divided by the above-described sum.

Presenting this mathematically, the “alternativeness score” (denoted as “A”) is:
A=(X*12,000)/(X*12,000+Y*200,000)

As an example the following parameters are used:

Size 123 bp

Divided by 3

Length of upstream conserved region: 73 bp

Length of downstream conserved region: 100 bp

Human-Mouse exon conservation: 96%

13 out of 243 (X=5.3%) alternative exons have these features, while 1/1753 (0.05%) constitutive exons have these features. 5.3%×12,000=636 and 0.05%×200,000=100.

Therefore, the alternativeness score A is: A=636/(636+100)=86%.

Using this alternativeness scoring, 4042 exons in the human genome exhibited a score of 100%, 749 additional exons exhibited a score between 90% to 100% and 2032 exons exhibited a score between 80% to 90%.

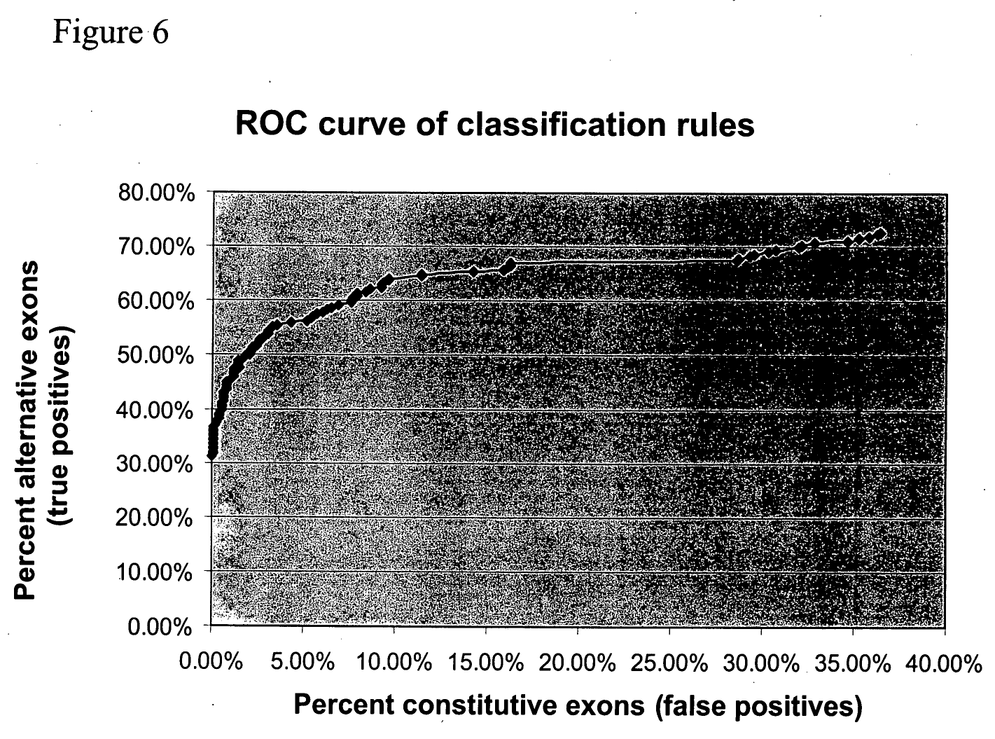

The classification rule that was chosen for the experimental verification retrieves alternatively spliced exons with a very high specificity (less than 0.3% false positive rate) but at the price of a relatively low sensitivity (32%). Other rules can be chosen in which sensitivity is higher, but naturally this would increase the false positive rate of the prediction. FIG. 6 presents a sensitivity versus false positive rate plot (ROC curve) for different rules selecting for increasing number of alternative exons from our test set of 243 exons. As shown in the figure, it is possible to employ a rule that would identify up to 73% of the alternative exons, but this rule would also retrieve 36% of the constitutively spliced exons (the upper limit of 73% is due to the Boolean nature of the “divisibility by 3” feature). Note, that since most of the exons in the human genome are constitutive, such a rule would have low predictability for exon skipping: Assuming, for example, that ˜10%, or 20,000 out of the ˜200,000 predicted exons in the human genome, are alternative, the probability that an exon identified by, the 73%:36% rule would really be alternative is only 18% (0.73*20,000/[0.73*20,000+0.36*180,000]). Therefore, preferably a rule is selected with close to zero false positives. The curve in FIG. 6 presents a variety of alternatives, and allows the selection of a % rule for a desired target specificity or sensitivity. For example, 50% sensitivity is achievable at about 1.8% false positive rate.

### Example 2

### Experimental Evidence for Putative Alternative Exons Uncovered Using the Methodology of the Present Invention

Biological relevance of computationally identified alternative exons in the absence of EST data support was determine according to RT-PCR results.

Experimental Procedures

RT-PCR—RT was done on total RNA samples. RT-PCR reactions were effected using random hexamer primer mix (Invitrogen) and Superscript II Reverse transcriptase (Invitrogen). Conditions used were as follows: denaturation at 70° C. (5 min), annealing on ice, RT at 37° C. (1 hour). “Hot-Star” Taq polymerase (Qiagen) was used in all reaction samples. Some reactions required addition of Q solution (Qiagen) to enhance the reaction. Reaction composition included: total volume of 25 μl, Taq Buffer ×10—2.5 μl, DNTPs (mix of 4) ×12.5—2 μl, Primers—0.5 μl of each (total 1 μl), cDNA—1 μl (1-2 ng/μl), Taq Enzyme—0.5 μl, Q solution (when needed) ×5—5 μl, H₂O was added to complete a final volume of 25 μl.

Primers are listed in Table 2, below.

TABLE 2

| | | | Predicted | Predicted |
|---|---|---|---|---|
| | | | product | product size |
| | Forward primer/ | Reverse Primer/ | size | of novel |
| Gene | SEQ ID NO: | SEQ ID NO: | (bp) | variant |
| EFNA | ACCGGCCTCACTCTCCAAA | TGGCTCGGCTGACTC | 287 | 206 |
| | TGG/1 | ATGTACGG/2 | | |
| EPHB1 | AAGCTCCAGCATTACAGC | ACCCTCCAGGCGAAT | 324 | 201 |
| | ACAGGCC/3 | GATGTTAGG/4 | | |
| FGF11 | CCAAGGTGCGACTGTGCG | GGTAGAGAGCAGAG | 344 | 233 |
| | G/5 | GCGTACAGGACG/6 | | |
| VLDLR | TGAGCCCCTGAAAGAGTG | TCTAAGCCAATCTTC | 324 | 198 |
| | TCATATAAACG/7 | CTGATGTCTCTTCG/8 | | |
| FSHR | CCTGCTCTACATCAACCCT | CCATAGCTAGGCAGG | 394 | skipping |
| | GAGGCC/9 | GAATGGATCC/10 | | 7: 325; |
| | | | | skipping |
| | | | | 8: 319; |
| | | | | skipping |
| | | | | 7&8: 250; |
| | | | | intron 7 |
| | | | | retention: |
| | | | | 505 |
| NOTCH2 | GAACACGGATGGCGCCTT | GGGGCAAAGTGTATC | 352 | 238 |
| | CC/11 | GATCACCCG/12 | | |
| NTRK2 | GGTCGGGAACATCTCTCGG | GCTCCCTTTTCAGAA | 400 | 211 |
| | TCTATGC/13 | CAATGTTATGTCGC/14 | | |
| PTPRZ1 | AAAAGATGCTGATGGGAT | TGCAGTCTGGAAGCA | 138 | 138 |
| | CCTGGC/15 | TTTCCTGCC/16 | | |
| VEGFC | CAGCACGAGCTACCTCAG | CACTGACAGGTCTCT | 351 | 199 |
| | CAAGACG/17 | TCATCCAGCTCC/18 | | |
| HPSE2 | TCACCTCGTGGACCAGAAT | ACTAAGGGCTGGCCA | 357 | 205 |
| | TTTAACCC/19 | TTCAGTTGC/20 | | |
| HGF | GGATCATCAGACACCACA | CGTGAGGATACTGAG | 302 | 183 |
| | CCGGC/21 | AATCCCAACGC/22 | | |

Reaction conditions were as follows: Activation of HotStar Taq—95° C. for 5 min; [denaturation—94° C. for 45 sec; annealing—Tm (specific for each set of primers)—4-5° C. for 45 sec; extension—72° C. for 1 min]×34 cycles]; Gap filling—72° C. for 10 min; storage—10° C. Forever.

Reaction products were separated on % a 2% agarose gel in TBE×5 at ˜150V. DNA was extracted from gel using a Qiaquick (Qiagen) kit, and DNA was sent out for direct sequencing using same primers.

Tissues and cell-lines—All samples were cDNA pools generated by RT-PCR. Sample 1. Cervix pool—included a pool of 3 cervix derived RNA samples. Samples were of mixed origin (tumor and normal). The cervix pool also included mRNA from HeLa cell-line (cervical cancer). Sample 2: Uterus pool—included a pool of 3 uterus derived RNA samples. Samples were of mixed origin (tumor and normal). Sample 3: Ovary pool—included a pool of 5 normal ovary derived RNA samples (Biochain www.biochain.com). The ovary pool was supplemented with two ovary samples of Mix origin (Tumor and Normal). Sample 4: Placenta—included one sample of Placenta derived RNA of a normal origin (Biochain). Sample 5: Breast Pool—included a pool of 3 breast derived RNA samples of mixed origin (i.e., 2 samples from a tumorous origin and one from a normal origin). Sample 6: Colon and intestine—included a pool of 5 colon derived RNA of mixed origin (tumor and normal). The pool was supplemented with one intestine (Normal) derived RNA sample. Sample 7: Pancreas—included one sample of normal pancreas derived RNA (Biochain). Sample 8: Liver and Spleen pool—included one sample of normal liver derived RNA (Biochain), one sample of normal spleen derived. RNA (Biochain) and one sample of HepG2 cell line (liver tumor) derived RNA. Sample 9: Brain pool—included a pool of normal brain derived RNA samples (Biochain). Sample 10: Prostate pool—included a pool of normal prostate derived RNA samples (Biochain). Sample 11: Testis pool—included a pool of normal testis derived RNA samples (Biochain). Sample 12: Kidney pool—included a pool of normal kidney derived RNA samples (Biochain). Sample 13: Thyroid pool—included a pool of normal thyroid derived RNA samples (Biochain—Normal). Sample 14: Assorted cell-line pool—included a pool of RNA samples from the following cell-lines: DLD, MiaPaCa, HT29, THP1, MCF7 (Obtained from the ATCC, USA).

Results

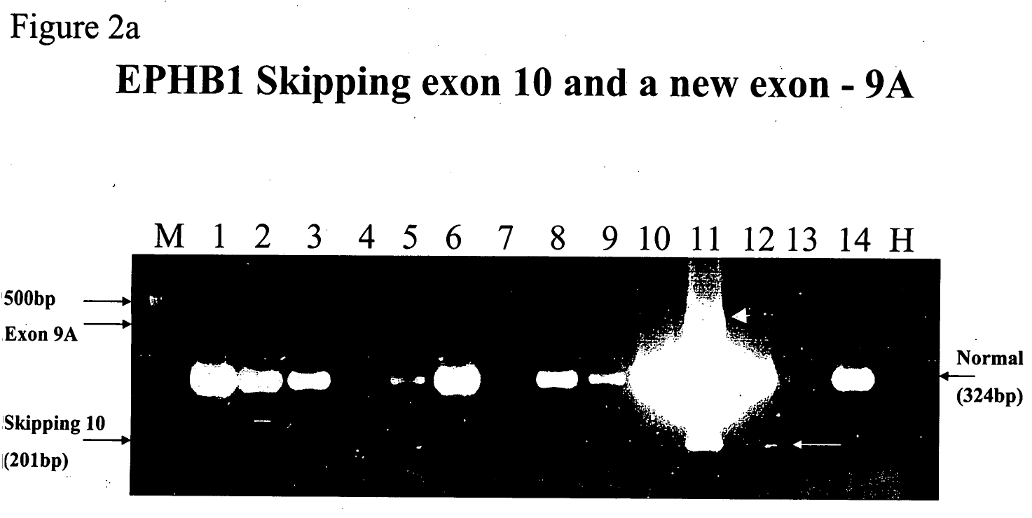

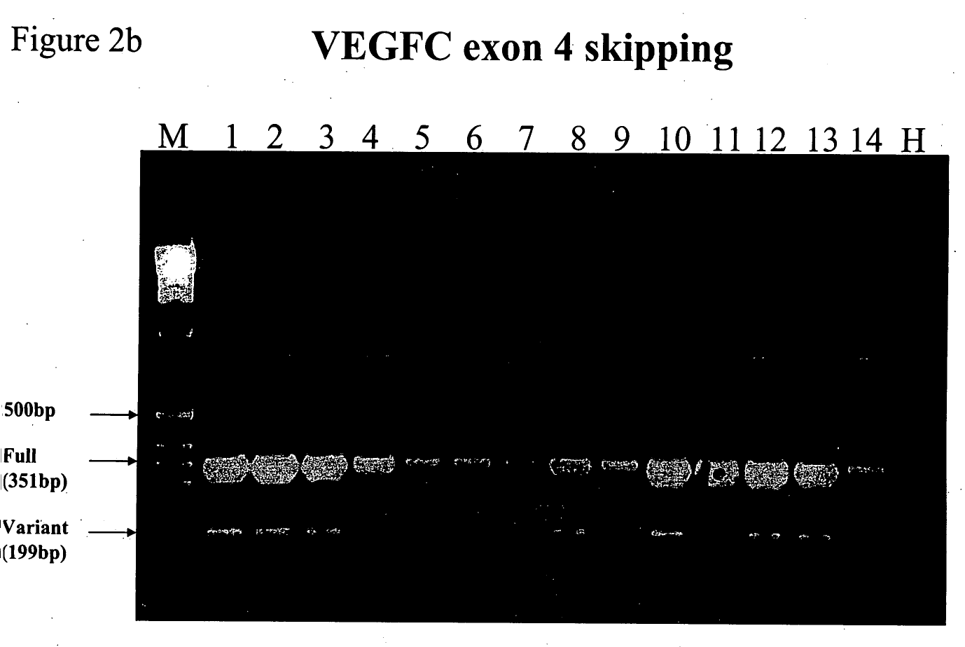

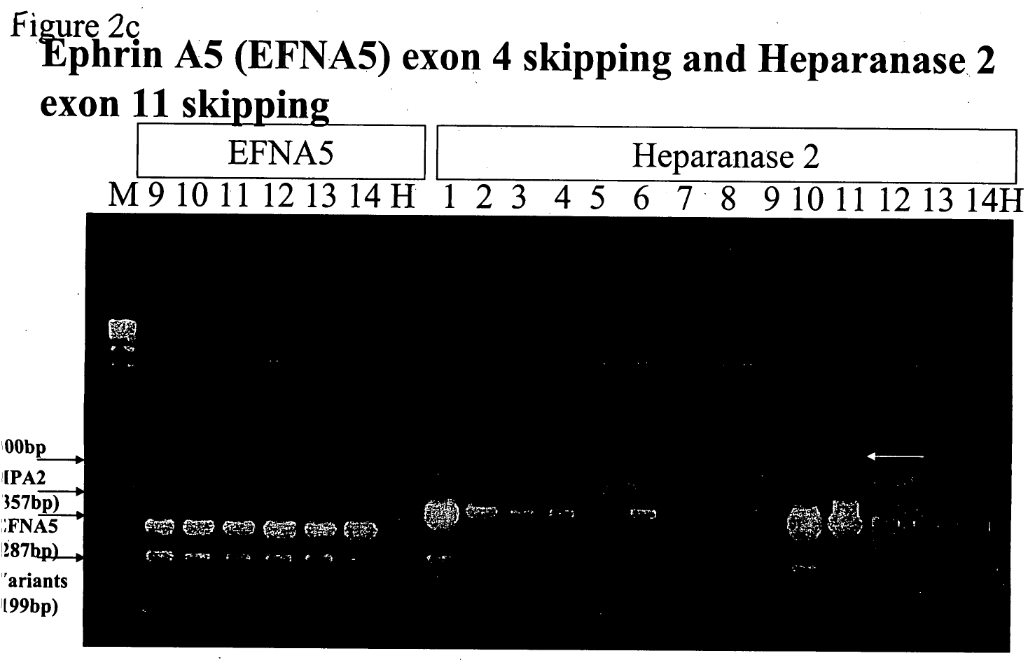

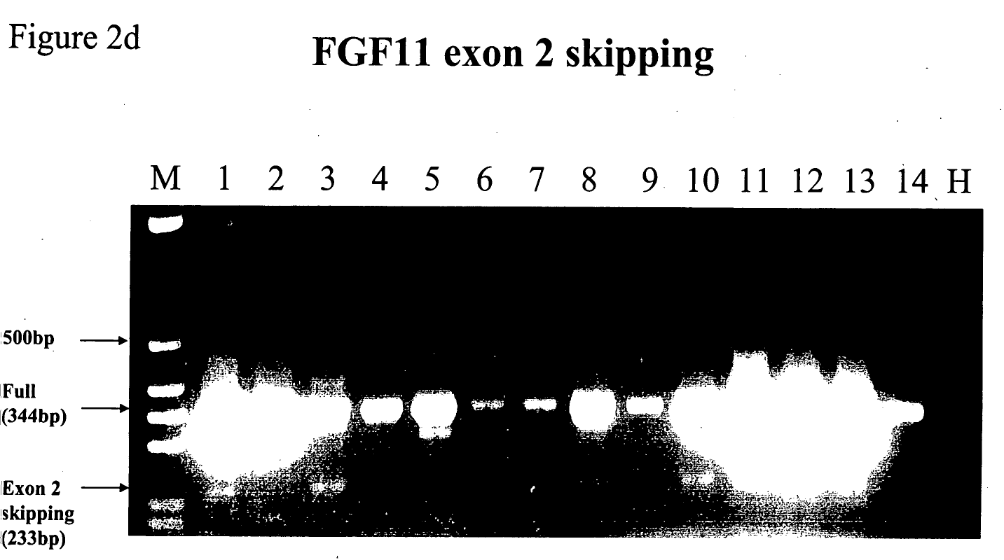

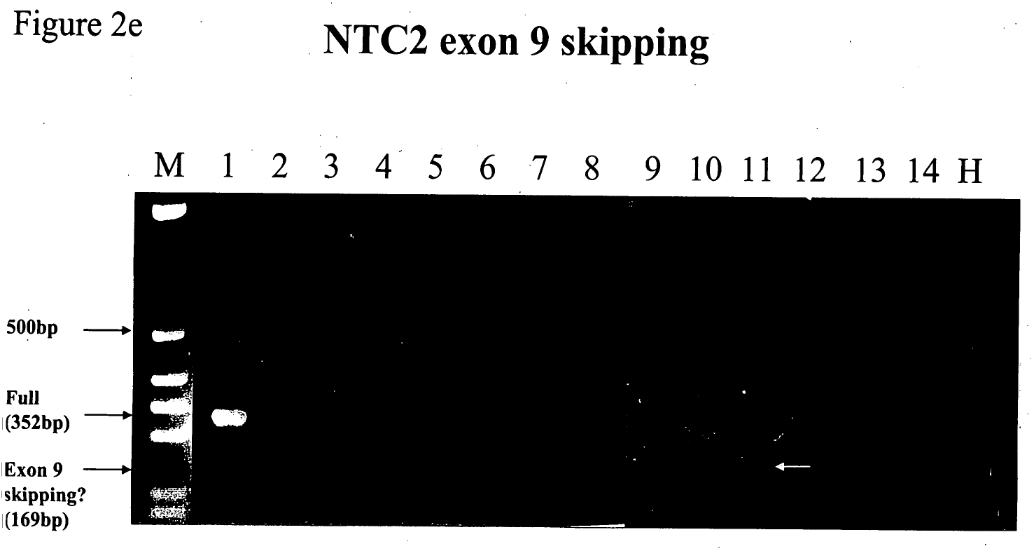

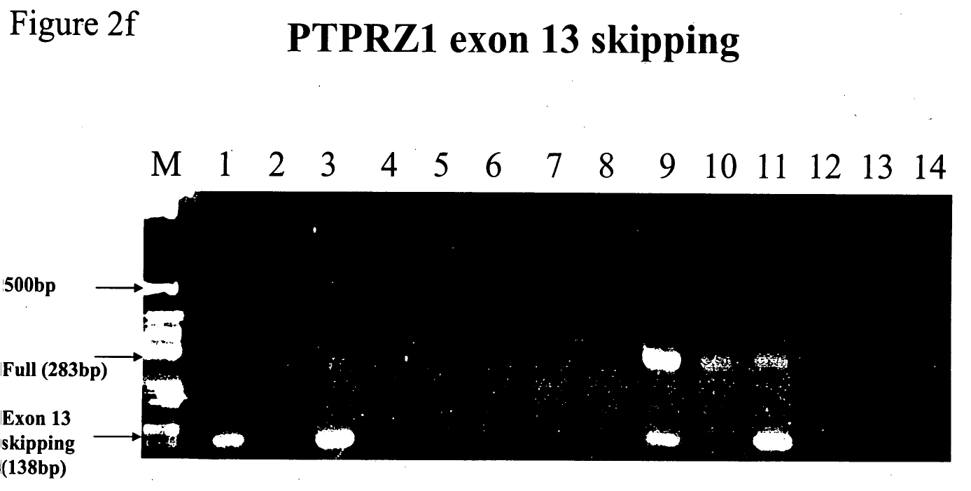

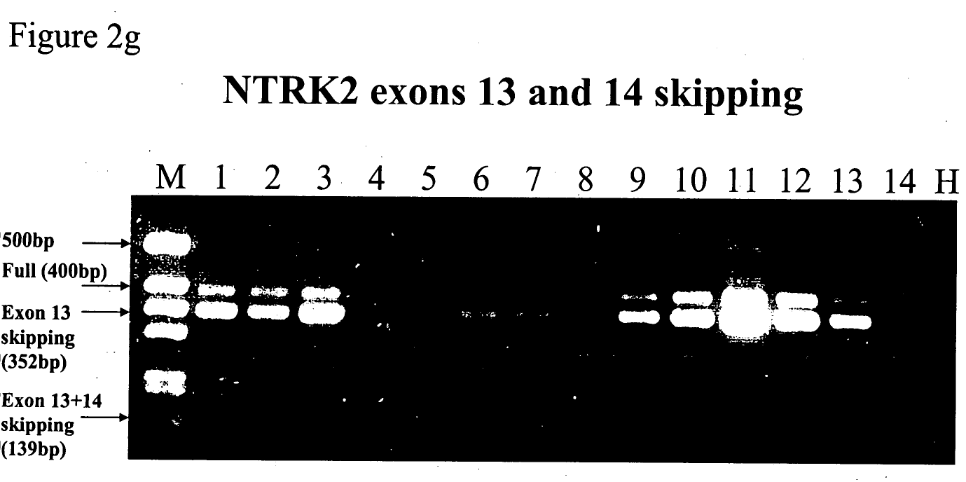

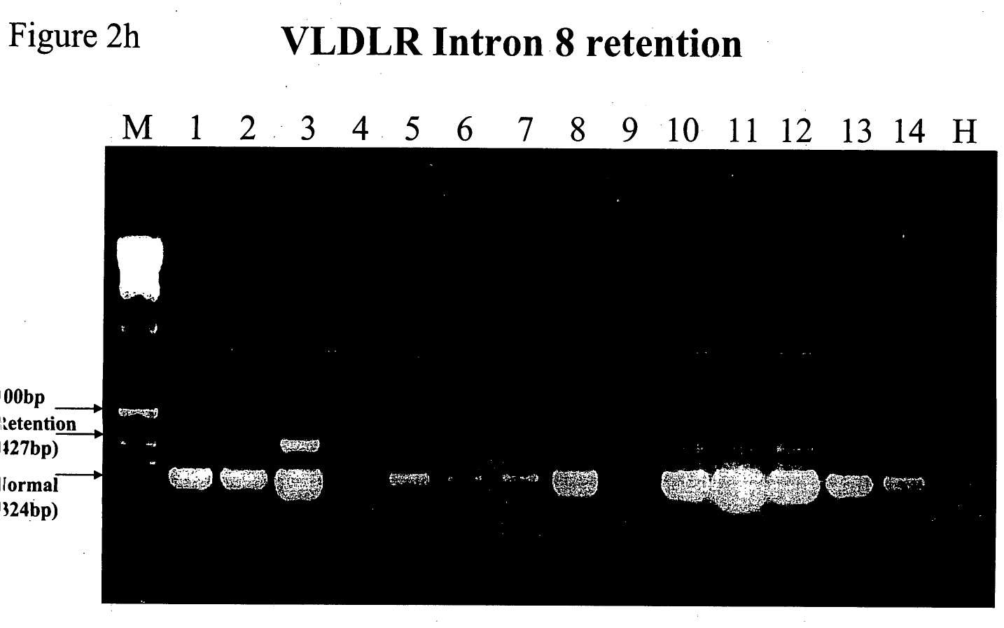

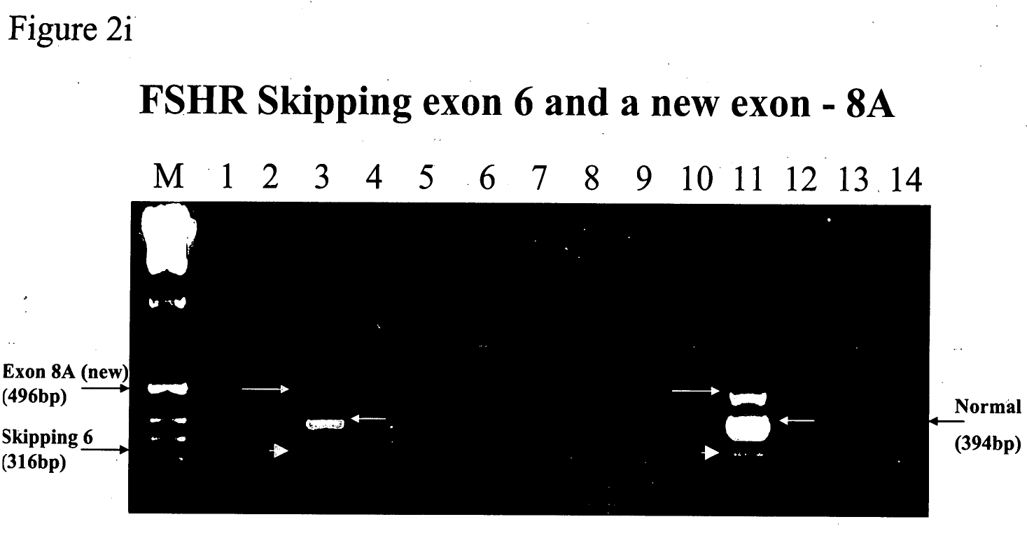

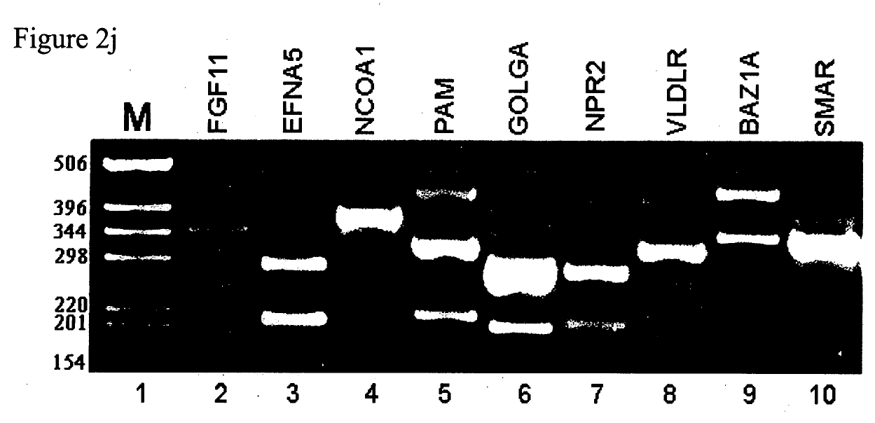

To show that candidate alternative exons for which no EST data exists are indeed alternative, 11 of them were randomly selected for experimental verification. For each of these exons, primers were designed from two flanking exons. RT-PCR reactions were carried out with RNA extractions of 14 different tissue types (FIGS. 2a-i). For 9 of these exons, a skipping splice variant was detected in at least one of the 14 tissues tested. In the tenth genie (VLDLR), it was predicted that exon 9 would be skipped; instead, the RT-PCR showed another type of alternative splicing—retention of intron 8. Only in one out of the 11 genes tested, the predicted skipping was not detected (skipping on exon 7 in FSHR).

In short, RT-PCR detected alternative splicing in 10 out of 11 predicted cases, in 9 of which this alternative splicing was an exon skipping event as predicted. This reflects a rate of success of at least 80%-90%. Moreover, the fact that the two predicted exon skipping events were not detected does not mean they do not exist, as they could still exist in a tissue other than the 14 that were tested, or in a particular embryonic developmental stage for example.

A similar protocol was followed for the experimental results in FIG. 2j, except that a different set of primers was used (see Table 8 below).

TABLE 8

Primers used for validation of
alternative exons.

| | | |
|---|---|---|
| Gene and | | |
| direction | Primer sequences | TM |

| | | |
|---|---|---|
| FGF11 | | |
| Forward | 5′-CCAAGGTGCGACTGTGCGG-3′ | 68° C. |
| FGF11 | | |
| Reverse | 5′-GGTAGAGAGCAGAGGCGTACAGGACG-3′ | 66° C. |
| EFNA5 | | |
| Forward | 5′-ACCGGCCTCACTCTCCAAATGG-3′ | 65° C. |
| EFNA5 | | |
| Reverse | 5′-TGGCTCGGCTGACTCATGTACGG-3′ | 67° C. |
| NCOA1 | | |
| Forward | 5′-AGGCAACACGACGAAATAGCCATACC-3′ | 66° C. |
| NCOA1 | | |
| Reverse | 5′-TCTGGCATAAGATGGTTCTCTGCCC-3′ | 65° C. |
| PAM | | |
| Forward | 5′-TGTCCCAGTGCCCGGG-3′ | 61° C. |
| PAM | | |
| Reverse | 5′-GGTGAAATCCACAGCTGACTTGG-3′ | 62° C. |
| GOLGA4 | | |
| Forward | 5′-TCAAGAGAACCTACTTAAGCGTTGTAAGG-3′ | 61° C. |
| GOLGA4 | | |
| Reverse | 5′-TGAGCAATTTCTTCTTCTTTCATTTCC-3′ | 61° C. |
| NPR2 | | |
| Forward | 5′-CATGTTTGGTGTTTCCAGCTTCC-3′ | 62° C. |
| NPR2 | | |
| Reverse | 5′-CGGGTCAGCTCAATGCGC-3′ | 62° C. |
| VLDLR | | |
| Forward | 5′-TGAGCCCCTGAAAGAGTGTCATATAAACG-3′ | 66° C. |
| VLDLR | | |
| Reverse | 5′-TCTAAGCCAATCTTCCTGATGTCTCTTCG-3′ | 66° C. |
| BAZ1A | | |
| Forward | 5′-TGCTCTGATGGTTTTGGAGTTCC-3′ | 61° C. |
| BAZ1A | | |
| Reverse | 5′-CGTTTTTGATATCTATACTTTGCATTTGC-3′ | 60° C. |
| SMARCD1 | | |
| Forward | 5′-CAGCCTTGTCCAAATATGATGCC-3′ | 61° C. |
| SMARCD1 | | |
| Reverse | 5′-AAACTCCCGCTCGTGAGGG-3′ | 61° C. |
| DICER1 | | |
| Forward | 5′-AACTCATTCAGATCTCAAGGTTGGG-3′ | 61° C. |
| DICER1 | | |
| Reverse | 5′-CCAGGTCAGTTGCAGTTTCAGC-3′ | 61° C. |
| HATB | | |
| Forward | 5′-AGGCTTCAGACCTTTTTGATGTGG-3′ | 62° C. |
| HATB | | |
| Reverse | 5′-CTTCCGCTGTAATATCAAGAACTGTAGG-3′ | 61° C. |
| PRKCM | | |
| Forward | 5′-AAGTACTGGGTTCTGGACAGTTTGG-3′ | 61° C. |
| PRKCM | | |
| Reverse | 5′-CTGGTTTGAGGTCACAGTGAACG-3′ | 61° C. |
| RNASE3L | | |
| Forward | 5′-CGGAGAATTTTTGTGTGAAAGGG-3′ | 61° C. |
| RNASE3L | | |
| Reverse | 5′-CCAGCTCCTCCCACTGAAGC-3′ | 61° C. |
| TIAM2 | | |
| Forward | 5′-AACGACAGTCAGGCCAACGG-3′ | 62° C. |
| TIAM2 | | |
| Reverse | 5′-CCAGAAACACCTTCTGAAACTCAAGC-3′ | 62° C. |
| MDA5 | | |
| Forward | 5′-AAATCTGGAGAAGGAGGTCTGGG-3′ | 61° C. |
| MDA5 | | |
| Reverse | 5′-CCACTCTGGTTTTTCCACTCCC-3′ | 61° C. |

Table 9 shows a description of the results obtained in the experiment (shown in FIG. 2j).

TABLE 9
Experimental validation of predicted alternatively spliced exons

| | | | Type of | |
|---|---|---|---|---|
| | Alt | PCR | alternative | |
| Gene | Exon^{a} | confirmed^{b} | confirmed^{c} | Gene Description |
| FGF11 | 2 | Yes | Skip | fibroblast growth factor 11 |
| EFNA5 | 4 | Yes | Skip | ephrin-A5 |
| NCOA1 | 8 | Yes | Skip | steroid nuclear receptor |
| | | | | coactivator |
| PAM | 22 | Yes | Skip | protein associated with Myc |
| | | | | mRNA |
| GOLGA4 | 9 | Yes | Skip | golgi autoantigen, golgin |
| | | | | subfamily a, 4 |
| NPR2 | 9 | Yes | Skip | natriuretic peptide receptor |
| | | | | B/guanylate cyclase B |
| VLDLR | 9 | Yes | Int Ret^{d} | very low density lipoprotein |
| | | | | receptor |
| BAZ1A | 12 | Yes | Alt 3′ss^{e} | bromodomain adjacent to zinc |
| | | | | finger domain protein 1A |
| SMARCD1 | 7 | Yes | Alt 3′ss^{f} | SWI/SNF related, matrix |
| | | | | associated, actin dependent |
| | | | | regulator of chromatin, subfamily |
| | | | | d, member 1 |
| PRKCM | 15 | No | | protein kinase C, mu |
| TIAM2 | 12 | No | | T-cell lymphoma invasion and |
| | | | | metastasis 2 |
| MDA5 | 4 | No | | melanoma differentiation |
| | | | | associated protein-5 |
| RNASE3L | 15 | No | | nuclear RNase III |
| HAT1 | 7 | No | | histone acetyltransferase 1 |
| DICER1 | 6 | No | | Dicer1, Dcr-1 homolog |
| | | | | (Drosophila) |

^{a}Serial number of exon (out of gene's exons) identified as alternative
^{b}For each predicted exons, primers were designed from its flanking exons and RT_PCR was conducted using total RNA from 14 different tissue types: cervix, uterus, ovary, placenta, breast, colon, pancreas, liver + spleen, brain, prostate, testis, kidney, thyroid, and assorted cell-lines. Products were sequenced, and alternative splicing was searched.
^{c}Type of alternative splicing: Skip, exon-skipping; Alt 3′ss, alternative 3′ splice site (acceptor); Int Ret., intron retention.
^{d}Retention of intron 8 (size 103 nucleotides) was detected in VLDLR.
^{e}Deletion of 86 nucleotides was detected on the 3′ end of exon 12 7 of BAZ1A.
^{f}Extension of 44 nucleotides was detected on the 3′ end of exon 12 of SMARCD1.

### Example 3

### Examples of Annotations for Selected Variants Uncovered Using the Teachings of the Present in Invention

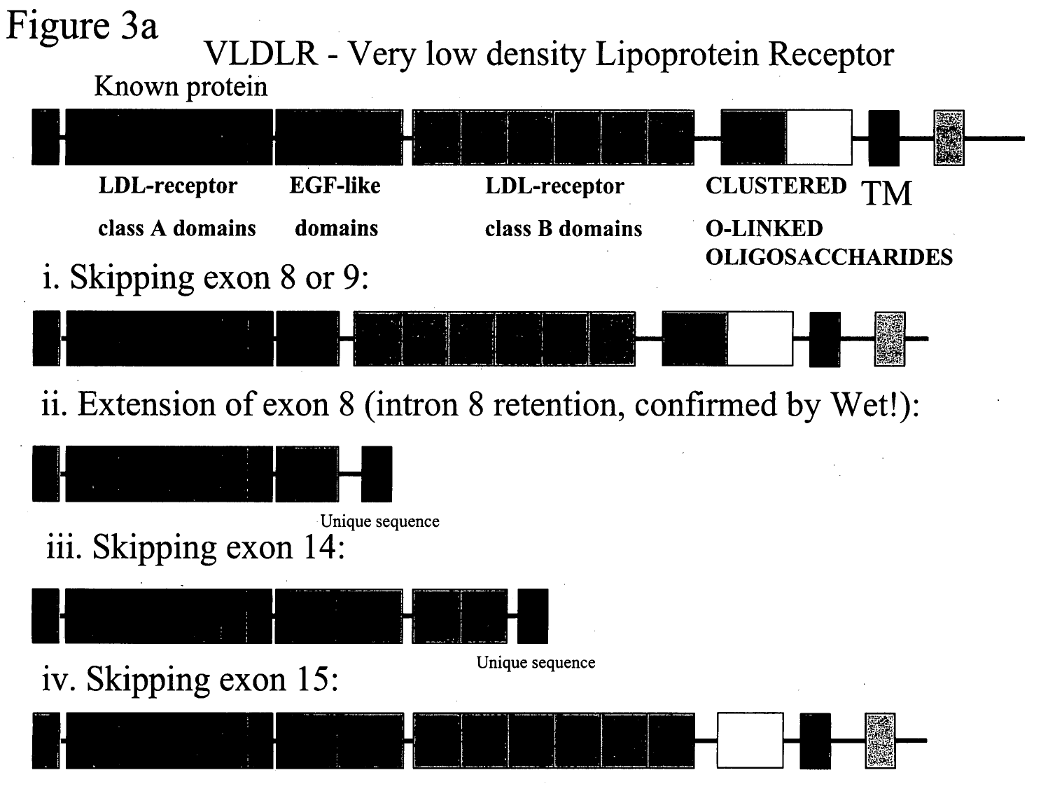

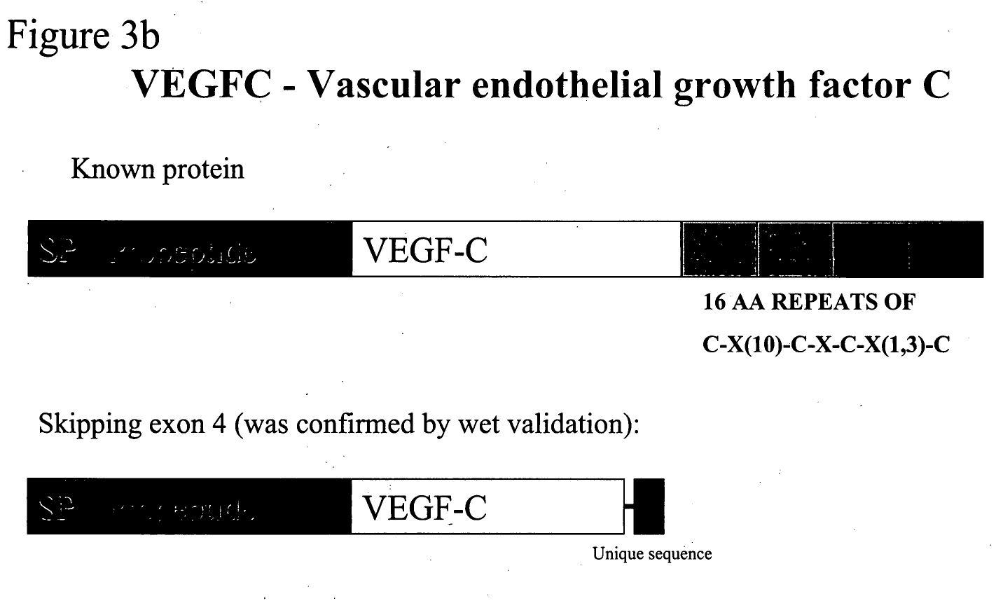

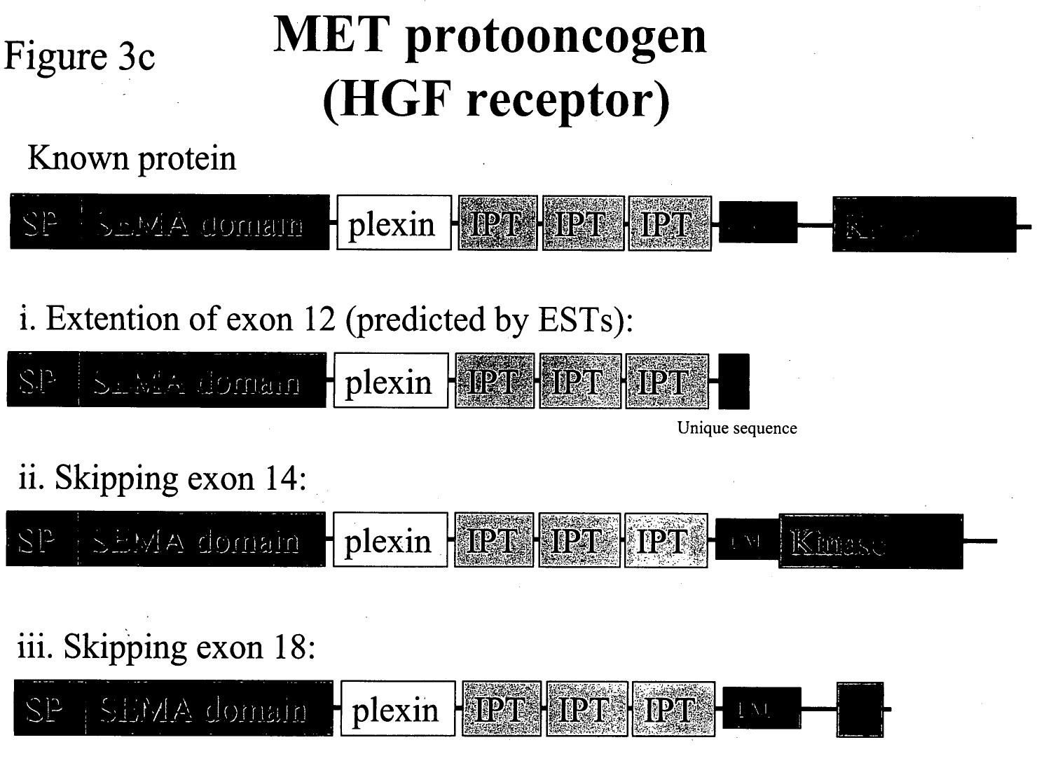

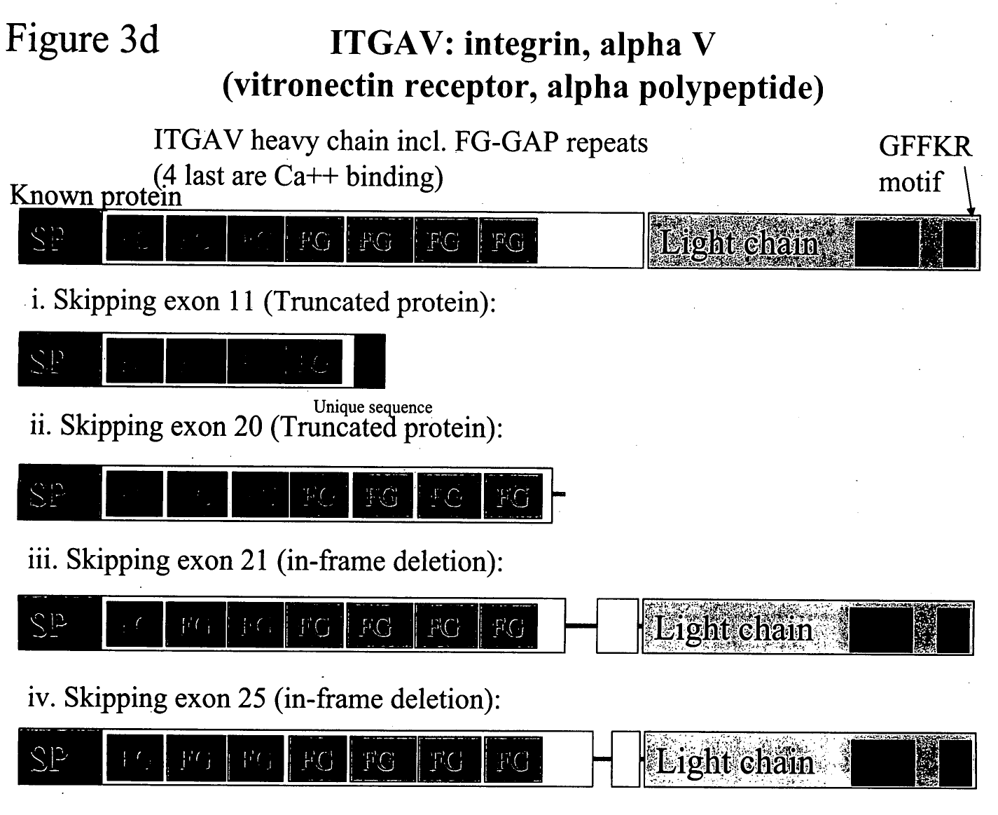

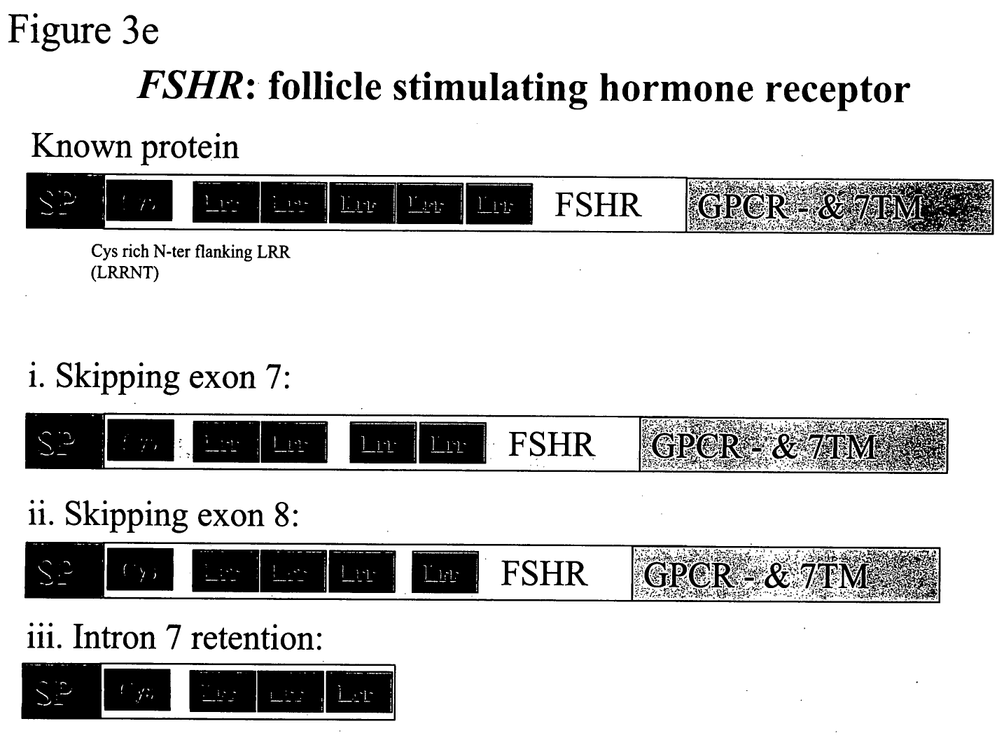

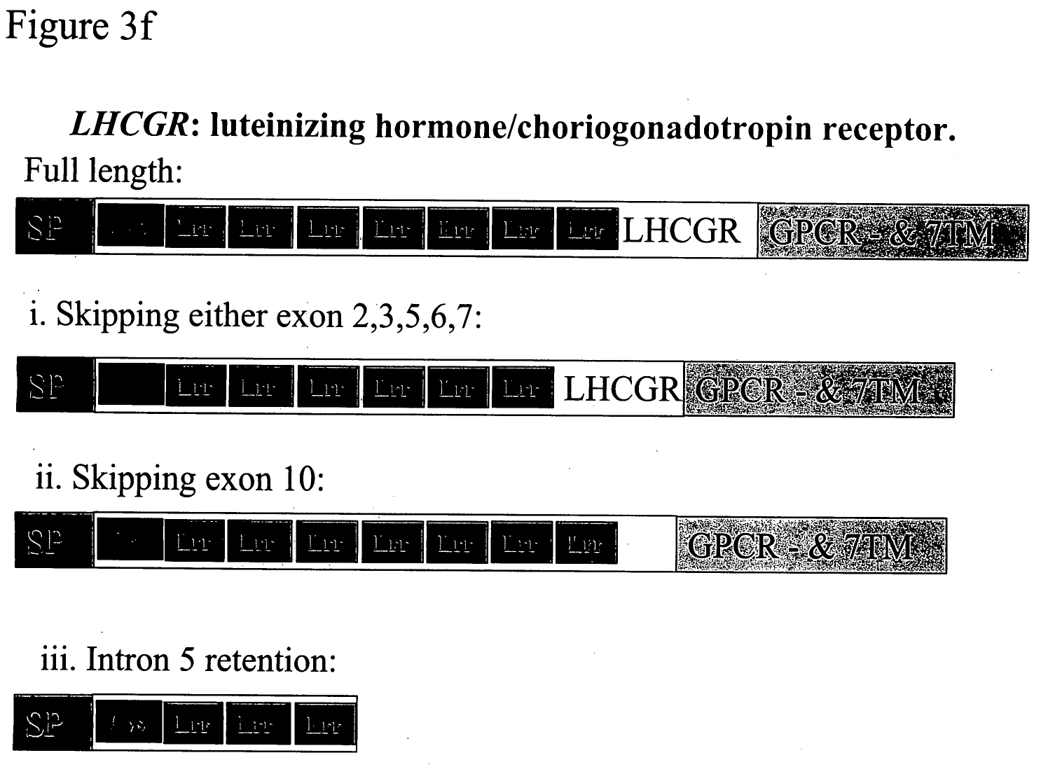

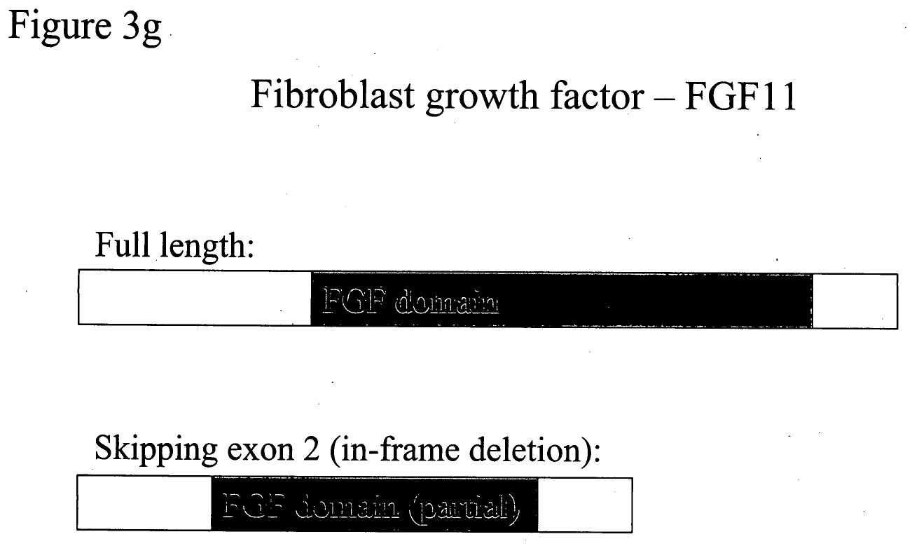

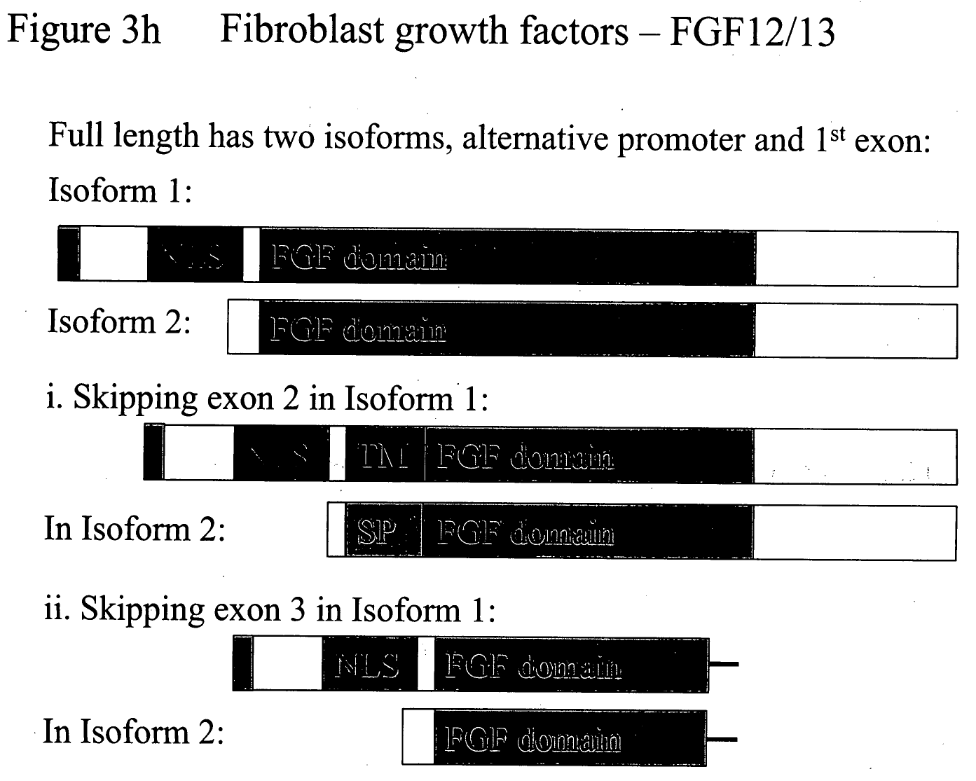

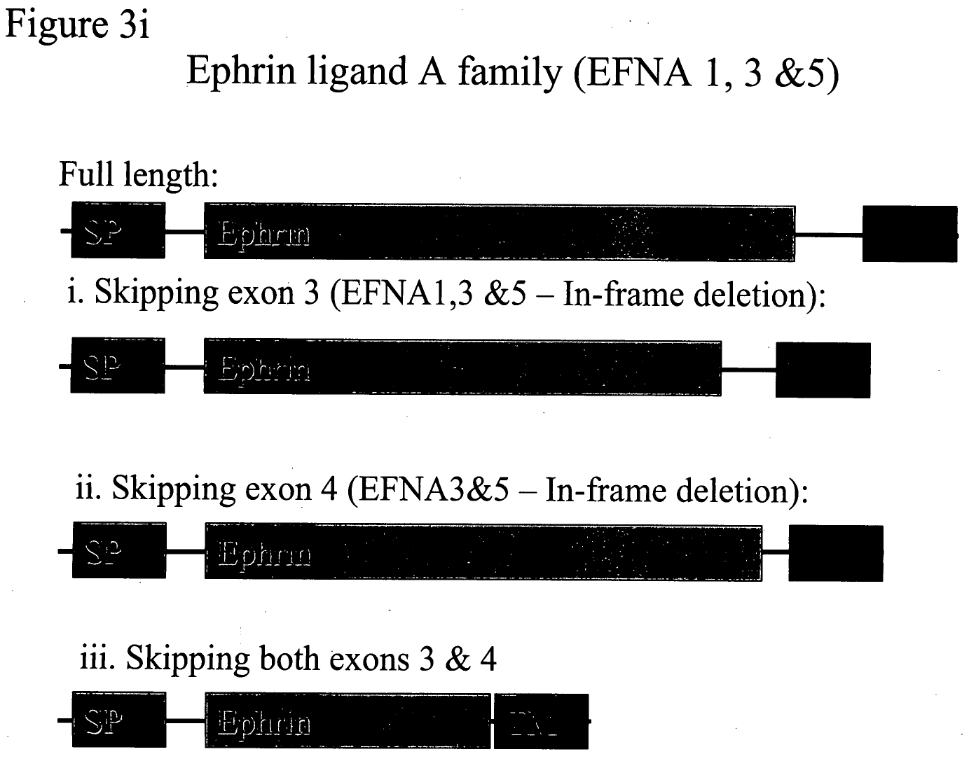

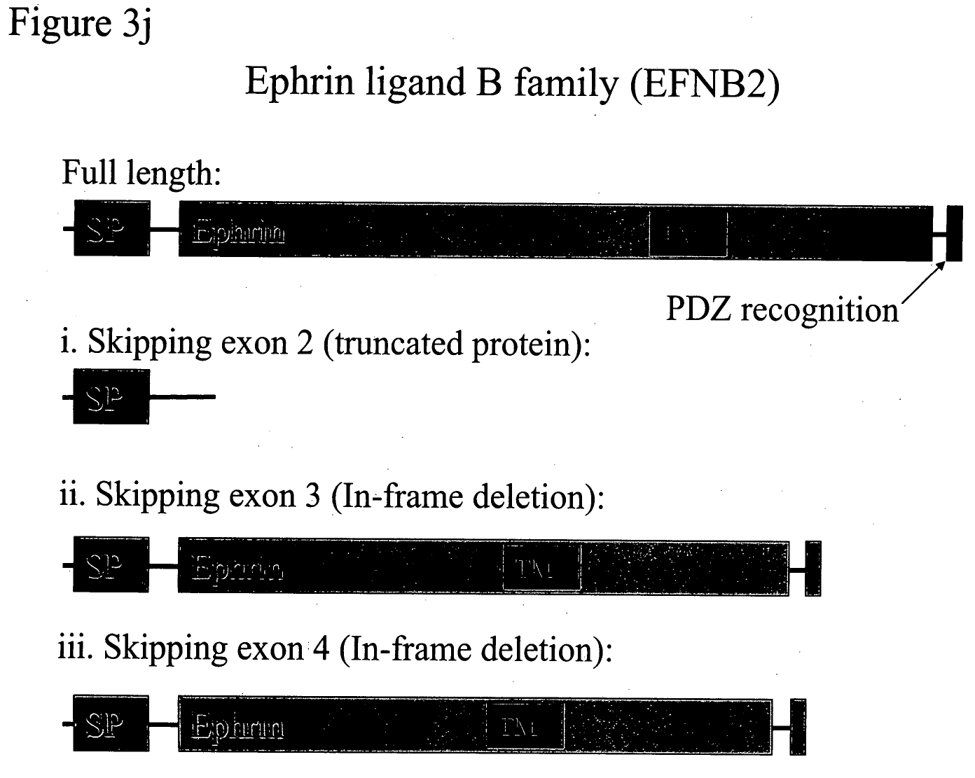

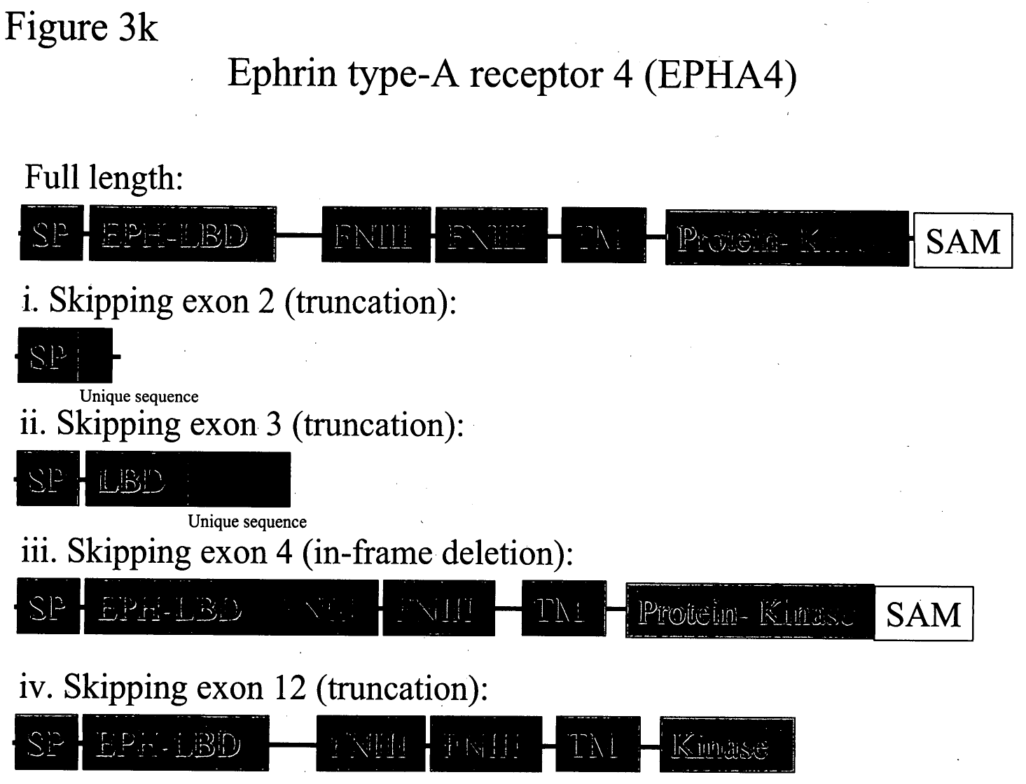

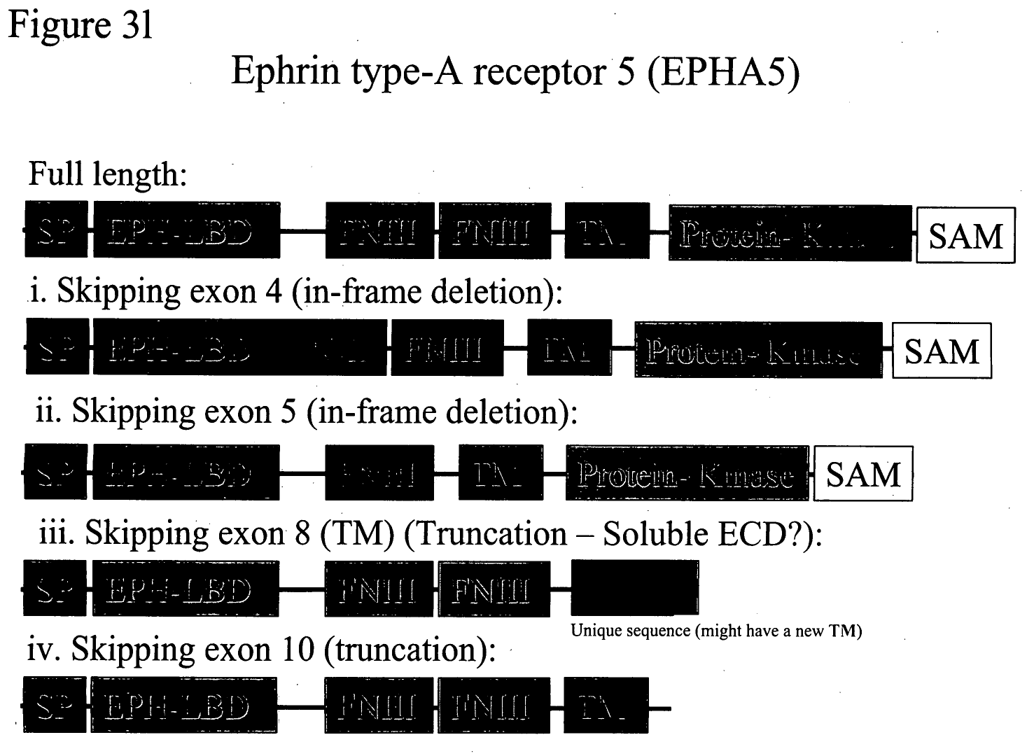

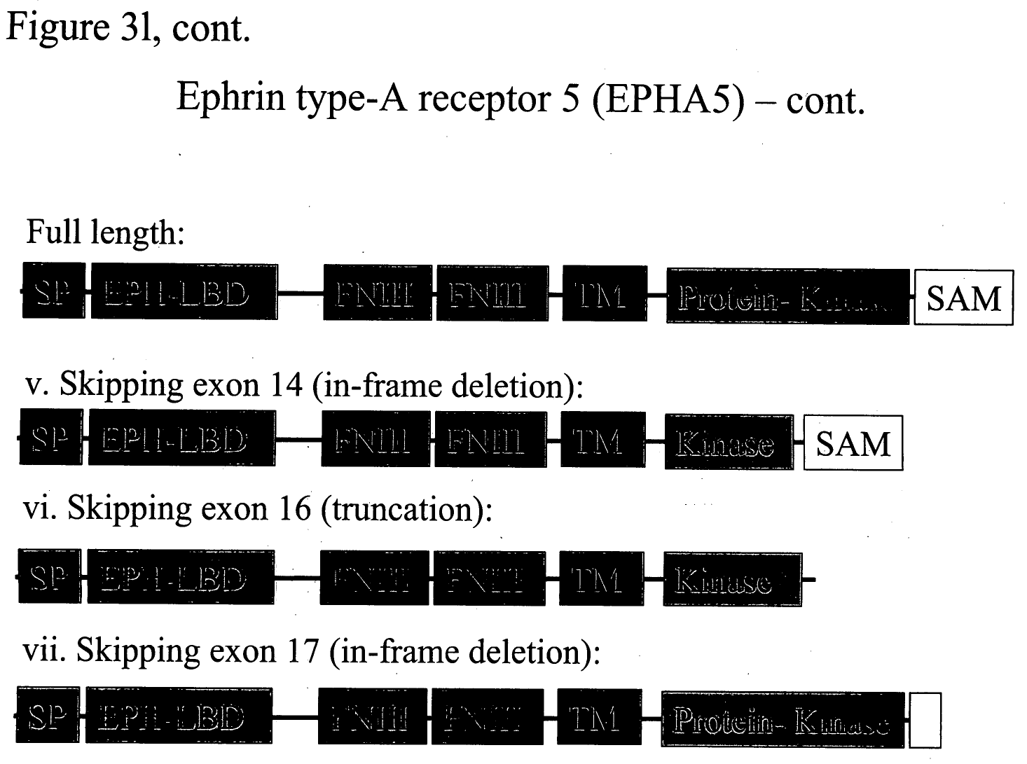

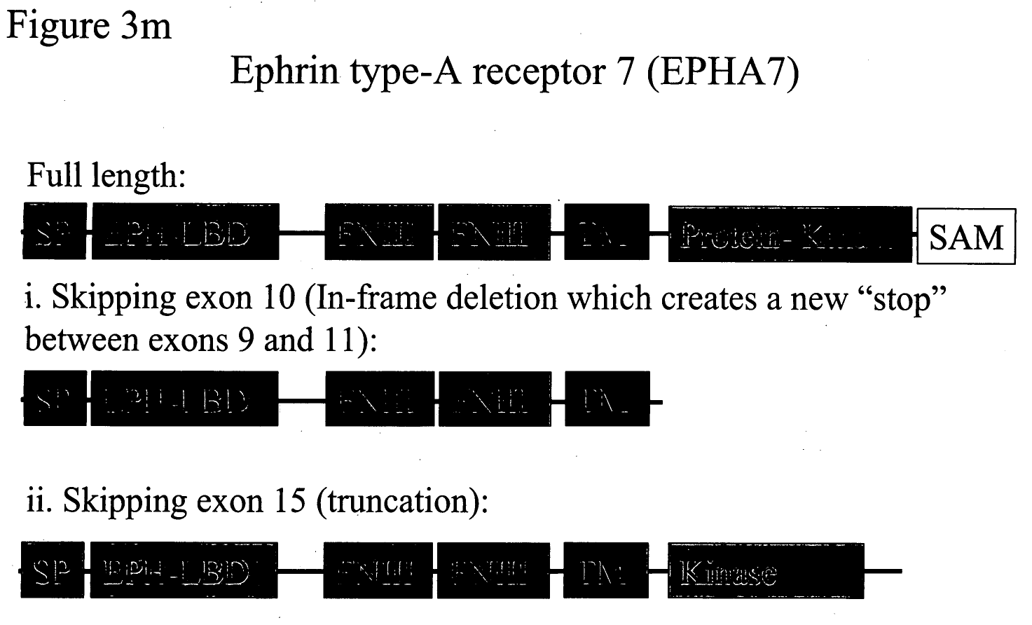

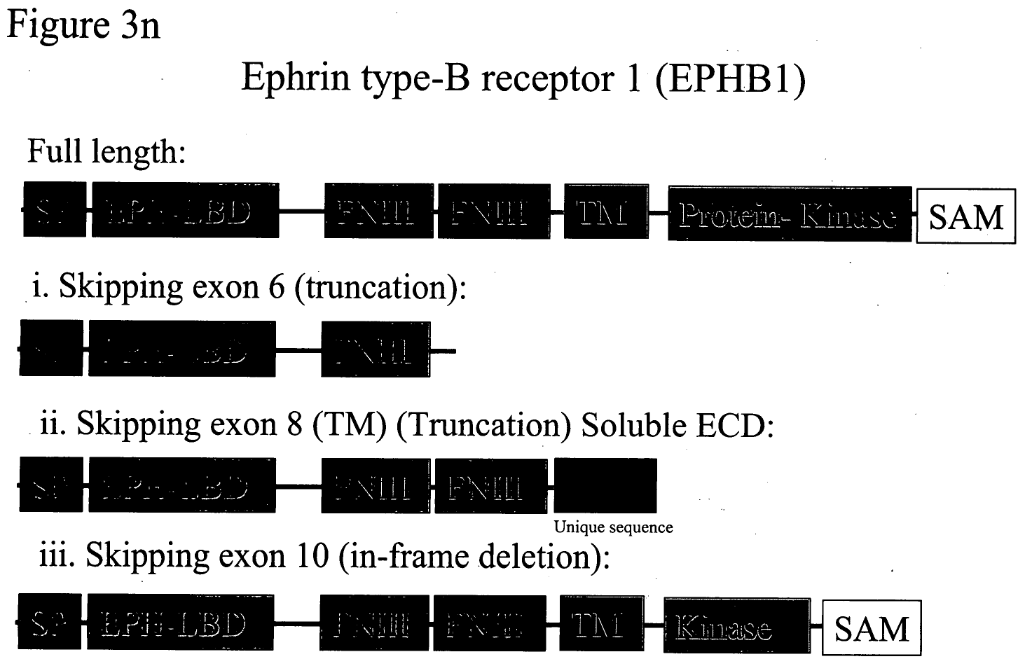

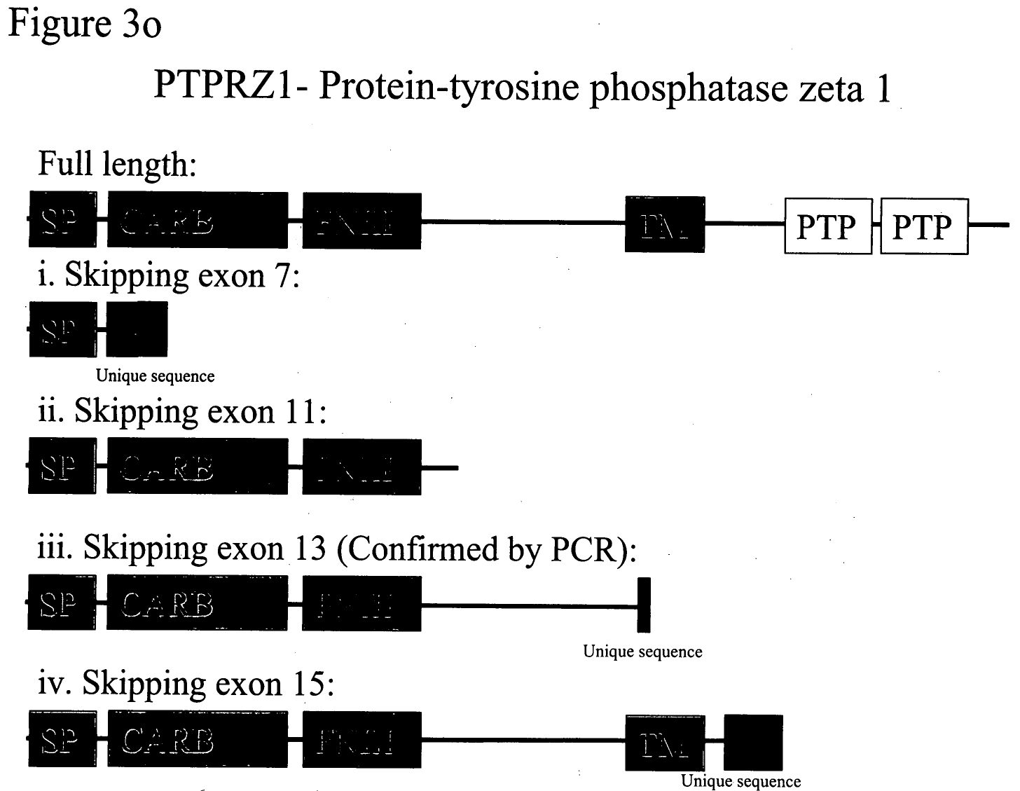

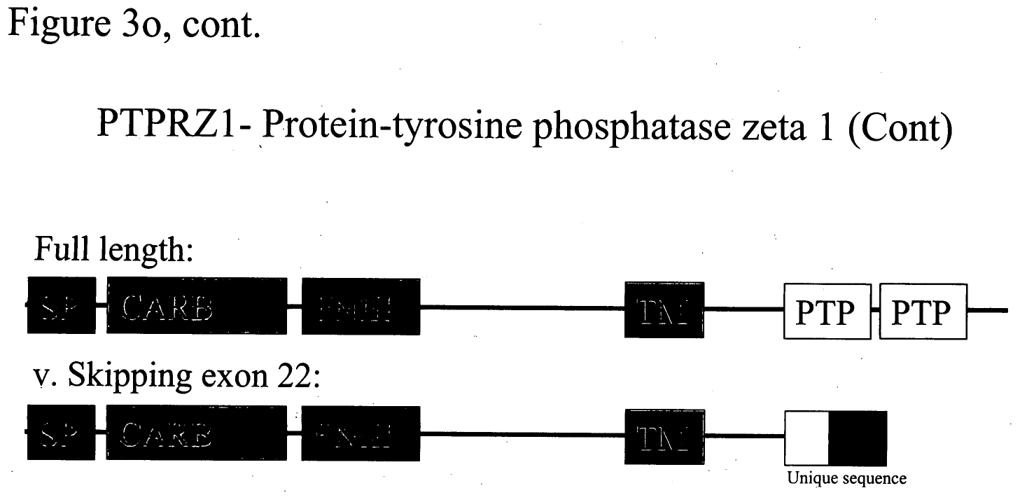

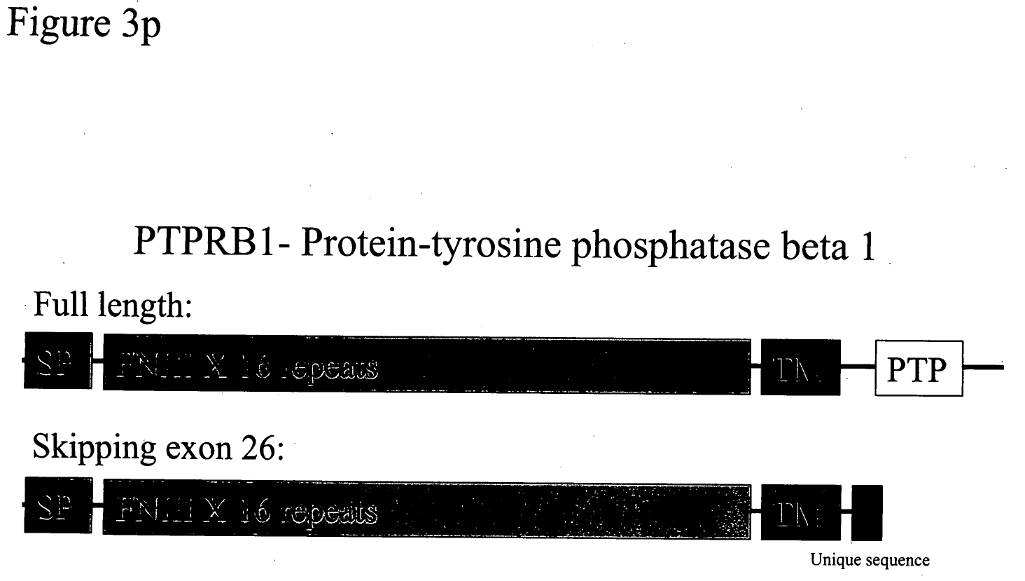

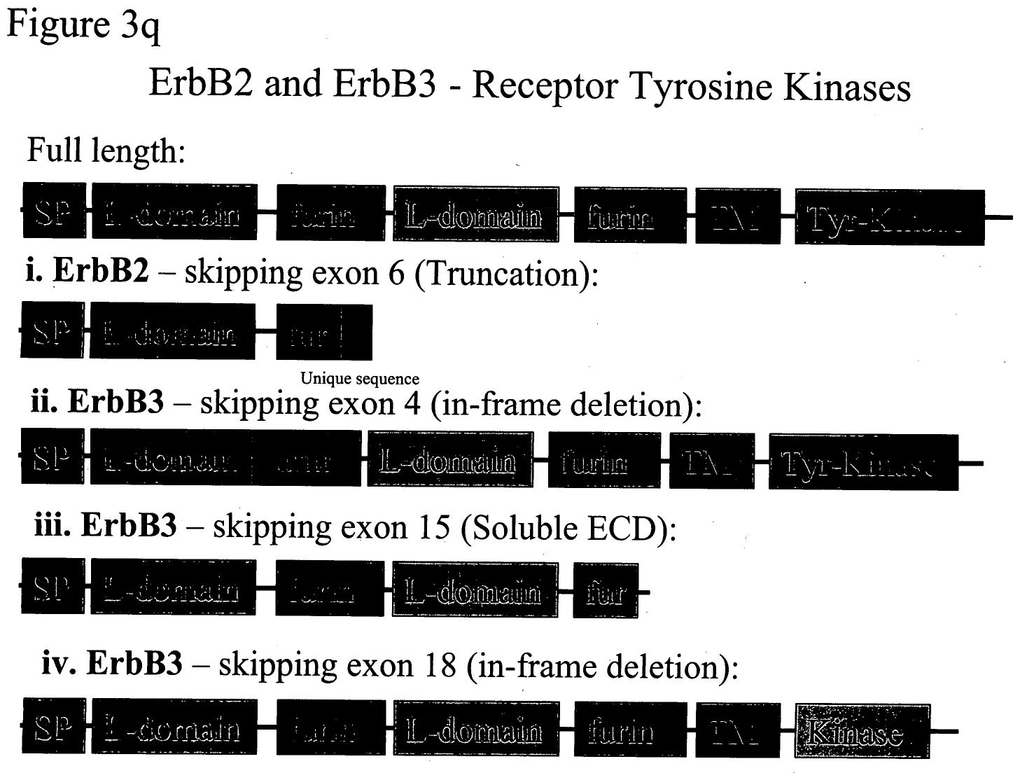

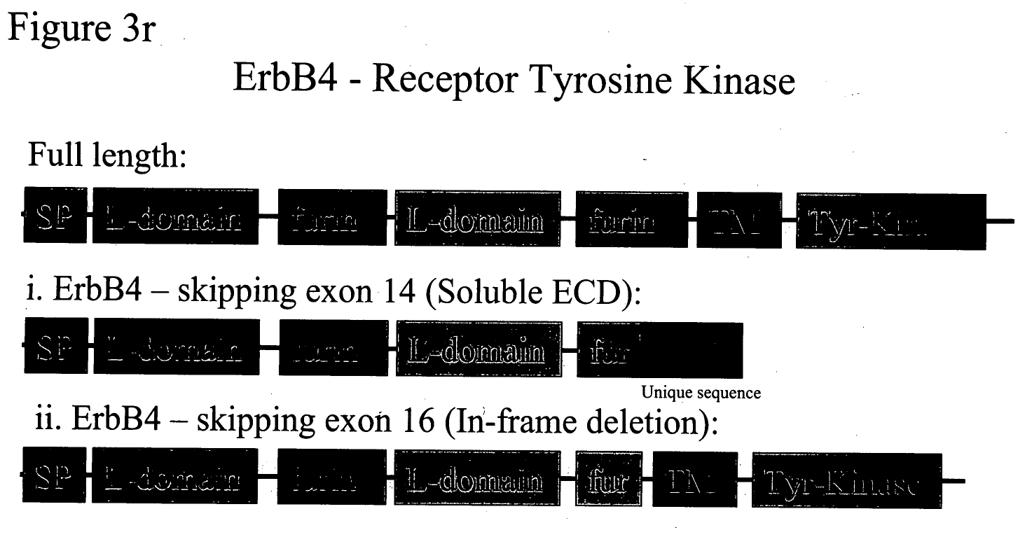

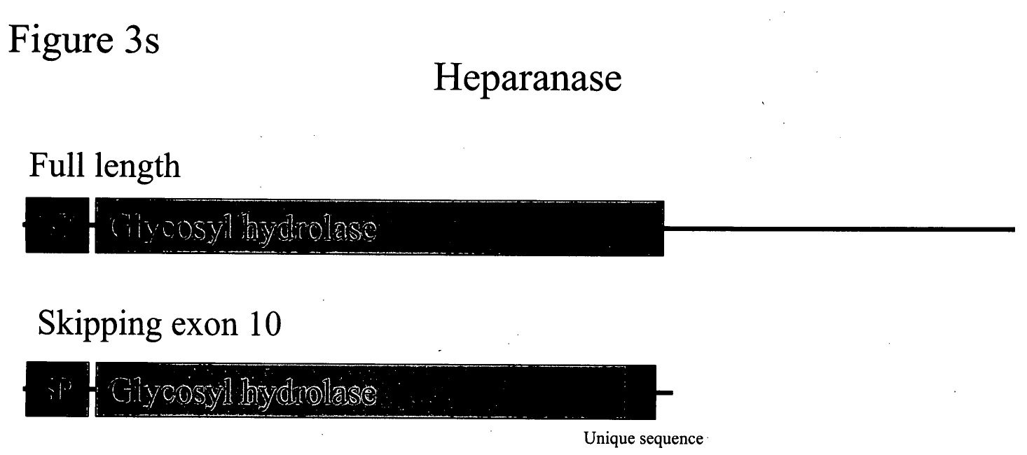

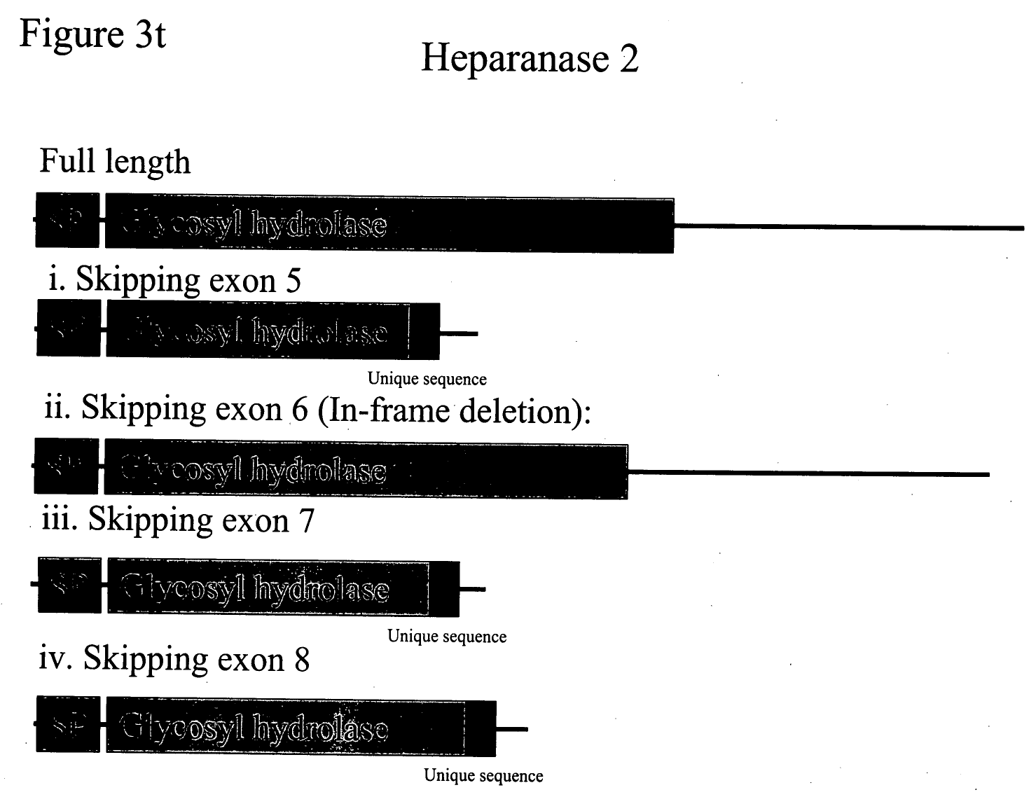

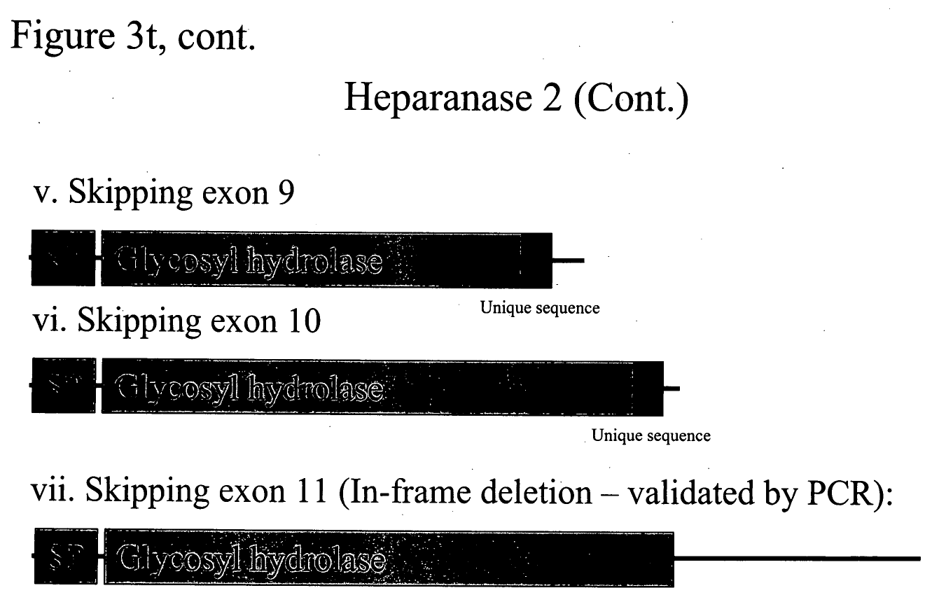

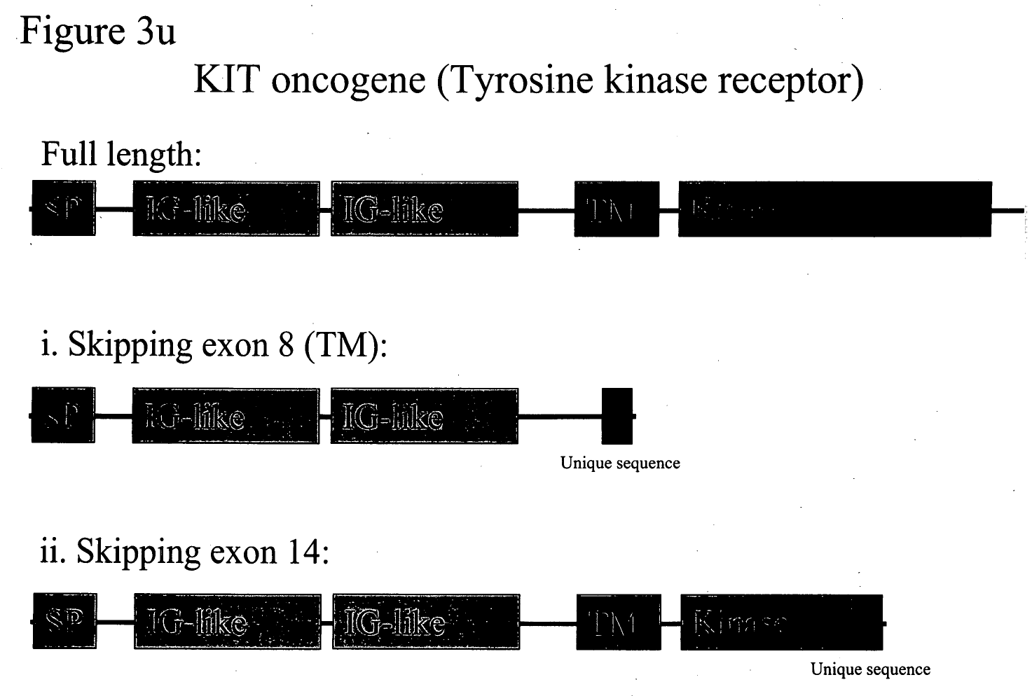

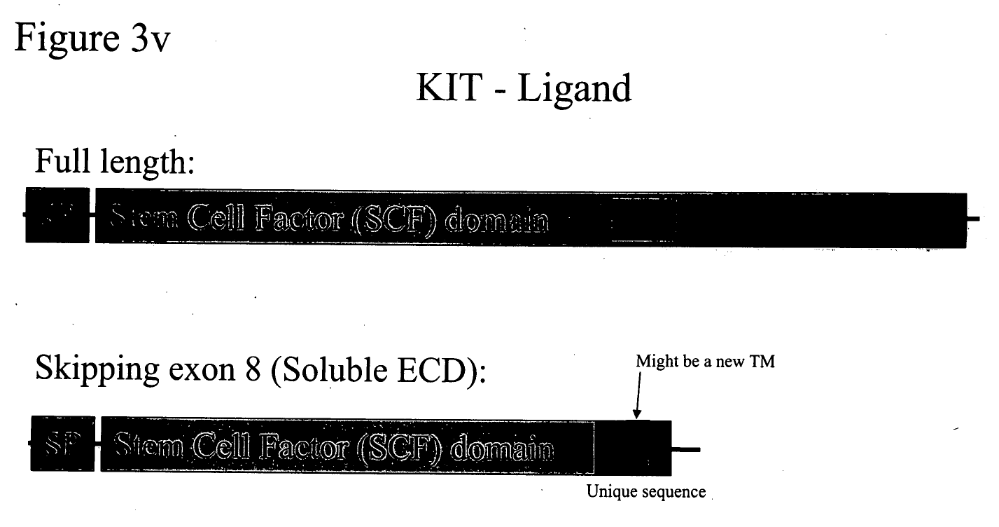

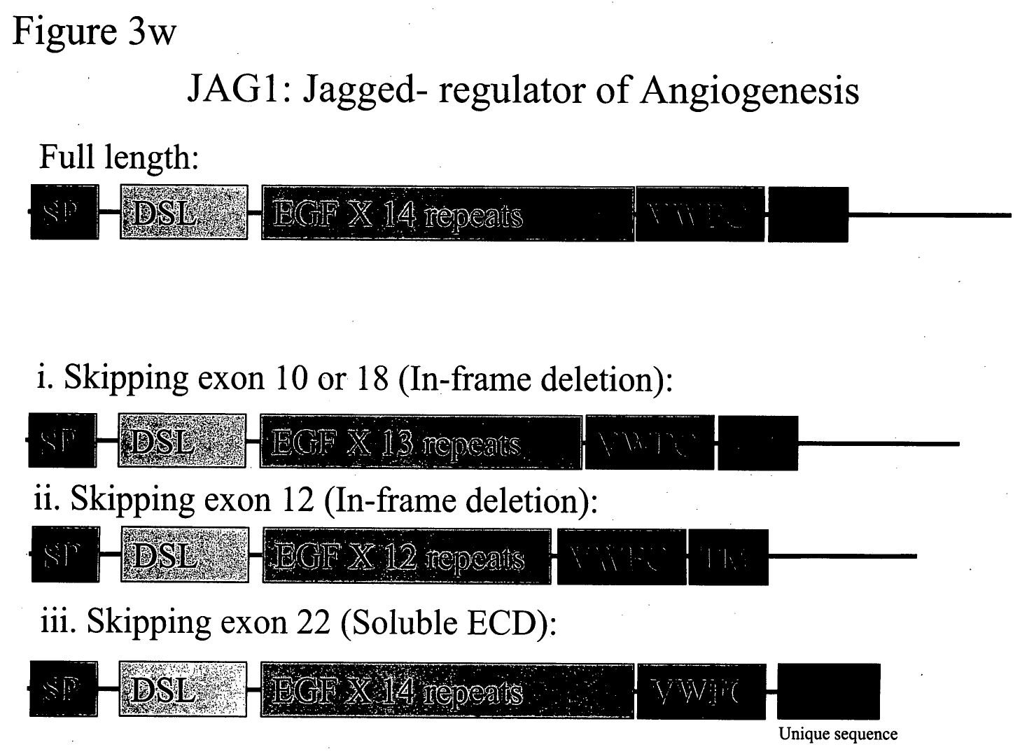

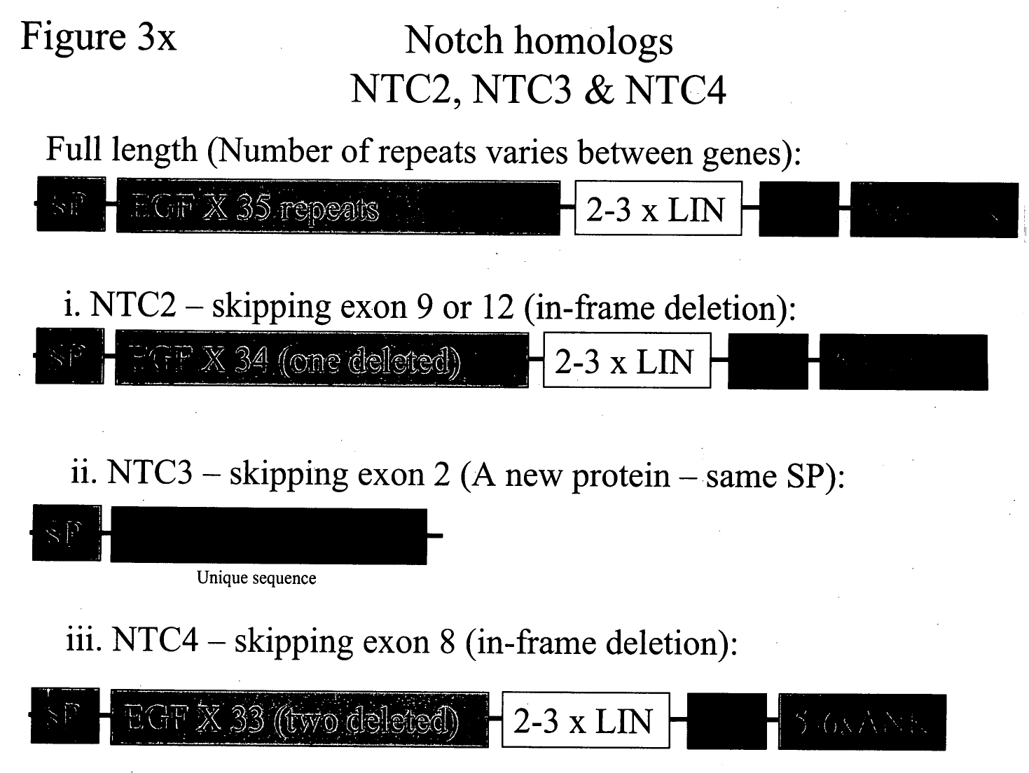

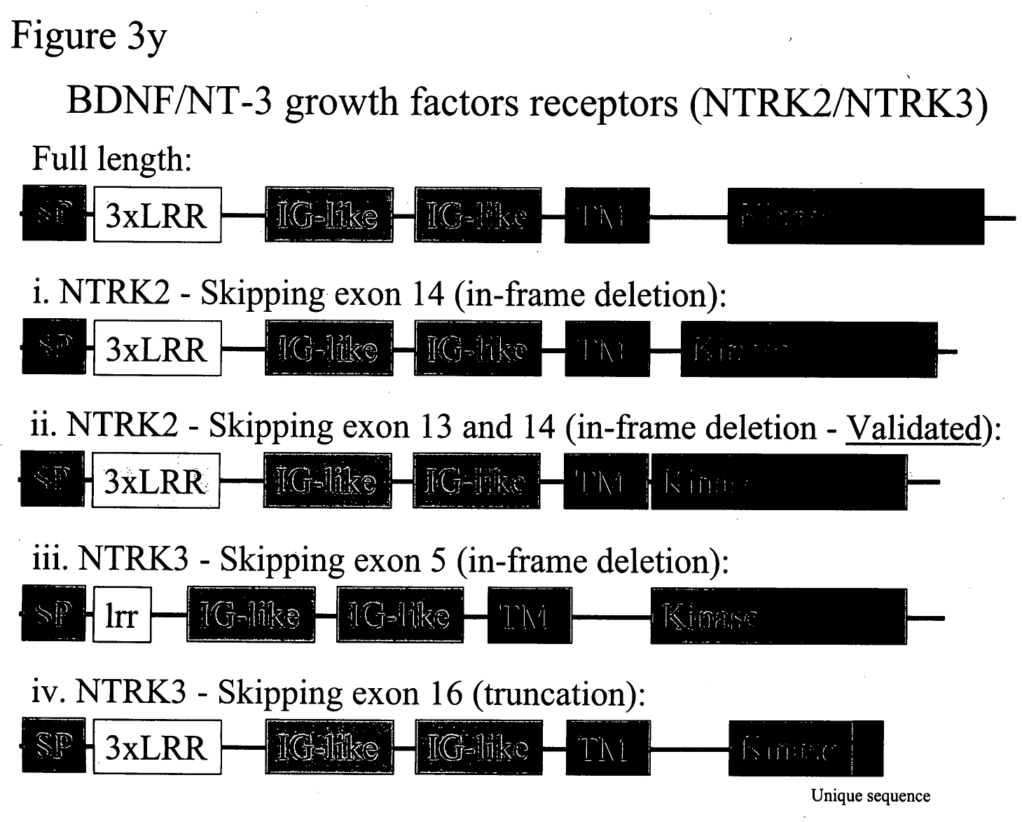

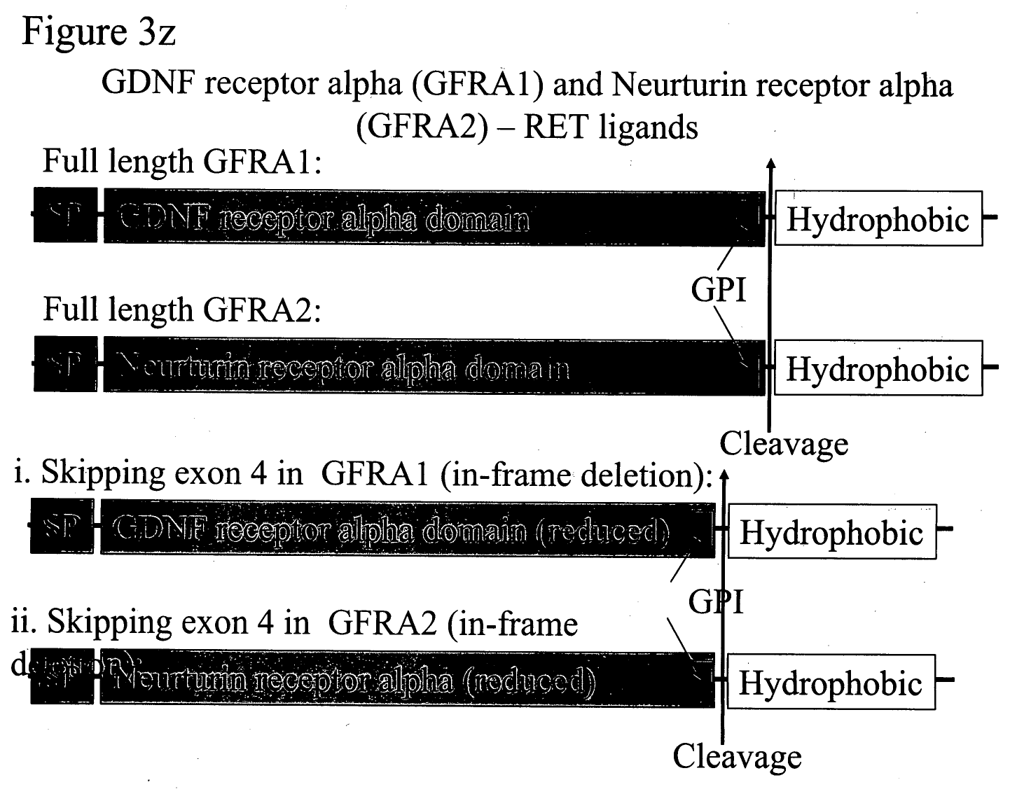

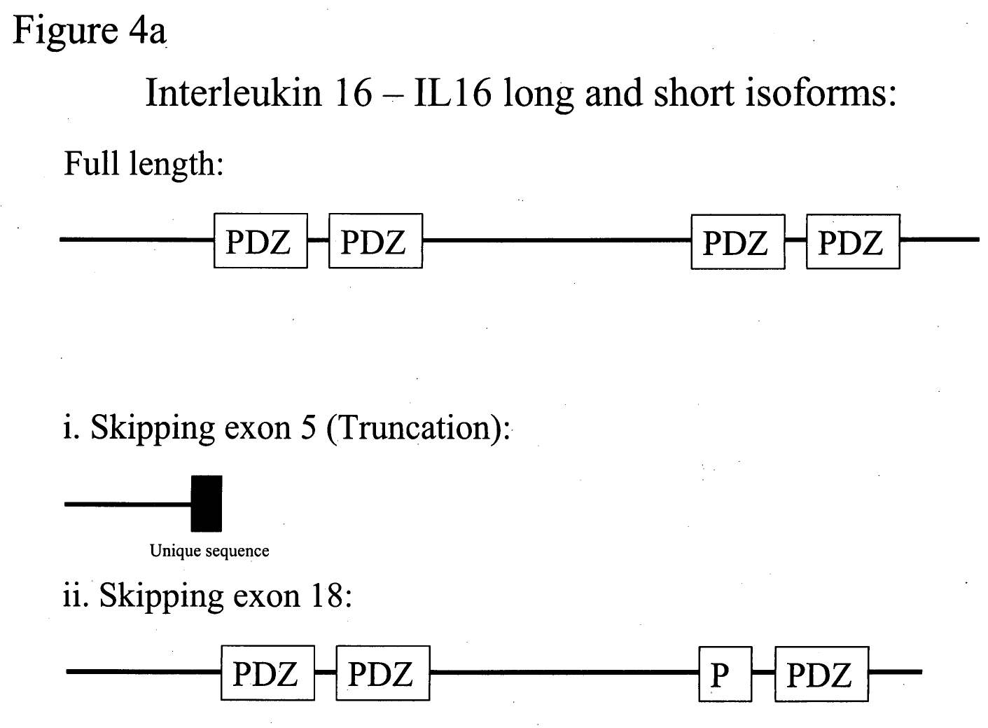

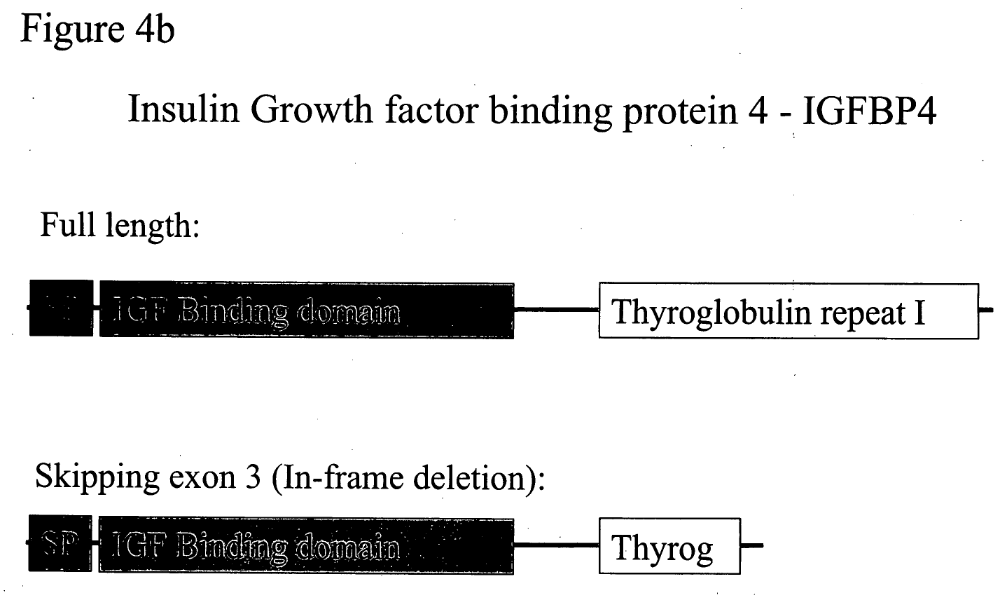

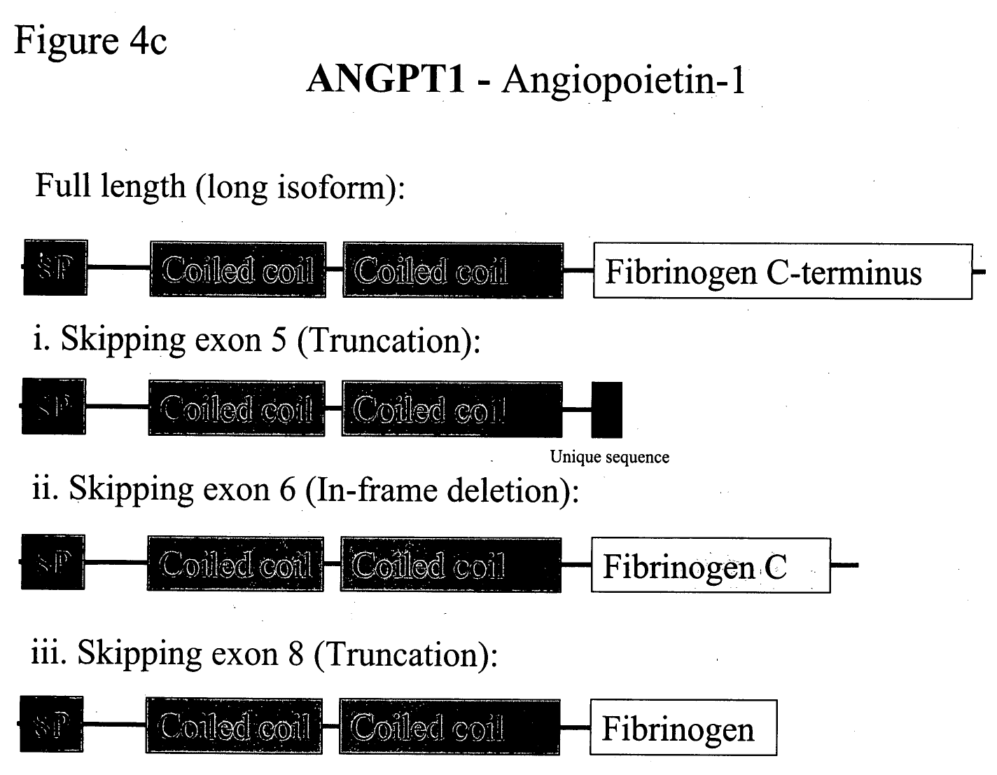

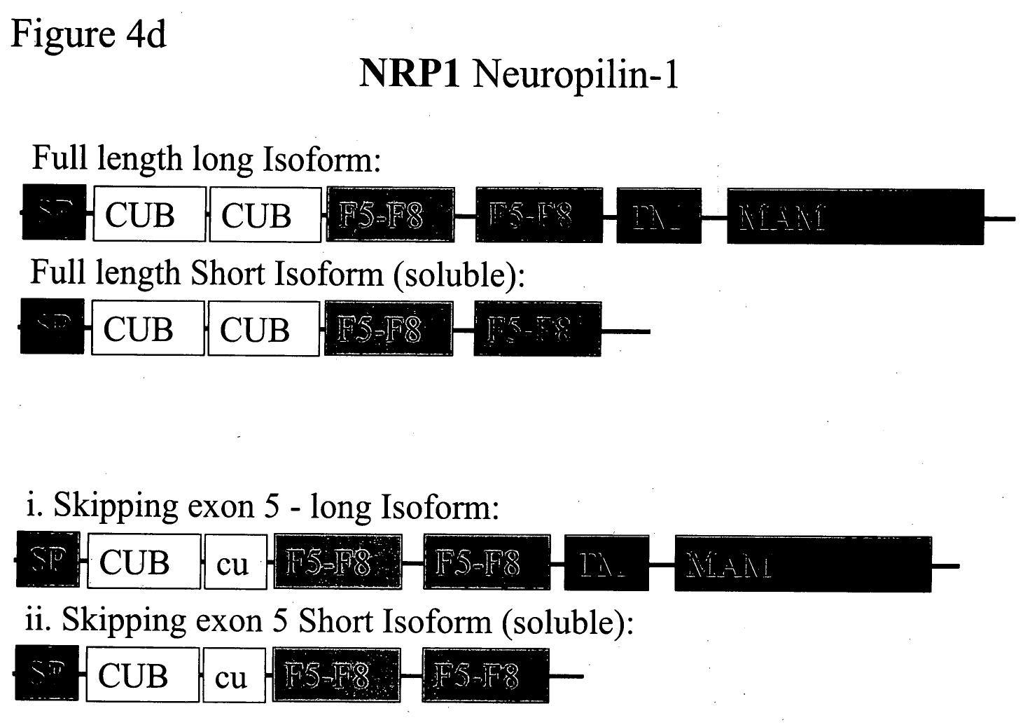

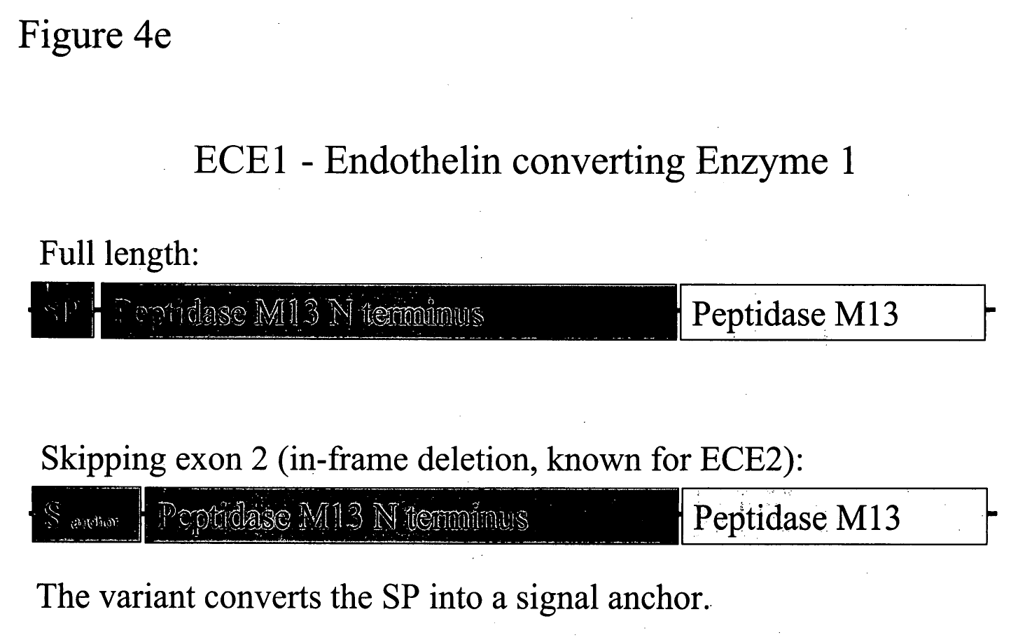

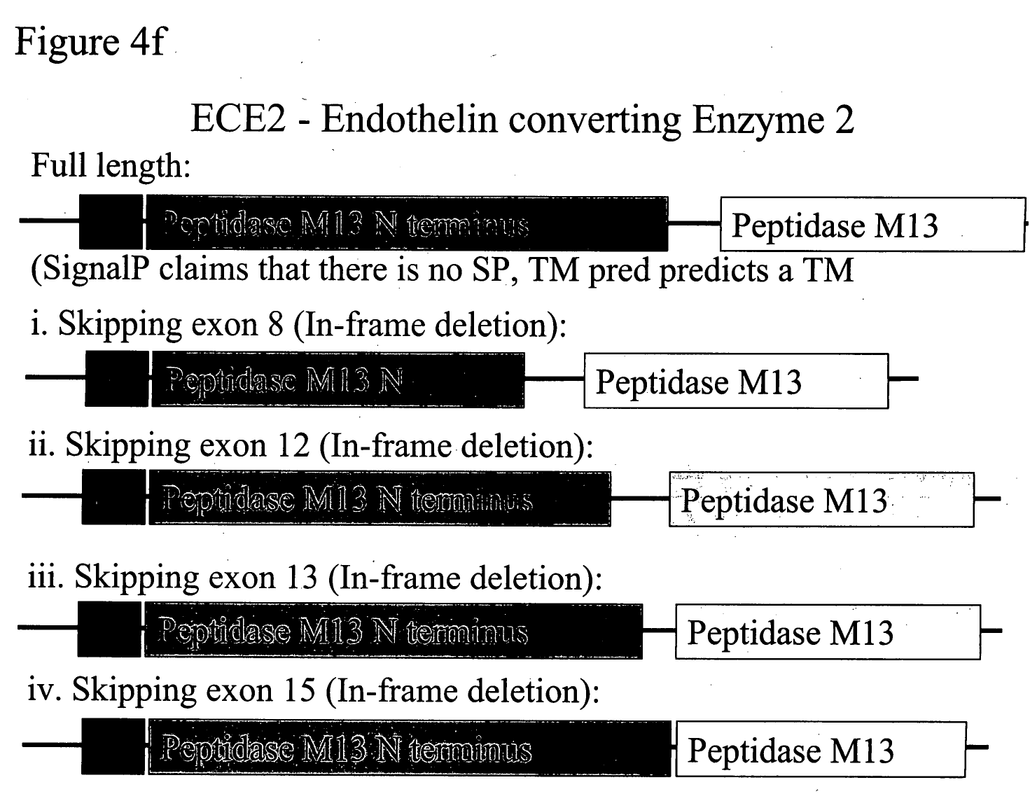

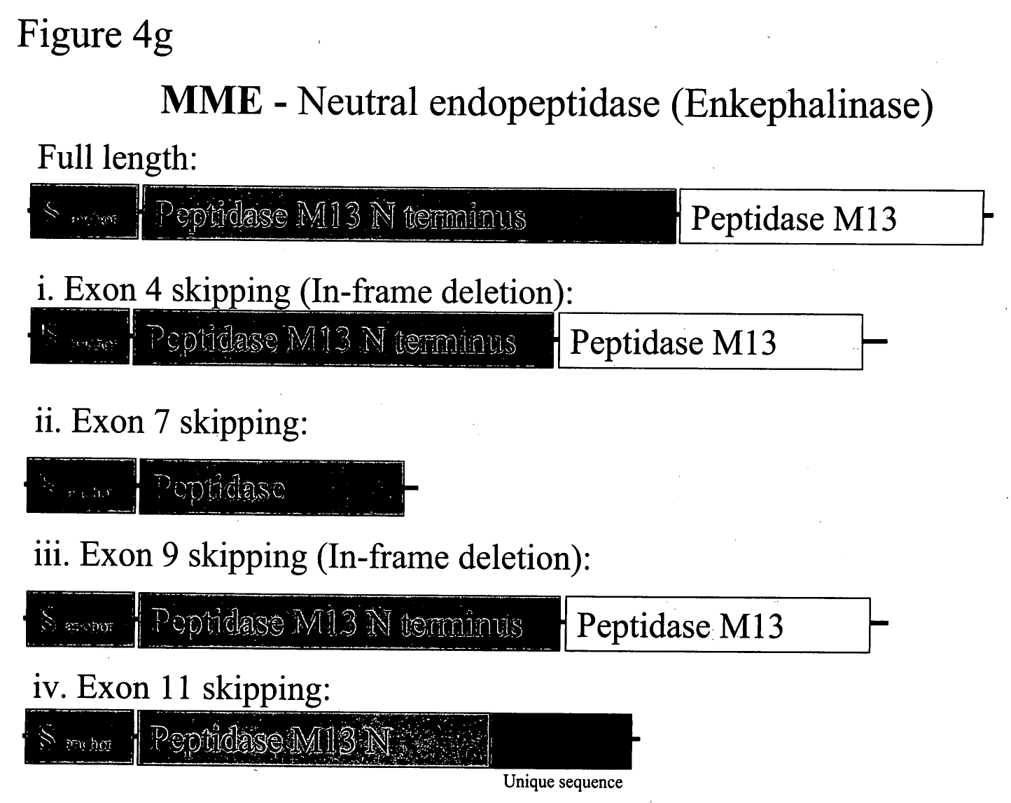

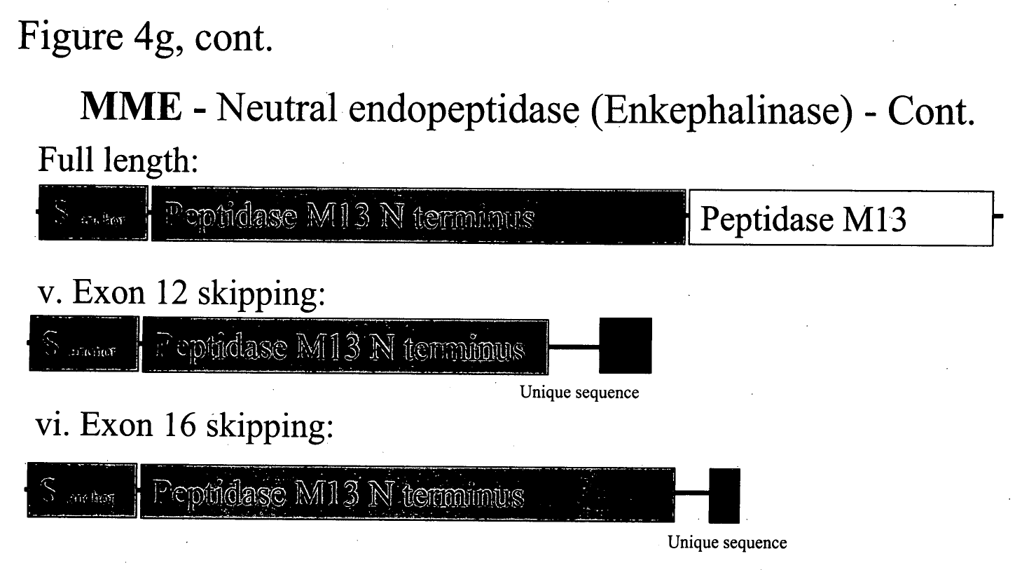

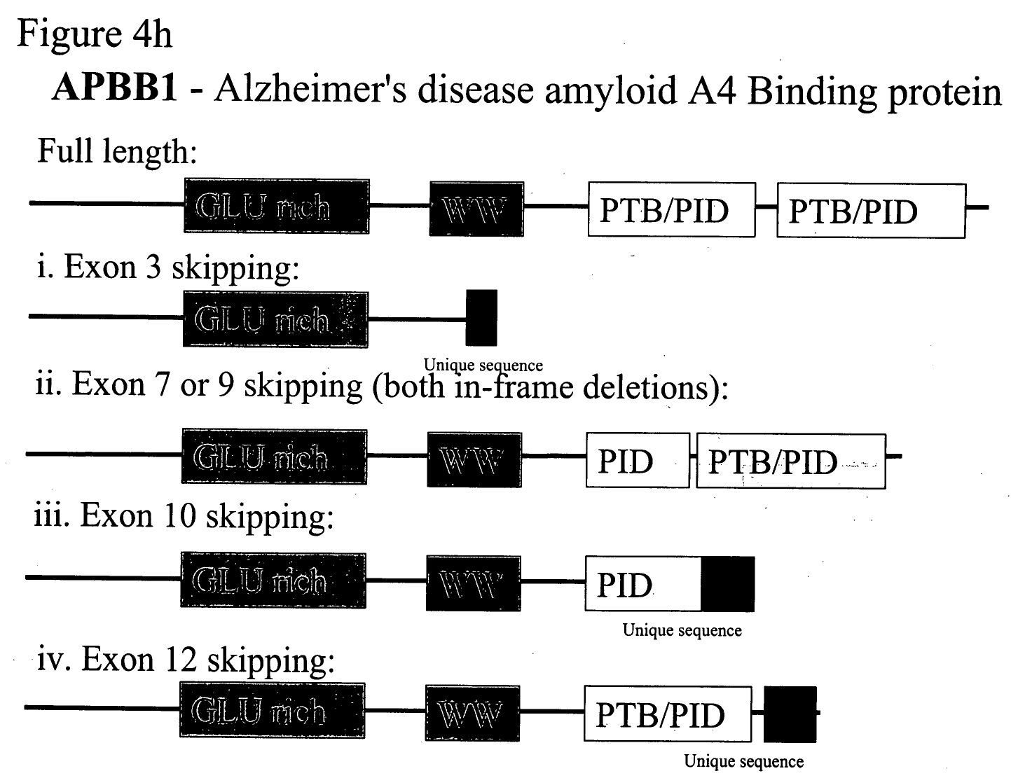

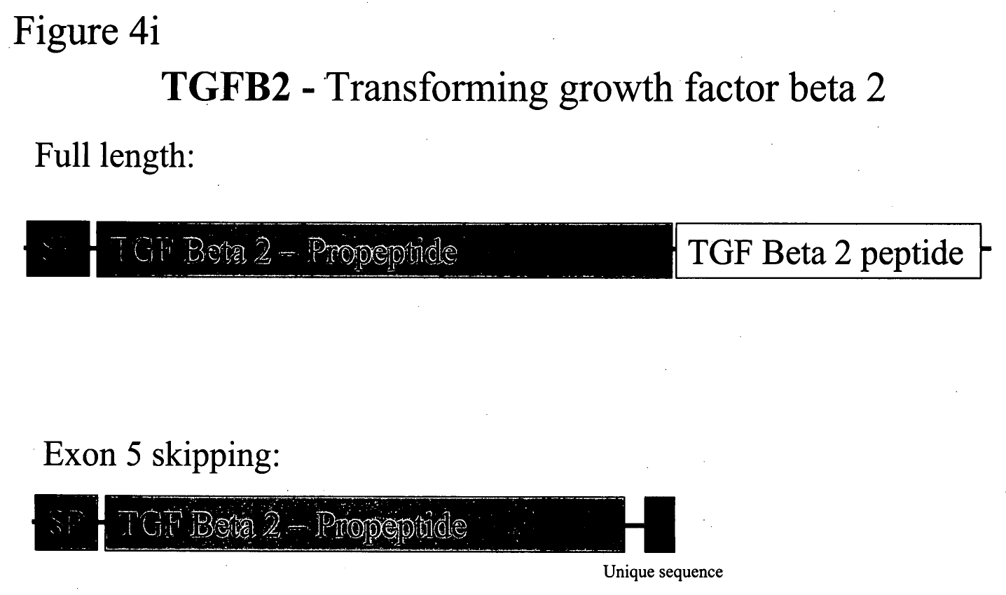

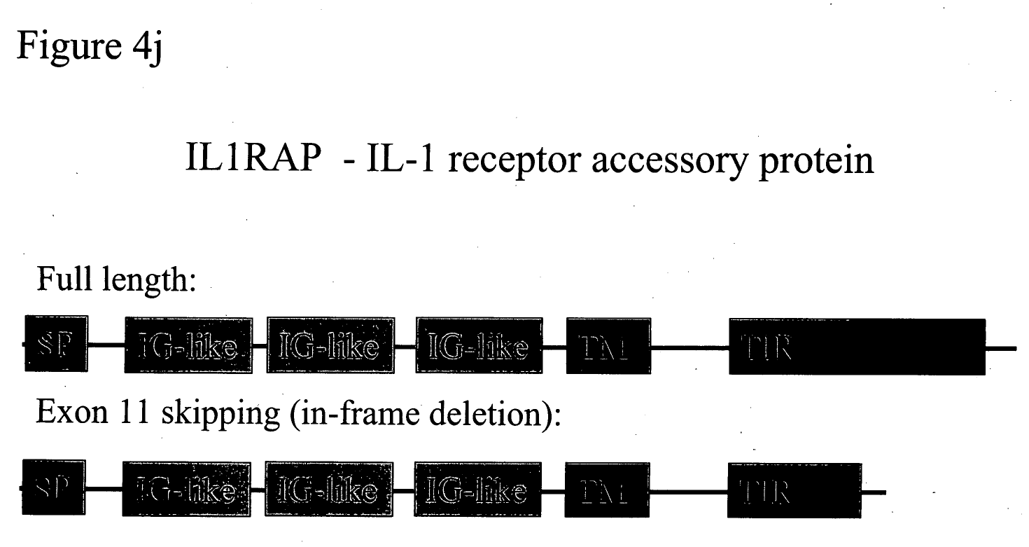

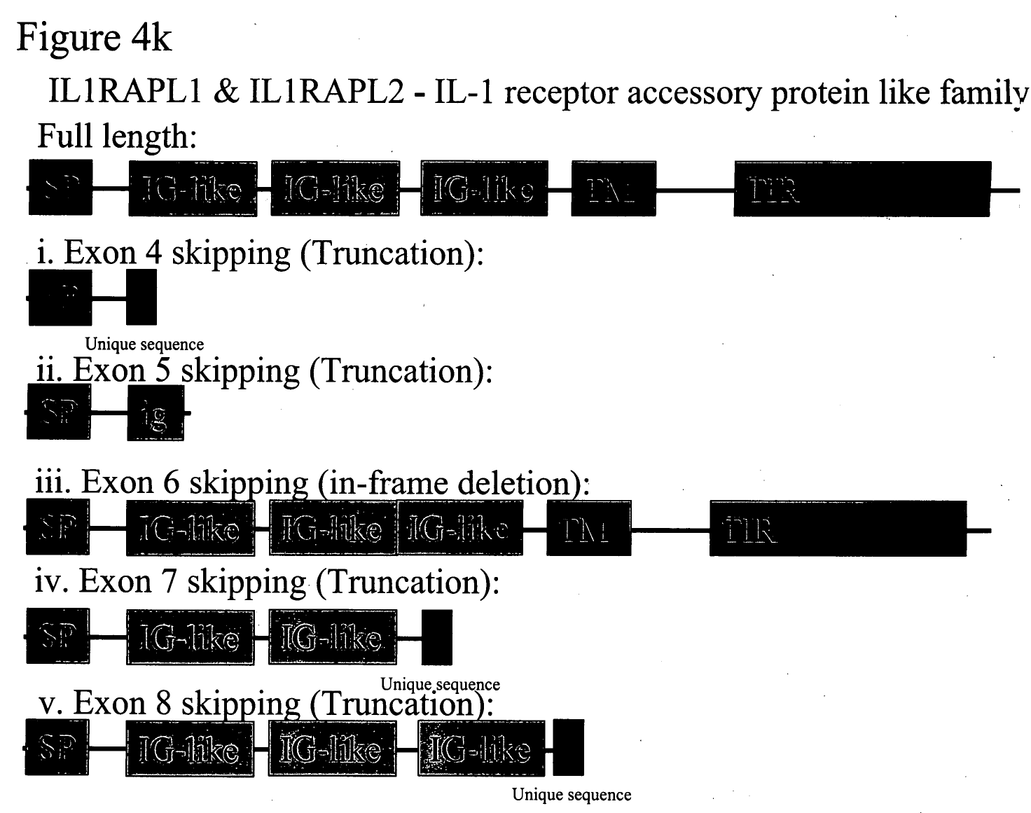

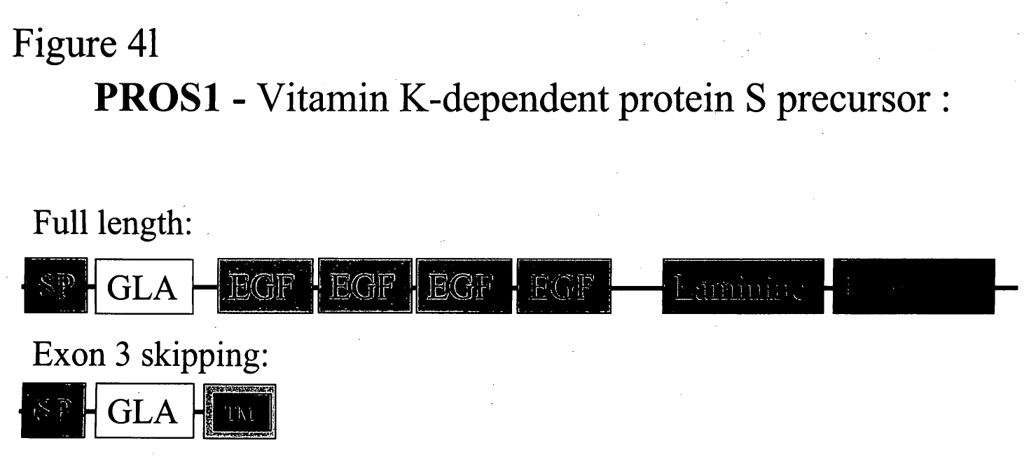

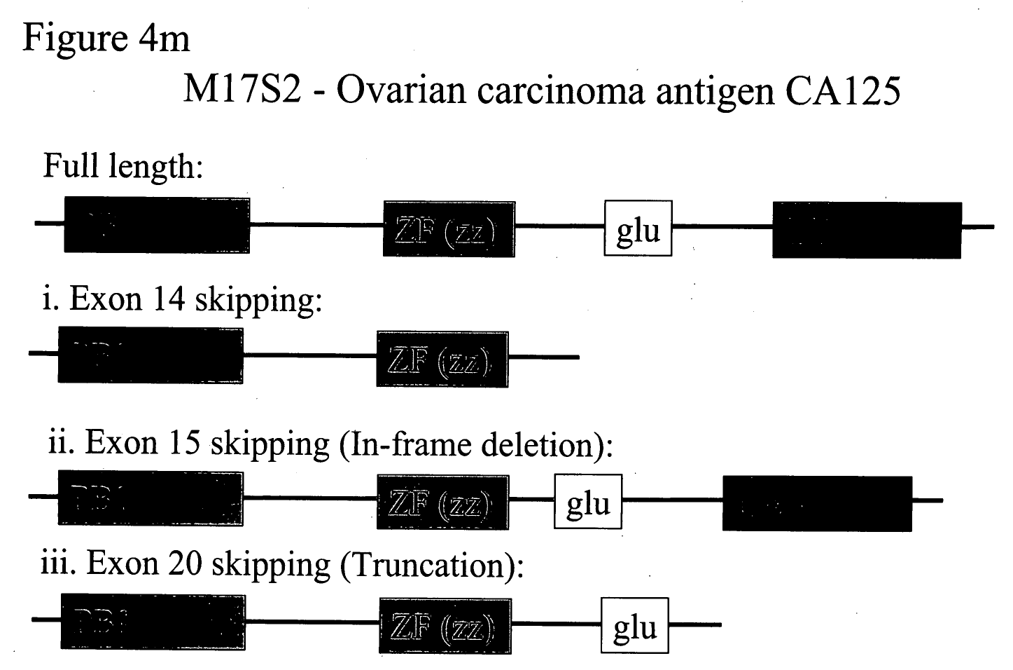

500 clinically relevant genes were scanned and manually annotated. These annotations are listed in Table 3, below. Protein structure of the below listed genes and corresponding splice variants are shown in FIGS. 3a-z and 4a-m.

TABLE 3

| | | | | | | Protein |
|---|---|---|---|---|---|---|
| | | | | | | Product - |
| | Gene name | | Mechanism of | CDs features (incl. | | SEQ ID |
| # | and Swissprot | Examples for indications | splicing | Unique sequence) | #pep_num | NOs: |
| 1 | VLDLR | Some variants could be used as soluble | Skipping exons: 8 | Deletion of EGF | 1 | 23, 273 |
| | Very low density | traps for LDL and as such to reduce risk | 9 | Deletion of EGF | 2 | 24, 274 |
| | Lipoprotein Receptor | of heart diseases, Vascular diseases and | 12 | Truncation - | 3 | 25, 275 |
| | LDVR_HUMAN | hypertension. It could also be used as: | | Soluble receptor | | |
| | | Anti hyperlipidemia | 14 | Truncation - | 4 | 26, 276 |
| | | Anti cholesterol | | soluble receptor | | |
| | | Anti gallstones | 15 | Deletion of EGF | 5 | 27, 277 |
| | | | Retention of intron | Truncation - Soluble | 6 | 28, 278 |
| | | | 8 - see FIG. 2i | receptor | | |
| | | | | Confirmed by | | |
| | | | | sequencing | | |
| 2 | VEGFC | Might be used as agonist for | Skipping exon 4 - | Truncates the protein | 7 | 29, 279 |
| | Vascular Endothelial | cardiovascular diseases and diabetes | see FIG. 2b | within VEGF | | |
| | Growth Factor | (agonist of VEGFR2); | | peptide. Probable | | |
| | VEGC_HUMAN | Might be an antagoinst to VEGF | | Elevation of VEGF2 | | |
| | | receptors | | specificity | | |
| | | and as such be used for treatment of | | Confirmed by | | |
| | | cancer, diabetes and Asthma. | | sequencing | | |
| | | Might also be used for Psoriasis. | | | | |
| 3 | FLT1 | Might be an antagonist to VEGF | Skipping exon | Deletion reduces | 8 | 30, 280 |
| | Vascular endothelial | receptors | 19 | Protein kinase | | |
| | growth factor receptor | and as such be used for treatment of | | domain | | |
| | 1 precursor | cancer, diabetes and Asthma. | | | | |
| | VGR1_HUMAN | Might also be used for Psoriasis. | | | | |
| 4 | KDR | Mostly the two first variants (which | Skipping exon | Truncates the protein | 9 | 31, 281 |
| | Vascular endothelial | might serve as a soluble/anchored decoy | 16(TM) | right before TM | | |
| | growth factor receptor | receptors for VEGF) | | (Soluble receptor) | | |
| | 2 precursor | might serve an antagonist to VEGF | 17 | Truncation deletes all | 10 | 32, 282 |
| | VGR2_HUMAN | receptors | | of the ICD | | |
| | | and as such be used for treatment of | 27 | Truncation doesn't | 11 | 33, 283 |
| | | cancer, diabetes and Asthma. | | affect domain | | |
| | | Might also be used for Psoriasis. | 28 | Truncation doesn't | 12 | 34, 284 |
| | | | | affect domain | | |
| | | | 29 | Truncation doesn't | 13 | 35, 285 |
| | | | | affect domain | | |
| 5 | ITAV | Might be used as Integrin antagonst: | Skipping exon | Truncation - Soluble | 14 | 36, 286 |
| | Integrin alpha-V | Would be used as anti-inflammatory | 11 | Receptor. | | |
| | precursor | (especially for GI), immunosuppressant, | 20 | Truncation - Soluble | 15 | 37,287 |
| | ITAV_Human | anti Asthma and anti cancer. | | Receptor. | | |
| | | | 21 | Deletion in heavy | 16 | 38, 288 |
| | | | | chain | | |
| | | | 25 | Deletion in heavy | 17 | 39, 289 |
| | | | | chain | | |
| 6 | MET | Soluble receptor might serve as MET | Skipping exon | Skipping TM - | 18 | 40, 290 |
| | (HGF receptor) | antagonist. | 12 | Soluble receoptor | | |
| | MET_Human | The variant might be involved in | | (evidence for | | |
| | | prevention of proliferation and | | extension) | | |
| | | prevention of metastases and cell | 14 | Deletion after TM - | 19 | 41, 291 |
| | | motility. It might be used for diabetes, | | may affect TM | | |
| | | skin conditions and for urological | 18 | Truncates most of the | 20 | 42, 292 |
| | | disorders. | | PK domain | | |
| 8 | FSHR | Soluble chain might serve as a | Skipping exon 7 | Deletion of LRR | 26 | 43, 293 |
| | Follicular stimulating | diagnostic marker for fertility and | 8 | Deletion of LRR | 27 | 44,294 |
| | hormone Receptor | menopausal disorders. | intron 7 retention | Truncation - Soluble | 28 | 45, 295 |
| | FSHR_Human | Both truncated forms could also be used | | extracellular Chain | | |
| | | as contraceptives. | Novel exon 8A | Truncation - Soluble | 29 | 46, 296 |
| | | Could also be used for mail fertility | (102 bp) | extracellular Chain - | | |
| | | diagnostic and treatment. | | A unique tail; | | |
| | | | | Validated by | | |
| | | | | sequencing | | |
| 9 | LSHR | Soluble chain might serve as a | Skipping exon 2 | Deletion LRR | 30 | 47, 297 |
| | Lutheizing hormone | diagnostic marker for fertility and | 3 | Deletion LRR | 31 | 48, 298 |
| | receptor | menopausal disorders. | 5 | Deletion LRR | 32 | 49, 299 |
| | LSHR_Human | Both truncated forms could also be used | 6 | Deletion LRR | 33 | 50, 300 |
| | | as contraceptives. | 7 | Deletion LRR | 34 | 51, 301 |
| | | Could also be used for mail fertility | 10 | Deletion LSHR | 35 | 52, 302 |
| | | diagnostic and treatment. | Intron 5 retention | Truncation - Soluble | 36 | 53, 303 |
| | | | | extracellular Chain | | |
| 10 | FGF11 | The soluble form might be used as | Skipping exon 2 - | In-frame Deletion of | 37 | 54, 304 |
| | Fibroblast growth | FGFR agonist/antagonist. Might be used | see FIG. 2d | 37 AA | | |
| | Factor | for treatment of Cancer, cardiovascular | | Validated by | | |
| | FGFB_HUMAN. | diseases and as a growth factor. | | seuqnecing | | |
| | | Deletion might cause Antagonist effect, | | | | |
| | | and thus be used for treatment of cancer | | | | |
| | | as well as diabetes and respiratory | | | | |
| | | conditions. | | | | |
| 11 | FGF12 | The soluble form might be used as | Skipping exon 2 | In-frame Deletion of | 38 | 55, 305 |
| | Fibroblast growth | FGFR agonist/antagonist. Might be used | long isdoform | 37 AA | | |
| | Factor | for treatment of Cancer, cardiovascular | | Soluble secreted form | | |
| | FGFC_HUMAN | diseases and as a growth factor. | Skipping exon 2 | In-frame Deletion of | 39 | 56, 306 |
| | | Deletion might cause Antagonist effect, | short isdoform | 37 AA | | |
| | | and thus be used for treatment of cancer | | Soluble secreted form | | |
| | | as well as diabetes and respiratory | | | | |
| | | conditions. | | | | |
| 12 | FGF13 | The soluble form might be used as | Skipping exon 2 | In-frame Deletion of | 40 | 57, 307 |
| | Fibroblast growth | FGFR agonist/antagonist. Might be used | long isdoform | 37 AA | | |
| | Factor | for treatment of Cancer, cardiovascular | | Soluble secreted form | | |
| | FGFD_HUMAN | diseases and as a growth factor. | Skipping exon 2 | In-frame Deletion of | 40a | 58, 308 |
| | | Deletion might cause Antagonist effect, | short isdoform | 37 AA | | |
| | | and thus be used for treatment of cancer | | Soluble secreted form | | |
| | | as well as diabetes and respiratory | Skipping exon 3 | Truncation of | 41 | 59, 309 |
| | | conditions. | long isdoform | protein. | | |
| | | | Skipping exon 3 | Truncation of | 41a | 60, 310 |
| | | | short isdoform | protein. | | |
| 13 | EFNA1 | Ephrin ligands and receptors have a | Skipping exon 3 | In-frame deletion - | 42 | 61, 311 |
| | Ephrin A | variety of roles in development and | | Reduction of Ephrin | | |
| | EFA1_human | cancer. | | domain | | |
| | | Variant's indication would be either | | | | |
| | | cause or prevent proliferation of certain | | | | |
| | | tissues - treatment of cancer as well as | | | | |
| | | wound healing and anti-inflammatory. | | | | |
| 14 | EFNA3 | Ephrin ligands and receptors have a | Skipping exon 3 | In-frame deletion - | 43 | 62, 312 |
| | Ephrin A | variety of roles in development and | | Reduction of Ephrin | | |
| | EFA3_human | cancer. | | domain. | | |
| | | Variant's indication would be either | 4 | In-frame deletion- | 44 | 63, 313 |
| | | cause or prevent proliferation of certain | | Redaction of Ephrin | | |
| | | tissues - treatment of cancer as well as | | domain. (supported | | |
| | | wound healing and anti-inflammatory. | | by 1 EST) | | |
| 15 | EFNA5 | Ephrin ligands and receptors have a | Skipping exon 3 - | In-frame deletion - | 45 | 64, 314 |
| | Ephrin A | variety of roles in development and | see | Reduction of Ephrin | | |
| | EFA5_human | cancer. | | domain. | | |
| | | Variant's indication would be either | 4 | In-frame deletion. | 46 | 65, 315 |
| | | cause or prevent proliferation of certain | | Reduction of Ephrin | | |
| | | tissues - treatment of cancer as well as | | domain. Validated by | | |
| | | wound healing and anti-inflammatory. | | sequencing | | |
| 16 | EFNB2 | Ephrin ligands and receptors have a | Skipping exon 2 | Truncation of most | 47 | 66, 316 |
| | Ephrin B | variety of roles in development and | | Ephrin domain. | | |
| | EFB2_Human | cancer. | | | | |
| | | Variant's indication would be either. | 3 | Reduction of Ephrin | 48 | 67, 317 |
| | | cause or prevent proliferation of certain | | domain. | | |
| | | tissues - treatment of cancer as well as | 4 | Reduction of | 49 | 68, 318 |
| | | wound healing and-anti-inflammatory. | | distance between | | |
| | | | | Ephrin domain and | | |
| | | | | TM | | |
| 17 | EPHA4 | Ephrin ligands and receptors have a | Skipping exon 2 | Truncation most of | 50 | 69, 319 |
| | Ephrin A receptor | variety of roles in development and | | the protein | | |
| | (Tyrosine Kinase) | cancer. | 3 | Truncation leaving | 51 | 70, 320 |
| | EPA4_Human | Variant's indication would be either | | LBD reduced and a | | |
| | | cause or prevent proliferation of certain | | long unique sequence | | |
| | | tissues - treatment of cancer as well as | 4 | Reducing distance | 52 | 71, 321 |
| | | wound healing and anti-inflammatory. | | LBD-FN III | | |
| | | | 12 | Truncation of SAM | 53 | 72, 322 |
| | | | | and most TK | | |
| 18 | EPHA5 | Ephrin ligands and receptors have a | Skipping exon 4 | Reducing distance | 54 | 73, 323 |
| | Ephrin A receptor | variety of roles in development and | | LBD-FN III | | |
| | (Tyrosine Kinase) | cancer. | | | | |
| | EPA5_Human | Variant's indication would be either | 5 | Abolishes the 1st FN | 55 | 74, 324 |
| | | cause or prevent proliferation of certain | | III | | |
| | | tissues - treatment of cancer as well as | 8 (TM) | Soluble ECD | 56 | 75, 325 |
| | | wound healing and anti-inflammatory. | | (Soluble receptor) | | |
| | | | | and a long unique | | |
| | | | | sequence | | |
| | | | 10 | Truncation of ICD | 57 | 76, 326 |
| | | | | (SAM and TK) | | |
| | | | 14 | Reducing Protein | 58 | 77, 327 |
| | | | | kinase domain | | |
| | | | 16 | Truncation of SAM | 59 | 78, 328 |
| | | | | and most Protein | | |
| | | | | kinase | | |
| | | | 17 | Reduces SAM | 60 | 79, 329 |
| | | | | domain | | |
| 19 | EPHA7 | Ephrin ligands and receptors have a | Skipping exon 10 | Deletion truncates | 61 | 80, 330 |
| | Ephrin A receptor | variety of roles in development and | | most of ICD | | |
| | (Tyrosine Kinase) | cancer. | 15 | Truncation of SAM | 62 | 81, 331 |
| | EPA7_Human | Variant's indication would be either | | and most of the | | |
| | | cause or prevent proliferation of certain | | Protein kinase. | | |
| | | tissues - treatment of cancer as well as | | | | |
| | | wound healing and anti-inflammatory. | | | | |
| 20 | EPHB1 | Ephrin ligands and receptors have a | Skipping exon 6 | Truncated Soluble | 63 | 82, 332 |
| | Ephrin B receptor | variety of roles in development and | | Receptor | | |
| | (Tyrosine Kinase) | cancer. | 8 (TM) | Truncation of ECD- | 64 | 83, 333 |
| | EPB1_Human | Variant's indication would be either | | Soluble Receptor; | | |
| | | cause or prevent proliferation of certain | | long Unique | | |
| | | tissues - treatment of cancer as well as | | sequence | | |
| | | wound healing and anti-inflammatory. | 10- see FIG. 2a | In-frame deletion | 65 | 84, 334 |
| | | | | Reduces Protein | | |
| | | | | kinase - Validated by | | |
| | | | | Sequencing | | |
| 21 | PTPRZ1 | Protein tyrosine phosphatase receotors | Skipping exon 7 | Truncation of most | 66 | 85, 335 |
| | Protein-tyrosine | have a variety of roles in development; | | protein domains | | |
| | phosphatase zeta | metabolism and cancer. Variant's | 11 | Truncation after 2^{nd} | 67 | 86, 336 |
| | PTPZ_Human | indication would be either cause or | | fibronectin | | |
| | | prevent proliferation of certain tissues - | 13 (TM)- | A soluble receptor - | 68 | 87, 337 |
| | | treatment of cancer as well as | see FIG. 2f | validated | | |
| | | cardiovascular disorders and diabetes | 15 | abolishing most of | 69 | 88, 338 |
| | | | | ICD Long Unique | | |
| | | | | sequence | | |
| | | | 16 | doesn't effect any | 70 | 89, 339 |
| | | | | domain | | |
| | | | 22 | abolishes 2nd PTP - | 71 | 90,340 |
| | | | | Long Unique | | |
| 22 | PTPRB | Protein tyrosine phosphatase receotors | Skipping exon 26 | Truncation abolishes | 72 | 91, 341 |
| | Protein-tyrosine | have a variety of roles in development, | | all ICD with a short | | |
| | phosphatase Beta | metabolism and cancer. Variant's | | unique sequence. | | |
| | PTPB_Human | indication would be either cause or | | | | |
| | | prevent proliferation of certain tissues - | | | | |
| | | treatment of cancer as well as | | | | |
| | | cardiovascular disorders and diabetes | | | | |
| 23 | KITLG | Agonist plays a role as antianaemic. | Skipping exon 8 | Truncating C-ter | 73 | 92, 342 |
| | KIT ligand: SCF/MGF | Secreted molecule might be a more | | including TM and | | |
| | SCF_Human | potent agonist for the receptor. | | ICD. Unique | | |
| | | Soluble form might also be used as an | | sequence might add | | |
| | | antagonist and thus prevent proliferation | | an alternative TM. | | |
| | | of blood cells in hematopoietic cancers. | | But may be soluble. | | |
| 24 | KIT | Agonist plays a role as antianaemic. | Skipping exon 8 | Truncation creates | 74 | 93, 343 |
| | KIT_Human | Soluble receptor might be used as an | | Soluble receptor | | |
| | | antagonist and thus prevent proliferation | 14 | Truncation reduces | 75 | 94, 344 |
| | | of blood cells in hematopoietic cancers. | | Protein Kinase | | |
| 25 | ErbB2 | Might serve as a diagnostic marker for | Skipping exon 6 | Truncation of most | 76 | 95, 345 |
| | Receptor Tyrosine | HER2 overexpressing cancer types. | | C-ter (leaving one L- | | |
| | Kinase | Might be used as an antagonist. | | domain and reduced | | |
| | ERB2_Human | | | furin-like domain) - | | |
| | | | | Soluble | | |
| 26 | ErbB3 | Since exon 15 and 18 skipping variants | Skipping exon 4 | Reducing distance L- | 77 | 96, 346 |
| | Receptor Tyrosine | encode soluble receptors which include | | domain - furin | | |
| | Kinase | the ligand binding domain, it is | 15 | Soluble ECD | 78 | 97, 347 |
| | ERB3_Human | suggested that such proteins may serve | | (reduced 2^{nd} furin) - | | |
| | | as antagonists for all EGFR family genes | | Soluble receptor | | |
| | | which undergo heterodimerization as | 18 | Deletion reduces | 79 | 98, 348 |
| | | part of their activation. | | Protein kinase | | |
| | | | | domain. | | |
| 27 | ErbB4 | Especially skipping exon 14 might serve | Skipping exon 14 | Soluble ECD | 80 | 99, 349 |
| | Receptor Tyrosine | as a good antagonist for all EGFR | | (reduced 2^{nd} furin) - | | |
| | Kinase | family genes. | | Soluble receptor | | |
| | ERB4_Human | Might serve as ERBB2 antagonist (also | 16 | Reducing 2^{nd} furin | 81 | 100, 350 |
| | | for EGFR, ERBB3 and ERBB4) | | like domain | | |
| 28 | NRG1 incl forms: | As many of the NRG1 isoforms serve as | HGR-α, HGR β1 | (Known in some | 82 | 101, 351 |
| | HGR-α, HGR-β1, | ErbB1/3/4 (EGFR family) ligands. | HGR β2 | isoforms, but not in | 83 | 102, 352 |
| | HGR-β2, | Most variants might be used as | HGR β3 | others): Deletion | 84 | 103, 353 |
| | HGR-β3, HGR-γ, | partial/full antagonists of these cancer | HGR γ | Reduces distance | 85 | 104, 354 |
| | HGR-GGF, NDF43 | related receptors | HGR-GGF, | between EGF - Ig | 86 | 105, 355 |
| | Neuregulin Variants | The indication might therefore be (in | NDF43 | like domain. | | |
| | NRG1_Human | some of the cases) for cancer treatment | Skipping exon 5 | Truncation abolishes | 87 | 106, 356 |
| | | and diagnosis. | HGR-β2, | NRG family domain. | 88 | 107, 357 |
| | | In some cases, some forms could serve | Skipping exon 8 | (Truncates HGR-β1 | | |
| | | as agonists, to enhance cell proliferation | HGR-β1 | to be like the shorter | 89 | 108, 358 |
| | | (especially for wound healing). | Skipping exon 9 | isoforms). | | |
| | | | HGR-α, | Truncation abolishes | 90 | 109, 359 |
| | | | HGR-β1, | NRG finnily domain. | | |
| | | | NDF43 | (Truncates HGR-β1 | | |
| | | | Skipping exon 7 | to be like the shorter | 91 | 110, 360 |
| | | | NDF43 | isoforms). | 92 | 111, 361 |
| | | | Skipping exon 12 | Truncation abolishes | 93 | 112, 362 |
| | | | HGR-β1 | NRG and EGF | 94 | 113, 363 |
| | | | Skipping exon 8 | domains | 95 | 114, 364 |
| | | | | (ln NDF43 adds a | | |
| | | | | long unique). | | |
| | | | | Truncates and adds a | | |
| | | | | long unique sequence | | |
| | | | | which is identical to | | |
| | | | | the HGR-β1 isoform, | | |
| | | | | and recreates the | | |
| | | | | NRG domain. | | |
| | | | | Reduces distance | | |
| | | | | between EGF and | | |
| | | | | NRG. | | |
| 29 | JAG1 | Has a known indication for | Skipping exon 10 | Deletion of 4th | 96 | 115, 365 |
| | Jagged-regulator of | atherosclerotic diseases. JAG1 | | EGF domain | | |
| | Angiogenesis | antagonist (especially. Soluble receptor) | 12 | Deletion of 5th & 6th | 97 | 116, 366 |
| | JAG1_Human | might serve in preventing/treating | | EGF domains | | |
| | | cardiovascular diseases and cancer. | 18 | Deletion of 12th | 98 | 117, 367 |
| | | | | EGF domain | | |
| | | | | (extention creates a | | |
| | | | | soluble receptor, but | | |
| | | | | is known) | | |
| | | | 22 | Truncation creates a | 99 | 118, 368 |
| | | | | soluble receptor with | | |
| | | | | a long unique | | |
| | | | | sequence. | | |
| 30 | NOTCH2 | NOTCH agonists are indicated for | Skipping exon 9 - | abolishes one EGF - | 100 | 119, 369 |
| | Neurogenic locus notch | AntiAsthma and immunosuppressants. | seeFIG. 2e | like repeat. | | |
| | homolog protein | Might also be diagnostic markers for | 12 | abolishes one EGF - | 101 | 120, 370 |
| | NTC2_Human | mental illnesses. | | like repeat. | | |
| 31 | NOTCH3 | NOTCH agonists are indicated for | Skipping exon 2 | Truncates entire | 102 | 121, 371 |
| | Neurogenic locus notch | AntiAstluna and immunosuppressants. | | protein leaving only | | |
| | homolog protein | Might also be diagnostic markers for | | SP with a long | | |
| | NTC3_Human | mental illnesses. | | different, unique, AA | | |
| | | | | sequence. | | |
| 32 | NOTCH4 | NOTCH agonists are indicated for | Skipping exon 8 | abolishes two EGF - | 103 | 122, 372 |
| | Neurogenic locus notch | AntoAsthma and immunosuppressants. | | like repeats | | |
| | homolog protein | Might also be diagnostic markers for | | | | |
| | NTC4_Human | mental illnesses. | | | | |
| 33 | NTRK2 | Agonist/partial agonist might play a role | Skipping exon | In-frame deletion, | 104 | 123, 373 |
| | BDNF/NT-3 growth | in CNS related diseases such as | 14 FIG. 2g | Doesn't affect a | | |
| | factor receptor | Parkinson, Alzheimer and other | | domain - Validated | | |
| | TRKB_HUMAN | disorders. As well as a memory | | by sequencing. | | |
| | | enhancer and neuroprotective. | | | | |
| | | Antagonist might also be a mental | | | | |
| | | treatment. | | | | |
| 34 | NTRK3 | Agonist/partial agonist might play a role | Skipping exon 5 | Deletion abolishes | 105 | 124, 374 |
| | NT-3 growth factor | in CNS related diseases such as | | two short LRRs | | |
| | receptor | Parkinson, Alzheimer and other | 16 | Truncation reduces | 106 | 125, 375 |
| | TRKC_HUMAN | disorders. As well as a memory | | the PK domain | | |
| | | enhancer and neuroprotective | | | | |
| | | Antagonist might also be a mental | | | | |
| | | treatment. | | | | |
| 35 | GFRA1 | Agonist might serve as a neuroprotective | Skipping exon 4 (3 | Reduces GDNF | 107 | 126, 376 |
| | RET ligand | agent. | in CDs) | receptor family | | |
| | GDNF receptor | Thus might have a role in preventing | | | | |
| | GDNR_HUMAN | Parkinson and other CNS related | | | | |
| | | disorders. | | | | |
| 36 | GFRA2 | Agonist might serve as a neuroprotective | Skipping exon 3 | Reduces GDNF | 108 | 127, 377 |
| | RET ligand | agent. | | receptor family | | |
| | GDNF receptor | Thus might have a role in preventing | | | | |
| | NRTR_Human | Parkinson and other CNS related | | | | |
| | | disorders. | | | | |
| 37 | IL16 - Long | Both agonist and antagonist might have | Skipping exon 5 | Truncates the | 109 | 128, 378 |
| | Interleukin 16 long | a role in treating cancer and | | protein, leaving no | | |
| | variant | inflammation, antagonist would be used | | domains | | |
| | IL16_human | for Asthma. | 18 (5 in shorter | Deletion reduces | 110 | 129, 379 |
| | | | isoform) | 3rd (1st) PDZ | | |
| | | | | domain | | |
| 38 | IGFBP4 | Might serve as an enhancer for Insulin | Skipping exon 3 | Deletion reduces | 111 | 130, 380 |
| | Insulin Growth factor | growth factor. Might thus have an affect | | Thyroglobulin type-1 | | |
| | binding protein | as a Growth hormone and on diseases | | repeat domain | | |
| | IBP4_Human | such as: | | | | |
| | | Osteoporosis and MS. | | | | |
| 39 | NRP1 | Much like VEGF and VEGFR genes, | Skipping exon 5 | Deletion reduces the | 112 | 131, 381 |
| | Neuropilin-1 precursor | indication for preventing angiogenesis | | CUB domain | | |
| | NRP1_HUMAN | (for treatment of cancer) and inducing | | | | |
| | | angiogenesis (for cardiovascular and | | | | |
| | | ischemia diseases). | | | | |
| 40 | FGF9 | The soluble form might be used as | Skipping exon 2 | Truncation reduces | 113 | 132, 382 |
| | Fibroblast growth | FGFR agonist/antagonist. Might be used | | FGF domain | | |
| | factor | for treatment of Cancer, cardiovascular | | (creating a unique | | |
| | FGF9_Human | diseases and as a growth factor. | | putative hydrophilic | | |
| | | Deletion might cause Antagonist effect, | | tail) | | |
| | | and thus be used for treatment of cancer | | | | |
| | | as well as diabetes and respiratory | | | | |
| | | conditions. | | | | |
| 41 | FGF10 | The soluble form might be used as | Skipping exon 2 | Truncation reduces | 114 | 133, 383 |
| | Fibroblast growth | FGFR agonist/antagonist. Might be used | | FGF domain | | |
| | factor | for treatment of Cancer, cardiovascular | | (creating a unique | | |
| | FGFA_Human | diseases and as a growth factor. | | putative hydrophilic | | |
| | | Deletion might cause Antagonist effect, | | tail) | | |
| | | and thus be used for treatment of cancer | | | | |
| | | as well as diabetes and respiratory | | | | |
| | | conditions. | | | | |
| 42 | FGF18 | The soluble form might be used as | Skipping exon 2 | Truncated protein | 115 | 134, 384 |
| | Fibroblast growth | FGFR agonist/antagonist. Might be used | 4 | Truncation reducing | 116 | 135, 385 |
| | factor | for treatment of Cancer, cardiovascular | | FGF domain | | |
| | FGFI_Human | diseases and as a growth factor. | | (creating a unique | | |
| | | Deletion might cause Antagonist effect, | | putative hydrophilic | | |
| | | and thus be used for treatment of cancer | | tail) | | |
| | | as well as diabetes and respiratory | | | | |
| | | conditions. | | | | |
| 43 | ANGPT1 | Agonist of Angiopoietin might serve for | Skipping exon 5 | Truncation of the | 117 | 136, 386 |
| | Angiopoietin-1 | therapy of cardiovascular diseases as | | Fibrinogen-C | | |
| | AGP1_HUMAN | well as cancer. | | terminal domain | | |
| | | | 6 | Deletion reduces | 118 | 137, 387 |
| | | | | Fibrinogen-C | | |
| | | | | terminal domain | | |
| | | | 8 (in long isoform) | Truncation reduces | 119 | 138, 388 |
| | | | | Fibrinogen-C | | |
| | | | | terminal domain | | |
| 45 | EDNRB | Antagonist would have a role in | Skipping exon 4 | reduction in the 7 | 128 | 139, 389 |
| | Endothelin B receptor | cardiovascular diseases. | | transmembrane | | |
| | ETBR_human | | | receptor (rhodopsin | | |
| | | | | family) domain | | |
| 46 | ECE1 | Antagonist would be useful in | Skipping exon 2 | Deletion would | 129 | 140, 390 |
| | Endothelin converting | respiratory diseases, it might have | | convert Signal | | |
| | Enzyme | diuretic effect and thus be used for | | Peptide to a Signal | | |
| | ECE1_HUMAN | hypertention and cardiovascular | | anchor. | | |
| | | diseases. | | | | |
| 47 | ECE2 | Antagonist would be useful in | Skipping exon 2 | Deletion would | 130 | 141, 391 |
| | Endothelin converting | respiratory diseases, it might have | | convert Signal | | |
| | Enzyme | diuretic effect and thus be used for | | Peptide to a Signal | | |
| | ECE2_HUMAN | hypertention and cardiovascular | | anchor. (Known) | | |
| | | diseases. | 8 | Deletion reduces | 131 | 142, 392 |
| | | | | M13 peptidase N | | |
| | | | 12 | Deletion reduces | 132 | 143, 393 |
| | | | | M13 peptidase N | | |
| | | | 13 | Deletion reduces | 133 | 144, 394 |
| | | | | M13 peptidase N | | |
| | | | 15 | Deletion reduces | 134 | 145, 395 |
| | | | | M13 peptidase C | | |
| 48 | ITGA2B | Might be used as Integrin antagonist: | Skipping exon 3 | Truncation abolishes | 135 | 146, 396 |
| | Integrin alpha-Iib | Indicated for cardiovascular diseases. | | most of the protein | | |
| | ITAB_Human | | | including most of | | |
| | | | | FG-GAP repeats (1 | | |
| | | | | EST skips exons 2-4) | | |
| 49 | MPL | Might be used as a diagnostic agent for | Skipping exon 2 | Truncation of most of | 136 | 147, 397 |
| | Thrombopoietin | hematological diseases, as well as | | the protein | | |
| | receptor | therapy as a growth factor and antiviral. | | | | |
| | TPOR_HUMAN | | | | | |
| 50 | CUL5 | Variants might be used as Vasopressin | Skipping exon 2 | Truncation reduces | 137 or 138 | 148 or |
| | Cullin homolog 5 | antagonists for treatment of Diabetes, | | the CULLIN domain | | 149/398 |
| | Vasopressin-activated | cardiovascular diseases (Diuretic for | 8 | Truncation reduces | 139 | 150, 399 |
| | calcium-mobilizing | hypertension) and as an antidepressant. | | the CULLIN domain | | |
| | receptor | | | | | |
| | VAC1_HUMAN | | | | | |
| 51 | HPA | As Agonist this protein might serve for | Skipping exon 10 | Truncation slightly | 140 | 151, 400 |
| | Heparanase | treatment of Cystic Fibrosis. | | reduces Glycosyl | | |
| | Q9Y251 | As antagonist it is indicated for Cancer | | hydrolase domain. | | |
| | | (anti metastatic), cardiovascular and MS. | | | | |
| 52 | HPSE2 | As Agonist this protein might serve for | Skipping 5 | Truncation reduces | 141 | 152, 401 |
| | Heparanase 2 | treatment of Cystic Fibrosis | | Glycosyl hydrolase | | |
| | Q8WWQ2 | As antagonist it is indicated for Cancer | | domain | | |
| | Q8WWQ1 | (anti metastatic), cardiovascular and MS. | 6 | Deletion reduces | 142 | 153, 402 |
| | | | | Glycosyl hydrolase | | |
| | | | | domain | | |
| | | | 7 | Truncation reduces | 143 | 154, 403 |
| | | | | Glycosyl hydrolase | | |
| | | | | domain | | |
| | | | 8 | Truncation reduces | 144 | 155, 404 |
| | | | | Glycosyl hydrolase | | |
| | | | | domain | | |
| | | | 9 | Truncation reduces | 145 | 156, 405 |
| | | | | Glycosyl hydrolase | | |
| | | | | domain | | |
| | | | 10 | Truncation reduces | 146 | 157, 406 |
| | | | | Glycosyl hydrolase | | |
| | | | | domain | | |
| | | | 11 | Deletion doesn't | 147 | 158, 407 |
| | | | | affect Glycosyl | | |
| | | | | hydrolase | | |
| 55 | MME | As an antagonist, these variant might be | Skipping exon 4 | Deletion reduces N- | 150 | 159, 408 |
| | Neutral endopeptidase | used for treatment of Hypertension (a | | ter M13 peptidase | | |
| | (Enkephalinase) | diuretic agent), as a cardiostimulant, as | 7 | Truncation reduces | 151 | 160, 409 |
| | NEP_HUMAN | antidepressant and for treatment of | | N-ter M13 peptidase | | |
| | | Migraine. | | and abolishes C-ter | | |
| | | | | M13 peptidase | | |
| | | | 9 | Deletion reduces N- | 152 | 161, 410 |
| | | | | ter M13 peptidase | | |
| | | | 11 | Truncation reduces | 153 | 162, 411 |
| | | | | N-ter M13 peptidase | | |
| | | | | and abolishes C-ter | | |
| | | | | M13 peptidase. | | |
| | | | 12 | Truncation reduces | 154 | 163, 412 |
| | | | | N-ter M13 peptidase | | |
| | | | | and abolishes C-ter | | |
| | | | | M13 peptidase. | | |
| | | | 16 | Truncation abolishes | 155 | 164, 413 |
| | | | | C-terminal M13 | | |
| | | | | peptidase. | | |
| 56 | APBB1 | Antagonist to the amiloid 4a might be | Skipping exon 3 | Truncation abolishes | 156 | 165, 414 |
| | Alzheimer's disease | used as a neuroprotective agent, to help | | most of the protein | | |
| | amyloid A4 binding | prevent/treat Alzheimer, Parkinson and | | (Extended EST) | | |
| | protein | other neurodegradative diseases. I might | 7 | Deletion reduces 1st | 157 | 166, 415 |
| | ABB1_HUMAN | also be used for hypertention, and as an | | PID domain | | |
| | | anti-inflammatory agent. | 9 | Deletion reduces 1st | 158 | 167, 416 |
| | | | | PID domain | | |
| | | | | (Extended EST) | | |
| | | | 10 | Truncation abolishes | 159 | 168, 417 |
| | | | | 2^{nd} PID reduces 1st | | |
| | | | | PID Domain | | |
| | | | 12 | Truncation abolishes. | 160 | 169, 418 |
| | | | | 2^{nd} PID domain - | | |
| | | | | Adds a Cys rich | | |
| | | | | unique sequence. | | |
| 57 | GDNF | Anti Parkinson. | Skipping exon 2 | Unknown as exon 2 | | 170, 419 |
| | GDNF_HUMAN | | | is last. | | |
| 58 | SCTR | Agonist has haemostatic affects | Skipping exon 10 | Truncation reduces 7 | 162 | 171, 420 |
| | Secretin receptor | (clotting) and some neurological | | transmembrane | | |
| | SCRC_HUMAN | functions. | | receptor (Secretin | | |
| | | | | family) (eliminates | | |
| | | | | last two TM) | | |
| 59 | RSU1 | Might have anti-cancer affect. | Skipping exon 6 | Truncation eliminates | 163 | 172, 421 |
| | Ras suppressor protein 1 | Might serve as a diagnostic marker. | | 3/7 LRR repeats. | | |
| | RSU1_human | | | | | |
| 60 | IL18R | Antagonist has an anti-inflammatory | Skipping exon 9 | Deletion abolishes all | 164 | 173, 422 |
| | Interleukine 18 | effect, might be useful for arthritis and | | of TIR domain | | |
| | receptor | MS. | | (NFkB activating) | | |
| | IR18_Human | | | | | |
| 61 | TGFB2 | Might only be used as a diagnostic | Skipping exon 5 | Truncation abolishes | 165 | 174, 423 |
| | Transforming growth | marker as the variant is basically the | | TGFB peptide and | | |
| | factor beta 2 | Propeptide, Might be used for cancer or | | slightly reduces propeptide. | | |
| | TGF2_Human | respiratory related diseases. | | | | |
| 62 | TIAF1 | An agonist might be used for anti cancer | Skipping exon 11 | Deletion (4AA) | 166 | 175, 424 |
| | (TGFB1-induced anti- | or as an immunosuppressant. | | reduces Myosin head | | |
| | apoptotic factor 1) | An antagonist mught be used for cancer, | | (motor domain) | | |
| | TIAF_HUMAN | Asthma, MS, Cardiovascular diseases | 25 | Deletion doesn't | 167 | 176, 425 |
| | | and respiratory | | affect a domain. | | |
| | | | 34 | Deletion doesn't | 168 | 177, 426 |
| | | | | affect a domain. | | |
| 63 | IL1RAP | Many indications associated with IL1 | Skipping exon 11 | Deletion reduces TIR | 169 | 178, 427 |
| | IL-1 receptor accessory | and IL1 family proteins | | domain | | |
| | protein | The most prevalent indication is as an | | | | |
| | O14915 | antagonist for anti-inflammatory | | | | |
| | | pusposes (Such as MS, Diabetes, Cancer | | | | |
| | | and Arthritis). As both agonist and | | | | |
| | | antagonist might be good for cancer, | | | | |
| | | cardiovascular diseases and | | | | |
| | | antiinflammatory. | | | | |
| 64 | IL1RAPL1 | Many indications associated with IL1 | Skipping exon 4 | Truncation | 170 | 179, 428 |
| | IL-1 receptor accessory | and IL1 family proteins. | | abolishes most of the | | |
| | protein like 1 | The most prevalent indication is as an | | protein | | |
| | Q9UJ53 | antagonist for anti-inflammatory | 5 | Truncation | 171 | 180, 429 |
| | | purposes (Such as MS, Diabetes, Cancer | | abolishes most of the | | |
| | | and Arthritis). As both agonist and | | protein | | |
| | | antagonist, might be good for cancer, | 6 | Deletion reduces | 172 | 181, 430 |
| | | cardiovascular diseases and | | distance: Ig2-3 | | |
| | | antiinflammatory. | | | | |
| | | | 7 | Truncation bolishes | 173 | 182, 431 |
| | | | | ICD and 1 Ig | | |
| | | | | (Soluble receptor) | | |
| | | | 8 | Truncation creates | 174 | 183, 432 |
| | | | | a soluble receptor | | |
| | | | | with 3 Ig-like | | |
| | | | | domains | | |
| 65 | IL1RAPL2 | Many indications associated with IL1 | Skipping exon 4 | Truncation | 175 | 184, 433 |
| | IL-1 receptor accessory | and IL1 family proteins. | | abolishes most of the | | |
| | protein like 2 | The most prevalent indication is as an | | protein. | | |
| | Q9NP60 | antagonist for anti-inflammatory | 5 | Truncation | 176 | 185, 434 |
| | | purposes (Such as MS, Diabetes, Cancer | | abolishes most of the | | |
| | | and Arthritis). As both agonist and | | protein | | |
| | | antagonist might be good for cancer, | 6 | Deletion reduces | 177 | 186, 435 |
| | | cardiovascular diseases and | | distance: Ig2-3 | | |
| | | antiinflammatory. | 7 | Truncation bolishes | 178 | 187, 436 |
| | | | | ICD and 1 Ig | | |
| | | | | (Soluble receptor) | | |
| | | | 8 | Truncation creates a | 179 | 188, 437 |
| | | | | soluble receptor with | | |
| | | | | 3 Ig-like domains | | |
| 66 | THBS1 | Can be used as an anticancer treatment | Skipping exon 4 | Truncation | 180 | 189, 438 |
| | Thrombospondin 1 | both as antagonist and as agonist. | | abolishes all domains | | |
| | precursor | Antagonist is useful against, | | but Thrombospondin | | |
| | TSP1_HUMAN | proliferation, and agonist as an anti- | | N-terminal-like | | |
| | | inflammatory. | | domain (reduced) | | |
| | | | 7 | Truncation | 181 | 190, 439 |
| | | | | abolishes all TSP and | | |
| | | | | EGF domains leaving | | |
| | | | | only the | | |
| | | | 9 | Thrombospondin N- | 182 | 191, 440 |
| | | | | terminal-like domain | | |
| | | | | and a reduced VWC. | | |
| | | | | A very long Unique | | |
| | | | | tail. | | |
| | | | 12 | Deletion abolishes | 183 | 192, 441 |
| | | | | 1st TSP1 repeat. | | |
| | | | | Deletion doesn't | | |
| | | | | affect a domain. | | |
| 67 | THBS4 | Can be used as an anticancer treatment | Skipping exon 15 | Truncation abolishes | 184 | 193, 442 |
| | Thrombospondin 4 | both as antagonist and as agonist. | | 6 TSP3 domain and | | |
| | precursor | Antagonist is useful against | | the entireTSO - C | | |
| | TSP4_HUMAN | proliferation, and agonist as an anti- | | domain. No Unique! | | |
| | | inflammatory, | | | | |
| 68 | PROS1 | Indication for blood clotting - might | Skipping exon 3 | Truncation of most | 185 | 194, 443 |
| | Vitamin K-dependent | serve as an antagonist for Fibrinogen, | | protein. Leaving only | | |
| | protein S precursor | and as a stimulant for TPA (anti | | SP and 77 AA as | | |
| | PRTS_HUMAN | clotting). | | reduced GLA | | |
| | | | | Domain. | | |
| 69 | VWF | Could serve as agonist and/or antagonist | Skipping exon 8 | Deletion abolishes | 186 | 195, 444 |
| | Von Willebrand factor | for clotting factor VIII. As such might | | the 1st TIL domain. | | |
| | precursor | be used for hematodynamic indications, | 13 | Trunaction abolishes | 187 | 196, 445 |
| | VWF_HUMAN | including anti-thrombosis and anti- | | all C-terminus of the | | |
| | | bleeding. | | protein including all | | |
| | | | | domains but two | | |
| | | | | WVD domains and | | |
| | | | | oneTIL | | |
| | | | 29 | Deletion doesn't | 188 | 197, 446 |
| | | | | affect a domain. | | |
| 70 | M17S2 Ovarian | A diagnostic marker for mostly Ovarian | Skipping exon 14 | Truncation doesn't | 189 | 198, 447 |
| | carcinoma antigen | cancer. The variants could be indicated | | affect a domain. | | |
| | CA125 | for other types of cancer. | 15 | Deletion doesn't | 190 | 199, 448 |
| | M172_HUMAN | | | affect a domain. | | |
| | | | 20 | No Unique | 191 | 200, 449 |

### Example 4

### Finding Novel Proteins Using Cross Species Homology

Mouse expressed sequences were aligned to the human genome. Alignments were filtered by a minimal length criterion, and remaining alignments were used to generate “corrected” expressed sequences (by concatenating the fragments of human genomic sequence to which a mouse expressed sequence aligned). These corrected sequences were clustered together with human expressed sequences and the resulting clusters were assembled and subjected to a process of transcript prediction. Within the set of resulting transcripts, transcripts were identified, which cannot be predicted using only human expressed sequences.

Specifically, the following method was performed:

1. Human, mouse and rat ESTs and cDNAs were obtained from NCBI GenBank versions 136 (Jun. 15, 2003) ftp://ftp.ncbi.nih.gov/genbank/release.notes/gb136.release.notes) and NCBI genome assembly of April 2003. Using the LEADS clustering and assembly system as described in Sorek et al. (2002), the expressed sequences were cleaned from repeats, vectors and immunoglobulins, and then aligned to the NCBI human genome reference build 33 (April 2003). The best genomic location was chosen for each human expressed sequence. The human sequences were clustered by genome location. Some clusters were separated in cases of suspected over-clustering or overlapping antisense clusters.

2. Mouse and rat expressed sequences may have more than one alignment to the human genome. All alignments were considered except those shorter than 50 base pairs and unspliced. For further analysis only alignments that overlap human clusters were selected.

3. Each mouse or rat alignment was replaced by the corresponding human DNA sequence, such that problems of low id entity alignments do not interfere with the analysis.

4. Human expressed sequences were grouped in each cluster with all the mouse/rat-originated sequences overlapping it. These groups were then assembled to form new hybrid clusters, taking into account alternative splicing.

5. A list of reliable transcripts was compiled for each of the clusters, filtering suspected intron contaminations and giving preference to canonical splice signals.

6. Alternative splicing events that are supported by non-human sequences only were searched. A list of the transcripts fat contains these events was then compiled.

7. Proteins for these transcripts were predicted.

### Example 5

### Annotation of Computationally Identified Alternatively Spliced Sequences

Newly uncovered naturally occurring transcripts were annotated using the GeneCarta (Compugen, Tel-Aviv, Israel) platform. The GeneCarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

Brief description of the methodology used to obtain annotative sequence information is summarized infra (for a detailed description see U.S. patent application Ser. No. 10/426,002, filed on Apr. 30, 2003 and owned in common with the present application, hereby incorporated by reference as if fully set forth herein).

The ontological annotation approach—An ontology refers to the body of knowledge in a specific knowledge domain or discipline such as molecular biology, microbiology, immunology, virology, plant sciences, pharmaceutical chemistry, medicine, neurology, endocrinology, genetics, ecology, genomics, proteomics, cheminformatics pharmacogenomics, bioinformatics, computer sciences, statistics, mathematics, chemistry, physics and artificial intelligence.

An ontology includes domain-specific concepts—referred to, herein, as sub-ontologies. A sub-ontology may be classified into smaller and narrower categories. The ontological annotation approach is effected as follows.

First, biomolecular (i.e., polynucleotide or polypeptide) sequences are computationally clustered according to a progressive homology range, thereby generating a plurality of clusters each being of a predetermined homology of the homology range.

Progressive homology is used to identify meaningful homologies among biomolecular sequences and to thereby assign new ontological annotations to sequences, which share requisite levels of homologies. Essentially, a biomolecular sequence is assigned to a specific cluster if displays a predetermined homology to at least one member of the cluster (i.e., single linkage). A “progressive homology range” refers to a range of homology thresholds, which progress via predetermined increments from a low homology level (e.g. 35%) to a high homology level (e.g. 99%).

Following generation of clusters, one or more ontologies are assigned to each cluster. Ontologies are derived from an annotation preassociated with at least one biomolecular sequence of each cluster; and/or generated by analyzing (e.g., text-mining) at least one biomolecular sequence of each cluster thereby annotating biomolecular sequences.

Sequence annotations obtained using the above-described methodologies and other approaches are disclosed in a data table in the file AnnotationForPatent.txt of the enclosed CD-ROM 1.

### Example 6

### Description of Data

Following is a description of the data table in “AnnotationForPatent.txt” file, on the attached CD-ROM1. The data table shows a collection of annotations for biomolecular sequences, which were identified according to the teachings of the present invention using transcript data based on GenBank versions Genbank version 136 (Jun. 15, 2003 ftp://ftp.ncbi.nih.gov/genbank/release.notes/gb136.release.notes.

Each feature in the data table is identified by “#”.

The sequences in this patent application are additional information to the Gencarta contigs. Therefore, all annotations that re in terms of Gencarta contigs were also assigned to the sequences in this patent that are derived from these contigs. Also, annotations that are applied by comparing proteins resulting from the same contig were adapted by comparing the sequences in this patent to the proteins from the originals Gencarta contig.

#INDICATION—This field designates the indications and therapies that the polypeptide of the present invention can be utilized for. The indications state the disorders/disease that the polypeptide can be used for and the therapy is the postulated mode of action of the polypeptide for the indication. For example, an indication can be “Cancer, general” while the therapy will be “Anticancer”. Each Gencarta contig was assigned a SWISSPROT and/or TremB1 human protein accession as described in section “Assignment of Swissprot/TremB1 accessions to Gencarta contigs” hereinbelow. The information contained in this field is the indication concatenated to the therapies that were accumulated for the SWISSPROT and/or TremB1 human protein from drug databases, such as PharmaProject (PJB Publications Ltd 2003 http://www.pjbpubs.com/cms.asp?pageid=340) and public databases, such as LocusLink (http://www.genelynx.org/cgi-bin/resource?res=locuslink) and Swissprot (http://www.ebi.ac.uk/swissprot/index.html). The field may comprise more than one term wherein a “,” separates each adjacent terms.

Example—#INDICATION Alopecia, general; Antianginal; Anticancer, immunological; Anticancer, other; Atherosclerosis; Buerger's syndrome; Cancer, general; Cancer, head and neck; Cancer, renal; Cardiovascular; Cirrhosis, hepatic; Cognition enhancer; Dermatological; Fibrosis, pulmonary; Gene therapy; Hepatic dysfunction; general Hepatoprotective; Hypolipaemic/Antiatherosclerosis; Infarction, cerebral; Neuroprotective; Ophthalmological; Peripheral vascular disease; Radio/chemoprotective; Recombinant growth factor; Respiratory; Retinopathy, diabetic; Symptomatic antidiabetic; Urological;

Assignment of Swissprot/TremB1 accessions to Gencarta contigs—Gencarta contigs were assigned a Swissprot/TremB1 human accession as follows. Swissprot/TremB1 data were parsed and for each Swissprot/TremB1 accession (excluding Swissprot/TremB1 that are annotated as partial or fragment proteins) cross-references to EMBL and Genbank were parsed. The alignment quality of the Swissprot/TremB1 protein to their assigned mRNA sequences was checked by frame+p2n alignment analysis. A good alignment was considered as heving the following properties:

(i) For partial mRNAs (those that in the mRNA description have the phrase “partial cds” or annotated as “3”, or “5”)—an overall identity of 97% coverage of 80% of the Swissprot/TremB1 protein.

(ii) All the rest were considered as full coding RNAs an for them overall identity of 97% identity and coverage of the Swissprot/TremB1 protein of over 95%.

The mRNAs were searched in the LEADS database for their corresponding contigs, and the contigs that included these mRNA sequences were assigned the Swissprot/TremB1 accession.

#PHARM—This field indicates possible pharmacological activities of the % polypeptide. Each Gencarta polypeptide was assigned a SWISSPROT and/or TremB1 human protein accession, as described above. The information contained in this field is the proposed pharmacological activity that was accumulated for the SWISSPROT and/or TremB1 human protein from drug databases such as PharmaProject (PJB Publications Ltd 2003 http://www.pjbpubs.com/cms.asp?pageid=340) and public databases, such as LocusLink and Swissprot. Note that in some cases this field can include opposite terms in cases where the protein can have contradicting activities—such as:

(i) Stimulant—inhibitor

(ii) Agonist—antagonist

(iii) Activator—inhibitor

(iv) Immunosuppressant—Immunostimulant

In these cases the pharmacology was indicated as “modulator”.

As used herein the term “modulator” refers to a molecule which inhibits (i.e., antagonist, inhibitor, suppressor) or activates (i.e., agonist, stimulant, activator) a downstream molecule to thereby modulate its activity.

For example, if the predicted polypeptide has potential agonistic/antagonistic effects (e.g. Fibroblast growth factor agonist and Fibroblast growth factor antagonist) then the annotation for this code will be “Fibroblast growth factor modulator”.

A documentated example for such contradicing activities has been described for the soluble tumor necrosis factor receptors [Mohler et al., J. Immunology 151, 1548-1561]. Essentially, Mohler and co-workers showed that soluble receptor can act both as a carrier of TNF (i.e., agonistic effect) and as an antagonist of TNF activity.

#THERAPEUTIC_PROTEIN—This field predicts a therapeutic role for a protein represented by the contig. A contig was assigned this field if there was information in the drug database or the public databases (e.g., described hereinabove) that this protein, or part thereof, is used or can be used as a drug. This field is accompanied by the swissprot accession of the therapeutic protein which this contig most likely represents. Example: #THERAPEUTIC_PROTEIN UROK_HUMAN

#DN represents information pertaining to transcripts, which contain altered functional interpro domains (further described hereinabove). The Interpro domain is either lacking in this protein (as compared to another expression product of the gene) or its score is decreased (i.e., includes sequence alteration within the domain when compared to another expression product of the gene). This field lists the description of the functional domain(s), which is altered in the respective splice variants.

As used herein the phrase “functional domain” refers to a region of a biomolecular sequence, which displays a particular function. This function may give rise to a biological, chemical, or physiological consequence which may be reversible or irreversible and which may include protein-protein interactions (e.g., binding interactions) involving the functional domain, a change in the conformation or a transformation into a different chemical state of the functional domain or of molecules acted upon by the functional domain, the transduction of an intracellular or intercellular signal, the regulation of gene or protein expression the regulation of cell growth or death, or the activation or inhibition of an immune response.

Method: the proteins were compared to the proteins in the relevant Gencarta contig by BLASTP analysis against each other. All proteins were also analysed by Interpro domain analysis software (Interpro default parameters, the analyses that were run are HMMPfam, HMMSmart, ProfileScan, FprintScan, and BlastProdom). Each pair of proteins that shared at least 20% coverage of one or the other with an identity of at least 80% were analysed by domain comparison. If the proteins share a common domain (same domain accession) and in one of the proteins this domain has a decreased score (escore of 20 magnitude for HMMPfam, HMMSmart, BlastProdom, FprintScan or Pscore difference of ProfileScan of 5), or lacking the domain contained in another protein in the same contig, the protein with the reduced score or without the domains annotated as having lost this interpro domain. This lack of domain can have a functional meaning in which the protein lacking it (or having some part of it missing) can either gain a function or lose a function (e.g., acting, at times, as dominant negative inhibitor of the respective protein). Interpro domains, which have no functional attributes, were omitted from this analysis. The domains that were omitted are:

IPR000694 Proline-rich region

IPR001611 Leucine-rich repeat

IPR001893 Cysteine rich repeat

IPR000372 Cysteine-rich flanking region, N-terminal

IPR000483 Cysteine-rich flanking region, C-terminal

IPR003591 Leucine-rich repeat, typical subtype

IPR003885 Leucine-rich repeat, cysteine-containing type

IPR006461 Uncharacterized Cys-rich domain

IPR006553 Leucine-rich repeat, cysteine-containing subtype

IPR007089 Leucine-rich repeat, cysteine-containing

The results of this analysis are denoted in terms of the Interpro domain that is missing or altered in the protein. Example: #DN IPR002110 Ankyrin.

A documented example is in an article describing two splice variant forms of guanylyl cyclase-B receptor (Tamura N and Garbers D L, J Biol Chem. 2003 Dec. 5; 278(49):48880-9. Epub 2003 Sep. 26). One variant of this receptor has a 25 amino acid deletion in the kinase homology domain and therefore it binds the ligand but fails to activate the cyclase. The other variant includes part of the extracellular binding domain and hence it fails to bind the ligand. Both variants, when co-expressed with the wild-type receptor act as dominant negative isoforms.

#SECRETED_FORM_OF_MEMBRANAL_PROTEINS_BY_PROLOC—This field indicates if the indicated protein is a secreted form of a membranal protein. Method: the proteins were compared to the proteins in the relevant Gencarta by BLASTP analysis against each other. The Proloc algorithm was applied to all the proteins. Each pair of proteins that shared at least 20% coverage of one or the other with an identity of at least 80% was further examined. A protein was considered a soluble form of a membranal protein (i.e., cognate protein) if it was shown to be a secreted protein (as further described below) while the cognate partner was a membranal protein.

A protein was considered secreted of the following properties.

(i) Proloc's highest subcellular localization prediction is EXTRACELLULAR.

(ii) Proloc's prediction of a signal peptide sequence is more reliable than the prediction of a lack of signal peptide sequence. Furthermore, no transmembrane regions are predicted in the non N-terminus part of the protein (following 30 N-terminal amino acids)

(iii) Proloc's prediction of only one transmembrane domain, which is localized to the N-terminus part of the protein (in a region less than the first 30 amino acids)

The cognate protein was considered to be an membranal protein if it obeyed at least one of the following rules:

(i) Proloc's highest subcellular localization prediction is either CELL_INTEGRAL_MEMBRANE, CELL_MEMBRANE E_ANCHORI, or CELL_MEMBRANE_ANCHORII.

(ii) Proloc's prediction of at least one transmembrane domain which is not in the N-terminus part of the protein (in a region greater than the first 30 amino acids)

The header in this method will be

#SECRETED_FORM_OF_MEMBRANNEL_PROTEINS_BY_PROLOC.

Example:

#SECRETED_FORM_OF_MEMBRANNEL_PROTEINS_BY_PROLOC

Example: AA290625 P2 #SECRETED_FORM_OF_MEMBRANNEL_PROTEINS

#MEMBRANE FORM_OF_SOLUBLE_PROTEINS_BY_PROLOC_—This fields denotes if the indicated protein is a membranal form of a secreted protein.

Method: the proteins were compared to the proteins in the relevant Gencarta by BLASTP analysis against each other. The Proloc algorithm was applied to all the proteins. Each pair of proteins that shared at least 20% coverage with an identity of at least 80% was further examined. A protein was considered a membranal form of a secreted protein if it was shown to be (i.e., annotated) a membranal protein and they other protein it was compared to (i.e., cognate) was a secreted protein.

A protein is annotated membranal if is had at least one of the following properties:

(i) Proloc's highest subcellular localization prediction is either CELL_INTEGRAL_MEMBRANE, CELL_MEMBRANE_ANCHORI, or CELL_MEMBRANE_ANCHORII.

(ii) Proloc's prediction of at least one transmembrane domain which is not in the N-terminus part of the protein (in a region greater than the first N-terminal 30 amino acids)

The cognate protein is considered secreted if it obeyed at least one of the following rules:

(i) Proloc's highest subcellular localization prediction is EXTRACELLULAR.

(ii) Proloc's prediction of the existence of a signal peptide sequence is more reliable than the prediction of a lack of signal peptide sequence and no transmembrane regions are predicted in the non N-terminus part of the protein (after its N-terminal 30 amino acids)

(iii) Proloc's prediction of only one transmembrane domain which is in the N-terminus part of the protein (in a region less than the N-terminal 30

The annotation will be in the form of this header, example:

AA176800_P7 #MEMBRANE_FORM_OF_SOLUBLE_PROTEINS_BY PROLOC.

GO annotations were predicted as described in “The ontological annotation approach” section hereinabove. Additions to the GO prediction, other than the GO engine will be described below. These additions are to the cellular component attribute and biological process.

Functional annotations of transcripts based on Gene Ontology (GO) are indicated by the following format.

“#GO_P”, annotations related to Biological Process,

“#GO_F”, annotations related to Molecular Function, and

“#GO_C”, annotations related to Cellular Component.

Proloc was used for protein subcellular localization prediction that assigns GO cellular component annotation to the protein. The localization terms were assigned GO entries.

For this assignment two main approaches were used: (i) the presence of known extracellular domain/s in a protein (as appears in Table 4); (ii) calculating putative transmembrane segments, if any, in the protein and calculating 2 p-values for the existence of a signal peptide. The latest is done by a search for a signal peptide at the N-terminal sequence of the protein generating a score. Running the program on real signal peptides and on N-terminal protein sequences that lack a signal peptide resulted in 2 score distributions the first is the score distribution of the real signal peptides, and the second is the score distribution of the N-terminal protein sequences that lack the signal peptide. Given a new, protein, ProLoc calculates its score and outputs the percentage of the scores that are higher than the current score, in the first distribution, as a first p-value (lower p-values mean more reliable signal peptide prediction) and the percentage of the scores that are lower than the current score, in the second distribution, as a second p-value (lower p-values mean more reliable non signal peptide prediction).

Assignment of an extracellular localization (GO_Acc 5576 GO_Desc extracellular) was also based, on Interpro domains. A list of Interpro domains that characterize secreted proteins was compiled. A Gencarta protein that had a hit to at least one of these domains was annotated with an extracellular GO annotation. The list of secreted Interpro domains is depicted in Table 4.

TABLE 4
List of Interpro Domains of Secreted Proteins

| | |
|---|---|
| IPR000874 | Bombesin-like peptide |
| IPR001693 | Calcitonin-like |
| IPR001651 | Gastrin/cholecystokinin peptide hormone |
| IPR000532 | Glucagon/GIP/secretin/VIP |
| IPR001545 | Gonadotropin, beta chain |
| IPR004825 | Insulin/IGF/relaxin |
| IPR000663 | Natriuretic peptide |
| IPR001955 | Pancreatic hormone |
| IPR001400 | Somatotropin hormone |
| IPR002040 | Tachykinin/Neurokinin |
| IPR006081 | Alpha defensin |
| IPR001928 | Endothelin-like toxin |
| IPR001415 | Parathyroid hormone |
| IPR001400 | Somatotropin hormone |
| IPR001990 | Chromogranin/secretogranin |
| IPR001819 | Chromogranin A/B |
| IPR002012 | Gonadotropin-releasing hormone |
| IPR001152 | Thymosin beta-4 |
| IPR000187 | Corticotropin-releasing factor, CRF |
| IPR001545 | Gonadotropin, beta chain |
| IPR000476 | Glycoprotein hormones alpha chain |
| IPR000476 | Glycoprotein hormones alpha chain |
| IPR001323 | Erythropoietin/thrombopoeitin |
| IPR001894 | Cathelicidin |
| IPR001894 | Cathelicidin |
| IPR001483 | Urotensin II |
| IPR006024 | Opioid neuropeptide precursor |
| IPR000020 | Anaphylatoxin/fibulin |
| IPR000074 | Apolipoprotein A1/A4/E |
| IPR001073 | Complement C1q protein |
| IPR000117 | Kappa casein |
| IPR001588 | Casein, alpha/beta |
| IPR001855 | Beta defensin |
| IPR001651 | Gastrin/cholecystokinin peptide hormone |
| IPR000867 | Insulin-like growth factor-binding protein, IGFBP |
| IPR001811 | Small chemokine, interleukin-8 like |
| IPR004825 | Insulin/IGF/relaxin |
| IPR002350 | Serine protease inhibitor, Kazal type |
| IPR000001 | Kringle |
| IPR002072 | Nerve growth factor |
| IPR001839 | Transforming growth factor beta (TGFb) |
| IPR001111 | Transforming growth factor beta (TGFb), N-terminal |
| IPR001820 | Tissue inhibitor of metalloproteinase |
| IPR000264 | Serum albumin family |
| IPR005817 | Wnt superfamily |

For each category the following features are optionally addressed:

“#GO_Acc” represents the accession number of the assigned GO entry, corresponding to the following “#GO_Desc” field.

“#GO_Desc” represents the description of the assigned GO entry, corresponding to the mentioned “#GO_Acc” field.

The assignment of Immune response GO annotation (#GO_Acc 6955# GO_Desc immune response) to Gencarta; transcripts and proteins was baseds on a homology to a viral protein, as described in U.S. Pat. Appl. No. 60/480,752.

“#CL” represents the confidence level of the GO assignment, when #CL1 is the highest and #CL5 is the lowest possible confidence level. This field appears only when the GO assignment is based on a Swissprot/TremB1 protein accession or Interpro accession and (not on Proloc predictions or viral proteins predictions). Preliminary confidence levels were calculated for all public proteins as follows:

PCL 1: a public protein that has a curated GO annotation,

PCL 2: a public protein that has over 85% identity to a public protein with a curated GO annotation,

PCL 3: a public protein that exhibits 50-85% identity to a public protein with a curated GO annotation,

PCL 4: a public protein that has under 50% identity to a public protein with a curated GO annotation.

For each Gencarta protein a homology search against all public proteins was done. If the Gencarta protein has over 95% identity to a public protein with PCL X than the Gencarta protein gets the same confidence level as the public protein. This confidence level is marked as “#CL X”. If the Gencarta protein has over 85% identity but not over 95% to a public protein with PCL X than the Gencarta protein gets a confidence level lower by 1 than the confidence level of the public protein. If the Gencarta protein has over 70% identity but not over 85% to a public protein with PCL X than the Gencarta protein gets a confidence level lower by 2 than the confidence level of the public protein. If the Gencarta protein has over 50% identity but not over 70% to a public protein with PCL X than the Gencarta protein gets a confidence level lower by 3 than the confidence level of the public protein. If the Gencarta protein has over 30% identity but not over 50% to a public protein with PCL X than the Gencarta protein gets a confidence level lower by 4 than the confidence level of the public protein.

A Gencarta protein may get confidence level of 2 also if it has a true interpro domain that is linked to a GO annotation http://www.geneontology.org/external2go/interpro2go/.

When the confidence level is above “1”, GO annotations of higher levels of the GO hierarchy are assigned (e.g. for “#CL 3” the GO annotations provided, is as appears plus the 2 GO annotations above it in the hierarchy).

“#DB” marks the database on which the GO assignment relies on. The “sp”, as in Example 10a, relates to SwissProt/TremB1 Protein knowledgebase, available from http://www.expasy.ch/sprot/. “InterPro”, as in Example 10c, refers to the InterPro combined database, available from http://www.ebi.ac.uk/interpro/, which contains information regarding protein families, collected from the following databases: SwissProt (http://www.ebi.ac.uk/swissprot/), Prosite (http://www.expasy.ch/prosite/), Pfam (http://www.sanger.ac.uk/Software/Pfam/), Prints (http://www.bioinf.man.ac.uk/dbbrowser/PRINTS/), Prodom (http://prodes.toulouse.inra.fr/prodom/), Smart (http://smart.embl-heidelberg.de/) and Tigrfams (http://www.tigr.org/TIGRFAMs/). PROLOC means the method used was Proloc based on statistics Proloc uses for predicting the subcellular localization of a protein #EN” represents the accession of the entity in the database (#DB), corresponding to the accession of the protein/domain why the GO was predicted. If the GO assignment is based on a protein from the SwissProt/TremB1 Protein database this field will have the locus name of the protein. Examples, “#DB sp #EN NRG2_HUMAN” means that the GO assignment in this case was based on a protein from the SwissProt/Tremb1 database, while the closest homologue (that has a GO assignment) to the assigned protein is depicted in SwissProt entry “NRG2_HUMAN “#DB interpro #EN IPR001609” means that GO assignment in this case was based on InterPro database, and the protein had an Interpro domain, IPR001609, that the assigned GO was based on. In Proloc predictions this field will have a Proloc annotation “#EN Proloc”. #GENE_SYMBOL—for each Gencarta contig a HUGO gene symbol was assigned in two ways:

(i) After assigning a Swissprot/TremB1 proteins to each contig (see Assignment of Swissprot/TremB1 accessions to Gencarta contigs) all the gene symbols that appear for the Swissprot entry were parsed and added as a Gene symbol annotation to the gene.

(ii) LocusLink information—LocusLink was downloaded from NCBI ftp)://ftp.ncbi.nih.gov/refseq/LocusLink/ (files loc2acc, loc2ref, and LL.out_hs). The data was integrated producing a file containing the gene symbol for every sequence. Gencarta contigs were assigned a gene symbol if they contain a sequence from this file that has a gene symbol

Example: #GENE_SYMBOL MMP15

#DIAGNOSTICS—KGencarta contigs representing known diagnostic markers (such as listed in Table 5, below) and all transcripts and proteins deriving from this contig will be assigned to this field and will get the above mentioned annotation followed by “as indicated in the Diagnostic markers table”.

TABLE 5

| | | |
|---|---|---|
| Test | Gencarta Contig | Comments |

Enzymes

| | | |
|---|---|---|
| GPT | R35137 (GPT glutamic-pyruvate | Also called ALT - alanine |
| | transaminase (alanine aminotransferase)) | aminotransferase. Standard liver |
| | Z24841 (GPT2 glutamic pyruvate | function test |
| | transaminase (alanine aminotransferase) | |
| | 2) | |
| GOT | M78228 (GOT1 glutamic-oxaloacetic | Also called AST - aspartate |
| | transaminase 1, soluble (aspartate | aminotransferase. Standard liver |
| | aminotransferase 1)) | function test |
| | M86145 (GOT2 glutamic-oxaloacetic | |
| | transaminase 2, mitochondrial (aspartate | |
| | aminotransferase 2) | |
| GGT | HUMGGTX (GGT1: gamma- | Liver disease |
| | glutamyltransferase 1) | |
| CPK | T05088 (CKB creatine kinase, brain) | Also called CK. Mostly used for muscle |
| | HUMCKMA (CKM creatine kinase, | pathologies. The MB variant is heart |
| | muscle) | specific and used in the diagnosis of |
| | H20196 (CKMT1 creatine kinase, | myocardial infarction |
| | mitochondrial 1 (ubiquitous)) | |
| | HUMSMCK (CKMT2 creatine kinase, | |
| | mitochondrial 2 (sarcomeric)) | |
| CPK-MB | T05088 (CKB creatine kinase, brain) | Cardiac problems - hetro-dimer of |
| | HUMCKMA (CKM creatine kinase, | CKB and CKM |
| | muscle) | |
| Alkaline | HSAPHOL-ALPL: alkaline phosphatase, | Bone related syndromes and liver |
| Phosphatase | liver/bone/kidney | diseases, mostly with biliary |
| | HUMALPHB-ALPI: alkaline | involvement |
| | phosphatase, intestinal | |
| | HUMALPP-ALPP: alkaline phosphatase, | |
| | placental (Regan isozyme) | |
| Amylase | AA367524 - (AMY1A: amylase, alpha | Blood/Urine. Pancreas related diseases |
| | 1A; salivary) | |
| | T10898 - (AMY2B: amylase, alpha 2B; | |
| | pancreatic and 2A) | |
| LDH | HSLDHAR (LDHA lactate | Lactate Dehydrogenase. Used for |
| | dehydrogenase A) | myocardial infarction diagnosis and |
| | M77886 (LDHB lactate dehydrogenase | neoplastic syndromes assessment. |
| | B) | |
| | HSU13680 (LDHC lactate dehydrogenase | |
| | C) | |
| | AA398148 (LDHL lactate dehydrogenase | |
| | A-like) | |
| | R09053 (LDHD lactate dehydrogenase D) | |
| G6PD | S58359 (G6PD glucose-6-phosphate | Glucose 6-phosphate dehydrogenase. |
| | dehydrogenase) | Levels measured when deficiency is |
| | | suspected (leading to susceptibility to |
| | | hemolysis) |
| Alpha1 | HUMA1ACM (SERPINA3 serine (or | Chronic lung diseases |
| antiTrypsin | cysteine) proteinase inhibitor, clade, A | |
| | (alpha-1 antiproteinase, antitrypsin), | |
| | member 3) | |
| | T10891 (AGT angiotensinogen (serine (or | |
| | cysteine) proteinase inhibitor, clade A | |
| | (alpha-1 antiproteinase, antitrypsin), | |
| | member | |
| | 8)) | |
| | R83168 (SERPINA6 serine (or cysteine) | |
| | proteinase inhibitor, clade A (alpha-1 | |
| | antiproteinase, antitrypsin), member 6) | |
| | HUMCINHP (SERPINA5 serine (or | |
| | cysteine) proteinase inhibitor, clade A | |
| | (alpha-1 antiproteinase, antitrypsin), | |
| | member 5) | |
| | HSA1ATCA (SERPINA1 serine (or | |
| | cysteine) proteinase inhibitor, clade A | |
| | (alpha-1 antiproteinase, antitrypsin), | |
| | member 1) | |
| | HUMKALLS (SERPINA4 serine (or | |
| | cysteine) proteinase inhibitor, clade A | |
| | (alpha-1 antiproteinase, antitrypsin), | |
| | member 4) | |
| | HUMTBG (SERPINA7 serine (or | |
| | cysteine) proteinase inhibitor, clade A | |
| | (alpha-1 antiproteinase, antitrypsin), | |
| | member 7) | |
| | T60354 (SERPINA10 serine (or cysteine) | |
| | proteinase inhibitor, clade A (alpha-1 | |
| | antiproteinase, antitrypsin), member 10) | |
| Renin | HSRENK (REN renin) | Some hypertension syndromes |
| Acid | HUMAAPA (ACP1: acid phosphatase 1, | Used to differentiate multiple myeloma |
| Phosphatase | soluble) | with other monoclonal gammopathies |
| | T48863 (ACP2: acid phosphatase 2, | of uncertain significance |
| | lysosomal) | |
| | HSMRACP5 (ACP5: acid phosphatase 5, | |
| | tartrate resistant) | |
| | T85211 (ACP6: lysophosphatidic acid | |
| | phosphatase) | |
| | HSPROSAP (ACPP: acid phosphatase, | |
| | prostate) | |
| | AA005037 (ACPT: acid phosphatase, | |
| | testicular) | |
| Beta | T11069 (GUSB glucuronidase, beta) | Used to differentiate multiple myeloma |
| glucoronidase | | with other monoclonal gammopathies |
| | | of uncertain significance |
| Aldolase | HSALDAR (ALDOA aldolase A, | Glycogen storage diseases |
| | fructose-bisphosphate) | |
| | HSALDOBR (ALDOB aldolase B, | |
| | fructose-bisphosphate) | |
| | M62176 (ALDOC aldolase C, fructose- | |
| | bisphosphate) | |
| Choline esterase | HUMCHEF (BCHE | Probably used for |
| | butyrylcholinesterase) | organophosphates/“nerve gases” |
| | F00931 (ACHE acetylcholinesterase (YT | intoxications |
| | blood group)) | |
| Pepsinogen | HUMPGCA PGC: progastricsin | (in the stomach), high in gastritis, low |
| | (pepsinogen C) | in pernicious anemia[ |
| ACE | HSACE (ACE: angiotensin I converting | Angiotensin-converting enzyme |
| | enzyme (peptidyl-dipeptidase A) 1) | Sarcoidosis |
| | AA397955 (ACE2: angiotensin I | |
| | converting enzyme (peptidyl-dipeptidase | |
| | A) 2) | |

Miscelleneous

| | | |
|---|---|---|
| Prion Protein | HUMPRP0A (PRNP prion protein (p27-30) | BSE diagnosis |
| | (Creutzfeld-Jakob disease, | |
| | Gerstmann-Straus | |
| | ler-Scheinker syndrome, fatal familial | |
| | insomnia)) | |
| | W73057 (PRND prion protein 2 (dublet)) | |
| Myelin basic | M78010 (MBP myelin basic protein) | In CSF. In Multiple sclerosis |
| protein | R13982 (MOBP myelin-associated | |
| | oligodendrocyte basic protein) | |
| Albumin | HSALB1 (ALB albumin) | Mostly liver function and failure of |
| | | intestine absorption |
| Prealbumin | HSALB1 (ALB albumin) | early diagnosis of malabsorption |
| Ferritin | HUMFERLS (FTL ferritin, light | Iron deficiency anemia |
| | polypeptide) | |
| | HUMFERHA (FTH1 ferritin, heavy | |
| | polypeptide 1) | |
| Transferrin | S95936 (TF transferrin) | Iron deficiency anemia |
| Haptoglobin | HUMHPA1B (HP haptoglobin) | Used in anemia states and neoplastic |
| | | syndromes |
| CRP | HSCREACT (CRP C-reactive protein, | C reactive protein. Associated with |
| | pentraxin-related) | active inflammation |
| AFP | D11581 (AFP alpha-fetoprotein) | Alpha Feto Protein. Used in pregnancy |
| | | for abnormalities screening and as a |
| | | cancer marker. |
| C3 | T40158 (C3 complement component 3) | Various auto-immune and allergy |
| | | syndromes |
| C4 | HSCOC4 (C4A complement component | Various auto-immune and allergy |
| | 4A; C4B complement component 4B) | syndromes |
| Ceruloplasmin | HSCP2 (CP ceruloplasmin (ferroxidase)) | Wilson's disease (liver disease) |
| Myoglobin | T11628 (MB myoglobin) | Rhabdomyolysis, Myocardial infarction |
| FABP | S67314 (FABP3: fatty acid binding | myoglobin and Fatty Acid Binding |
| | protein 3, muscle and heart) | |
| | D11754 (FABP1 liver-L-FABP-fatty | |
| | acid binding protein 1) | |
| | AW605378 (FABP2: fatty acid binding | |
| | protein 2, intestinal) | |
| | HUMALBP (FABP4: fatty acid binding | |
| | protein 4, adipocyte) | |
| | T06152 (FABP5: fatty acid binding | |
| | protein 5 (psoriasis-associated) | |
| | HSI15PGN1 (FABP6: fatty acid binding | |
| | protein 6, ileal (gastrotropin) | |
| | R60348 (FABP7: fatty acid binding | |
| | protein 7, brain) | |
| Troponin I | HUMTROPNIN (TNNI2 troponin I, | Acute myocardial infarction |
| | skeletal, fast) | |
| | Z25083 (TNNI1 troponin I, skeletal, | |
| | slow) | |
| | HUMTROPIA (TNNI3 troponin I, | |
| | cardiac) | |
| Beta-2- | HSB2MMU (B2M beta-2-microglobulin) | |
| microglobulin | | |
| Macroglobin | M62177 (A2M: alpha-2-macroglobulin) | Elevated in inflammation |
| Alpha-1 | T72188 (A1BG: alpha-1-B glycoprotein) | Elevated in inflammation and tumors, |
| glycoprotein | | |
| Apo A-I | HUMAPOAIP (APOA1: apolipoprotein | Risk for coronary artery disease |
| | A-I) | |
| Apo B-100 | HSAPOBR2 (APOB: apolipoprotein B | Atherosclerotic heart disease |
| | (including Ag(x) antigen)) | |
| Apo E | T61627 (APOE: apolipoprotein E) | diagnosis of Type III |
| | | hyperlipoproteinemia, evaluate a |
| | | possible genetic component to |
| | | atherosclerosis, or to help confirm a |
| | | diagnosis of late onset AD |
| CF gene | HUMCFTRM (CFTR: cystic fibrosis | Cystic fibrosis disease (a DNA test - |
| | transmembrane conductance regulator, | blood sample) |
| | ATP-binding cassette (sub-family C, | |
| | member 7)) | |
| PSEN1 gene | T89701 (PSEN1: presenilin 1 (Alzheimer | Early onset of familial AD (a DNA test - |
| | disease 3)) | blood sample) |

Hormones

| | | |
|---|---|---|
| Erythropoietin | HSERPR (EPO erythropoietin) | Hardly used for diagnosis. Used as |
| | | treatment |
| GH | HSGROW1 (GH1 growth hormone 1) | Growth Hormone. Endocrine |
| | HUMCS2 (GH2 growth hormone 2) | syndromes |
| TSH | AV745295 (TSHB thyroid stimulating | Part of thyroid functions tests |
| | hormone, beta) | |
| betaHCG | R27266 (CGB5 chorionic | Pregnancy, malignant syndromes in |
| | gonadotropin, beta polypeptide 5) | men and women |
| LH | HUMCGBB50 (LHB luteinizing | Part of standard hormonal profile for |
| | hormone beta polypeptide) | fertility, gynecological syndromes and |
| | | endocrine syndromes |
| FSH | AV754057 (FSHB follicle stimulating | Part of standard hormonal profile for |
| | hormone, beta polypeptide) | fertility, gynecological syndromes and |
| | | endocrine syndromes |
| TBG | S40807 (TG thyroglobulin) | Thyroxin binding globulin. Thyroid |
| | | syndromes |
| Prolactin | HSLACT (PRL prolactin) | Various endocrine syndromes |
| Thyroglobulin | S40807 (TG thyroglobulin) | Follow up of thyroid cancer patients |
| PTH | HSTHYR (PTH parathyroid hormone) | Parathyroid Hormone. Syndromes of |
| | | calcium management |
| Insulin/Pre Insulin | HSPPI (INS insulin) | Diabetes |
| Gastrin | HSGAST (GAS gastrin) | Peptic ulcers |
| Oxytocin | HUMOTCB (OXT oxytocin, prepro- | Endocrine syndromes related to |
| | (neurophysin I)) | lactation |
| AVP | HUMVPC (AVP arginine vasopressin | Arginine Vasopressin. Endocrine |
| | (neurophysin II, antidiuretic hormone, | syndromes related to the osmotic |
| | diabetes | pressure of body fluids |
| | insipidus, neurohypophyseal)) | |
| ACTH | HUMPOMCMTC (POMC: | Secreted from the anterior pituitary |
| | proopiomelanocortin | gland. Regulation of cortisol. |
| | (adrenocorticotropin/beta-lipotropin/ | Abnormalities are indicative of |
| | alpha-melanocyte stimulating | Cushing's disease, addison's disease |
| | hormone/beta-melanocyte stimulating | and adrenal tumors |
| | hormone/beta-endorphin)) | |
| BNP | HUMNATPEP (NPPB: natriuretic | Heart failure |
| | peptide precursor B) | |

Blood Clotting

| | | |
|---|---|---|
| Protein C | S50739 (PROC protein C (inactivator of | Inherited Clotting disorders |
| | coagulation factors Va and VIIIa)) | |
| Protein S | HSSPROTR (PROS1 protein S (alpha)) | Inherited Clotting disorders |
| Fibrinogen | D11940 (FGA: fibrinogen, A alpha | Clotting disorders |
| | polypeptide) | |
| | HUMFBRB (FGB: fibrinogen, B beta | |
| | polypeptide) | |
| | T24021 (FGG: fibrinogen, gamma | |
| | polypeptide) | |
| Factors 2, 5, 7, | HUMPTHROM (F2 coagulation factor II | Inherited Clotting disorders |
| 9, 10, 11, 12, 13 | (thrombin)) | |
| | HUMTFPC (F3 coagulation factor III | |
| | (thromboplastin, tissue factor)) | |
| | HUMF5A (F5 coagulation factor V | |
| | (proaccelerin, labile factor)) | |
| | M78203 (F7 coagulation factor VII (serum | |
| | prothrombin conversion accelerator)) | |
| | HUMF8C (F8 coagulation factor VIII, | |
| | procoagulant component (hemophilia A)) | |
| | HUMCFIX (F9 coagulation factor IX (plasma | |
| | thromboplastic component, Christmas disease, | |
| | hemophilia B)) | |
| | HUMCFX (F10: coagulation factor X) | |
| | HUMEXI (F11 coagulation factor XI (plasma | |
| | thromboplastin antecedent)) | |
| | HUMCFXIIA (F12 coagulation factor XII | |
| | (Hageman factor)) | |
| | HUMFXIIIA (F13A1 coagulation factor XIII, | |
| | A1 polypeptide) | |
| | R28976 (F13B coagulation factor XIII, B | |
| | polypeptide) | |
| vWF | HUMVWF (VWF von Willebrand factor) | Von Willebrand factor. Inherited |
| | | Clotting disorders |
| Antithrombin | T62060 (SERPINC1 serine (or cysteine) | Inherited Clotting disorders |
| III | proteinase inhibitor, clade C (antithrombin), | |
| | member 1) | |

Cancer Markers

| | | |
|---|---|---|
| AFP | D11581 (AFP alpha-fetoprotein) | Pregnancy, testicular cancer and |
| | | hepatocellular cancer |
| CA125 | HSIAI3B (M17S2 membrane component, | Ovarian cancer |
| | chromosome 17, surface marker 2 (ovarian | |
| | carcinoma antigen CA125)) | |
| CA-15-3 | HSMUC1A (MUC1 mucin 1, transmembrane) | Breast cancer |
| CA-19-9 | HSAFUTF (FUT3: fucosyltransferase 3 | Gastrointestinal cancer, pancreatic |
| | (galactoside 3(4)-L-fucosyltransferase, Lewis | cancer |
| | blood group included)) | |
| CEA | T10888 HUMCEA (CEACAM3 | Carcinoembryonic Antigen. |
| | carcinoembryonic antigen-related cell adhesion | Colorectal cancer |
| | molecule 3) | |
| PSA | HSCDN9 (KLK3: kallikrein 3, (prostate | |
| | specific antigen)) | |
| PSMA | HUMPSM (FOLH1: folate hydrolase | |
| | (prostate-specific membrane antigen) 1) | |
| TPA, TATI, | HSPSTI (SPINK1: serine protease inhibitor, | Ovarian cancer |
| OVX1, LASA, | Kazal type 1) | |
| CA54/81 | | |
| BRCA 1 | H90415 (BRCA1: breast cancer 1, early onset) | |
| BRCA 2 | H47777 (BRCA2: breast cancer 2, early onset) | Breast cancer (ovarian cancer). |
| HER2/Neu | S57296 (ERBB2: v-erb-b2 erythroblastic | Breast cancer |
| | leukemia viral oncogene homolog 2, | |
| | neuro/glioblastoma derived oncogene homolog | |
| | (avian)) | |
| Estrogen | HSERG5UTA (ESR1: estrogen receptor 1) | Breast cancer |
| receptor | HSRINAERB (ESR2: estrogen receptor 2 (ER | |
| | beta)) | |
| Progesterone | T09102 (PGRMC1: progesterone receptor | Breast cancer |
| | membrane component 1) | |
| | Z32891 (PGRMC2: progesterone receptor | |
| | membrane component 2) | |

Note:
(i) Small portion of these “markers” are also drug targets, whether already for approved drugs (such as alpha1 antiTrypsin) or under development (e.g., GOT).
(ii) Some of these “markers” are also used as therapeutic proteins (e.g., Erythropoietin).
(iii) All markers are found in the blood/serum unless otherwise specified.

1. #DISEASE_RELATED_CLINICAL_PHENOTYPE—This field denotes the possibility of using biomolecular sequences of the present invention for the diagnosis and/or treatment of genetic diseases such as listed in the following URL: http://www.geneclinics.org/servlet/access?id=8888891&key=X9D790O5re1Az&db=genetests&res=&fcn=b&grp=g&genesearch=true&testtype=both&1s=1&type=e&qry=&submit=Search and in Table 6, below is list includes genetic diseases and genes which may be used for the detection and/or treatment thereof. As such, newly uncovered variants of these genes, including novel SNPs or mutations may be used for improved diagnosis and/or treatment when used singly or in combination with the previously described genes. For example, in genetic diseases where the diseased phenotype has a different splice variant of the than the healthy phenotype, like that seen in Thalasemia and in Duchenne Macular Dystrophy, the novel splice variants might discriminate between healthy and diseased phenotype.

Another example is in cases of autosomal recessive genetic diseases. Some of the sequences in genebank were sequenced from malfunctioning alleles derived from healthy carriers of the disease, and therefore contain the mutation that leads to the disease. Identification of novel SNPs predicted based on sequence alignment can assist in identifying disease-causing mutations.

TABLE 6

| Gencarta Contig | Gene Symbol | Disease |
|---|---|---|
| HSCFTRMA | CFTR | Congenital Bilateral Absence of the Vas Deferens; |
| | | Cystic Fibrosis |
| HUMCFTRM | CFTR | Congenital Bilateral Absence of the Vas Deferens; |
| | | Cystic Fibrosis |
| HUMFGFR3 | FGFR3 | Achondroplasia; Crouzon Syndrome with Acanthosis |
| | | Nigricans; FGFR-Related Craniosynostosis Syndromes; |
| | | Hypochondroplasia; Muenke Syndrome; Severe |
| | | Achondroplasia with Developmental Delay and |
| | | Acanthosis Nigricans (SADDAN); Thanatophoric |
| | | Dysplasia |
| HSU11690 | FGD1 | Aarskog Syndrome |
| HSCA1III | COL3A1 | Ehlers-Danlos Syndrome, Vascular Type |
| HUMCOL2A1B | COL2A1 | Achondrogenesis Type 2; Kniest Dysplasia; |
| | | Spondyloepimetaphyseal Dysplasia, Strudwick Type; |
| | | Sponclyloepiphyseal Dysplasia, Congenita; Stickler |
| | | Syndrome; Stickler Syndrome Type I |
| R68817 | APRT | Adenine Phosphoribosyltransferase Deficiency |
| HUMAMPD1 | AMPD1 | Adenosine Monophosphate Deaminase 1 |
| M62124 | PXR1 | Zellweger Syndrome Spectrum |
| HSXLALDA | ABCD1 | Adrenoleukodystrophy, X-Linked |
| T28718 | BTK | X-Linked Agammaglobulinemia |
| R91110 | IL2RG | X-Linked Severe Combined Immunodeficiency |
| HUMPEDG | OCA2 | Oculocutaneous Albinism Type 2 |
| HSU01873 | TYR | Oculocutaneous Albinism Type 1 |
| HSOA1MRNA | OA1 | Ocular Albinism, X-Linked |
| R14843 | TYRP1 | Oculocutaneous Albinism Type 3 (TRP1 Related) |
| HSALDAR | ALDOA | Aldolase A Deficiency |
| T40633 | HBA1 | Alpha-Thalassemia |
| T40633 | HBA2 | Alpha-Thalassemia; Hemoglobin Constant Spring |
| HSU09820 | ATRX | Alpha-Thalassemia X-Linked Mental Retardation |
| | | Syndrome |
| HUMCOL4A5 | COL4A5 | Alport Syndrome; Alport Syndrome, X-Linked |
| T61627 | APOE | Apolipoprotein E Genotyping; Familial Combined |
| | | Hyperlipidemia; Hyperlipoproteinemia Type III |
| T89701 | PSEN1 | Alzheimer Disease Type 3; Early-Onset Familial |
| | | Alzheimer Disease |
| R05822 | PSEN2 | Alzheimer Disease Type 4; Early-Onset Familial |
| | | Alzheimer Disease |
| HSTTRM | TTR | Transthyretin Amyloidosis |
| T23978 | SOD1 | Amyotrophic Lateral Sclerosis |
| HUMANDREC | AR | Androgen Insensitivity Syndrome; Spinal and Bulbar |
| | | Muscular Atrophy |
| Z19491 | UBE3A | Angelman Syndrome |
| HUMPAX6AN | PAX6 | Aniridia; Anophthalmia; Isolated Aniridia; Peters |
| | | Anomaly; Peters Anomaly with Cataract; Wilms |
| | | Tumor-Aniridia-Genital Anomalies-Retardation |
| | | Syndrome |
| HUMKGFRA | FGFR2 | Apert Syndrome; Beare-Stevenson Syndrome; Crouzon |
| | | Syndrome; FGFR-Related Craniosynostosis Syndromes; |
| | | Jackson-Weiss Syndrome; Pfeiffer Syndrome Type 1, |
| | | 2, and 3 |
| HSU03272 | FBN2 | Congenital Contractural Arachnodactyly |
| Z19459 | AMCD1 | Arthrogryposis Multiplex Congenita, Distal, Type I |
| T88756 | ATM | Ataxia-Telangiectasia |
| H30056 | BBS1 | Bardet-Biedl Syndrome |
| Z25009 | BBS2 | Bardet-Biedl Syndrome |
| T64876 | BBS4 | Bardet-Biedl Syndrome |
| N27125 | PTCH | Nevoid Basal Cell Carcinoma Syndrome |
| N31453 | VMD2 | Best Vitelliform Macular Dystrophy |
| HUMHBB3E | HBB | Beta-Thalassemia; Hemoglobin E; Hemoglobin S Beta- |
| | | Thalassemia; Hemoglobin SC; Hemoglobin SD; |
| | | Hemoglobin SO; Hemoglobin SS; Sickle Cell Disease |
| H53763 | BLM | Bloom Syndrome |
| N22283 | EYA1 | Branchiootorenal Syndrome |
| H90415 | BRCA1 | BRCA1 and BRCA2 Hereditary Breast/Ovarian Cancer; |
| | | BRCA1 Hereditary Breast/Ovarian Cancer |
| H47777 | BRCA2 | BRCA1 and BRCA2 Hereditary Breast/Ovarian Cancer; |
| | | BRCA2 Hereditary Breast/Ovarian Cancer |
| Z33575 | SOX9 | Campomelic Dysplasia |
| S67156 | ASPA | Canavan Disease |
| T52465 | CPS1 | Carbamoylphosphate Synthetase I Deficiency |
| HSVD3HYD | CYP27A1 | Cerebrotendinous Xanthomatosis |
| S66705 | MPZ | Charcot-Marie-Tooth Neuropathy Type 1; Charcot- |
| | | Marie-Tooth Neuropathy Type 1B; Congenital |
| | | Hypomyelination |
| HSGAS3MR | PMP22 | Charcot-Marie-Tooth Neuropathy Type 1; Charcot- |
| | | Marie-Tooth Neuropathy Type 1A; Charcot-Marie- |
| | | Tooth Neuropathy Type 1E; Hereditary Neuropathy |
| | | with Liability to Pressure Palsies |
| T93208 | PMP22 | Charcot-Marie-Tooth Neuropathy Type 1; Charcot- |
| | | Marie-Tooth Neuropathy Type 1A; Charcot-Marie- |
| | | Tooth Neuropathy Type 1E; Hereditary Neuropathy |
| | | with Liability to Pressure Palsies |
| HSGAPJR | GJB1 | Charcot-Marie-Tooth Neuropathy Type X |
| HSXCGD | CYBB | Chronic Granulomatous Disease |
| S67289 | CYBB | Chronic Granulomatous Disease |
| HSASD | ASS | Citrullinemia |
| HUMPAX2A | PAX2 | Anophthalmia; Renal-Coloboma Syndrome |
| HUMP45C21 | CYP21A2 | 21-Hydroxylase Deficiency |
| S74720 | NR0B1 | Complex Glycerol Kinase Deficiency; Dosage- |
| | | Sensitive Sex Reversal; Isolated X-Linked Adrenal |
| | | Hypoplasia Congenita; X-Linked Adrenal Hypoplasia |
| | | Congenita |
| HSKERTRNS | TGM1 | Autosomal Recessive Congenital Ichthyosis |
| BF928311 | CPO | Hereditary Coproporphyria |
| HSCPPOX | CPO | Hereditary Coproporphyria |
| HUMTGFBIG | TGFBI | Avellino Corneal Dystrophy; Granular Corneal |
| | | Dystrophy; Lattice Corneal Dystrophy Type I |
| R08437 | MSX2 | Craniosynostosis Type II; Parietal Foramina 1 |
| HUMPRP0A | PRNP | Prion Diseases |
| T08652 | DRPLA | DRPLA |
| Z46151 | DRPLA | DRPLA |
| HSWT1 | WT1 | Denys-Drash Syndrome; Wilms Tumor; Wilms Tumor- |
| | | Aniridia-Genital Anomalies-Retardation Syndrome; |
| | | WT1-Related Disorders |
| HUMWT1X | WT1 | Denys-Drash Syndrome; Wilms Tumor; Wilms Tumor- |
| | | Aniridia-Genital Anomalies-Retardation Syndrome; |
| | | WT1-Related Disorders |
| M78080 | ATP2A2 | Darier Disease |
| Z30219 | DCR | Down Syndrome Critical Region |
| T11279 | DKC1 | Dyskeratosis Congenita |
| T08131 | DYT1 | Early-Onset Primary Dystonia (DYT1) |
| T50729 | ED1 | Hypohidrotic Ectodermal Dysplasia; Hypohidrotic |
| | | Ectodermal Dysplasia, X-Linked |
| HUMPA1V | COL5A1 | Ehlers-Danlos Syndrome, Classic Type |
| HUMLYSYL | PLOD | Ehlers-Danlos Syndrome, Kyphoscoliotic Form |
| HSCOLIA | COL1A2 | Ehlers-Danlos Syndrome, Arthrochalasia Type; |
| | | Osteogenesis Imperfecta |
| HUMCG1PA1 | COL1A1 | Ehlers-Danlos Syndrome, Arthrochalasia Type; |
| | | Osteogenesis Imperfecta |
| Z30171 | TAZ | 3-Methylglutaconic Aciduria Type 2; Cardiomyopathy; |
| | | Dilated Cardiomyopathy; Endocardial Fibroelastosis; |
| | | Familial Isolated Noncompaction of Left Ventrical |
| | | Myocardium |
| Z39302 | TAZ | 3-Methylglutaconic Aciduria Type 2; Cardiomyopathy; |
| | | Dilated Cardiomyopathy; Endocardial Fibroelastosis; |
| | | Familial Isolated Noncompaction of Left Ventrical |
| | | Myocardium |
| HUMKERK5A | KRT5 | Epidermolysis Bullosa Simplex |
| R72295 | KRT14 | Epidermolysis Bullosa Simplex |
| HUMKTEP2A | KRT1 | Epidermolytic Hyperkeratosis; Nonepidermolytic |
| | | Palmoplantar Hyperkeratosis |
| HUMK10A | KRT10 | Epidermolytic Hyperkeratosis |
| M78482 | CHS1 | Chediak-Higashi Syndrome |
| HSTCD1 | CHM | Choroideremia |
| HSAGALAR | GLA | Fabry Disease |
| T79651 | GLA | Fabry Disease |
| HUMF5A | F5 | Factor V Leiden Thrombophilia; Factor V R2 Mutation |
| | | Thrombophilia |
| HUMFXI | F11 | Factor XI Deficiency |
| M79108 | APC | Colon Cancer (APC I1307K related); Familial |
| | | Adenomatous Polyposis |
| T10619 | IKBKAP | Familial Dysautonomia |
| HUMFMR1 | FMR1 | Fragile X Syndrome |
| M78417 | FMR2 | FRAXE Syndrome |
| R06415 | FRDA | Friedreich Ataxia |
| HSALDOBR | ALDOB | Hereditary Fructose Intolerance |
| HUMALFUC | FUCA1 | Fucosidosis |
| M85904 | FH | Fumarate Hydratase Deficiency |
| H85361 | ABCA4 | Age-Related Macular Degeneration; Retinitis |
| | | Pigmentosa, Autosomal Recessive; Stargardt Disease 1 |
| R31596 | GALK1 | Galactokinase Deficiency |
| T53762 | GALT | Galactosemia |
| HUMGCB | GBA | Gaucher Disease |
| T48672 | GBA | Gaucher Disease |
| HSGCRAR | NR3C1 | Glucocorticoid Resistance |
| S58359 | G6PD | Glucose-6-Phosphate Dehydrogenase Deficiency |
| HSGKTS1 | GK | Glycerol Kinase Deficiency |
| HSRNAGLK | GK | Glycerol Kinase Deficiency |
| U01120 | G6PC | Glycogen Storage Disease Type Ia |
| HUMGAAA | GAA | Glycogen Storage Disease Type II |
| F00985 | AGL | Glycogen Storage Disease Type III |
| HUMHGBE | GBE1 | Glycogen Storage Disease Type IV |
| HSPHOSR1 | PYGM | Glycogen Storage Disease Type V |
| D12179 | PYGL | Glycogen Storage Disease Type VI |
| HSHMPFK | PFKM | Glycogen Storage Disease Type VII |
| HUMGLI3A | GLI3 | GLI3-Related Disorders; Greig Cephalopolysyndactyly |
| | | Syndrome; Pallister-Hall Syndrome |
| F09335 | ATP2C1 | Hailey-Hailey Disease |
| M62210 | CCM1 | Angiokeratoma Corporis Diffusum with Arteriovenous |
| | | Fistulas; Familial Cerebral Cavernous Malformation |
| T59431 | HFE | HFE-Associated Hereditary Hemochromatosis |
| HSALK1A | ACVRL1 | Hereditary Hemorrhagic Telangiectasia |
| HUMENDO | ENG | Hereditary Hemorrhagic Telangiectasia |
| HUMF8C | F8 | Hemophilia A |
| HUMFVIII | F8 | Hemophilia A |
| HUMCFIX | F9 | Hemophilia B |
| HSU03911 | MSH2 | Hereditary Non-Polyposis Colon Cancer |
| Z24775 | MLH1 | Hereditary Non-Polyposis Colon Cancer |
| HSRETTT | RET | Hirschsprung Disease; Multiple Endocrine Neoplasia |
| | | Type 2 |
| HUMSHH | SHH | Holoprosencephaly 3 |
| N81026 | TBX5 | Holt-Oram Syndrome |
| M78262 | CBS | Homocystinuria |
| T06035 | IDS | Mucopolysaccharidosis Type II |
| T03828 | HD | Huntington Disease |
| H27612 | IDUA | Mucopolysaccharidosis Type I |
| M62205 | GFAP | Alexander Disease |
| HUMCD40L | TNFSF5 | Hyper IgM Syndrome, X-Linked |
| HUMPTHROM | F2 | Prothrombin G20210A Thrombophilia |
| T61466 | MTHFR | MTHFR Deficiency; MTHFR Thermolabile Variant |
| HUMSKM1A | SCN4A | Hyperkalemic Periodic Paralysis Type 1; Hypokalemic |
| | | Periodic Paralysis; Hypokalemic Periodic Paralysis |
| | | Type 2; Myotonia Congenita, Dominant; |
| | | Paramyotonia Congenita |
| HSU09784 | CACNA1S | Hypokalemic Periodic Paralysis; Hypokalemic Periodic |
| | | Paralysis Type 1; Malignant Hyperthermia |
| | | Susceptibility |
| HUMLPLAA | LPL | Familial Lipoprotein Lipase Deficiency |
| HUMPEX | PHEX | Hypophosphatemic Rickets, X-Linked Dominant |
| M78626 | STS | Ichthyosis, X-Linked |
| R56102 | IKBKG | Incontinentia Pigmenti |
| Z39843 | IVD | Isovaleric Acidemia |
| S60085S1 | KAL1 | Kallmann Syndrome, X-Linked |
| T55061 | KEL | Kell Antigen Genotyping |
| HUMGALC | GALC | Krabbe Disease |
| HUMZFPSREB | ZNF9 | Myotonic Dystrophy Type 2 |
| Z19342 | KIF1B | Charcot-Marie-Tooth Neuropathy Type 2 |
| T11351 | NPC2 | Niemann-Pick Disease Type C |
| Z39096 | NDRG1 | Charcot-Marie-Tooth Neuropathy Type 4 |
| AA984421 | PRX | Charcot-Marie-Tooth Neuropathy Type 4; Charcot- |
| | | Marie-Tooth Neuropathy Type 4F |
| HUMRETGC | GUCY2D | Leber Congenital Amaurosis |
| HSU18991 | RPE65 | Leber Congenital Amaurosis; Retinitis Pigmentosa, |
| | | Autosomal Recessive |
| C16899 | MTND6 | Leber Hereditary Optic Neuropathy; Mitochondrial |
| | | Disorders; Mitochondrial DNA-Associated Leigh |
| | | Syndrome and NARP |
| AA069417 | MTND4 | Leber Hereditary Optic Neuropathy; Mitochondrial |
| | | Disorders; Mitochondrial DNA-Associated Leigh |
| | | Syndrome and NARP |
| HUMCYP3A | MTND4 | Leber Hereditary Optic Neuropathy; Mitochondrial |
| | | Disorders; Mitochondrial DNA-Associated Leigh |
| | | Syndrome and NARP |
| HSCPHC22 | MTND1 | Leber Hereditary Optic Neuropathy; Mitochondrial |
| | | Disorders; Mitochondrial DNA-Associated Leigh |
| | | Syndrome and NARP |
| HUMHPRT | HPRT1 | Lesch-Nyhan Syndrome |
| HUMLHHCGR | LHCGR | Leydig Cell Hypoplasia/Agenesis; Male-Limited |
| | | Precocious Puberty |
| HSP53 | TP53 | Li-Fraumeni Syndrome |
| Z19198 | HADHB | Long Chain 3-Hydroxyacyl-CoA Dehydrogenase |
| | | Deficiency |
| M79018 | HADHA | Long Chain 3-Hydroxyacyl-CoA Dehydrogenase |
| | | Deficiency |
| W93500 | KCNQ1 | Atrial Fibrillation; Jervell and Lange-Nielsen |
| | | Syndrome; LQT 1; Romano-Ward Syndrome |
| S62085 | OCRL | Lowe Syndrome |
| T48981 | FBN1 | Marfan Syndrome |
| HUMASFB | ARSB | Mucopolysaccharidosis Type VI |
| M62202 | GNAS | Albright Hereditary Osteodystrophy; McCune-Albright |
| | | Syndrome; Osseus Heteroplasia, Progressive |
| N46342 | SACS | ARSACS |
| T81605 | FANCD2 | Fanconi Anemia |
| H47777 | FANCD1 | Fanconi Anemia |
| T23877 | ACADM | Medium Chain Acyl-Coenzyme A Dehydrogenase |
| | | Deficiency |
| AA906866 | PARK2 | Parkin Type of Juvenile Parkinson Disease |
| BE140729 | GJB4 | Erythrokeratodermia Variabilis |
| HSU26727 | CDKN2A | Familial Malignant Melanoma |
| T47218 | SPINK5 | Netherton Syndrome |
| HSMNKMBP | ATP7A | ATP7A-Related Copper Transport Disorders |
| R37821 | SHFM4 | Ectrodactyly |
| M78183 | GSN | Amyloidosis V |
| HSARYA | ARSA | Chromosome 22q13.3 Deletion Syndrome; |
| | | Metachromatic Leukodystrophy |
| S68531 | COL10A1 | Metaphyseal Chondrodysplasia, Schmid Type |
| T59742 | CACNA1A | Episodic Ataxia Type 2; Familial Hemiplegic Migraine; |
| | | Spinocerebellar Ataxia Type 6 |
| HSCP2 | HPS3 | Hermansky-Pudlak Syndrome; Hermansky-Pudlak |
| | | Syndrome 3 |
| R21301 | HPS3 | Hermansky-Pudlak Syndrome; Hermansky-Pudlak |
| | | Syndrome 3 |
| HUMBGALRP | GLB1 | GM1 Gangliosidosis; Mucopolysaccharidosis Type |
| | | IVB |
| HSU12507 | KCNJ2 | Andersen Syndrome |
| R28488 | MEN1 | Multiple Endocrine Neoplasia Type 1 |
| HUMCOMP | COMP | COMP-Related Multiple Epiphyseal Dysplasia; |
| | | Multiple Epiphyseal Dysplasia, Dominant; |
| | | Pseudoachondroplasia |
| H30258 | COL9A2 | Multiple Epiphyseal Dysplasia, Dominant |
| T48133 | EXT1 | Hereditary Multiple Exostoses; Multiple Exostoses, |
| | | Type I |
| T06129 | EXT2 | Hereditary Multiple Exostoses; Multiple Exostoses, |
| | | Type II |
| T05624 | LAMA2 | Congenital Muscular Dystrophy with Merosin |
| | | Deficiency |
| HSDYSTIA | DMD | Duchenne/Becker Muscular Dystrophy; |
| | | Dystrophinopathies; X-Linked Dilated |
| | | Cardiomyopathy |
| HSSTA | EMD | Emery-Dreifuss Muscular Dystrophy, X-Linked |
| HSU20165 | BMPR2 | Primary Pulmonary Hypertension |
| M79239 | CAPN3 | Calpainopathy; Limb-Girdle Muscular Dystrophies, |
| | | Autosomal Recessive |
| HSU34976 | SGCG | Gamma-Sarcoglycanopathy; Limb-Girdle Muscular |
| | | Dystrophies, Autosomal Recessive; |
| | | Sarcoglycanopathies |
| HUMADHA | SGCA | Alpha-Sarcoglycanopathy; Limb-Girdle Muscular |
| | | Dystrophies, Autosomal Recessive; |
| | | Sarcoglycanopathies |
| Z25374 | SGCB | Beta-Sarcoglycanopathy; Limb-Girdle Muscular |
| | | Dystrophies, Autosomal Recessive; |
| | | Sarcoglycanopathies |
| N29439 | SGCD | Delta-Sarcoglycanopathy; Dilated Cardiomyopathy; |
| | | Limb-Girdle Muscular Dystrophies, Autosomal |
| | | Recessive; Sarcoglycanopathies |
| N56180 | CASQ2 | Catecholaminergic Ventricular Tachycardia, |
| | | Autosomal Recessive |
| T23560 | CHRNB2 | Nocturnal Frontal Lobe Epilepsy, Autosomal Dominant |
| HSCHRNA44 | CHRNA4 | Nocturnal Frontal Lobe Epilepsy, Autosomal Dominant |
| M78654 | CHRNA4 | Nocturnal Frontal Lobe Epilepsy, Autosomal Dominant |
| T86329 | CDH23 | Usher Syndrome Type 1 |
| D11677 | PABPN1 | Oculopharyngeal Muscular Dystrophy |
| AW449267 | PCDH15 | Usher Syndrome Type 1 |
| HUMCLC | CLCN1 | Myotonia Congenita, Dominant; Myotonia |
| | | Congenita, Recessive |
| S86455 | DMPK | Myotonic Dystrophy Type 1 |
| T70260 | MTM1 | Myotubular Myopathy, X-Linked |
| T12579 | LMX1B | Nail-Patella Syndrome |
| HSTRKT1 | TPM3 | Nemaline Myopathy |
| HUMTROPCK | TPM3 | Nemaline Myopathy |
| Z19248 | NEB | Nemaline Myopathy |
| AF030626 | AVPR2 | Nephrogenic Diabetes Insipidus; Nephrogenic Diabetes |
| | | Insipidus, X-Linked |
| AA780862 | NPHS1 | Congenital Finnish Nephrosis |
| T08860 | ABCC8 | ABCC8-Related Hyperinsulinism; Familial |
| | | Hyperinsulinism |
| AA679741 | KCNJ11 | Familial Hyperinsulinism; KCNJ11-Related |
| | | Hyperinsulinism |
| M77935 | NF1 | Neurofibromatosis 1 |
| HSMEORPRA | NF2 | Neurofibromatosis 2 |
| T08995 | CLN3 | CLN3-Related Neuronal Ceroid-Lipofuscinosis; |
| | | Neuronal Ceroid-Lipofuscinoses |
| T72120 | CLN2 | CLN2-Related Neuronal Ceroid-Lipofuscinosis; |
| | | Neuronal Ceroid-Lipofuscinoses |
| T41059 | GRHPR | Hyperoxaluria, Primary, Type 2 |
| HUMGCRFC | FCGR3A | Neutrophil Antigen Genotyping |
| R21657 | NPC1 | Niemann-Pick Disease Type C; Niemann-Pick Disease |
| | | Type C1 |
| M77961 | SMPD1 | Niemann-Pick Disease Due to Sphingomyelinase |
| | | Deficiency |
| T87256 | SUOX | Sulfocysteinuria |
| D79813 | SOST | SOST-Related Sclerosing Bone Dysplasias |
| T94707 | MATN3 | Multiple Epiphyseal Dysplasia, Dominant |
| HSCOL9AL | COL9A1 | Multiple Epiphyseal Dysplasia, Dominant |
| S69208 | TNNT1 | Nemaline Myopathy |
| Z19459 | TPM2 | Nemaline Myopathy |
| D11793 | SLC2A1 | Glucose Transporter Type 1 Deficiency Syndrome |
| HSCHRX | NDP | Norrie Disease |
| T62791 | OPA1 | Optic Atrophy 1 |
| Z24812 | OFD1 | Oral-Facial-Digital Syndrome Type I |
| HUMOTC | OTC | Ornithine Transcarbamylase Deficiency |
| R66505 | MKKS | Bardet-Biedl Syndrome; McKusick-Kaufman |
| | | Syndrome |
| Z19438 | CHAC | Choreoacanthocytosis |
| HUMRDSA | RDS | Patterned Dystrophy of Retinal Pigment Epithelium; |
| | | Retinitis Pigmentosa, Autosomal Dominant |
| Z30072 | PLP1 | Hereditary Spastic Paraplegia, X-Linked; PLP- |
| | | Related Disorders |
| HSFGR1IG | FGFR1 | FGFR-Related Craniosynostosis Syndromes; Pfeiffer |
| | | Syndrome Type 1, 2, and 3 |
| HUMPHH | PAH | Phenylalanine Hydroxylase Deficiency |
| HSKITCR | KIT | Gastrointestinal Stromal Tumor; Piebaldism |
| HSGROW1 | GH1 | Pituitary Dwarfism I |
| F00079 | GHR | Pituitary Dwarfism II |
| HSPIT1 | POU1F1 | Pituitary-Specific Transcription Factor Defects (PIT1) |
| T58874 | SDHD | Familial Nonchromaffin Paragangliomas |
| HUMINTB3 | ITGB3 | Integrin, Beta 3; Platelet Antigen Genotyping |
| T09245 | PKD1 | Polycystic Kidney Disease 1, Autosomal Dominant; |
| | | Polycystic Kidney Disease, Autosomal Dominant |
| T55657 | PKD2 | Polycystic Kidney Disease 2, Autosomal Dominant; |
| | | Polycystic Kidney Disease, Autosomal Dominant |
| T77325 | PKD2 | Polycystic Kidney Disease 2, Autosomal Dominant; |
| | | Polycystic Kidney Disease, Autosomal Dominant |
| W27963 | PKD2 | Polycystic Kidney Disease 2, Autosomal Dominant; |
| | | Polycystic Kidney Disease, Autosomal Dominant |
| R05352 | PKHD1 | Polycystic Kidney Disease, Autosomal Recessive |
| M77871 | PCLD | Polycystic Liver Disease |
| M78097 | UROD | Porphyria Cutanea Tarda |
| HUMPBG | HMBS | Acute Intermittent Porphyria |
| HUMRODSA | UROS | Congenital Erythropoietic Porphyria |
| T10891 | AGT | Angiotensinogen |
| T67463 | CTSK | Pycnodysostosis |
| M77954 | PDHA1 | Pyruvate Dehydrogenase Deficiency, X-linked |
| Z19400 | PHYH | Refsum Disease, Adult |
| R07476 | PEX1 | Zellweger Syndrome Spectrum |
| Z24965 | RCA1 | Renal Cell Carcinoma |
| H37900 | RHO | Retinitis Pigmentosa, Autosomal Dominant; Retinitis |
| | | Pigmentosa, Autosomal Recessive |
| T24020 | RB1 | Retinoblastoma |
| Z44098 | RS1 | X-Linked Juvenile Retinoschisis |
| HSRH30A | RHCE | Rh C Genotyping; Rh E Genotyping |
| S57971 | RHCE | Rh C Genotyping; Rh E Genotyping |
| T89255 | RHCE | Rh C Genotyping; Rh E Genotyping |
| R60192 | PEX7 | Refsum Disease, Adult; Rhizomelic |
| | | Chondrodysplasia Punctata Type 1 |
| HUMMLC1AA | MLC1 | Megalencephalic Leukoencephalopathy with |
| | | Subcortical Cysts |
| M79106 | MLC1 | Megalencephalic Leukoencephalopathy with |
| | | Subcortical Cysts |
| T64905 | PITX2 | Anophthalmia; Peters Anomaly; Rieger Syndrome |
| Z41163 | CREBBP | Rubinstein-Taybi Syndrome |
| HSBHLH | TWIST1 | Saethre-Chotzen Syndrome |
| F00367 | EIF2B1 | Childhood Ataxia with Central Nervous System |
| | | Hypomyelination/Vanishing White Matter |
| Z20030 | EIF2B2 | Childhood Ataxia with Central Nervous System |
| | | Hypomyelination/Vanishing White Matter |
| Z41323 | EIF2B3 | Childhood Ataxia with Central Nervous System |
| | | Hypomyelination/Vanishing White Matter |
| Z17882 | EIF2B4 | Childhood Ataxia with Central Nervous System |
| | | Hypomyelination/Vanishing White Matter |
| R13846 | EIF2B5 | Childhood Ataxia with Central Nervous System |
| | | Hypomyelination/Vanishing White Matter; Cree |
| | | Leukoencephalopathy |
| T03917 | HEXB | Sandhoff Disease |
| HUMSRYA | SRY | XX Male Syndrome; XY Gonadal Dysgenesis |
| HUMSCAD | ACADS | Short Chain Acyl-CoA Dehydrogenase Deficiency |
| HSALAS2R | ALAS2 | Sideroblastic Anemia, X-Linked |
| T47846 | GPC3 | Simpson-Golabi-Behmel Syndrome |
| T11069 | GUSB | Mucopolysaccharidosis Type VII |
| T08813 | SPG3A | Hereditary Spastic Paraplegia, Dominant; SPG 3 |
| Z40639 | SPG3A | Hereditary Spastic Paraplegia, Dominant; SPG 3 |
| M77964 | SPG4 | Hereditary Spastic Paraplegia, Dominant; SPG 4 |
| N36808 | SMN1 | Spinal Muscular Atrophy |
| Z38265 | SMN1 | Spinal Muscular Atrophy |
| T06490 | SCA1 | Spinocerebellar Ataxia Type 1 |
| T55469 | SCA2 | Spinocerebellar Ataxia Type 2 |
| Z41764 | SCA2 | Spinocerebellar Ataxia Type 2 |
| T61453 | MJD | Spinocerebellar Ataxia Type 3 |
| HUMELASF | ELN | Cutis Laxa, Autosomal Dominant; Supravalvular |
| | | Aortic Stenosis |
| T05970 | HEXA | Hexosaminidase A Deficiency |
| M79184 | THRB | Thyroid Hormone Resistance |
| Z20729 | TCOF1 | Treacher Collins Syndrome |
| R48739 | TRPS1 | Trichorhinophalangeal Syndrome Type I |
| T77655 | TSC1 | Tuberous Sclerosis 1; Tuberous Sclerosis Complex |
| M78940 | TSC2 | Tuberous Sclerosis 2; Tuberous Sclerosis Complex |
| HSFAA | FAH | Tyrosinemia Type I |
| T39510 | TBX3 | Ulnar-Mammary Syndrome |
| HUMM7AA | MYO7A | Usher Syndrome Type 1 |
| W22160 | USH1C | Usher Syndrome Type 1 |
| T08506 | ACADVL | Very Long Chain Acyl-CoA Dehydrogenase |
| | | Deficiency |
| HUMHIPLIND | VHL | Von Hippel-Lindau Syndrome |
| HUMVWF | VWF | Von Willebrand Disease |
| HSU02368 | PAX3 | Waardenburg Syndrome Type I |
| H80461 | WRN | Werner Syndrome |
| HUMWND | ATP7B | Wilson Disease |
| T40645 | WAS | WAS-Related Disorders |
| HSLAL | LIPA | Wolman Disease |
| HSASL1 | ASL | Argininosuccinicaciduria |
| HSAGAGENE | AGA | Aspartylglycosaminuria |
| T88756 | ATD | Asphyxiating Thoracic Dystrophy |
| Z19164 | ASAH | Farber Disease |
| HUMALD | FBP1 | Fructose 1,6 Bisphosphatase Deficiency |
| HSLDHAR | LDHA | Lactate Dehydrogenase Deficiency |
| M77886 | LDHB | Lactate Dehydrogenase Deficiency |
| HSU13680 | LDHC | Lactate Dehydrogenase Deficiency |
| Z46189 | MAN2B1 | Alpha-Mannosidosis |
| M79249 | MANBA | Beta-Mannosidosis |
| H26723 | GALNS | Mucopolysaccharidosis Type IVA |
| H23053 | SLC26A4 | DFNB 4; Enlarged Vestibular Aqueduct Syndrome; |
| | | Nonsyndromic Hearing Loss and Deafness, Autosomal |
| | | Recessive; Pendred Syndrome |
| HSPGK1 | PGK1 | Phosphoglycerate Kinase Deficiency |
| HSU08818 | MET | Papillary Renal Carcinoma |
| M79231 | PRCC | Papillary Renal Carcinoma |
| T08200 | GNS | Mucopolysaccharidosis Type IIID |
| HUMNAGB | NAGA | Schindler Disease |
| T08881 | NEU1 | Mucolipidosis I |
| R81783 | SLC17A5 | Free Sialic Acid Storage Disorders |
| HUMAUTONH | MTATP6 | Mitochondrial Disorders; Mitochondrial DNA- |
| | | Associated Leigh Syndrome and NARP |
| F09306 | SCA7 | Spinocerebellar Ataxia Type 7 |
| AF248482 | DAZ | Y Chromosome Infertility |
| HSU21663 | DAZ | Y Chromosome Infertility |
| T47024 | JAG1 | Alagille Syndrome |
| HSRYRRM1 | RBMY1A1 | Y Chromosome Infertility |
| HSRYRRM2 | RBMY1A1 | Y Chromosome Infertility |
| HSVD3R | VDR | Osteoporosis; Rickets-Alopecia Syndrome |
| T40157 | FMO3 | Trimethylaminuria |
| HUMPHOSLIP | PPGB | Galactosialidosis |
| HUMPPR | PPGB | Galactosialidosis |
| H22222 | FANCC | Fanconi Anemia |
| D12009 | RPS6KA3 | Coffin-Lowry Syndrome |
| M78282 | PTEN | PTEN Hamartoma Tumor Syndrome (PHTS) |
| M78802 | FY | Duffy Antigen Genotyping |
| HSU04270 | KCNH2 | LQT 2; Romano-Ward Syndrome |
| T19733 | SCN5A | Brugada Syndrome; LQT 3; Romano-Ward Syndrome |
| HSTFIIDX | TBP | Spinocerebellar Ataxia Type17 |
| HUMKCHA | KCNA1 | Episodic Ataxia Type 1 |
| HSU78110 | NRTN | Hirschsprung Disease |
| HSET3AA | EDN3 | Hirschsprung Disease |
| Z17351 | ECE1 | Hirschsprung Disease |
| T47284 | DHCR7 | Smith-Lemli-Opitz Syndrome |
| HUMXIHB | HBZ | Alpha-Thalassemia |
| HSCP2 | CP | Aceruloplasminemia |
| N25320 | CLN6 | CLN6-Related Neuronal Ceroid-Lipofuscinosis; |
| | | Neuronal Ceroid-Lipofuscinoses |
| T11340 | NBS1 | Nijmegen Breakage Syndrome |
| Z40114 | NBS1 | Nijmegen Breakage Syndrome |
| HSU03688 | CYP1B1 | Glaucoma, Recessive (Congenital); Peters Anomaly |
| D62980 | MYOC | Glaucoma, Dominant (Juvenile Onset) |
| T98453 | NAGLU | Mucopolysaccharidosis Type IIIB |
| AA779817 | RUNX2 | Cleidocranial Dysplasia |
| HUMCBFA | RUNX2 | Cleidocranial Dysplasia |
| HSMARENO | MEFV | Familial Mediterranean Fever |
| F02180 | PHKB | Phosphorylase Kinase Deficiency of Liver and Muscle |
| D11905 | HPS1 | Hermansky-Pudlak Syndrome; Hermansky-Pudlak |
| | | Syndrome 1 |
| R95987 | CRX | Retinitis Pigmentosa, Autosomal Dominant |
| T05762 | EVC | Ellis-van Creveld Syndrome |
| T12126 | FLNA | Frontometaphyseal Dysplasia; Melnick-Needles |
| | | Syndrome; Otopalatodigital Syndrome; Periventricular |
| | | Heterotopia, X-Linked |
| T60913 | EBP | Chondrodysplasia Punctata, X-Linked Dominant |
| HSHNF4 | HNF4A | Maturity-Onset Diabetes of the Young Type I |
| HUMBGLUKIN | GCK | Familial Hyperinsulinism; GCK-Related |
| | | Hyperinsulinism; Maturity-Onset Diabetes of the |
| | | Young Type II |
| M62026 | GCK | Familial Hyperinsulinism; GCK-Related |
| | | Hyperinsulinism; Maturity-Onset Diabetes of the |
| | | Young Type II |
| R94860 | CIAS1 | Chronic Infantile Neurological Cutaneous and Articular |
| | | Syndrome; Familial Cold Urticaria; Muckle-Wells |
| | | Syndrome |
| T08221 | SMARCAL1 | Schimke Immunoosseous Dysplasia |
| T95621 | SLC25A15 | Hyperornithinemia-Hyperammonemia- |
| | | Homocitrullinuria Syndrome |
| HUMOATC | OAT | Ornithine Aminotransferase Deficiency |
| R08989 | MLYCD | Malonyl-CoA Decarboxylase Deficiency |
| T20008 | PMM2 | Congenital Disorders of Glycosylation |
| HSRPMI | MPI | Congenital Disorders of Glycosylation |
| HSSRECV6 | MGAT2 | Congenital Disorders of Glycosylation |
| T91755 | MGAT2 | Congenital Disorders of Glycosylation |
| HSCPTI | CPT1A | Carnitine Palmitoyltransferase IA (liver) Deficiency |
| HUMCPT | CPT2 | Carnitine Palmitoyltransferase II Deficiency |
| HSA1ATCA | SERPINA1 | Alpha-1-Antitrypsin Deficiency |
| N36808 | SMN2 | Spinal Muscular Atrophy |
| Z38265 | SMN2 | Spinal Muscular Atrophy |
| HUMACADL | ACADL | Long Chain Acyl-CoA Dehydrogenase Deficiency |
| Z25247 | CACT | Carnitine-Acylcarnitine Translocase Deficiency |
| HUMETFA | ETFA | Glutaricacidemia Type 2 |
| HSETFBS | ETFB | Glutaricacidemia Type 2 |
| S69232 | ETFDH | Glutaricacidemia Type 2 |
| T09377 | MEB | Muscle-Eye-Brain Disease |
| Z40427 | G6PT1 | Glycogen Storage DiseaseType Ib |
| AI002801 | SLC14A1 | Kidd Genotyping |
| Z19313 | SLC14A1 | Kidd Genotyping |
| HUMPGAMM | PGAM2 | Phosphoglycerate Mutase Deficiency |
| H86930 | MPP4 | Retinitis Pigmentosa, Autosomal Recessive |
| HSU14910 | RGR | Retinitis Pigmentosa, Autosomal Recessive |
| AA775466 | CARD15 | Crohn Disease |
| AA306952 | GAN | Giant Axonal Neuropathy |
| T99245 | CLCN5 | Dent Disease |
| T23537 | NR3C2 | Pseudohypoaldosteronism Type 1, Dominant |
| HSLASNA | SCNN1A | Pseudohypoaldosteronism Type 1, Recessive |
| H26938 | SCNN1B | Pseudoaldosteronism; Pseudohypoaldosteronism Type |
| | | 1, Recessive |
| HUMGAMM | SCNN1G | Pseudoaldosteronism; Pseudohypoaldosteronism Type |
| | | 1, Recessive |
| HSP450AL | CYP11B2 | Familial Hyperaldosteronism Type 1; Familial |
| | | Hypoaldosteronism Type 2 |
| HUMCYPADA | CYP11B1 | Familial Hyperaldosteronism Type 1 |
| AF017089 | COL11A1 | Stickler Syndrome; Stickler Syndrome Type II |
| HUMCA1XIA | COL11A1 | Stickler Syndrome; Stickler Syndrome Type II |
| HUMA2XICOL | COL11A2 | Stickler Syndrome |
| S61523 | PIGA | Paroxysmal Nocturnal Hemoglobinuria |
| T58881 | PHKA2 | Glycogen Storage Disease Type IX |
| Z39614 | DHAPAT | Rhizomelic Chondrodysplasia Punctata Type 2 |
| N89899 | SH2D1A | Lymphoproliferative Disease, X-Linked |
| HUMUGT1FA | UGT1A1 | Gilbert Syndrome |
| HUMNC1A | COL7A1 | Epidermolysis Bullosa Dystrophica, Bart Type; |
| | | Epidermolysis Bullosa Dystrophica, Cockayne- |
| | | Touraine Type; Epidermolysis Bullosa Dystrophica, |
| | | Hallopeau-Siemens Type; Epidermolysis Bullosa |
| | | Dystrophica, Pasini Type; Epidermolysis Bullosa, |
| | | Pretibial. |
| T49684 | ITGB4 | Epidermolysis Bullosa Letalis with Pyloric Atresia |
| S66196 | ITGA6 | Epidermolysis Bullosa Letalis with Pyloric Atresia |
| T10988 | LAMC2 | Epidermolysis Bullosa Junctional, Herlitz-Pearson |
| | | Type |
| HUMLAMAA | LAMA3 | Epidermolysis Bullosa Junctional, Herlitz-Pearson |
| | | Type |
| Z24848 | LAMA3 | Epidermolysis Bullosa Junctional, Herlitz-Pearson |
| | | Type |
| T10484 | LAMB3 | Epidermolysis Bullosa Junctional, Disentis Type; |
| | | Epidermolysis Bullosa Junctional, Herlitz-Pearson |
| | | Type |
| HUMBP180AA | COL17A1 | Epidermolysis Bullosa Junctional, Disentis Type |
| M78889 | PLEC1 | Epidermolysis Bullosa with Muscular Dystrophy |
| Z38659 | SLC22A5 | Carnitine Deficiency, Systemic |
| T85099 | CTNS | Cystinosis |
| W27253 | CNGA3 | Achromatopsia; Achromatopsia 2 |
| HSU66088 | SLC5A5 | Thyroid Hormonogenesis Defect I |
| HUMTEKRPTK | TEK | Venous Malformation, Multiple Cutaneous and |
| | | Mucosal |
| R69741 | SLC26A2 | Achondrogenesis Type 1B; Atelosteogenesis Type 2; |
| | | Diastrophic Dysplasia; Multiple Epiphyseal Dysplasia, |
| | | Recessive |
| Z46092 | PEX10 | Zellweger Syndrome Spectrum |
| S55790 | COL4A3 | Alport Syndrome; Alport Syndrome, Autosomal |
| | | Recessive |
| HSCOL4A4 | COL4A4 | Alport Syndrome; Alport Syndrome, Autosomal |
| | | Recessive |
| T10559 | SHFM3 | Ectrodactyly |
| T93670 | FANCA | Fanconi Anemia |
| H47777 | FANCB | Fanconi Anemia |
| AA542822 | FANCE | Fanconi Anemia |
| HUMPSPB | PSAP | Metachromatic Leukodystrophy |
| HUMSAPA1 | PSAP | Metachromatic Leukodystrophy |
| S69686 | PSAP | Metachromatic Leukodystrophy |
| AA252786 | NCF1 | Chronic Granulomatous Disease |
| HUMNCF1A | NCF1 | Chronic Granulomatous Disease |
| HSTGFB1 | TGFB1 | Camurati-Engelmann Disease |
| R24242 | CYBA | Chronic Granulomatous Disease |
| HUMLNOXF | NCF2 | Chronic Granulomatous Disease |
| S41458 | PDE6B | Retinitis Pigmentosa, Autosomal Recessive |
| R21727 | DYSF | Dysferlinopathy; Limb-Girdle Muscular Dystrophies, |
| | | Autosomal Recessive |
| AF055580 | USH2A | Usher Syndrome Type 2; Usher Syndrome Type 2A |
| N36632 | MITF | Waardenburg Syndrome Type II; Waardenburg |
| | | Syndrome Type IIA |
| M78027 | MYH9 | DFNA 17; Epstein Syndrome; Fechtner Syndrome; |
| | | May-Hegglin Anomaly; Sebastian Syndrome |
| Z40194 | HPS4 | Hermansky-Pudlak Syndrome |
| AA333774 | GP1BA | Platelet Antigen Genotyping |
| M79110 | GP1BB | Platelet Antigen Genotyping |
| HUMGPIIBA | ITGA2B | Platelet Antigen Genotyping |
| T29174 | ITGA2 | Glycoprotein 1a Deficiency; Platelet Antigen |
| | | Genotyping |
| HSGST4 | GSTM1 | Lung Cancer |
| AA338271 | CHEK2 | Li-Fraumeni Syndrome |
| T78869 | CHEK2 | Li-Fraumeni Syndrome |
| T03839 | SH3BP2 | Cherubism |
| T67412 | IRF6 | IRF6-Related Disorders |
| AB037973 | FGF23 | Hypophosphatemic Rickets, Dominant |
| T60199 | FBLN5 | Cutis Laxa, Autosomal Recessive |
| T03890 | ARX | ARX-Related Disorders |
| M79175 | NSD1 | Sotos Syndrome |
| T07860 | NSD1 | Sotos Syndrome |
| M79181 | COH1 | Cohen Syndrome |
| MIHS75KDA | NDUFS1 | Leigh Syndrome (nuclear DNA mutation); |
| | | Mitochondrial Respiratory Chain Complex I |
| | | Deficiency |
| T09312 | NDUFV1 | Leigh Syndrome (nuclear DNA mutation); |
| | | Mitochondrial Respiratory Chain Complex I |
| | | Deficiency |
| AA399371 | SALL4 | Acrorenoocular Syndrome; Okihiro Syndrome |
| HUMA8SEQ | TIMP3 | Pseudoinflammatory Fundus Dystrophy |
| Z40623 | GDAP1 | Charcot-Marie-Tooth Neuropathy Type 4; Charcot- |
| | | Marie-Tooth Neuropathy Type 4A |
| AA128030 | FOXL2 | Blepharophimosis, Epicanthus Inversus, Ptosis |
| HUMCRTR | SLC6A8 | Creatine Deficiency Syndrome, X-Linked |
| T08882 | JPH3 | Huntington Disease-Like 2 |
| T07283 | SNRPN | Autistic Disorder; Pervasive, Developmental Disorders |
| Z38837 | SPR | Sepiapterin Reductase Deficiency (SR) |
| HUMANTIR | AGTR1 | Angiotensin II Receptor, Type 1 |
| T46961 | SEPN1 | Congenital Muscular Dystrophy with Early Spine |
| | | Rigidity; Multiminicore Disease |
| Z43954 | TRIM32 | Limb-Girdle Muscular Dystrophies, Autosomal |
| | | Recessive |
| Z19219 | TTID | Limb-Girdle Muscular Dystrophies, Autosomal |
| | | Dominant |
| HSECADH | CDH1 | Hereditary Diffuse Gastric Cancer |
| Z41199 | WFS1 | Nonsyndromic Low-Frequency Sensorineural Hearing |
| | | Loss; Wolfram Syndrome |
| HUMLORAA | LOR | Progressive Symmetric Erythrokeratoderma |
| Z38324 | HR | Alopecia Universalis; Papular Atrichia |
| T09039 | RYR1 | Central Core Disease of Muscle; Malignant |
| | | Hyperthermia Susceptibility; Multiminicore Disease |
| T10442 | GALE | Galactose Epimerase Deficiency |
| D82541 | PDB2 | Paget Disease of Bone |
| HSU20759 | CASR | Autosomal Dominant Hypocalcemia; Familial |
| | | Hypocalciuric Hypercalcemia, Type I; Familial |
| | | Isolated Hypoparathyroidism; Neonatal Severe Primary |
| | | Hyperparathyroidism |
| AA071082 | SALL1 | Townes-Brocks Syndrome |
| T81692 | EDAR | Hypohidrotic Ectodermal Dysplasia; Hypohidrotic |
| | | Ectodermal Dysplasia, Autosomal |
| HUMHPA1B | HP | Anhaptoglobinemia |
| HSU01922 | TIMM8A | Deafness-Dystonia-Optic Neuronopathy Syndrome |
| HUMHSDI | HSD3B2 | Prostate Cancer |
| HSU05659 | HSD17B3 | Prostate Cancer |
| Z38915 | NPHP4 | Nephronophthisis 4; Senior-Loken Syndrome |
| HSC1INHR | SERPING1 | Hereditary Angioneurotic Edema |
| D62739 | BBS7 | Bardet-Biedl Syndrome |
| T64266 | SLC7A7 | Lysinuric Protein Intolerance |
| S52028 | CTH | Cystathioninuria |
| Z30254 | EFEMP1 | Doyne Honeycomb Retinal Dystrophy; Patterned |
| | | Dystrophy of Retinal Pigment Epithelium |
| D59254 | ELOVL4 | Stargardt Disease 3 |
| S43856 | GCH1 | Dopa-Responsive Dystonia; GTP Cyclohydrolase 1- |
| | | Deficient DRD; GTP Cyclohydrolase-1 Deficiency |
| | | (GTPCH) |
| M78468 | PAFAH1B1 | 17-Linked Lissencephaly |
| M78473 | PAFAH1B1 | 17-Linked Lissencephaly |
| S51033 | MID1 | Opitz Syndrome, X-Linked |
| Z40343 | MID1 | Opitz Syndrome, X-Linked |
| HUM6PTHS | PTS | Pyruvoyltetrahydropterin Synthase Deficiency |
| M62103 | CIRH1A | North American Indian Childhood Cirrhosis |
| HSDHPR | QDPR | Dihydropteridine Reductase Deficiency (DHPR) |
| T23665 | FKRP | Congenital Muscular Dystrophy Type 1C; Limb-Girdle |
| | | Muscular Dystrophies, Autosomal Recessive |
| T60498 | LRPPRC | Leigh Syndrome, French-Canadian Type |
| HSACHRA | CHRNA1 | Congenital Myasthenic Syndromes |
| HSACHRB | CHRNB1 | Congenital Myasthenic Syndromes |
| HSACHRG | CHRND | Congenital Myasthenic Syndromes |
| HSACETR | CHRNE | Congenital Myasthenic Syndromes |
| HSACRAP | RAPSN | Congenital Myasthenic Syndromes |
| M78334 | COLQ | Congenital Myasthenic Syndromes |
| S56138 | CHAT | Congenital Myasthenic Syndromes |
| D11584 | SDHC | Familial Nonchromaffin Paragangliomas |
| HSPSTI | SPINK1 | Hereditary Pancreatitis |
| HSSPROTR | PROS1 | Protein S Heerlen Variant |
| HUMLAP | ITGB2 | Leukocyte Adhesion Deficiency, Type 1 |
| T12572 | ADAMTS13 | Familial Thrombotic Thrombocytopenia Purpura |
| HUMCOMIIP | SDHB | Carotid Body Tumors and Multiple Extraadrenal |
| | | Pheochromocytomas |
| NM005912 | MC4R | Obesity |
| HUMPAX8A | PAX8 | Congenital Hypothyroidism |
| AA037119 | FOXE1 | Bamforth-Lazarus Syndrome; Congenital |
| | | Hypothyroidism |
| AV754057 | FSHB | Isolated Follicle Stimulating Hormone Deficiency |
| HUMHOMEOA | PCBD | Pterin-4a Carbinolamine Dehydratase Deficiency |
| | | (PCD) |
| HSTHR | TH | Dopa-Responsive Dystonia; Tyrosine Hydroxylase- |
| | | Deficient DRD |
| AA219596 | ZIC3 | Heterotaxy Syndrome |
| HSU20324 | CSRP3 | Dilated Cardiomyopathy |
| HUMPHLAM | PLN | Dilated Cardiomyopathy |
| F10219 | ALMS1 | Alstrom Syndrome |
| T06612 | VCL | Dilated Cardiomyopathy |
| AF388366 | USH3A | Usher Syndrome Type 3 |
| Z40797 | SGCE | Myoclonus-Dystonia |
| T08448 | RAB7 | Charcot-Marie-Tooth Neuropathy Type 2 |
| D12383 | GARS | Charcot-Marie-Tooth Neuropathy Type 2 |
| Z36734 | HRPT2 | HRPT2-Related Disorders |
| H19914 | EDARADD | Hypohidrotic Ectodermal Dysplasia; Hypohidrotic |
| | | Ectodermal Dysplasia, Autosomal |
| T08852 | PPT1 | Neuronal, Ceroid-Lipofuscinoses; PPT1-Related |
| | | Neuronal Ceroid-Lipofuscinosis |
| HUMDRA | SLC26A3 | Familial Chloride Diarrhea |
| R16324 | AGPAT2 | Berardinelli-Seip Congenital Lipodystrophy |
| Z38569 | BSCL2 | Berardinelli-Seip Congenital Lipodystrophy |
| W28410 | OPN1MW | Blue-Mono-Cone-Monochromatic Type Colorblindness |
| T27896 | OPN1LW | Blue-Mono-Cone-Monochromatic Type Colorblindness |
| AI469991 | PHOX2A | Congenital Fibrosis of Extraocular Muscles |
| HSFSTHR | FSHR | Premature Ovarian Failure, Autosomal Recessive |
| HSLPH | LCT | Hypolactasia, Adult Type |
| Z41000 | BCS1L | Gracile Syndrome; Mitochondrial Respiratory Chain |
| | | Complex III Deficiency |
| HSCGJP | GJA1 | Oculodentodigital Dysplasia |
| HSPERFP1 | PRF1 | Familial Hemophagocytic Lymphohistiocytosis 2 |
| M78112 | GLUD1 | Familial Hyperinsulinism; GLUD1-Related |
| | | Hyperinsulinism |
| W79230 | RAX | Anophthalmia |
| AF041339 | PITX3 | Anophthalmia |
| AA151708 | HESX1 | Anophthalmia |
| HSSOXB | SOX3 | Anophthalmia; Mental Retardation, X-Linked, with |
| | | Growth Hormone Deficiency |
| HUMHMGBOX | SOX2 | Anophthalmia |
| HSGM2APA | GM2A | GM2 Activator Deficiency |
| Z19280 | GLC1E | Glaucoma, Dominant (Adult Onset) |
| T20165 | PHF6 | Borjeson-Forssman-Lehmann Syndrome |
| Z40394 | CMT4B2 | Charcot-Marie-Tooth Neuropathy Type 4 |
| HUMIHH | IHH | Brachydactyly Type A1 |
| HUMCDPK | CDK4 | Familial Malignant Melanoma |
| T39355 | SBDS | Shwachman-Diamond Syndrome |
| HSHMPLK | MPL | Amegakaryocytic Thrombocytopenia, Congenital |
| Z38860 | TRIM37 | Mulibrey Nanism |
| M62027 | DTNA | Familial Isolated Noncompaction of Left Ventrical |
| | | Myocardium |
| Z39175 | DDB2 | Xeroderma Pigmentosum |
| T09329 | MUTYH | MYH-Associated Polyposis |
| HUMAPA | APP | Alzheimer Disease Type 1; Early-Onset Familial |
| | | Alzheimer Disease |
| M79090 | GSS | 5-Oxoprolinuria |
| Z26981 | OXCT | 3-Oxoacid CoA Transferase |
| D12046 | PMS1 | Hereditary Non-Polyposis Colon Cancer |
| T08186 | PMS2 | Hereditary Non-Polyposis Colon Cancer |
| R00471 | MSH6 | Hereditary Non-Polyposis Colon Cancer |
| T60457 | NDUFS4 | Leigh Syndrome (nuclear DNA mutation); |
| | | Mitochondrial Respiratory Chain Complex I |
| | | Deficiency |
| D30864 | NDUFS8 | Leigh Syndrome (nuclear DNA mutation) |
| M78107 | SDHA | Leigh Syndrome (nuclear DNA mutation) |
| R15290 | NTDUFS7 | Leigh Syndrome (nuclear DNA mutation) |
| HUMPCBA | PC | Pyruvate Carboxylase Deficiency |
| W32719 | AASS | Hyperlysinemia |
| T23789 | PEX3 | Zellweger Syndrome Spectrum |
| T09086 | STK11 | Peutz-Jeghers Syndrome |
| T87335 | HAL | Histidmemia |
| Z19082 | ALDH4A1 | Hyperprolinemia, Type II |
| Z25227 | MADH4 | Juvenile Polyposis Syndrome |
| M78130 | XPB | Xeroderma Pigmentosum |
| T08987 | XPD | Xeroderma Pigmentosum |
| D81449 | XPF | Xeroderma Pigmentosum |
| HSXPGAA | XPG | Xeroderma Pigmentosum |
| HSAUHMR | AUH | 3-Methylglutaconic Aciduria Type 1 |
| T19530 | MMAB | Methylmalonicaciduria |
| Z40169 | MMAA | Methylmalonicaciduria |
| T93695 | BCAT1 | Hyperleucine-Isoleucinemia |
| Z41266 | BCAT2 | Hyperleucine-Isoleucinemia |
| HSU03506 | SLC1A1 | Dicarboxylicaminoaciduria |
| R88591 | PRODH | Hyperprolinemia, Type I |
| T05380 | EPM2A | Progressive Myoclonus Epilepsy, Lafora Type |
| T27227 | FANCF | Fanconi Anemia |
| Z41736 | FANCG | Fanconi Anemia |
| R66178 | ED4 | Ectodermal Dysplasia, Margarita Island Type |
| L25197 | KCNE1 | Jervell and Lange-Nielsen Syndrome; LQT 5; Romano- |
| | | Ward Syndrome |
| HUMUMOD | UMOD | Familial Nephropathy with Gout; Medullary Cystic |
| | | Kidney Disease 2 |
| HSU66583 | CRYGD | Cataract, Crystalline Aculeiform |
| HSPHR | PTHR1 | Chondrodysplasia, Blomstrand Type |
| T97980 | MTRR | Homocystinuria-Megaloblastic Anemia |
| S60710 | ADSL | Adenylosuccinase deficiency |
| Z38216 | SLC25A19 | Amish Lethal Microcephaly |
| T11501 | DBH | Dopamine Beta-Hydroxylase Deficiency |
| H11439 | NLGN3 | Autistic Disorder; Pervasive Developmental Disorders |
| R12551 | NLGN4 | Autistic Disorder; Pervasive Developmental Disorders |
| M78212 | ATP1A2 | Familial Hemiplegic Migraine |
| T96957 | SPCH1 | Severe Speech Delay |
| AI266171 | PHOX2B | Congenital Central Hypoventilation Syndrome |
| BG723199 | DSG4 | Localized Autosomal Recessive Hypotrichosis |
| T46918 | HSD11B2 | Apparent Mineralocorticoid Excess Syndrome |
| HUMFERLS | FTL | Hyperferritinemia Cataract Syndrome |
| HUMCKRASA | KRAS2 | Familial Pancreatic Cancer |
| S39383 | PTPN11 | LEOPARD Syndrome; Noonan Syndrome |
| HUMSTAR | STAR | Cholesterol Desmolase Deficiency |
| Z20453 | STAR | Cholesterol Desmolase Deficiency |
| HUMVPC | AVP | Neurohypophyseal Diabetes Insipidus |
| M62144 | MECP2 | Rett Syndrome |
| HSCA2VR | COL5A2 | Ehlers-Danlos Syndrome, Classic Type |
| HUMGENX | TNXB | Ehlers-Danlos-like Syndrome Due to Tenascin-X |
| | | Deficiency |
| R02385 | TNXB | Ehlers-Danlos-like Syndrome Due to Tenascin-X |
| | | Deficiency |
| T39901 | LITAF | Charcot-Marie-Tooth Neuropathy Type 1 |
| AA621310 | FOXE3 | Anophthalmia |
| H18132 | CFC1 | Heterotaxy Syndrome |
| R36719 | EBAF | Heterotaxy Syndrome |
| HSACTIIRE | ACVR2B | Heterotaxy Syndrome |
| T52017 | CRELD1 | Heterotaxy Syndrome |
| D11851 | LMNA | Dilated Cardiomyopathy; Emery-Dreifuss Muscular |
| | | Dystrophy, Autosomal Dominant; Familial Partial |
| | | Lipodystrophy, Dunnigan Type; Hutchinson-Gilford |
| | | Progeria Syndrome; Limb-Girdle Muscular |
| | | Dystrophies, Autosomal Dominant; Mandibuloacral |
| | | Dysplasia |
| D12062 | DSP | Cardiomyopathy, Dilated, with Woolly Hair and |
| | | Keratoderma; Keratosis Palmoplantaris Striata |
| H99382 | MSH3 | Hereditary Non-Polyposis Colon Cancer |
| AW205295 | NOG | Multiple Synostoses Syndrome |
| AA135181 | GJB3 | Erythrokeratodermia Variabilis |
| F10278 | PEO1 | Mitochondrial DNA Deletion Syndromes |
| M62022 | MASS1 | Febrile Seizures |
| Z42549 | UQCRB | Mitochondrial Respiratory Chain Complex III |
| | | Deficiency |
| HUMEGR2A | EGR2 | Charcot-Marie-Tooth Neuropathy Type 1; Charcot- |
| | | Marie-Tooth Neuropathy Type 1D; Charcot-Marie- |
| | | Tooth Neuropathy Type 4; Charcot-Marie-Tooth |
| | | Neuropathy Type 4E |
| HSFLT4X | FLT4 | Milroy Congenital Lymphedema |
| Z28459 | PEX26 | Zellweger Syndrome Spectrum |
| HUMRPS24A | RPS19 | Diamond-Blackfan Anemia |
| T11633 | RPS19 | Diamond-Blackfan Anemia |
| HSACMHCP | MYH7 | Dilated Cardiomyopathy; Familial Hypertrophic |
| | | Cardiomyopathy |
| Z25920 | TNNT2 | Dilated Cardiomyopathy; Familial Hypertrophic |
| | | Cardiomyopathy |
| HUMTRO | TPM1 | Dilated Cardiomyopathy; Familial Hypertrophic |
| | | Cardiomyopathy |
| Z18303 | MYBPC3 | Dilated Cardiomyopathy; Familial Hypertrophic |
| | | Cardiomyopathy |
| H5U09466 | COX10 | Leigh Syndrome (nuclear DNA mutation) |
| S72487 | ECGF1 | Mitochondrial Neurogastrointestinal Encephalopathy |
| | | Syndrome |
| M62196 | KIF5A | Hereditary Spastic Paraplegia, Dominant |
| T07578 | KIF5A | Hereditary Spastic Paraplegia, Dominant |
| D11648 | HSPD1 | Hereditary Spastic Paraplegia, Dominant |
| T47330 | SOX18 | Hypotrichosis-Lymphedema-Telangiectasia Syndrome |
| AA448334 | CAV3 | Caveolinopathy; Limb-Girdle Muscular Dystrophies, |
| | | Autosomal Dominant |
| AW071529 | ALX4 | Parietal Foramina 2 |
| M61973 | CD2AP | Focal Segmental Glomerulosclerosis |
| W21801 | NR2E3 | Enhanced S-Cone Syndrome |
| Z20305 | TREM2 | PLOSL |
| T05421 | ANK2 | LQT 4; Romano-Ward Syndrome |
| HUMROR2A | ROR2 | ROR2-Related Disorders |
| Z25920 | CMD1D | Dilated Cardiomyopathy |
| AA887962 | HLXB9 | Currarino Syndrome |
| R00281 | ALDH5A1 | Succinic Semialdehyde Dehydrogenase Deficiency |
| HSPCCAR | PCCA | Propionic Acidemia |
| N43992 | DLL3 | Spondylocostal Dysostosis, Autosomal Recessive; |
| | | Syndactyly, Type IV |
| Z39790 | MUT | Methylmalonicaciduria |
| HUMARGL | ARG1 | Argininemia |
| HUMRENBAT | SLC3A1 | Cystinuria |
| T80665 | SLC7A9 | Cystinuria |
| T27286 | HGD | Alkaptonuria |
| HUMBCKDH | BCKDHA | Maple Syrup Urine Disease |
| HUMBCKDHA | BCKDHB | Maple Syrup Urine Disease |
| HSTRANSP | DBT | Maple Syrup Urine Disease |
| Z44722 | HLCS | Holocarboxylase Synthetase Deficiency |
| Z38396 | BTD | Biotinidase Deficiency |
| T48178 | POMT1 | Walker-Warburg Syndrome |
| T28737 | GJB2 | DFNA 3 Nonsyndromic Hearing Loss and Deafness; |
| | | DFNB 1 Nonsyndromic Hearing Loss and Deafness; |
| | | GJB2-Related DFNA 3 Nonsyndromic Hearing Loss |
| | | and Deafness; GJB2-Related DFNB 1 Nonsyndromic |
| | | Hearing Loss and Deafness; Nonsyndromic Hearing |
| | | Loss and Deafness, Autosomal Dominant; |
| | | Nonsyndromic Hearing Loss and Deafness, Autosomal |
| | | Recessive; Vohwinkel Syndrome |
| T05861 | COCH | DFNA 9 (COCH); Nonsyndromic Hearing Loss and |
| | | Deafness, Autosomal Dominant |
| HSBRN4 | POU3F4 | DFN 3 |
| HSU21938 | TTPA | Ataxia with Vitamin E Deficiency (AVED) |
| T93783 | KIAA1985 | Charcot-Marie-Tooth Neuropathy Type 4 |
| BE735997 | SANS | Usher Syndrome Type 1 |
| AA548783 | HOXD13 | Syndactyly, Type II |
| R33750 | HOXA13 | Hand-Foot-Uterus Syndrome |
| HUMPP | GLDC | GLDC-Related Glycine Encephalopathy; Glycine |
| | | Encephalopathy |
| F04230 | AMT | AMT-Related Glycine Encephalopathy; Glycine |
| | | Encephalopathy |
| T54795 | DECR | 2,4-Dienoyl-CoA Reductase Deficiency |
| R07295 | ACAT1 | Ketothiolase Deficiency |
| S70578 | ACAT1 | Ketothiolase Deficiency |
| HUMMEVKIN | MVK | Hyper IgD Syndrome; Mevalonicaciduria |
| T11245 | HMGCL | 3-Hydroxy-3-Methylglutaryl-Coenzyme A Lyase |
| | | Deficiency |
| Z41427 | GCDH | Glutaricacidemia Type 1 |
| HSSHOXA | SHOX | Langer Mesomelic Dwarfism; Leri-Weill |
| | | Dyschondrosteosis; Short Stature |
| HUMDOPADC | DDC | Aromatic L-Amino Acid Decarboxylase Deficiency |
| HSCOL3A4 | COL6A3 | Limb-Girdle Muscular Dystrophies, Autosomal |
| | | Dominant |
| HSCOL1A4 | COL6A1 | Limb-Girdle Muscular Dystrophies, Autosomal |
| | | Dominant |
| HSCOL2C2 | COL6A2 | Limb-Girdle Muscular Dystrophies, Autosomal |
| | | Dominant |
| H16770 | RECQL4 | Rothmund-Thomson Syndrome |
| H11473 | SGSH | Mucopolysaccharidosis Type IIIA |
| H67137 | MCCC1 | 3-Methylcrotonyl-CoA Carboxylase Deficiency |
| R88931 | MCCC2 | 3-Methylcrotonyl-CoA Carboxylase Deficiency |
| Z24865 | TCAP | Dilated Cardiomyopathy; Limb-Girdle Muscular |
| | | Dystrophies, Autosomal Recessive |
| M86030 | DCX | DCX-Related Malformations |
| HUMACTASK | ACTA1 | Nemaline Myopathy |
| HSDGIGLY | DSG1 | Keratosis Palmoplantaris Striata |
| HSRETSA | SAG | Retinitis Pigmentosa, Autosomal Recessive |
| HSAPHOL | ALPL | Hypophosphatasia |
| N73784 | XPA | Xeroderma Pigmentosum |
| T28958 | XPC | Xeroderma Pigmentosum |
| N69543 | POLH | Xeroderma Pigmentosum |
| T54103 | POLH | Xeroderma Pigmentosum |
| H56484 | CKN1 | Cockayne Syndrome |
| Z38185 | ERCC6 | Cockayne Syndrome |
| F07041 | PI12 | Familial Encephalopathy with Neuroserpin Inclusion |
| | | Bodies |
| AA633404 | KCNE2 | LQT 6; Romano-Ward Syndrome |
| HSTITINC2 | CMD1G | Dilated Cardiomyopathy |
| N99115 | NPHP1 | Nephronophthisis 1; Senior-Loken Syndrome |
| HUMELANAA | ELA2 | ELA2-Related Neutropenia |
| S67325 | PCCB | Propionic Acidemia |
| HSGA7331 | M1S1 | Corneal Dystrophy, Gelatinous Drop-Like |
| HSACE | ACE | Angiotensin I Converting Enzyme 1 |
| S49816 | TSHR | Congenital Hypothyroidism; Familial Non- |
| | | Autoimmune Hyperthyroidism |
| Z30221 | VMGLOM | Multiple Glomus Tumors |
| H88042 | COL9A3 | Multiple Epiphyseal Dysplasia, Dominant |
| M78119 | ADA | Adenosine Deaminase Deficiency |
| T55785 | GAMT | Guanidinoacetate Methyltransferase Deficiency |
| HUMCST4BA | CSTB | Myoclonic Epilepsy of Unverricht and Lundborg |
| S73196 | AQP2 | Nephrogenic Diabetes Insipidus; Nephrogenic Diabetes |
| | | Insipidus, Autosomal |
| HSU76388 | NR5A1 | XY Sex Reversal with Adrenal Failure |
| HSCPHC22 | MTRNR1 | MTRNR1-Related Hearing Loss and Deafness |
| H21596 | PPARG | Diabetes Mellitus with Acanthosis Nigricans and |
| | | Hypertension |
| D56550 | FOXC1 | Anophthalmia; Rieger Syndrome |
| M78868 | AP3B1 | Hermansky-Pudlak Syndrome |
| T47068 | NOTCH3 | CADASIL |
| HSHMF1C | TCF1 | Maturity-Onset Diabetes of the Young Type III |
| AF049893 | IPF1 | Maturity-Onset Diabetes of the Young Type IV |
| HSU30329 | IPF1 | Maturity-Onset Diabetes of the Young Type IV |
| HSVHNF1 | TCF2 | Maturity-Onset Diabetes of the Young Type V |
| HUMLDLRFMT | LDLR | Familial Hypercholesterolemia |
| HSAPOBR2 | APOB | Familial Hypercholesterolemia Type B |
| T78010 | ABCB7 | Sideroblastic Anemia and Ataxia |
| AF076215 | PROP1 | PROP1-Related Combined Pituitary Hormone |
| | | Deficiency |
| S99468 | ALAD | Acute Hepatic Porphyria |
| T61818 | ABCC2 | Dubin-Johnson Syndrome |
| HUMLCAT | LCAT | Lecithin Cholesterol Acyltransferase Deficiency |
| Z38510 | HADHSC | Short Chain 3-Hydroxyacyl-CoA Dehydrogenase |
| | | Deficiency, Liver |
| AF041240 | PPOX | Variegate Porphyria |
| T77011 | PPOX | Variegate Porphyria |
| Z40014 | ALDH10 | Sjogren-Larsson Syndrome |
| S79867 | KRT16 | Nonepidermolytic Palmoplantar Hyperkeratosis; |
| | | Pachyonychia Congenita |
| HUMKER56K | KRT6A | Pachyonychia Congenita |
| HSKERELP | KRT17 | Pachyonychia Congenita; Steatocystoma Multiplex |
| R11850 | KRT6B | Pachyonychia Congenita |
| S69510 | KRT9 | Epidermolytic Palmoplantar Keratoderma |
| HSCYTK | KRT13 | White Sponge Nevus of Cannon |
| T92918 | KRT4 | White Sponge Nevus of Cannon |
| S54769 | SPG7 | Hereditary Spastic Paraplegia, Recessive; SPG 7 |
| T50707 | FECH | Erythropoietic Protoporphyria |
| HUMPOMM | PXMP3 | Zellweger Syndrome Spectrum |
| R05392 | PEX6 | Zellweger Syndrome Spectrum |
| Z38759 | PEX12 | Zellweger Syndrome Spectrum |
| R14480 | PEX16 | Zellweger Syndrome Spectrum |
| R10031 | PEX13 | Zellweger Syndrome Spectrum |
| R13532 | PXF | Zellweger Syndrome Spectrum |
| Z30136 | AGPS | Rhizomelic Chondrodysplasia Punctata Type 3 |
| HSU07866 | ACOX | Pseudoneonatal Adrenoleukodystrophy |
| N63143 | ALG6 | Congenital Disorders of Glycosylation |
| HSTNFR1A | TNFRSF1A | Familial Hibernian Fever |
| AA018811 | RP1 | Retinitis Pigmentosa, Autosomal Dominant |
| HSG11 | RP1 | Retinitis Pigmentosa, Autosomal Dominant |
| T07942 | RP1 | Retinitis Pigmentosa, Autosomal Dominant |
| H28658 | PRPF31 | Retinitis Pigmentosa, Autosomal Dominant |
| T07062 | PRPF8 | Retinitis Pigmentosa, Autosomal Dominant |
| T05573 | RP18 | Retinitis Pigmentosa, Autosomal Dominant |
| HUMNRLGP | NRL | Retinitis Pigmentosa, Autosomal Dominant |
| T87786 | CRB1 | Retinitis Pigmentosa, Autosomal Recessive |
| H92408 | TULP1 | Retinitis Pigmentosa, Autosomal Recessive |
| S42457 | CNGA1 | Retinitis Pigmentosa, Autosomal Recessive |
| H30568 | PDE6A | Retinitis Pigmentosa, Autosomal Recessive |
| M78192 | RLBP1 | Retinitis Pigmentosa, Autosomal Recessive; Retinitis |
| | | Pigmentosa, Autosomal Recessive, Bothnia Type |
| T10761 | SLC4A4 | Proximal Renal Tubular Acidosis with Ocular |
| | | Abnormalities |
| N64339 | GJB6 | DFNA 3 Nonsyndromic Hearing Loss and Deafness; |
| | | DFNB 1 Nonsyndromic Hearing Loss and Deafness; |
| | | GJB6-Related DFNB 1 Nonsyndromic Hearing Loss |
| | | and Deafness; GJB6-Related DFNA 3 Nonsyndromic |
| | | Hearing Loss and Deafness; Hidrotic Ectodermal |
| | | Dysplasia 2; Nonsyndromic Hearing Loss and |
| | | Deafness, Autosomal Dominant; Nonsyndromic |
| | | Hearing Loss and Deafness, Autosomal Recessive |
| T67968 | MAT1A | Isolated Persistent Hypermethioninemia |
| HUMUMPS | UMPS | Oroticaciduria |
| HSPNP | NP | Purine Nucleoside Phosphorylase Deficiency |
| AB006682 | AIRE | Autoimmune Polyendocrinopathy Syndrome Type 1 |
| BE871354 | JUP | Naxos Disease |
| T08214 | JUP | Naxos Disease |
| F00120 | DES | Dilated Cardiomyopathy |
| R28506 | MOCS1 | Molybdenum Cofactor Deficiency |
| T70309 | MOCS2 | Molybdenum Cofactor Deficiency |
| T08212 | SNCA | Parkinson Disease |
| R99091 | ABCC6 | Pseudoxanthoma Elasticum |
| T69749 | ABCC6 | Pseudoxanthoma Elasticum |
| AA207040 | PRG4 | Arthropathy Camptodactyly Syndrome |
| T07189 | PRG4 | Arthropathy Camptodactyly Syndrome |
| F07016 | OPPG | Osteoporosis Pseudoglioma Syndrome |
| H27782 | SCO2 | Fatal Infantile Cardioencephalopathy due to COX |
| | | Deficiency |
| S54705S1 | PRKAR1A | Carney Complex |
| Z25903 | SCA10 | Spinocerebellar Ataxia Type10 |
| AA592984 | WISP3 | Progressive Pseudorheumatoid Arthropathy of |
| | | Childhood |
| Z39666 | MCOLN1 | Mucolipidosis IV |
| HSEMX2 | EMX2 | Familial Schizencephaly |
| HUMSP18A | SFTPB | Pulmonary Surfactant Protein B Deficiency |
| T10596 | ATP8B1 | Benign Recurrent Intrahepatic Cholestasis; Progressive |
| | | Familial Intrahepatic Cholestasis; Progressive Familial |
| | | Intrahepatic Cholestasis 1 |
| U46845 | CYP27B1 | Pseudovitamin D Deficiency Rickets |
| Z21585 | MAPT | Frontotemporal Dementia with Parkinsonism-17 |
| HSPPD | HPD | Tyrosinemia Type III |
| HUMUGT1FA | UGT1A | Crigler-Najjar Syndrome |
| R20880 | SLC19A2 | Thiamine-Responsive Megaloblastic Anemia |
| | | Syndrome |
| H42203 | TFAP2B | Char Syndrome |
| Z30126 | RYR2 | Catecholaminergic Ventricular Tachycardia, |
| | | Autosomal Dominant |
| HSSPYRAT | AGXT | Hyperoxaluria, Primary, Type 1 |
| T80758 | SEDL | Spondyloepiphyseal Dysplasia Tarda, X-Linked |
| T89449 | SEDL | Spondyloepiphyseal Dysplasia Tarda, X-Linked |
| AA373083 | FOXC2 | Lymphedema with Distichiasis |
| HUMPROP2AB | SCA12 | Spinocerebellar Ataxia Type12 |
| Z30145 | ACTC | Dilated Cardiomyopathy |
| HS1900 | GDNF | Hirschsprung Disease |
| M62223 | NEFL | Charcot-Marie-Tooth Neuropathy Type 1F/2E; |
| | | Charcot-Marie-Tooth Neuropathy Type 2; Charcot- |
| | | Marie-Tooth Neuropathy Type 2E/1F |
| T10920 | SERPINE1 | Plasminogen Activator Inhibitor I |
| HSNCAML1 | L1CAM | Hereditary Spastic Paraplegia, X-Linked; L1 |
| | | Syndrome |
| T11074 | L1CAM | Hereditary Spastic Paraplegia, X-Linked; L1 Syndrome |
| HUMHPROT | GCSH | Glycine Encephalopathy |
| HSTATR | TAT | Tyrosinemia Type II |
| Z19514 | CPT1B | Carnitine Palmitoyltransferase IB (muscle) Deficiency |
| HSALK3A | BMPR1A | Juvenile Polyposis Syndrome |
| T78581 | CLN5 | CLN5-Related Neuronal Ceroid-Lipofuscinosis; |
| | | Neuronal Ceroid-Lipofuscinoses |
| N32269 | CLN8 | CLN8-Related Neuronal Ceroid-Lipofuscinosis; |
| | | Neuronal Ceroid-Lipofuscinoses |
| HSU44128 | SLC12A3 | Gitelman Syndrome |
| AI590292 | NPHS2 | Focal Segmental Glomerulosclerosis; Steroid-Resistant |
| | | Nephrotic Syndrome |
| M62209 | ACTN4 | Focal Segmental Glomerulosclerosis |
| H53423 | CNGB3 | Achromatopsia; Achromatopsia 3 |
| HSEPAR | HCI | Hemangioma, Hereditary |
| R14741 | ZIC2 | Holoprosencephaly 5 |
| H84264 | SIX3 | Anophthalmia; Holoprosencephaly 2 |
| T10497 | TGIF | Holoprosencephaly 4 |
| Z30052 | USP9Y | Y Chromosome Infertility |
| N85185 | DBY | Y Chromosome Infertility |
| T11164 | SPTLC1 | Hereditary Sensory Neuropathy Type I |
| T68440 | GNE | GNE-Related Myopathies; Sialuria, French Type |
| HSPROPERD | PFC | Properdin Deficiency, X-Linked |
| T46865 | SURF1 | Leigh Syndrome (nuclear DNA mutation) |
| AI015025 | VAX1 | Anophthalmia |
| BM727523 | VAX1 | Anophthalmia |
| AA310724 | SIX6 | Anophthalmia |
| R37821 | TP63 | TP63-Related Disorders |
| AF091582 | ABCB11 | Progressive Familial Intrahepatic Cholestasis |
| HUMHOX7 | MSX1 | Hypodontia, Autosomal Dominant; Tooth-and-Nail |
| | | Syndrome |
| R15034 | CACNB4 | Episodic Ataxia Type 2 |
| T52100 | TYROBP | PLOSL |
| F09012 | MTMR2 | Charcot-Marie-Tooth Neuropathy Type 4 |
| T08510 | APTX | Ataxia with Oculomotor Apraxia; Ataxia with |
| | | Oculomotor Apraxia 1 |
| HUMHAAC | HF1 | Hemolytic-Uremic Syndrome |
| C16899 | MTND5 | Leber Hereditary Optic Neuropathy; Mitochondrial |
| | | DNA-Associated Leigh Syndrome and NARP |

#DRUG_DRUG_INTERACTION: refers to proteins involved in a biological process which mediates the interaction between at least two consumed drugs. Novel splice variants of known protein is involved in interaction between drugs may be used, for example, to modulate such drug-drug interactions. Examples of proteins involved in drug-drug interactions are presented in Table 7 together with the corresponding internal gene contig name, enabling to allocate the new splice variants within the data files “proteins.fasta” and “transcripts.fasta” in the attached CD-ROM1 and “proteins” and “transcripts” files in the attached CD-ROM2.

TABLE 7

| Contig | Gene Symbol | Description |
|---|---|---|
| HUMANTLA | SLC3A2 | 4f2 cell-surface antigen heavy chain |
| Z43093 | HTR6 | 5-hydroxytryptamine 6 receptor |
| HSXLALDA | ABCD1 | Adrenoleukodystrophy protein |
| R35137 | GPT | Alanine aminotransferase |
| D11683 | ALDH1 | Aldehyde dehydrogenase, cytosolic |
| T53833 | AOX1 | Aldehyde oxidase |
| HUMAGP1A | ORM1 | Alpha-1-acid glycoprotein 1 |
| HUMAGP1A | ORM2 | Alpha-1-acid glycoprotein 2 |
| HUMABPA | ABP1 | Amiloride-sensitive amine oxidase [copper-containing] |
| S62734 | MAOB | Amine oxidase [flavin-containing] b |
| AA526963 | SLC6A14 | Amino acid transporter b0+ |
| HSAE2 | SLC4A2 | Anion exchange protein 2 |
| M78110 | SLC4A3 | Anion exchange protein 3 |
| M78052 | ABCB2 | Antigen peptide transporter 1 |
| HUMMHCIIAB | ABCB3 | Antigen peptide transporter 2 |
| F02693 | APOD | Apolipoprotein d |
| M62234 | ASNA1 | Arsenical pump-driving ATPase |
| HUMNORTR | NAT1 | Arylamine n-acetyltransferase 1 |
| T67129 | NAT1 | Arylamine n-acetyltransferase 1 |
| AI262683 | NAT2 | Arylamine n-acetyltransferase 2 |
| Z39550 | ABCB9 | ATP-binding cassette protein abcb9 |
| Z44377 | ABCA1 | ATP-binding cassette, sub-family a, member 1 |
| M78056 | ABCA2 | ATP-binding cassette, sub-family a, member 2 |
| M85498 | ABCA3 | ATP-binding cassette, sub-family a, member 3 |
| T79973 | ABCB6 | ATP-binding cassette, sub-family b, member 6, mitochondrial |
| T78010 | ABCB7 | ATP-binding cassette, sub-family b, member 7, mitochondrial |
| R89046 | ABCB8 | ATP-binding cassette, sub-family b, member 8, mitochondrial |
| H64439 | ABCD2 | ATP-binding cassette, sub-family d, member 2 |
| M85760 | ABCD3 | ATP-binding cassette, sub-family d, member 3 |
| Z21904 | ABCD4 | ATP-binding cassette, sub-family d, member 4 |
| Z39977 | ABCG1 | ATP-binding cassette, sub-family g, member 1 |
| Z45628 | ABCG2 | ATP-binding cassette, sub-family g, member 2 |
| T80665 | SLC7A9 | B(0, +)-type amino acid transporter 1 |
| AF091582 | ABCB11 | Bile salt export pump |
| Z38696 | BLMH | Bleomycin hydrolase |
| T08127 | BNPI | Brain-specific na-dependent inorganic phosphate cotransporter |
| F00545 | SLC12A2 | Bumetanide-sensitive sodium-(potassium)-chloride cotransporter 2 |
| HSU07969 | CDH17 | Cadherin-17 |
| T10238 | SLC25A12 | Calcium-binding mitochondrial carrier protein aralar1 |
| Z40674 | SLC25A13 | Calcium-binding mitochondrial carrier protein aralar2 |
| T61818 | ABCC2 | Canalicular multispecific organic anion transporter 1 |
| T39953 | ABCC3 | Canalicular multispecific organic anion transporter 2 |
| HUMCRE | CBR1 | Carbonyl reductase [nadph] 1 |
| AA320697 | CBR3 | Carbonyl reductase [nadph] 3 |
| F03362 | COMT | Catechol o-methyltransferase, membrane-bound form |
| T11004 | COMT | Catechol o-methyltransferase, membrane-bound form |
| T39368 | SLC7A4 | Cationic amino acid transporter-4 |
| S74445 | RBP5 | Cellular retinol-binding protein iii |
| T55952 | RBP5 | Cellular retinol-binding protein iii |
| HSU39905 | SLC18A1 | Chromaffin granule amine transporter |
| R52371 | SLC35A1 | Cmp-sialic acid transporter |
| D20754 | CNT3 | Concentrative nucleoside transporter 3 |
| HSMNKMBP | ATP7A | Copper-transporting ATPase 1 |
| HUMWND | ATP7B | Copper-transporting ATPase 2 |
| HUMCFTRM | ABCC7 | Cystic fibrosis transmembrane conductance regulator |
| F10774 | SLC7A11 | Cystine/glutamate transporter |
| HUMCYPADA | CYP11B1 | Cytochrome P450 11B1, mitochondrial |
| HUMARM | CYP19 | Cytochrome P450 19 |
| HUMCYP145 | CYP1A1 | Cytochrome P450 1A1 |
| R21282 | CYP26 | Cytochrome P450 26 |
| AF209774 | CYP2A13 | Cytochrome P450 2A13 |
| HSC45B2C | CYP2A6 | Cytochrome P450 2A6 |
| HSC45B2C | CYP2A7 | Cytochrome P450 2A7 |
| HSP452B6 | CYP2B6 | Cytochrome P450 2B6 |
| HUM2C18 | CYP2C18 | Cytochrome P450 2C18 |
| HSCP450 | CYP2C19 | Cytochrome P450 2C19 |
| HUM2C18 | CYP2C19 | Cytochrome P450 2C19 |
| HUMCYPAX | CYP2C8 | Cytochrome P450 2C8 |
| HSCP450 | CYP2C9 | Cytochrome P450 2C9 |
| HSP450 | CYP2D6 | Cytochrome P450 2D6 |
| M77918 | CYP2E1 | Cytochrome P450 2E1 |
| HUMCYPIIF | CYP2F1 | Cytochrome P450 2F1 |
| H09076 | CYP2J2 | Cytochrome P450 2J2 |
| R07010 | CYP39A1 | Cytochrome P450 39A1 |
| HUMCYPHLP | CYP3A3 | Cytochrome P450 3A3 |
| HUMCYPHLP | CYP3A4 | Cytochrome P450 3A4 |
| AA416822 | CYP3A43 | Cytochrome P450 3A43 |
| HUMCYP3A | CYP3A5 | Cytochrome P450 3A5 |
| T82801 | CYP3A7 | Cytochrome P450 3A7 |
| HSCYP4AA | CYP4A11 | Cytochrome P450 4A11 |
| S67580 | CYP4A11 | Cytochrome P450 4A11 |
| HUMCP45IV | CYP4B1 | Cytochrome P450 4B1 |
| T98002 | CYP4F12 | Cytochrome P450 4F12 |
| AA377259 | CYP4F2 | Cytochrome P450 4F2 |
| AI400898 | CYP4F8 | Cytochrome P450 4F8 |
| HSU09178 | DPYD | Dihydropyrimidine dehydrogenase [nadp+] |
| W03174 | DPYD | Dihydropyrimidine dehydrogenase [nadp+] |
| HUMFMO1 | FMO1 | Dimethylaniline monooxygenase [n-oxide forming] 1 |
| HSFLMON2R | FMO2 | Dimethylaniline monooxygenase [n-oxide forming] 2 |
| T64494 | FMO2 | Dimethylaniline monooxygenase [n-oxide forming] 2 |
| T40157 | FMO3 | Dimethylaniline monooxygenase [n-oxide forming] 3 |
| HSFLMON2R | FMO4 | Dimethylaniline monooxygenase [n-oxide forming] 4 |
| D12220 | FMO5 | Dimethylaniline monooxygenase [n-oxide forming] 5 |
| H25503 | HET | Efflux transporter like protein |
| T12485 | HET | Efflux transporter like protein |
| M78151 | EPHX1 | Epoxide hydrolase 1 |
| T66884 | SLC29A1 | Equilibrative nucleoside transporter 1 |
| HSHNP36 | SLC29A2 | Equilibrative nucleoside transporter 2 |
| T08444 | SLC1A3 | Excitatory amino acid transporter 1 |
| HSU01824 | SLC1A2 | Excitatory amino acid transporter 2 |
| HSU03506 | SLC1A1 | Excitatory amino acid transporter 3 |
| F07883 | SLC1A6 | Excitatory amino acid transporter 4 |
| N39099 | SLC1A7 | Excitatory amino acid transporter 5 |
| F00548 | SLC2A9 | Facilitative glucose transporter family member glut9 |
| T95337 | SLC27A1 | Fatty acid transport protein |
| Z44099 | SLC27A1 | Fatty acid transport protein |
| HUMALBP | FABP4 | Fatty acid-binding protein, adipocyte |
| S67314 | FABP3 | Fatty acid-binding protein, heart |
| AW605378 | FABP2 | Fatty acid-binding protein, intestinal |
| L25227 | SLC19A1 | Folate transporter 1 |
| HSI15PGN1 | FABP6 | Gastrotropin |
| Z40427 | G6PT1 | Glucose 5-phosphate transporter |
| D11793 | SLC2A1 | Glucose-transporter type 1, erythrocyte/brain |
| N27535 | SLC2A10 | Glucose transporter type 10 |
| T52633 | SLC2A11 | Glucose transporter type 11 |
| HUMLGTPA | SLC2A2 | Glucose transporter type 2, liver |
| HUMLGTPA | SLC2A2 | Glucose transporter type 2, liver |
| T07239 | SLC2A3 | Glucose transporter type 3, brain |
| HUMIRGT | SLC2A4 | Glucose transporter type 4, insulin-responsive |
| M62105 | SLC2A5 | Glucose transporter type 5, small intestine |
| T59518 | SLC2A8 | Glucose transporter type 8 |
| HUMLGTH1 | GSTA1 | Glutathione s-transferase a1 |
| HUMLGTH1 | GSTA2 | Glutathione s-transferase a2 |
| T98291 | GSTA3 | Glutathione s-transferase a3-3 |
| Z21581 | GSTA4 | Glutathione s-transferase a4-4 |
| HSGST4 | GSTM1 | Glutathione s-transferase mu 1 |
| D31291 | GSTM2 | Glutathione s-transferase mu 2 |
| HSGST4 | GSTM2 | Glutathione s-transferase mu 2 |
| T08311 | GSTM3 | Glutathione s-transferase mu 3 |
| HUMGSTM4B | GSTM4 | Glutathione s-transferase mu 4 |
| HUMGSTM5 | GSTM5 | Glutathione s-transferase mu 5 |
| T05391 | GSTP1 | Glutathione s-transferase p |
| Z32822 | GSTT1 | Glutathione s-transferase theta 1 |
| R08187 | GSTT2 | Glutathione s-transferase theta 2 |
| Z25318 | GSTK1 | Glutathione s-transferase, mitochondrial |
| H03163 | SLC37A1 | Glycerol-3-phosphate transporter |
| AA363955 | SLC5A7 | High affinity choline transporter |
| HSRRMRNA | SLC7A1 | High-affinity cationic amino acid transporter-1 |
| R22196 | SLC31A1 | High-affinity copper uptake protein 1 |
| AA918012 | SLC10A2 | Ileal sodium/bile acid transporter |
| F00840 | SLC7A5 | Large neutral amino acid transporter small subunit 1 |
| M79133 | SLC7A5 | Large neutral amino acid transporter small subunit 1 |
| Z38621 | SLC7A8 | Large neutral amino acids transporter small subunit 2 |
| HUMCARAA | CES1 | Liver carboxylesterase |
| S52379 | CES1 | Liver carboxylesterase |
| T55488 | SLC21A6 | Liver-specific organic anion transporter |
| W78748 | SLC5A4 | Low affinity sodium-glucose cotransporter |
| T54842 | SLC7A2 | Low-affinity cationic amino acid transporter-2 |
| T87799 | ABCA7 | Macrophage abc transporter |
| Z17844 | LRP | Major vault protein |
| Z24885 | GSTZ1 | Maleylacetoacetate isomerase |
| T39939 | MT1A | Metallothionein-IA |
| R99207 | MT1B | Metallothionein-IB |
| T39939 | MT1E | Metallothionein-IE |
| D11725 | MT1F | Metallothionein-IF |
| S68949 | MT1G | Metallothionein-IG |
| S68954 | MT1G | Metallothionein-IG |
| HSFMET | MT1H | Metallothionein-IH |
| S52379 | MT2A | Metallothionein-II |
| M78846 | MT3 | Metallothionein-III |
| AA570216 | MT1K | Metallothionein-IK |
| S68954 | MT1K | Metallothionein-IK |
| D11725 | MT1L | Metallothionein-IL |
| HSPP15 | MT1L | Metallothionein-IL |
| HSPP15 | MT1R | Metallothionein-IR |
| NM032935 | MT4 | Metallothionein-IV |
| HUMGST | MGST1 | Microsomal glutathione s-transferase 1 |
| H59104 | MGST2 | Microsomal glutathione s-transferase 2 |
| T47062 | MGST3 | Microsomal glutathione s-transferase 3 |
| SSMPCP | SLC25A3 | Mitochondrial phosphate carrier protein |
| H39996 | SULT1A3 | Monoamine-sulfating phenol sulfotransferase |
| HUMARYTRAB | SULT1A3 | Monoamine-sulfating phenol sulfotransferase |
| M62141 | SLC16A1 | Monocarboxylate transporter 1 |
| H90048 | SLC16A6 | Monocarboxylate transporter 2 |
| F02520 | SLC16A2 | Monocarboxylate transporter 3 |
| AI005004 | SLC16A8 | Monocarboxylate transporter 4 |
| T59354 | SLC16A3 | Monocarboxylate transporter 5 |
| R22416 | SLC16A4 | Monocarboxylate transporter 6 |
| T78890 | SLC16A5 | Monocarboxylate transporter 7 |
| F01173 | SLC16A7 | Monocarboxylate transporter 8 |
| Z41819 | ABCB1 | Multidrug resistance protein 1 |
| HUMMDR3 | ABCB4 | Multidrug resistance protein 3 |
| SATHRMRP | ABCC1 | Multidrug resistance-associated protein 1 |
| R00050 | ABCC4 | Multidrug resistance-associated protein 4 |
| M78673 | ABCC5 | Multidrug resistance-associated protein 5 |
| R99091 | ABCC6 | Multidrug resistance-associated protein 6 |
| T69749 | ABCC6 | Multidrug resistance-associated protein 6 |
| D11495 | DIA4 | Nad(p)h dehydrogenase [quinone] 1 |
| HUMNRAMP | SLC11A1 | Natural resistance-associated macrophage protein 1 |
| Z38360 | SLC11A2 | Natural resistance-associated macrophage protein 2 |
| HUMASCT1A | SLC1A4 | Neutral amino acid transporter a |
| T10696 | SLC1A5 | Neutral amino acid transporter b(0) |
| HUMRENBAT | SLC3A1 | Neutral and basic amino acid transport protein rbat |
| HSU08021 | NNMT | Nicotinamide n-methyltransferase |
| T87759 | SLC22A4 | Novel organic cation transporter 1 |
| Z41935 | SLC15A2 | Oligopeptide transporter, kidney isoform |
| HSU21936 | SLC15A1 | Oligopeptide transporter, small intestine isoform |
| M62053 | OAT1 | Organic anion transporter 1 |
| H18607 | OAT3 | Organic anion transporter 3 |
| R16970 | OAT4 | Organic anion transporter 4 |
| T39111 | SLC21A9 | Organic anion transporter b |
| Z41576 | SLC21A11 | Organic anion transporter oATP-d |
| T23657 | SLC21A12 | Organic anion transporter oATP-e |
| Z21041 | SLC21A14 | Organic anion transporting polypeptide 14 |
| H75435 | SLC21A8 | Organic anion transporting polypeptide 8 |
| HSU77086 | SLC22A1 | Organic cation transporter 1 |
| HSOCTK | SLC22A2 | Organic cation transporter 2 |
| T53187 | SLC22A3 | Organic cation transporter 3 |
| H30224 | ORCTL4 | Organic cation transporter like 4 |
| H25503 | ORCTL2 | Organic cation transporter-like 2 |
| Z38659 | SLC22A5 | Organic cation/carnitine transporter 2 |
| AB010438 | ORCTL3 | Organic-cation transporter like 3 |
| T95621 | ORNT1 | Ornithine transporter |
| AA398593 | ORNT2 | Ornithine transporter 2 |
| R79412 | NTT5 | Orphan sodium- and chloride-dependent neurotransmitter |
| | | transporter ntt5 |
| H82347 | NTT73 | Orphan sodium- and chloride-dependent neurotransmitter |
| | | transporter ntt73 |
| Z43484 | NTT73 | Orphan sodium- and chloride-dependent neurotransmitter |
| | | transporter ntt73 |
| Z44749 | SLC25A17 | Peroxisomal membrane protein pmp34 |
| HUMARYLSUL | SULT1A1 | Phenol-sulfating phenol sulfotransferase 1 |
| HUMARYLSUL | SULT1A2 | Phenol-sulfating phenol sulfotransferase 2 |
| D12243 | RBP4 | Plasma retinol-binding protein |
| HUMATPAD | ATP12A | Potassium-transporting ATPase alpha chain 2 |
| Z40030 | ATP8A1 | Potential phospholipid-transporting ATPase ia |
| T10596 | FIC1 | Potential phospholipid-transporting ATPase ic |
| T86800 | SLC31A2 | Probable low-affinity copper uptake protein 2 |
| Z41717 | PTGIS | Prostacyclin synthase |
| S78220 | PTGS1 | Prostaglandin g/h synthase 1 |
| HUMENDOSYN | PTGS2 | Prostaglandin g/h synthase 2 |
| T85296 | SLC21A2 | Prostaglandin transporter |
| M62053 | SLC22A6 | Renal organic anion transport protein 1 |
| HSU26209 | SLC13A2 | Renal sodium/dicarboxylate cotransporter |
| Z40774 | SLC13A2 | Renal sodium/dicarboxylate cotransporter |
| HSNAPI1 | SLC17A1 | Renal sodium-dependent phosphate transport protein 1 |
| HUMNAPI3X | SLC34A1 | Renal sodium-dependent phosphate transport protein 2 |
| H85361 | ABCA4 | Retinal-specific ATP-binding cassette transporter |
| S74445 | CRABP1 | Retinoic acid-binding protein i, cellular |
| HUMCRABP | CRABP2 | Retinoic acid-binding protein ii, cellular |
| HUMCRBP | RBP1 | Retinol-binding protein i, cellular |
| S57153 | RBP1 | Retinol-binding protein i, cellular |
| T07054 | RBP2 | Retinol-binding protein ii, cellular |
| T63266 | RBP2 | Retinol-binding protein ii, cellular |
| HUMBGT1R | SLC6A12 | Sodium- and chloride-dependent betaine transporter |
| HUMCRTR | SLC6A8 | Sodium- and chloride-dependent creatine transporter 1 |
| R20043 | SLC6A13 | Sodium- and chloride-dependent gaba transporter 2 |
| S70609 | SLC6A9 | Sodium- and chloride-dependent glycine transporter 1 |
| AA625644 | SLC6A5 | Sodium- and chloride-dependent glycine transporter 2 |
| M78677 | SLC6A6 | Sodium- and chloride-dependent taurine transporter |
| T10761 | SLC4A4 | Sodium bicarbonate cotransporter nbc1 |
| AA452802 | NBC4 | Sodium bicarbonate cotransporter nbc4a |
| HUMCNC | SLC8A1 | Sodium/calcium exchanger 1 |
| R20720 | SLC8A2 | Sodium/calcium exchanger 2 |
| T07666 | SLC8A3 | Sodium/calcium exchanger 3 |
| T07666 | SLC8A3 | Sodium/glucose cotransporter 1 |
| HUMSGLCT | SLC5A2 | Sodium/glucose cotransporter 2 |
| S83549 | SLC9A2 | Sodium/hydrogen exchanger 2 |
| HSU66088 | SLC5A5 | Sodium/iodide cotransporter |
| HSU62966 | SLC28A1 | Sodium/nucleoside cotransporter 1 |
| AA358822 | SLC28A2 | Sodium/nucleoside cotransporter 2 |
| HUMNTCP | SLC10A1 | Sodium/taurocholate cotransporting polypeptide |
| HSGAT1MR | SLC6A1 | Sodium-and chloride-dependent gaba transporter 1 |
| F05686 | SLC6A11 | Sodium-and chloride-dependent gaba transporter 3 |
| AA604857 | SVCT1 | Sodium-denpendent vitamin c transporter 1 |
| T27309 | SVCT2 | Sodium-denpendent vitamin c transporter 2 |
| S44626 | SLC6A3 | Sodium-dependent dopamine transporter |
| Z39412 | NADC3 | Sodium-dependent high-affinity dicarboxylate transporter |
| T77525 | SLC5A6 | Sodium-dependent multivitamin transporter |
| HUMNORTR | SLC6A2 | Sodium-dependent noradrenaline transporter |
| HSZ83953 | SLC17A3 | Sodium-dependent phosphate transport protein 3 |
| R06460 | SLC17A3 | Sodium-dependent phosphate transport protein 3 |
| HSZ83953 | SLC17A4 | Sodium-dependent phosphate transport protein 4 |
| R09122 | SLC17A4 | Sodium-dependent phosphate transport protein 4 |
| H40741 | SLC6A7 | Sodium-dependent proline transporter |
| HSSERT | SLC6A4 | Sodium-dependent serotonin transporter |
| T64950 | SLC21A3 | Sodium-independent organic anion transporter |
| M79233 | EPHX2 | Soluble epoxide hydrolase |
| Z39813 | SLC25A18 | Solute carrier |
| HUMSTAR | STAR | Steroidogenic acute regulatory protein |
| Z20453 | STAR | Steroidogenic acute regulatory protein |
| R69741 | SLC26A2 | Sulfate transporter |
| T08860 | ABCC8 | Sulfonylurea receptor 1 |
| R73927 | ABCC9 | Sulfonylurea receptor 2 |
| T84623 | SULT1C1 | Sulfotransferase 1C1 |
| R58632 | SULT1C2 | Sulfotransferase 1C2 |
| HSVMT | SLC18A2 | Synaptic vesicle amine transporter |
| AF080246 | TRAG3 | Taxol resistant associated protein 3 |
| R20880 | SLC19A2 | Thiamine transporter 1 |
| HSU44128 | SLC12A3 | Thiazide-sensitive sodium-chloride cotransporter |
| S62904 | TPMT | Thiopurine s-methyltransferase |
| HSPBX2 | G17 | Transporter protein |
| T62038 | G17 | Transporter protein |
| R53836 | SLC35A3 | UDP n-acetylglucosamine transporter |
| T60594 | SLC35A2 | UDP-galactose translocator |
| HUMUGT1FA | UGT1 | UDP-glucuronosyltransferase 1-1, microsomal |
| HUMUGT1FA | UGT1A10 | UDP-glucuronosyltransferase 1A10 |
| HUMUGT1FA | UGT1A7 | UDP-glucuronosyltransferase 1A7 |
| HUMUGT1FA | UGT1A8 | UDP-glucuronosyltransferase 1A8 |
| HUMUGT1FA | UGT1A9 | UDP-glucuronosyltransferase 1A9 |
| HSUGT2BIO | UGT2B10 | UDP-glucuronosyltransferase 2B10, microsomal |
| HSUDPGT | UGT2B11 | UDP-glucuronosyltransferase 2B11, microsomal |
| N70316 | UGT2B11 | UDP-glucuronosyltransferase 2B11, microsomal |
| HSU08854 | UGT2B15 | UDP-glucuronosyltransferase 2B15, microsomal |
| T24450 | UGT2B17 | UDP-glucuronosyltransferase 2B17, microsomal |
| HSUDPGT | UGT2B4 | UDP-glucuronosyltransferase 2B4, microsomal |
| HUMUDPGTA | UGT2B7 | UDP-glucuronosyltransferase 2B7, microsomal |
| AI002801 | SLC14A1 | Urea transporter, erythrocyte |
| Z19313 | SLC14A1 | Urea transporter, erythrocyte |
| AI002801 | SLC14A2 | Urea transporter, kidney |
| HSU09210 | SLC18A3 | Vesicular acetylcholine transporter |
| HUMKCHB | KCNA4 | Voltage-gated potassium channel protein kv1.4 |
| R09608 | XDH | Xanthine dehydrogenase/oxidase |
| T64266 | SLC7A7 | Y + 1 amino acid transporter 1 |
| T10628 | SLC30A1 | Zinc transporter 1 |
| AA322641 | SLC30A4 | Zinc transporter 4 |

#EXONS_SKIPPED: This field details alternatively spliced exons identified according to the teachings of the present invention and their deletion to create the biomolecular sequences of the present invention. This field is marked by #EXONS_SKIPPED and thereafter the names of exons (for example: #EXONS_SKIPPED C15NT010194P1split49_{—}294009_{—}294072). C15NT010194P1split49_{—}294009_{—}294072 specifies the name of the exon of the present invention.

### Example 7

### Proteins and Diseases

The following sections list examples of proteins (subsection i), based on their molecular function, which participate in variety of diseases (listed in subsection ii), which diseases can be diagnosed/treated using the biomolecular sequences uncovered by the present invention.

The present invention is of biomolecular sequences, which can be classified to functional groups based on known activity of homologous sequences. This functional group classification, allows the identification of diseases and conditions, which may be diagnosed and treated based on the novel sequence information and annotations of the present invention.

This functional group classification includes the following groups:

Proteins Involved in Drug-Drug Interactions:

The phrase “proteins involved in drug-drug interactions” refers to proteins involved in a biological process which mediates the interaction between at least two consumed drugs.

Pharmaceutical compositions including such proteins or protein encoding sequences, antibodies directed against such proteins or polynucleotides capable of altering expression of such proteins, may be used to modulate drug-drug interactions. Antibodies and polynucleotides such as PCR primers and molecular probes designed to identify such proteins or protein encoding sequences may be used for diagnosis of such drug-drug interactions.

Examples of these conditions include, but are not limited to the cytochrom P450 protein family, which is involved in the metabolism of many drugs. Examples of proteins, which are involved in drug-drug interactions are presented in Table 7.

Proteins Involved in the Metabolism of a Pro-Drug to a Drug:

The phrase “proteins involved in the metabolism of a pro-drug to a drug” refers to proteins that activate an inactive pro-drug by chemically chaining it into a biologically active compound. Preferably, the metabolizing enzyme is expressed in the target tissue thus reducing systemic side effects.

Pharmaceutical compositions including such proteins or protein encoding sequences, antibodies directed against such proteins or polynucleotides capable of altering expression of such proteins, may be used to modulate the metabolism of a pro-drug into drug. Antibodies and polynucleotides such as PCR primers and molecular probes designed to identify such proteins or protein encoding sequences may be used for diagnosis of such conditions.

Examples of these proteins include, but are not limited to esterases hydrolyzing the cholesterol lowering drug simvastatin into its hydroxy acid active form.

MDR Proteins:

The phrase “MDR proteins” refers to Multi Drug Resistance proteins that are responsible for the resistance of a cell to a range of drugs, usually by exporting these drugs outside the cell. Preferably, the MDR proteins are ABC binding cassette proteins. Preferably, drug resistance is associated with resistance to chemotherapy.

Pharmaceutical compositions including such proteins or protein encoding sequences, antibodies directed against such proteins or polynucleotides capable of altering expression of such proteins, may be used to treat diseases in which the transport of molecules and macromolecules such as neurotransmitters, hormones, sugar etc. is abnormal leading to various pathologies. Antibodies and polynucleotides such as PCR primers and molecular probes designed to identify such proteins or protein encoding sequences may be used for diagnosis of such diseases.

Examples of these proteins include, but are not limited to the multi-drug resistant transporter MDR1/P-glycoprotein, the gene product of MDR1, which belongs to the ATP-binding cassette (ABC) superfamily of membrane transporters and increases the resistance of malignant cells to therapy by exporting the therapeutic agent out of the cell.

Hydrolases Acting on Amino Acids:

The phrase “hydrolases acting on amino acids” refers to hydrolases acting on a pair of amino acids.

Pharmaceutical compositions including such proteins or protein encoding sequences, antibodies directed against such proteins or polynucleotides capable of altering expression of such proteins, may be used to treat diseases in which the transfer of a glycosyl chemical group from one molecule to another is abnormal thus, a beneficial effect may be achieved by modulation of such reaction. Antibodies and polynucleotides such as PCR primers and molecular probes designed to identify such proteins or protein encoding sequences may be used for diagnosis of such diseases.

Examples of such diseases include, but are not limited to reperfusion of clotted blood vessels by TPA (Tissue Plasminogen Activator) which converts the abundant, but inactive, zymogen plasminogen to plasmin by hydrolyzing a single ARG-VAL bond in plasminogen.

Transaminases:

The term “transaminases” refers to enzymes transferring an amine group from one compound to another.

Pharmaceutical compositions including such proteins or protein encoding sequences, antibodies directed against such proteins or polynucleotides capable of altering expression of such proteins, may be used to treat diseases in which the transfer of an amine group from one molecule to another is abnormal thus, a beneficial effect may be achieved by modulation of such reaction. Antibodies and polynucleotides such as PCR primers and molecular probes designed to identify such proteins or protein encoding sequences may be used for diagnosis of such diseases.

Examples of such transaminases include, but are not limited to two liver enzymes, frequently used as markers for liver function—SGOT (Serum Glutamic-Oxalocetic Transaminase—AST) and SGPT (Serum Glutamic-Pyruvic Transaminase—ALT).

Immunoglobulins:

The term “immunoglobulins” refers to proteins that are involved in the immune and complement systems such as antigens and autoantigens, immunoglobulins, MHC and HLA proteins and their associated proteins.

Pharmaceutical compositions including such proteins or protein encoding sequences, antibodies directed against such proteins or polynucleotides capable of altering expression of such proteins, may be used to treat diseases involving the immune system such as inflammation, autoimmune diseases, infectious diseases, and cancerous processes. Antibodies and polynucleotides such as PCR primers and molecular probes designed to identify such proteins or protein encoding sequences may be used for diagnosis of such diseases.

Examples of such diseases and molecules that may be target for diagnostics include, but are not limited to members of the complement family such as C3 and C4 that their blood level is used for evaluation of autoimmune diseases and allergy state and C1 inhibitor that its absence is associated with angioedema. Thus, new variants of these genes are expected to be markers for similar events. Mutation in variants of the complement family may be associated with other immunological syndromes, such as increased bacterial infection that is associated with mutation in C3. C1 inhibitor was shown to provide safe and effective inhibition of complement activation after reperfused acute myocardial infarction and may reduce myocardial injury [Eur. Heart J. 2002, 23 (21):1670-7], thus its variant may have the same or improved effect.

Transcription Factor Binding:

The phrase “transcription factor binding” refers to proteins involved in transcription process by binding to nucleic acids, such as transcription factors, RNA and DNA binding proteins, zinc fingers, helicase, isomerase, histones, and nucleases.

Pharmaceutical compositions including such proteins or protein encoding sequences, antibodies directed against such proteins or polynucleotides capable of altering expression of such proteins may be used to treat diseases involving transcription factors binding proteins. Such treatment may be based on transcription factor that can be used to for modulation of gene expression associated with the disease. Antibodies and polynucleotides such as PCR primers and molecular probes designed to identify such proteins for protein encoding sequences may be used for diagnosis of such diseases.

Examples of such diseases include, but are not limited to breast cancer associated with ErbB-2 expression that was shown to be successfully modulated by a transcription factor [Proc. Natl. Acad. Sci. USA. 2000, 97(4):1495-500]. Examples of novel transcription factors used for therapeutic protein production include, but are not limited to those described for Erythropoietin production [J. Biol. Chem. 2000, 275(43):33850-60; J. Biol. Chem. 2000, 275(43):33850-60] and zinc fingers protein transcription factors (ZFP-TF) variants [J. Biol. Chem. 2000, 275(43) 33850-60].

Small GTPase Regulatory/Interacting Proteins:

The phrase “Small GTPase regulatory/interacting proteins” refers to proteins capable of regulating or interacting with GTPase such as RAB escort protein, guanyl-nucleotide exchange factor, guanyl-nucleotide exchange factor adaptor, GDP-dissociation inhibitor, GTPase inhibitor, GTPase activator, guanyl-nucleotide releasing factor, GDP-dissociation stimulator, regulator of G-protein signaling, RAS interactor, RHO interactor, RAB interactor, and RAL interactor.

Pharmaceutical compositions including such proteins or protein encoding sequences, antibodies directed against such proteins or polynucleotides capable of altering expression of such proteins, may be used to treat diseases in which G-proteases meditated signal-transduction is abnormal, either as a cause, or as a result of the disease. Antibodies and polynucleotides such as PCR primers and molecular the disease. Antibodies and polynucleotides such as PCR primers and molecular probes designed to identify such proteins or protein encoding sequences may be used for diagnosis of such diseases.

Examples of such diseases include, but are not limited to diseases related to prenylation. Modulation of prenylation was shown to affect therapy of diseases such as osteoporosis, ischemic heart disease, and inflammatory processes. Small GTPases regulatory/interacting proteins rare major component in the prenylation post translation modification, and are required to the normal activity of prenylated proteins. Thus, their variants may be used for therapy of prenylation associated diseases.

Calcium Binding Proteins:

The phrase “calcium binding proteins” refers to proteins involve in calcium binding, preferably, calcium binding proteins, ligand binding or carriers, such as diacylglycerol kinase, Calpain, calcium-dependent protein serine/threonine phosphatase, calcium sensing proteins, calcium storage proteins.

Pharmaceutical compositions including such proteins or protein encoding sequences, antibodies directed against such proteins or polynucleotides capable of altering expression of such proteins, may be used to treat calcium involved diseases. Antibodies and polynucleotides such as PCR primers and molecular probes designed to identify such proteins or protein encoding sequences may be used for diagnosis of such diseases.

Examples of such diseases include, but are not limited to diseases related to hypercalcemia, hypertension, cardiovascular disease, muscle diseases, gastro-intestinal diseases, uterus relaxing and uterus. An example for therapy use of calcium binding proteins variant may be treatment of emergency cases of hypercalcemia, with secreted variants of calcium storage proteins.

Oxidoreductase:

The term “oxidoreductase” refers to enzymes that catalyze the removal of hydrogen atoms and electrons from the compounds on which they act. Preferably, oxidoreductases acting on the following groups, of donors: CH—OH, CH—CH, CH—NH2, CH—NH; oxidoreductases acting on NADH or NADPH, nitrogenous compounds, sulfur group of donors, heme group, hydrogen group, diphenols and related substances as donors; oxidoreductases acting on peroxide as acceptor, superoxide radicals as acceptor, oxidizing metal ions, CH2 groups; oxidoreductases acting on reduced ferredoxin as donor; oxidoreductases acting on reduced flavodoxin as donor and oxidoreductases acting on the aldehyde or oxo group of donors.

Pharmaceutical compositions including such proteins or protein encoding sequences, antibodies directed against such proteins or polynucleotides capable of altering expression of such proteins, may be used to treat diseases caused by abnormal activity of oxidoreductases. Antibodies and polynucleotides such as PCR primers and molecular probes designed to identify such proteins or protein encoding sequences may be used for diagnosis of such diseases.

Examples of such diseases include, but are not limited to malignant and autoimmune diseases in which the enzyme DHFR (DiHydroFolateReductase) that participates in folate metabolism and essential for de novo glycine and purine synthesis is the target for the widely used drug Methotrexate (MTX).

Receptors:

The term “receptors” refers to protein-binding sites on a cell's surface or interior, that recognize and binds to specific messenger molecule leading to a biological response, such as signal transducers, complement receptors, ligand-dependent nuclear receptors, transmembrane receptors, GPI-anchored membrane-bound receptors, various coreceptors, internalization rectors, receptors to neurotransmitters, hormones and various other effectors and ligands.

Pharmaceutical compositions including such proteins or protein encoding sequences, antibodies directed against such proteins or polynucleotides capable of altering expression of such proteins, may be used to treat diseases caused by abnormal activity of receptors, preferably, receptors to neurotransmitters, hormones and various other effectors and ligands. Antibodies and polynucleotides such as PCR primers and molecular probes designed to identify such proteins or protein encoding sequences may be used for diagnosis of such diseases.

Examples of such diseases include, but are not limited to, chronic myelomonocytic leukemia caused by growth factor β receptor deficiency [Rao D. S., et al., (2001) Mol. Cell. Biol., 21(22):7796-806], thrombosis associated with protease-activated receptor deficiency [Sambrano G. R., et al., (2001) Nature, 413(6851):26-73, hypercholesterolemia associated with low density lipoprotein receptor deficiency [Koivisto U. M., et al., (2001) Cell, 105(5) 575-85], familial Hibernian fever associated with tumor necrosis factor receptor deficiency [Simon A., et al., (2001) Ned Tijdschr Geneeskd, 145(2):77-8], colitis associated with immunoglobulin E receptor expression [Dombrowicz D., et al. (2001) J. Exp. Med., 193(1):25-34], and alagille syndrome associated with Jagged1 [Stankiewicz P. et al., (2001) Am. J. Med. Genet., 103(2):166-71], breast cancer associated with mutated BRCA2 and androgen. Therapeutic applications of nuclear receptors variants may be based on secreted version of receptors such as the thyroid nuclear receptor that by binding plasma free thyroid hormone to reduce its levels may have a therapeutic effect in cases of thyrotoxicosis. A secreted version of glucocorticoid nuclear receptor, by binding plasma free cortisol, thus, reducing, may have a therapeutic effect in cases of Cushing's disease (a disease associated with high cortisole levels in the plasma).

Another example of a secreted variant of a receptor is a secreted form of the TNF receptor, which is used to treat conditions in which reduction of TNF levels is of benefit including Rheumatoid, Arthritis, Juvenile Rheumatoid Arthritis, Psoriatic Arthritis and Ankylosing Spondylitis.

Protein Serine/Threonine Kinases:

The phrase “protein serine/threonine kinases” refers to proteins which phosphorylate serine/threonine residues, mainly involved in signal transduction, such as transmembrane receptor protein serine/threonine kinase, 3-phosphoinositide-dependent protein kinase, DNA-dependent protein kinase, G-protein-coupled receptor phosphorylating protein kinase, SNF1A/AMP-activated protein kinase, casein kinase, calmodulin regulated protein kinase, cyclic-nucleotide dependent protein kinase, cyclin-dependent protein kinase, eukaryotic translation initiation factor 2a kinase, galactosyltransferase-associated kinase, glycogen synthase kinase 3, protein kinase C, receptor signaling protein see/threonine kinase, ribosomal protein S6 kinase, and IkB kinase.

Pharmaceutical compositions including such proteins or protein encoding sequences, antibodies directed against such proteins or polynucleotides capable of altering expression of such proteins may be used treat diseases ameliorated by a modulating kinase activity. Antibodies and polynucleotides such as PCR primers and molecular probes designed to identify such proteins or protein encoding sequences may be used for diagnosis of such diseases.

Examples of such diseases include, but are not limited to schizophrenia 5-HT(2A) serotonin receptor is the principal molecular target for LSD-like hallucinogens and atypical antipsychotic drugs. It hs been shown that a major mechanism for the attenuation of this receptor signaling following agonist activation typically involves the phosphorylation of serine and/or threonine residues by various kinases. Therefore, serine/threonine kinases specific for the 5-HT(2A) serotonin receptor may serve as drug targets for a disease such as schizophrenia. Other diseases that may be treated through serine/thereonine kinases modulation are Peutz-Jeghers syndrome (PJS, a rare autosomal-dominant disorder characterized by hamartomatous polyposis of the gastrointestinal tract and melanin pigmentation of the skin and mucous membranes [Hum. Mutat. 2000, 16(1):23-30], breast cancer [Oncogene. 1999, 18(35):4968-73], Type 2 diabetes insulin resistance [Am. J. Cardiol. 2002, 90(5A):11G-18G], and fanconi anemia [Blood. 2001, 98(13):3650-7].

Channel/Pore Class Transporters:

The phrase “Channel/pore class transporters” refers to proteins that mediate the transport of molecules and macromolecules across membranes, such as α-type channels, porins, and pore-forming toxins.

Pharmaceutical compositions including such proteins or protein encoding sequences, antibodies directed against such proteins or polynucleotides capable of altering expression of such proteins, may be used to treat diseases in which the transport of molecules and macromolecules are abnormal, therefore leading to various pathologies. Antibodies and polynucleotides such as PCR primers and molecular probes designed to identify such proteins or protein encoding sequences may be used for diagnosis of such diseases.

Examples of such diseases include, but are not limited to diseases of the nerves system such as Parkinson, diseases of the hormonal system, diabetes and infectious diseases such as bacterial and fungal infections. For example, α-hemolysin, is a protein product of S. aureus which creates ion conductive pores in the cell membrane, thereby deminishing its integrity.

Hydrolases, Acting on Acid Anhydrides:

The phrase “hydrolases, acting on acid anhydrides” refers to hydrolytic enzymes that are acting on acid anhydrides, such as hydrolases acting on acid anhydrides in phosphorus containing anhydrides or in sulfonyl-containing anhydrides, hydrolases catalyzing transmembrane movement of substances, and involved in cellular and subcellular movement.

Pharmaceutical compositions including such proteins or protein encoding sequences, antibodies directed against such proteins or polynucleotides capable of altering expression of such proteins may be used to treat diseases in which the hydrolase-related activities are abnormal. Antibodies and polynucleotides such as PCR primers and molecular probes designed to identify such proteins or protein encoding sequences may be used for diagnosis of such diseases.

Examples of such diseases include, but are not limited to glaucoma treated with carbonic anhydrase inhibitors (e.g. Dorzolamide), peptic ulcer disease treated with H(⁺)K(⁺)ATPase inhibitors that were shown to affect disease by blocking gastric carbonic anhydrase (e.g. Omeprazole).

Transferases, Transferring Phosphorous-Containing Groups:

The phrase “transferases, transferring phosphorus-containing groups” refers to enzymes that catalyze the transfer of phosphate from one molecule to another, such as phosphotransferases using the following groups as acceptors: alcohol group, carboxyl group, nitrogenous group, phosphate, phosphotransferases with regeneration of donors catalyzing intramolecular transfers; phosphotransferases; nucleotidyltransferase; and phosphotransferases for other substituted phosphate groups.

Pharmaceutical compositions including such proteins or protein encoding sequences, antibodies directed against such proteins or polynucleotides capable of altering expression of such proteins may be used to treat diseases in which the transfer of a phosphorous containing functional group to a modulated moiety is abnormal. Antibodies and polynucleotides such as PCR primers and molecular probes designed to identify such proteins or protein encoding sequences may be used for diagnosis of such diseases.

Examples of such diseases include, but are not limited to acute MI [Ann. Emerg. Med. 2003, 42(3):343-750], Cancer, [Oral. Dis. 2003, 9(3):119-28; J. Surg. Res. 2003, 113(1):102-8] and Alzheimer's disease [Am. J. Pathol. 2003, 163(3):845-58]. Examples for possible utilities of such transferases for drug improvement include, but are not limited to aminoglycosides treatment (antibiotics) to which resistance is mediated by aminoglycoside phosphotransferases [Front. Biosci. 1999, 1; 4:D9-21]. Using aminoglycoside phosphotransferases variants or inhibiting these enzymes may reduce aminoglycosides resistance. Since aminoglycosides can be toxic to some patients, proving the expression of aminoglycoside phosphotransferases in a patient can deter from treating him with aminoglycosides and risking the patient in vain.

Phosphoric Monoester Hydrolases:

The phrase “phosphoric monoester hydrolases” refers to hydrolytic enzymes that are acting on ester bonds, such as nuclease, sulfuric ester hydrolase, carboxylic ester hydrolase, thiolester hydrolase, phosphoric monoester hydrolase, phosphoric diester hydrolase, triphosphoric monoester hydrolase, diphosphoric monoester hydrolase, and phosphoric triester hydrolase.

Pharmaceutical compositions including such proteins or protein encoding sequences, antibodies directed against such proteins or polynucleotides capable of altering expression of such proteins, may be used to treat diseases in which the hydrolytic cleavage of a covalent bond with accompanying addition of water (—H being added to one product of the cleavage and —OH to the other), is abnormal. Antibodies and polynucleotides such as PCR primers and molecular probes designed to identify such proteins or protein encoding sequences may be used for diagnosis of such diseases.

Examples of such diseases include, but are not limited to diabetes and CNS diseases such as Parkinson and cancer.

Enzyme Inhibitors:

The term “enzyme inhibitors” refers to inhibitors and suppressors of other proteins and enzymes, such as inhibitors of kinases, phosphatases, chaperones, guanylate cyclase, DNA gyrase, ribonuclease, proteasome inhibitors, diazepam-binding inhibitor, ornithine decarboxylase, inhibitors, dUTP pyrophosphatase inhibitor, phospholipase inhibitor, proteinase inhibitor, protein biosynthesis inhibitors, and amylase inhibitors.

Pharmaceutical compositions including such proteins or protein encoding sequences, antibodies directed against such proteins or polynucleotides capable of altering expression of such proteins, may be used to treat diseases in which beneficial effect may be achieved by modulating the activity of inhibitors and suppressors of proteins and enzymes. Antibodies and polynucleotides such as PCR primers and molecular probes designed to identify such proteins or protein encoding sequences may be used for diagnosis of such diseases.

Examples of such diseases include, but are not limited to α-1 antitrypsin (a natural serine proteases, which protects the lung and liver from proteolysis) deficiency associated with emphysema, COPD and liver chirosis α-1 antitrypsin is also used for diagnostics in cases of unexplained liver and lung disease. A variant of this enzyme may act as protease inhibitor or a diagnostic target for related diseases.

Electron Transporters:

The term “Electron transporters” refers to ligand binding or carrier proteins involved in electron transport such as flavin-containing electron transporter, cytochromes, electron donors, electron acceptors, electron carriers, and cytochrome-c oxidases.

Pharmaceutical compositions including such proteins or protein encoding sequences, antibodies directed against such proteins or polynucleotides capable of altering expression of such proteins, may be used to treat diseases in which beneficial effect may be achieved by modulating the activity of electron transporters. Antibodies and polynucleotides such as PCR primers and molecular probes designed to identify such proteins or protein encoding sequences may be used for diagnosis of such diseases.

Examples of such diseases include, but are not limited to cyanide toxicity, resulting from cyanide binding to ubiquitous metalloenzymes rendering them inactive, and interfering with the electron transport. Novel electron transporters to which cyanide can bind may serve as drug targets for new cyanide antidotes.

Transferases, Transferring Glycosyl Groups:

The phrase “transferases, transferring glycosyl groups” refers to enzymes that catalyze the transfer of a glycosyl chemical group from one molecule to another such as murein lytic endotransglycosylate E, and sialyltransferase.

Pharmaceutical compositions including such proteins or protein encoding sequences, antibodies directed against such proteins or polynucleotides capable of altering expression of such proteins, may be used to treat diseases in which the transfer of a glycosyl chemical group is abnormal. Antibodies and polynucleotides such as PCR primers and molecular probes designed to identify such proteins or protein encoding sequences may be used for diagnosis of such diseases.

Ligases, Forming Carbon-Oxygen Bonds:

The phrase “ligases, forming carbon-oxygen bonds” refers to enzymes that catalyze the linkage between carbon and oxygen such as ligase forming aminoacyl-tRNA and related compounds.

Pharmaceutical compositions including such proteins or protein encoding sequences, antibodies directed against such proteins or polynucleotides capable of altering expression of such proteins, may be used to treat diseases in which the linkage between carbon and oxygen in an energy dependent process is abnormal. Antibodies and polynucleotides such as PCR primers and molecular probes designed to identify such proteins or protein encoding sequences may be used for diagnosis of such diseases.

Ligases:

The term “ligases” refers to enzymes that catalyze the linkage of two molecules, generally utilizing ATP as the energy donor, also called synthetase. Examples for ligases are enzymes such as β-alanyl-dopamine hydrolase, carbon-oxygen bonds forming ligase, carbon-sulfur bonds forming ligase, carbon-nitrogen bonds forming ligase, carbon-carbon bonds forming ligase, and phosphoric ester bonds forming ligase.

Pharmaceutical compositions including such proteins or protein encoding sequences, antibodies directed against such proteins or polynucleotides capable of altering expression of such proteins, may be used to treat diseases in which the joining together of two molecules in an energy dependent process is abnormal. Antibodies and polynucleotides such as PCR primers and molecular probes designed to identify such proteins or protein encoding sequences may be used for diagnosis of such diseases.

Examples of such diseases include, but are not limited to neurological disorders such as Parkinson's disease [Science. 2003, 302(5646):819-22; J. Neurol. 2003, 250 Suppl. 3:III25—III29] or epilepsy [Nat. Genet. 2003, 35(2):125-7], cancerous diseases [Cancer Res. 2003, 63(17):5428-37; Lab. Invest. 2003, 83(9):1255-65], renal diseases [Am. J. Pathol. 2003, 163(4):1645-523, infectious diseases [Arch. Virol. 2003, 148(9):1851-62] and fanconi anemia [Nat. Genet. 2003, 35(2):165-70].

Hydrolases, Acting on Glycosyl Bonds:

The phrase “hydrolases, acting on glycosyl bonds” refers to hydrolytic enzymes that are acting on glycosyl bonds such as hydrolases hydrolyzing N-glycosyl compounds, S-glycosyl compounds, and O-glycosyl compounds.

Pharmaceutical compositions including such proteins or protein encoding sequences, antibodies directed against such proteins or polynucleotides capable of altering expression of such proteins, may be used to treat diseases in which the hydrolase-related activities are abnormal. Antibodies and polynucleotides such as PCR primers and molecular probes designed to identify such proteins of protein encoding sequences may be used for diagnosis of such diseases.

Examples oft such diseases include cancerous diseases [J. Natl. Cancer Inst. 2003, 95(17):1263-5; Carcinogenesis. 2003, 24(7):1281-2; author reply 1283] vascular diseases [J. Thorac. Cardiovasc. Surg. 2003, 126(2):344-57], gastrointestinal diseases such as colitis [J. Immunol. 2003, 171(3):1556-63] or liver fibrosis [World J. Gastroenterol. 2002, 8(5):901-7].

Kinases:

The term “kinases” refers to enzymes which phosphorylate serine/threonine or tyrosine residues, mainly involved in signal transduction. Examples for kinases include enzymes such as 2-amino-4-hydroxy-6-hydroxymethyldihydropteridine pyrophosphokinase, NAD(⁺) kinase, acetylglutamate kinase, adenosine kinase, adenylate kinase, adenylsulfate kinase, arginine kinase, aspartate kinase, choline kinase, creatine kinase, cytidylate kinase, deoxyadenosine kinase, deoxycytidine kinase, deoxyguanosine, kinase, dephospho-CoA kinase, diacylglycerol kinase, dolichol kinase, ethanolamine kinase, galactokinase, glucokinase, glutamate 5-kinase, glycerol kinase, glycerone kinase, guanylate kinase, hexokinase, homoserine kinase, hydroxyethylthiazole kinase, inositol/phosphatidylinositol kinase, ketohexokinase, mevalonate kinase, nucleoside-diphosphate kinase, pantothenate kinase, phosphoenolpyruvate carboxykinase, phosphoglycerate kinase, phosphomevalonate kinase, protein kinase, pyruvate dehyrogenase (lipoamide) kinase, pyruvate kinase, ribokinase, ribose-phosphate pyrophosphokinase, selenide, water dikinase, shikimate kinase, thiamine pyrophosphokinase, thymidine kinase, thymidylate kinase, uridine kinase, xylulokinase, 1D-myo-inositol-trisphosphate 3-kinase, phosphofructokinase, pyridoxal kinase, shinganine kinase, riboflavin kinase, 2-dehydro-3-deoxygalactonokinase, 2-dehydro-3-deoxygluconokinase, 4-diphosphocytidyl-2C-methyl-D-erythritol-kinase, GTP pyrophosphokinase, L-fuculokinase, L-ribulokinase, L-xylulokinase, isocitrate dehydrogenase (NADP⁺) kinase, acetate kinase, allose kinase, carbamate kinase, cobinamide kinase, diphosphate-purine nucleoside kinase, fructokinase, glycerate kinase, hydroxymethylpyrimidine kinase, hygromycin-B kinase, inosine kinase, kanamycin kinase, phosphomethylpyrimidine kinase, phosphoribulokinase, polyphosphate kinase, propionate kinase, pyruvate, water dikinase, rhamnulokinase, tagatose-6-phosphate kinase, tetraacyldisaccharide 4′-kinase, thiamine-phosphate kinase, undecaprenol kinase, uridylate kinase, N-acylmannosamine kinase, D-erythro-sphingosine kinase.

Pharmaceutical compositions including such proteins or protein encoding sequences, antibodies directed against such proteins or polynucleotides capable of altering expression of such proteins, may be used to treat diseases which may be ameliorated by a modulating kinase activity. Antibodies and polynucleotides such as PCR primers and molecular probes designed to identify such proteins or protein encoding sequences may be used for diagnosis of such diseases.

Examples of such diseases include, but are not limited to, acute lymphoblastic leukemia associated with spleen tyrosine kinase deficiency [Goodman P. A., et al., (2001) Oncogene, 20(30):3969-78], ataxia telangiectasia associated with ATM kinase deficiency [Boultwood J., (2001) J. Clin. Pathol., 54(7):512-6], congenital haemolytic anaemia associated with erythrocyte pyruvate kinase deficiency [Zanella A., et al., (2001) Br. J. Haematol., 113(1):43-8], mevalonic aciduria caused by mevalonate kinase deficiency [Houten S. M., et al., (2001) Eur. J. Hum. Genet., 9(4):253-9], and acute myelogenous leukemia associated with over-expressed death-associated protein kinase [Guzman M. L., et al., (2001) Blood, 97(7):2177-9].

Nucleotide Binding:

The term “nucleotide binding” refers to ligand binding or carrier proteins, involved in physical interaction with a nucleotide, preferably, any compound consisting of a nucleoside that is esterified with [ortho]phosphate or an oligophosphate at any hydroxyl group on the glycose moiety, such as purine nucleotide binding proteins.

Pharmaceutical compositions including such proteins or protein encoding sequences, antibodies directed against such proteins or polynucleotides capable of altering expression of such proteins, may to be used to treat diseases that are associated with abnormal nucleotide binding. Antibodies and polynucleotides such as PCR primers and molecular probes designed to identify such proteins or protein encoding sequences may be used for diagnosis of such diseases.

Examples of such diseases include, but are not limited to Gout (a syndrome characterized by high urate level in the blood). Since urate is a breakdown metabolite of purines, reducing purines serum levels could have a therapeutic effect in Gout disease.

Tubulin Binding:

The term “tubulin binding” refers to binding proteins that bind tubulin such as microtubule binding proteins.

Pharmaceutical co positions including such proteins or protein encoding sequences, antibodies directed against such proteins or polynucleotides capable of altering expression of such proteins, may be used to treat diseases which are associated with abnormal tubulin activity or structure. Binding the products of the genes of this family, or antibodies reactive therewith, can modulate a plurality of tubulin activities as well as change microtubulin structure. Antibodies and polynucleotides such as PCR primers and molecular probes designed to identity such proteins or protein encoding sequences may be used for diagnosis of such diseases.

Examples of such diseases include, but are not limited to, Alzheimer's disease associated with t-complex polypeptide 1 deficiency [Schuller E., et al., (2001) Life Sci., 69(3):263-70], neurodegeneration associated with apoE deficiency [Masliah E., et al., (1995) Exp. Neurol., 136(2):107-22], progressive axonopathy associated with disfuctional neurofilaments [Griffiths I. R., et al., (1989) Neuropathol. Appl. Neurobiol., 15(1):63-74], familial frontotemporal dementia associated with tau deficiency [astor P., et al., (2001) Ann. Neurol., 49(2):263-7], and colon cancer suppressed by APC White R. L., (1997) Pathol. Biol. (Paris), 45(3):2404]. En example for a drug whose target is tubulin is the anticancer drug—Taxol. Drugs having similar mechanism of action (interfering with tubulin polymerization) may be developed based on tubulin binding proteins.

Receptor Signaling Proteins:

The phrase “receptor signaling proteins” refers to receptor proteins involved in signal transduction such as receptor signaling protein serine/threonine kinase, receptor signaling protein tyrosine kinase, receptor signaling protein tyrosine phosphatases, aryl hydrocarbon receptor nuclear translocator, hematopoeitin interferon-class (D200-domain) cytokine receptor signal transducer, transmembrane receptor protein tyrosine kinase signaling protein, transmembrane receptor protein serine/threonine kinase signaling protein, receptor signaling protein serine/threonine kinase signaling protein, receptor signaling protein serine/threonine phosphatase signaling protein, small GTPase regulatory/interacting protein, receptor signaling protein tyrosine kinase signaling protein, and receptor signaling protein serine/threonine phosphatase.

Pharmaceutical compositions including such proteins or protein encoding sequences, antibodies directed against such proteins or polynucleotides capable of altering expression of such proteins, may be used to treat diseases in which the signal-transduction is abnormal, either as a cause, or as a result of the disease. Antibodies and polynucleotides such as PCR primers and molecular probes designed to identify such proteins or protein encoding sequences may be used for diagnosis of such diseases.

Examples of such diseases include, but are not limited to, complete hypogonadotropic hypogonadism associated with GnRH receptor deficiency [Kottler M. L., et a., (2000) J. Clin. Endocrinol. Metab., 85(9):3002-8], severe combined immunodeficiency disease associated with IL-7 receptor deficiency [Puel A. and Leonard W. J., (2000) Curr. Opin. Immunol., 12(4):468-7], schizophrenia associated N-methyl-D-aspartate receptor deficiency [Mohn A. R., et al., (1999) Cell, 98(4):427-36], Yesinia-associated arthritis associated with tumor necrosis factor receptor p55 deficiency [Zhao Y. X., et al., (1999) Arthritis Rheum., 42(8):1662-72], and Dwarfism of Sindh caused by growth hormone-releasing hormone receptor deficiency [aheshwari H. G., et al., (1998) J. Clin. Endocrinol. Metab., 83(11):4065-74].

Molecular Function Unknown:

The phrase “molecular function unknown” refers to various proteins with unknown molecular function, such as cell surface antigens.

Pharmaceutical compositions including such proteins or protein encoding sequences, antibodies directed against such proteins or polynucleotides capable of altering expression of such proteins, may be used to treat diseases in which regulation of the recognition, or participation or bind of cell surface antigens to other moieties may have therapeutic effect. Antibodies and polynucleotides such as PCR primers and molecular probes designed to identify su proteins or protein encoding sequences may be used for diagnosis of such diseases.

Examples of such diseases include, but are not limited to, autoimmune diseases, various infectious diseases, cancer diseases which involve non cell surface antigens recognition and activity.

Enzyme Activators:

The term “enzyme activators” refers to enzyme regulators such as activators of kinases, phosphatases, sphingolipids, chaperones, guanylate cyclase, tryptophan hydroxylase, proteases, phospholipases, caspases, proprotein convertase 2 activator, cyclin-dependent protein kinase 5 activator superoxide-generating NADPH oxidase activator, sphingomyelin phosphodiesterase activator, monophenol monooxygenase activator, proteasome activator, and GTPase activator.

Pharmaceutical compositions including such proteins or protein encoding sequences, antibodies directed against such proteins or polynucleotides capable of altering expression of such proteins, may be used to treat diseases in which beneficial effect may be achieved by modulating the activity of activators of proteins and enzymes. Antibodies and polynucleotides such as PCR primers and molecular probes designed to identify such proteins or protein encoding sequences nay be used for diagnosis of such diseases.

Examples of such diseases include, but are not limited to all complement related diseases, as most complement proteins activate by cleavage other complement proteins.

Transferases, Transferring One-Carbon Groups:

The phrase “transferases, transferring one-carbon groups” refers enzymes that catalyze the transfer of a one-carbon chemical group from one molecule to another such as methyltransferase, amidinotransferase, hydroxymethyl-, formyl-, and related transferase, carboxyl- and carbamoyltransferase.

Pharmaceutical compositions including such proteins or protein encoding sequences, antibodies directed against such proteins or polynucleotides capable of altering expression of such proteins, may be used to treat diseases in which the transfer of a one-carbon chemical group from one molecule to another is abnormal so that a beneficial effect may be achieved by modulation of such reaction. Antibodies and polynucleotides such as PCR primers and molecular probes signal to identify such proteins or protein encoding sequences may be used for diagnosis of such diseases.

Transferases:

The term “transferases” refers to enzymes that catalyze the transfer of a chemical group, preferably, a phosphate or amine from one molecule to another. It includes enzymes such as transferases, transferring one-carbon groups, aldehyde or ketonic groups, acyl groups, glycosyl groups, alkyl or aryl (other than methyl) groups, nitrogenous, phosphors-containing groups, sulfur-containing groups, lipoyltransferase, deoxycytidyl transferases.

Pharmaceutical compositions including such proteins or protein encoding sequences, antibodies directed against such proteins or polynucleotides capable of altering expression of such proteins, may be used to treat diseases in which the transfer of a chemical group from one molecule to another is abnormal. Antibodies and polynucleotides such as PCR primers and molecular probes designed to identify such proteins or protein encoding sequences may be used for diagnosis of such diseases.

Examples of such diseases include, but are not limited to cancerous diseases such as prostate cancer [Urology. 2003, 62(5 Suppl 1):55-62] or lung cancer [Invest. New Drugs. 2003, 21(4):435-43; JAMA. 2003, 22; 290(16):2149-58], psychiatric disorders [Am. J. Med. Genet. 2003, 15; 123B(1):64-9], colorectal disease such as Crohn's disease [Dis. Colon Rectum. 2003, 46(11):1498-507] or celiac diseases [N Engl. J. Med. 2003, 349(17):1673-4; author reply 1673-4], neurological diseases such as Parkinson's disease [J. Chem Neuroanat. 2003, 26(2):143-51], Alzheimer disease [Hum. Mol. Genet. 2003 21] or Charcot-Marie-Tooth Disease [Mol. Biol. Evol. 2003 31].

Chaperones:

The term “chaperones” refers to functional classes of unrelated families of proteins that assist the correct non-covalent assembly of other polypeptide-containing structures in vivo, but are not components of these assembled structures when they a performing their normal biological function. The group of chaperones include proteins such as ribosomal chaperone, peptidylprolyl isomerase, lectin-binding chaperone, nucleosome assembly chaperone, chaperonin ATPase, cochaperone, heat shock protein, HSP70/HSP9 organizing protein, fimbrial, chaperone, metallochaperone, tubulin folding, and HSC70-interacting protein.

Pharmaceutical compositions including such proteins or protein encoding sequences, antibodies directed against such proteins or polynucleotides capable of altering expression of “such proteins, may be used to treat diseases which are associated with abnormal protein activity, structure, degradation or accumulation of proteins. Antibodies and polynucleotides such as PCR primers and molecular probes designed to identity such proteins or protein encoding sequences may be used for diagnosis of such diseases.

Examples of such diseases include, but are not limited to neurological syndromes [J. Neuropathol. Exp. Neurol. 2003, 62(7):751-64; Antioxid Redox Signal. 2003, 5(3):337-48; J. Neurochem. 2003, 86(2):394-404], neurological diseases such as Parkinson's disease [Hum. Genet. 2003, 6; Neurol Sci. 2003, 24(3):159-60; J. Neurol. 2003, 250 Suppl. 3:III25-III29] ataxia [J. Hum. Genet. 2003; 48(8):415-9] or Alzheimer diseases [J. Mol. Neurosci. 2003, 20(3):283-6; J. Alzheimers Dis. 2003, 5(3):171-7], cancerous diseases [Semin. Oncol. 2003, 30(5):709-16], prostate cancer [Semin. Oncol. 2003, 30(5):709-16] metabolic diseases [J. Neurochem. 2003, 87(1):248-56], infectious diseases, such as prion infection [EMBO J. 2003, 22(20):5435-5445]. Chaperones may, be also used for manipulating therapeutic proteins binding to their receptors therefore, improving their therapeutic effect.

Cell Adhesion Molecule:

The phrase “cell adhesion molecule” refers to proteins that serve adhesion molecules between adjoining cells such as membrane-associated protein with guanylate kinase activity, cell adhesion receptor, neuroligin, calcium-dependent cell adhesion molecule, selectin, calcium-independent cell adhesion molecule, and extracellular matrix protein.

Pharmaceutical compositions including such proteins or protein encoding sequences, antibodies directed against such proteins or polynucleotides capable of altering expression of such proteins, may be used to treat diseases in which adhesion between adjoining cells is involved, typically conditions in which the adhesion is abnormal. Antibodies and polynucleotides such as PCR primers and molecular probes designed to identify such proteins or protein encoding sequences may be used for diagnosis of such diseases.

Examples of such diseases include, but are not limited to cancer in which abnormal adhesion may cause and enhance the process of metastasis and abnormal growth and development of various tissues in which modulation adhesion among adjoining cells can improve the condition. Leucocyte-endothelial interactions characterized by adhesion molecules involved in interactions between cells lead to a tissue injury and ischemia reperfusion disorders in which activated signals generated during ischemia may trigger an exuberant inflammatory response during reperfusion provoking greater tissue damage than initial ischemic insult [Crit. Care Med. 2002, 30(5 Suppl):S214-9]. The blockade of leucocyte-endothelial adhesive interactions has the potential to reduce vascular and tissue injury. This blockade may be achieved using a soluble variant of the adhesion molecule.

States of septic shock and ARDS involve large recruitment of neutrophil cells to the damaged tissues. Neutrophil cells to bind to the endothelial cells in the target tissues through adhesion molecules. Neutrophils possess multiple effector mechanisms that can produce endothelial and lung tissue injury, and interfere with pulmonary gas transfer by disruption of surfactant activity [Eur. J. Surg. 2002, 168(4):204-14]. In such cases, the use of soluble variant of the adhesion molecule may decrease the adhesion of neutrophils to the damaged tissues.

Examples of such diseases include, but are not limited to, Wiskott-Aldrich syndrome associated with WAS deficiency [Westerberg L., et al., (2001) Blood, 98(4):1086-94], asthma associated with intercellular adhesion molecule-1 deficiency [Tang M. L. and Fiscus L. C., (2001) Pulm. Pharmacol. Ther., 14(3):203-10], intra-atrial thrombogenesis associated with increased von Willebrand factor activity [Fukuchi M., et al., (2001) J. Am. Coil. Cardiol., 37(5):1436-42], junctional, epidermolysis bullosa associated with laminin 5-β-3 deficiency [Robbins P. B., et al., (2001) Proc. Natl. Acad. Sci., 98(9):5193-8], and hydrocephalus caused by neural adhesion molecule L1 deficiency [Rolf B., et al., (2001) Brain Res., 891(1-2):247-52].

Motor Proteins:

The term “motor proteins” refers to proteins that generate force or energy by the hydrolysis of ATP and that function in the production of intracellular movement or transportation. Examples of such proteins include microfilament motor, axonemal motor, microtubule motor, and kinetochore motor (dynein, kinesin, or myosin).

Pharmaceutical compositions including such proteins or protein encoding sequences, antibodies directed against such proteins or polynucleotides capable of altering expression of such proteins, may be used to treat diseases in which force or energy generation is impaired. Antibodies and polynucleotides such as PCR primers and molecular probes designed to identify such proteins or protein encoding sequences may be used for diagnosis of such diseases.

Examples of such diseases include, but are not limited to, malignant diseases where microtubules are drug targets for a family of anticancer drugs such, as myodystrophies and myopathies [Tends Cell Biol. 2002, 12(12):585-91], neurological disorders [Neuron. 2003, 25; 40(1): 25-40; Trends Biochem. Sci. 2003, 28(10):558-65; Med. Genet. 2003, 40(9):671-5], and hearing impairment [Trends Biochem. Sci. 2003, 28(10):558-65].

Defense/Immunity Proteins:

The term “defense/immunity proteins” refers to protein that are involved in the immune and complement systems such as acute-phase response proteins, antimicrobial peptides, antiviral response proteins, blood coagulation factors, complement components, immunoglobulins, major histocompatibility complex antigens and opsonins.

Pharmaceutical compositions including such proteins or protein encoding sequences, antibodies directed against such proteins or polynucleotides capable of altering expression of such proteins, may be used to treat diseases involving the immunological system including inflammation, autoimmune diseases, infectious diseases, as well as cancerous processes or diseases which are manifested by abnormal coagulation processes, which may include abnormal bleeding or excessive coagulation. Antibodies and polynucleotides such as PCR primers and molecular probes designed to identify such proteins or protein encoding sequences may be used for diagnosis of such diseases.

Examples of such diseases include, but are not limited to, late (C5-9) complement component deficiency associated with opsonin receptor allotypes [Fijen C. A., et al., (2000) Clin. Exp. Immunol., 120(2):338-45], combined immunodeficiency associated with defective expression of MHC class II genes [Griscelli, C., et al., (1989) Immununodefic. Rev. 1(2):135-53], loss of antiviral activity of CD4 T cells caused by neutralization of endogenous TNFα [Pavic I., et al., (1993) J. Gen. Virol., 74 (Pt 10):2215-23], autoimmune diseases associated with natural resistance-associated macrophage protein, deficiency [Evans C. A., et al., (2001) Neurogenetics, 3(2):69-78], Epstein-Barr virus-associated lymphoproliferative disease inhibited by combined GM-CSF and IL-2 therapy; [Baiocchi R. A., et al., (2001) J. Clin. Invest., 108(6):887-94] and sepsis in which activate protein C is therapeutic protein itself.

Intracellular Transporters:

The term “intracellular transporters” refers to proteins that mediate the transport of molecules and macromolecules inside the cell, such as intracellular nucleoside transporter, vacuolar assembly proteins, vesicle transporters, vesicle fusion proteins, type II protein secretors.

Pharmaceutical compositions including such proteins or protein encoding sequences, antibodies directed against such proteins or polynucleotides capable of altering expression of such proteins, may be used to treat diseases in which the transport of molecules and macromolecules is abnormal leading to various pathologies. Antibodies and polynucleotides such as PCR primers and molecular probes designed to identify such proteins or protein encoding sequences may be used for diagnosis of such diseases.

Transporters:

The term “transporters” refers to proteins that mediate the transport of molecules and macromolecules, such as channels, exchangers, and pumps. Transporters include proteins such as: amine/polyamine transporter, lipid transporter, neurotransmitter transporter, organic acid transporter, oxygen transporter, water transporter, carriers, intracellular transports, protein transporters, ion transporters, carbohydrate transporter, polyol transporter, amino acid transporters, vita cofactor transporters, siderophore transporter, drug transporter, channel/pore class transporter, group translocator, auxiliary transport proteins, permeases, murein transporter, organic alcohol transporter, nucleobase, nucleoside, and nucleotide and nucleic acid transporters.

Pharmaceutical compositions including such proteins or protein encoding sequences, antibodies directed against such proteins or polynucleotides capable of altering expression of such proteins, may be used to treat diseases in which transport of molecules and macromolecules such as neurotransmitters, hormones, sugar etc. is impaired leading to various pathologies. Antibodies and polynucleotides such as PCR primers and molecular probes designed to identify such proteins or protein encoding sequences may be used for diagnosis of such diseases.

Examples of such diseases include, but are not limited to, glycogen storage disease caused by glucose-6-phosphate transporter deficiency [Hiraiwa H., and Chou J. Y. (2001) DNA Cell Biol., 20(8):447-53], tangier disease associated with ATP-binding cassette transporter-1 deficiency [McNeish J., et al., (2000) Proc. Natl. Acad. Sci., 97(8):4245-50], systemic primary carnitine deficiency associated with organic cation transporter deficiency [Tang N. L., et al., (1999) Hum. Mol. Genet., 8(4):655-60], Wilson disease associated with copper-transporting ATPases deficiency [Payne A. S., et al., (1998) Proc. Natl. Acad. Sci. 95(18):10854-9], and atelosteogenesis associated with diastrophic dysplasia sulphate transporter deficiency [Newbury-Ecob R., (1998) J. Med. Genet., 35(1):49-53], Central Nervous system diseases treated by inhibiting neurotransmitter transporter (e.g. Depression, treated with serotonin transporters inhibitors—Prozac), and Cystic fibrosis mediated by the chloride channel CFTR. Other transporter related diseases are cancer [Oncogene. 2003, 22(38):6005-12] and especially cancer resistant to treatment [Oncologist. 2003, 8(5):411-24; J. Med. Invest. 2003, 50(3-4):126-35], infectious diseases, especially fungal infections [Annu. Rev. Phytopathol. 2003, 41:641-67], neurological diseases, such as Parkinson [FASEB J. 2003, Sep. 4 [Epub ahead of print]], and cardiovascular diseases, including hypercholesterolemia [Am. J. Cardiol. 2003, 92(4B):10K-16K].

There are about 30 membrane transporter genes linked to a known genetic clinical syndrome. Secreted versions of splice variants of transporters may be therapeutic as the case with soluble receptors. These transporters may have the capability to bind the compound in the serum they would normally bind on the membrane. For example, a secreted form AT-T7B, a transporter involved in Wilson's disease, is expected to bind plasma Copper, therefore have a desired therapeutic effect in Wilson's disease.

Lyases:

The term “lyases” refers to enzymes that catalyze the formation of double bonds by removing chemical groups from a substrate without hydrolysis or catalyze the addition of chemical groups to double bonds. It includes enzymes such as carbon-carbon lyase, carbon-oxygen lyase, carbon-nitrogen lyase, carbon-sulfur lyase, carbon-halide lyase, and phosphorus-oxygen lyase.

Pharmaceutical compositions including such proteins or protein encoding sequences, antibodies directed against such proteins or polynucleotides capable of alternating expression of such proteins, may be used to treat diseases in which the double bonds formation catalyzed by these enzymes is impaired. Antibodies and polynucleotides such as PCR primers and molecular probes designed to identify such proteins or protein encoding sequences may be used for diagnosis of diseases.

Examples of such diseases include, but are not limited to, autoimmune diseases [JAMA. 2003, 290(13):1721-8; JAMA. 2003, 290(13):1713-20], diabetes [Diabetes. 2003, 52(9):2274-8], neurological disorders such as epilepsy: [J. Neurosci. 2003, 23(24):8471-9], Parkinson [J. Neurosci. 2003, 23(23):8302-9; Lancet. 2003, 362(9385):712] or Creutzfeldt-Jakob disease [Clin. Neurophysiol. 2003, 114(9):1724-8], and cancerous diseases [J. Pathol. 2003, 201(1):37-45; J. Pathol. 2003, 201(1):37-45; Cancer Res. 2003, 63(16):4952-9; Eur. J. Cancer. 2003, 39(13):1899-903].

Actin Binding Proteins:

The phrase “actin binding proteins” refers to proteins binding actin as actin cross-linking, actin bundling, F-actin capping, lactin monomer binding, actin lateral binding, actin depolymerizing, actin monomer sequestering, actin filament severing, actin modulating, membrane associated actin binding, actin thin filament length regulation, and actin polymerizing proteins.

Pharmaceutical compositions including such proteins or protein encoding sequences, antibodies directed against such proteins or polynucleotides capable of altering expression of such proteins, may be used to treat diseases in which actin binding is impaired. Antibodies and polynucleotides such as PCR primers and molecular probes designed to identify such proteins or protein encoding sequences may be used for diagnosis of such diseases.

Examples of such diseases include, but are not limited to, neuromuscular diseases such as muscular dystrophy [Neurology. 2003, 61(3):404-6], Cancerous diseases [Urology. 2003, 61(4):845-50; J. Cutan. Pathol. 2002, 29(7):430; Cancer. 2002, 94(6):1777-86; Clin. Cancer Res. 2001, 7(8):2415-24; Breast Cancer Res. Treat. 2001, 65(1):11-21], renal diseases such as glomerulonephritis [J. Am. Soc. Nephrol. 2002, 13(2):322-31; Eur. J. Immunol. 2001, 31(4):1221-7], and gastrointestinal diseases such as Crohn's disease [J. Cell Physiol. 2000, 182(2):303-9].

Protein Binding Proteins:

The phrase “protein binding proteins” refers to proteins involved in diverse biological functions through binding other proteins. Examples of such biological function include intermediate filament binding, LIM-domain binding, LLR-domain binding, clathrin binding, ARF binding, vinculin binding, KU70 binding, troponin C binding PDZ-domain binding, SH3-domain binding, fibroblast growth factor binding, membrane-associated protein with guanylate kinase activity interacting, Wnt-protein binding, DEAD/H-box RNA helicase binding β-amyloid binding, myosin binding, TATA-binding protein binding DNA topoisomerase I binding, polypeptide hormone binding, RHO binding, FH1-domain binding, syntaxin-1 binding, HSC70-interacting, transcription factor binding, metarhodopsin binding, tubulin binding, JUN kinase binding, RAN protein binding, protein signal sequence binding, importin α export receptor, poly-glutamine tract binding, protein carrier, β-catenin binding, protein C-terminus binding, lipoprotein binding, cytoskeletal protein binding protein, nuclear localization sequence binding, protein phosphatase 1 binding, adenylate cyclase binding, eukaryotic initiation factor 4E binding, calmodulin binding, collagen binding, insulin-like growth factor binding, lamin binding, profilin binding, tropomyosin binding, actin binding, peroxisome targeting sequence binding, SNARE binding, and cyclin binding.

Pharmaceutical compositions including such proteins or protein encoding sequences, antibodies directed against such proteins or polynucleotides capable of altering expression of such proteins, may be used to treat diseases which are associated with impaired protein binding. Antibodies and polynucleotides such as PCR primers and molecular probes designed to identify such proteins or protein encoding sequences may be used for diagnosis of such diseases.

Examples of such diseases include, but are not limited to, neurological and psychiatric diseases [J. Neurosci. 2003, 23(25):8788-99; Neurobiol. Dis. 2003, 14(1):146-56; J. Neurosci. 2003, 23(17):6956-64; Am. J. Pathol. 2003, 163(2):609-19], and cancerous diseases [Cancer Res. 2003, 63(15):4299-304; Semin. Thromb. Hemost. 2003, 29(3):247-58; Proc. Natl. Acad. Sci. USA. 2003, 100(16):9506-11].

Ligand Binding or Carrier Proteins:

The phrase “ligand binding or carrier proteins” refers to proteins involved in diverse biological function such as: pyridoxal phosphate binding, carbohydrate binding, magnesium binding, amino acid binding, cyclosporin A binding, nickel binding, chlorophyll binding, biotin biding, penicillin binding, selenium binding, tocopherol binding, binding, oxygen transporters, electron transporter, steroid binding, juvenile hormone binding, retinoid binding, heavy metal binding, calcium binding, protein binding, glycosaminoglycan binding, folate binding, odorant binding, lipopolysaccharide binding and nucleotide binding.

Pharmaceutical compositions including such proteins or protein encoding sequence, antibodies directed against such or polynucleotides capable of altering expression of such proteins, may be used to treat diseases which are associated with impaired function of these proteins. Antibodies and polynucleotides such as PCR primers and molecular probes designed to identify such proteins or protein encoding sequences may be used for diagnosis of such diseases.

Examples of such diseases include, but are not limited to, neurological disorders [J. Med. Genet. 2003, 40(10):733-40; J. Neuropathol. Exp. Neurol. 2003, 62(9):968-75; J. Neurochem. 2003, 87(2):427-36], autoimmune diseases (N. Engl. J. Med. 2003, 349(16):1526-33; JAMA. 2003, 290(13):1721-8]; gastroesophageal reflux disease [Dig. Dis. Sci. 2003, 48(9):1832-8], cardiovascular diseases [J. Vasc. Surg 2003, 38(4):827-32], cancerous diseases [Oncogene. 2003, 22(43):6699-703; Br. J. Haematol. 2003, 123(2):288-96], respiratory diseases [Circulation. 2003, 108(15):1839-44], and ophtalmic diseases [Ophthalmology. 2003, 110(10):2040-4; Am. J. Ophthalmol. 2003, 136(4): 729-32].

ATPases:

The term “ATPases” refers to enzymes that catalyze the hydrolysis of ATP to ADP, releasing energy that is used in the cell. This group include enzymes such as plasma membrane cation-transporting ATPase, ATP-binding cassette (ABC) transporter, magnesium-ATPase, hydrogen-/sodium-translcating ATPase or ATPase translocating any other elements, arsenite-transporting ATPase, protein-transporting ATPase, DNA translocase, P-type ATPase, and hydrolase, acting on acid anhydrides involved in cellular and subcellular movement.

Pharmaceutical compositions including such proteins or protein encoding sequences, antibodies directed against such proteins or polynucleotides capable of altering expression of such proteins, may be used to treat diseases which are associated with impaired conversion of the hydrolysis of ATP to ADP or resulting energy use. Antibodies and such and molecular probes designed to identify such proteins or protein encoding sequences may be used for diagnosis of such diseases.

Examples of such diseases include, but are not limited to, infectious diseases such as helicobacter pylori ulcers [BMC Gastroenterol. 2003, Nov. 6], Neurological, muscular and psychiatric diseases [Int. J. Neurosci. 2003, 13(12):1705-1717; Int. J. Neurosci. 2003, 113(11):1579-1591; Ann. Neurol. 2003, 54(4):494-500], Amyotrophic Lateral Sclerosis [Other Motor Neuron Discord. 2003 4(2):96-9], cardiovascular diseases [J. Nippon. Med. Sch. 2003, 70(5):384-92; Endocrinology. 2003, 144(10):478-83], metabolic diseases [Mol. Pathol. 2003, 56(5):302-4; Neurosci. Lett. 2003, 350(2):105-8], and peptic ulcer disease treated with inhibitors of the gastric H⁺—K⁺ ATPase (e.g. Omeprazole) responsible for acid secretion in the gastric mucosa.

Carboxylic Ester Hydrolases:

The phrase carboxylic ester hydrolases” refers to hydrolytic enzymes acting on carboxylic ester bonds such as N-acetylglucosaminylphosphatidylinositol deacetylase, 2-acetyl-1-alkylglycerophosphocholine esterase, aminoacyl-tRNA hydrolase, arylesterase, carboxylesterase, cholinesterase, gluconolactonase, sterol esterase, acetylesterase, carboxymethylenebutenolidase, protein-glutamate methylesterase, lipase, and 6-phosphogluconolactonase.

Pharmaceutical compositions including such proteins or protein encoding sequences, antibodies directed against such proteins or polynucleotides capable of altering expression of such proteins, may be used to treat diseases in which the hydrolytic cleavage of a covalent bond with accompanying addition of water (—H being added to one product of the cleavage and —OH to the other) is abnormal so that a beneficial effect may be achieved by modulation of such reaction. Antibodies and polynucleotides such as PCR primers and molecular probes designed to identify such proteins or protein encoding sequences may be used for diagnosis of such diseases.

Examples of such diseases include, but are not limited to, autoimmune neuromuscular disease Myasthenia Gravis, treated with cholinesterase inhibitors.

Hydrolase, Acting on Ester Bonds:

The phrase “hydrolase, acting on ester bonds” refers to hydrolytic enzymes acting on ester bonds such as nucleases, sulfuric ester hydrolase, carboxylic ester hydrolases, thiolester hydrolase, phosphoric monoester hydrolase, phosphoric diester hydrolase, triphosphoric monoester hydrolase, diphosphoric monoester hydrolase, and phosphoric triester hydrolase.

Pharmaceutical compositions including such proteins or protein encoding sequences, antibodies directed against such proteins or polynucleotides capable of altering expression of such proteins, may be used to treat diseases in which the hydrolytic cleavage of a covalent bond with accompanying addition of water (—H being added to one product of the cleavage and —OH to the other), is abnormal. Antibodies and polynucleotides such as PCR primers and molecular probes designed to identify such proteins or protein encoding sequences may be used for diagnosis of such diseases.

Hydrolases:

The term “hydrolases” refers to hydrolytic enzymes such as GPI-anchor transamidase, peptidases, hydrolases, acting on ester bonds, glycosyl bonds, ether bonds, carbon-nitrogen (but not peptide) bonds, acid anhydrides, acid carbon-carbon bonds, acid halide-bonds, acid phosphorus-nitrogen bonds, acid sulfur-nitrogen bonds, acid carbon-phosphorus bonds acid sulfur-sulfur bonds.

Pharmaceutical compositions including such proteins or protein encoding sequences, antibodies directed against such proteins or polynucleotides capable of altering expression of such proteins, may be used to treat diseases in which the hydrolytic cleavage of a covalent bond with accompanying addition of water (—H being added to one product of the cleavage and —OH to the other) is abnormal. Antibodies and polynucleotides such as PCR primers and molecular probes designed to identify such proteins or protein encoding sequences may be used for diagnosis of such diseases.

Examples of such diseases include, but are not limited to, cancerous diseases [Cancer. 2003, 98(9):1842-8; Cancer. 2003, 98(9):1822-9], neurological diseases such as Parkinson diseases [J. Neurol. 2003, 250 Suppl 3:III15-III24; J. Neurol. 2003, 250 Suppl 3:III2-III10], endocrinological diseases such as pancreatitis [Pancreas. 2003, 27(4):291-6] or childhood genetic diseases [Eur. J. Pediatr. 1997, 156(12):935-8], coagulation diseases [BMJ. 2003, 327(7421):974-7], cardiovascular diseases [Ann. Intern. Med. 2003, October 139(8):670-82], autoimmunity diseases [J. Med. Genet. 2003, 40(10):761-6] and metabolic diseases [Am. J. Hum. Genet. 2001, 69(5):1002-12].

Enzymes:

The term “enzymes” refers to naturally occurring or synthetic macromolecular substance composed mostly of protein, that catalyzes, to various degree of specificity, at least one (bio)chemical reactions at relatively low temperatures. The action of RNA that has catalytic activity (ribozyme) is often also regarded as enzymatic. Nevertheless, enzymes are mainly “proteinaceous and are often easily inactivated by heating or by protein-denaturing agents. The substances upon which they act are known as substrates, for which the enzyme possesses a specific binding or active site.

The group of enzymes include various proteins possessing enzymatic activities such as mannosylphosphate transferase, para-hydroxybenzoate:polyprenyltransferase, rieske iron-sulfur protein, imidazoleglycerol-phosphate synthase, sphingosine hydroxylase, tRNA 2′-phosphotransferase, sterol C-24(28) reductase, C-8 sterol isomerase, C-22 sterol desaturase, C-14 sterol reductase, C-3 sterol dehydrogenase (C-4 sterol decarboxylase), 3-keto sterol reductase, C-4 methyl sterol oxidase, dihydroricotinamide riboside quinone reductase, glutamate phosphate reductase, DNA repair enzyme, telomerase, α-ketoacid dehydrogenase, β-alanyl-dopamine synthase, RNA editase, aldo-keto reductase, alkylbase DNA glycosidase, glycogen debranching enzyme, dihydropterin deaminase, dihydropterin oxidase, dimethylnitrosamine demethylase, ecdysteroid UDP-glucosyl/UDP glucuronosyl transferase, glycine cleavage system, helicase, histone deacetylase, mevaldate reductase, monooxygenase, poly(AP-ribose) glycohydrolase, pyruvate dehydrogenase, serine esterase, sterol carrier protein X-related thiolase, transposase, tyramine-β hydroxylase, para-aminobenzoic acid (PABA) synthase, glu-tRNA(gln) amidotransferase, molybdopterin cofactor sulfurase, lanosterol 14-α-demethylase, aromatase, 4-hydroxybenzoate octaprenyltransferase 7,8-dihydro-8-oxoguanine-triphosphatase, CDP-alcohol phosphotransferase, 2,5-diamino-6-(ribosylamino)-4(3H)-pyrimidonone 5′-phosphate deaminase, diphosphoinositol polyphosphate phosphohydrolase, γ-glutamyl carboxylase, small protein conjugating enzyme, small protein activating enzyme, 1-deoxyxylulose-5-phosphate synthase, 2′-phosphotransferase, 2-octoprenyl-3-methyl-6-methoxy-1,4-benzoquinone hydroxylase, 2C-Methyl-D-erythritol 2,4-cyclodiphosphate synthase, 3,4 dihydroxy-2-butanone-4-phosphate synthase, 4-amino-4-deoxychorismate lyase, 4-diphosphocytidyl-2C-methyl-D-erythritol synthase, ADP-L-glycero-D-manno-heptose synthase, D-erythro-7,8-dihydroneopterin triphosphate 2′-epimerase, N-ethylmaleimide reductase, O-antigen ligase, O-antigen polymerase, UDP-2,3-diacylglucosamine hydrolase, arsenate reductase, carnitine racemase, cobalamin [5′-phosphate] synthase, cobinamide phosphate, guanylyltransferase, enterobactin synthetase, enterochelin esterase, enterochelin synthetase, glycolate oxidase, integrase, lauroyl transferase, peptidoglycan synthase, phosphopantetheinyltransferase, phosphoglucosamine mutase, phosphoheptose isomerase, quinolinate synthase, siroheme synthase, N-acylmannosamine-6-phosphate 2-epimerase, N-acetyl-anhydromuramoyl-L-alanine amidase, carbon-phosphorous lyase, heme-copper terminal oxidase, disulfide oxidoreductase, phthalate dioxygenase reductase, sphingosine-1-phosphate lyase, molybdopterin oxidoreductase, dehydrogenase, NADPH oxidase, naringenin-chalcone synthase, N-ethylammeline chlorohydrolase, polyketide synthase, aldolase, kinase, phosphatase, CoA-ligase, oxidoreductase, transferase, hydrolase, lyase, isomerase, ligase, ATPase, sulfhydryl oxidase, lipoate-protein ligase, δ-1-pyrroline-5-carboxyate synthetase, lipoic acid synthase, and tRNA dihydrouridine synthase.

Pharmaceutical compositions including such proteins or protein encoding sequences, antibodies directed against such proteins or polynucleotides capable of altering expression of such proteins, may be used to treat diseases which can be ameliorated by modulating the activity of various enzymes which are involved both in enzymatic processes inside cells as well as in cell signaling. Antibodies and polynucleotides such as PCR primers and molecular probes designed to identify such proteins or protein encoding sequences may be used for diagnosis of such diseases.

Cytoskeletal Proteins:

The term “cytoskeletal proteins” refers to proteins involved in the structure formation of the cytoskeleton.

Pharmaceutical compositions including such proteins or protein encoding sequences, antibodies directed against such proteins or polynucleotides capable of altering expression of such proteins, may be used to treat diseases which are caused or due to abnormalities in cytoskeleton, including cancerous cells, and diseased cells such as cells that do not propagate, grow or function normally. Antibodies and polynucleotides such as PCR primers and molecular probes designed to identify such proteins or protein encoding sequences may be used for diagnosis of such diseases.

Examples of such diseases include, but are not limited to, liver diseases such as cholestatic diseases [Lancet. 2003, 362(9390):1112-9], vascular diseases [J. Cell Biol. 2003, 162(6):1111-22], endocrinological diseases [Cancer Res. 2003, 63(16):4836-41], neuromuscular disorders such as muscular dystrophy [Neuromuscul. Discord. 2003, 13(7-8):579-88], or myopathy [Neuromuscul. Discord. 2003, 13(6):456-67] neurological disorders such as Alzheimer's disease [J. Alzheimers Dis. 2003, 5(3):209-28], cardiac disorders [J. Am. Col. Cardiol. 2003, 42(2):319-27], skin disorders [J. Am. Coll. Cardiol. 2003, 42(2):319-27], and cancer [Proteomics. 2003, 3(6):979-90].

Structural Proteins:

The term “structural proteins” refers to proteins involved in the structure formation of the cell, such as structural proteins of ribosome, cell wall structural proteins, structural proteins of cytoskeleton, extracellular matrix structural proteins, extracellular matrix glycoproteins, amyloid proteins, plasma proteins, structural proteins of eye lens, structural protein of chorion (sensu Insecta), structural protein of cuticle (sensu Insecta), puparial glue protein (sensu Diptera), structural proteins of bone, yolk proteins, structural proteins of muscle, structural protein of vitelline membrane (sensu Insecta), structural proteins of peritrophic membrane (sensu Insecta), and structural proteins of nuclear pores.

Pharmaceutical compositions including such proteins or protein encoding sequences, antibodies directed-against such proteins or polynucleotides capable of altering expression of such proteins, may be used to treat diseases which are caused by abnormalities in cytoskeleton, including cancerous cells, and diseased cells such as cells that do not propagate, grow or function normally. Antibodies and polynucleotides such as PCR primers and molecular probes designed to identify such proteins or protein encoding sequences may be used for diagnosis of such diseases.

Examples of such diseases include, but are not limited to, blood vessels diseases such as aneurysms [Cardiovasc. Res. 2003, 60(1):205-13], joint diseases [Rheum. Dis. Clin. North Am. 2003, 29(3):6311-45], muscular diseases such as a muscular dystrophies [Curr. Opin. Clin. Nutr. Metab. Care. 2003, 6(4):435-9], neuronal diseases such as encephalitis [Neurovirol. 2003, 9(2):274-83], retinitis pigmentosa [Dev. Ophthalmol. 2003, 37:109-25], and infectious diseases [J. Virol. Methods. 2003, 109(1):75-83; FEMS Immunol. Med. Microbiol. 2003, 35(2):125-30; J. Exp. Med. 2003, 197(5):633-42].

Ligands:

The term “ligands” Prefers to proteins that bind to another chemical entity to form a larger complex, involved in various biological processes, such as signal transduction, metabolism, growth and differentiation, etc. This group of proteins includes opioid peptides, baboon receptor ligand, branchless receptor ligand, breathless receptor ligand, ephrin, frizzled receptor ligand, frizzled-2 receptor ligand, heartless receptor ligand, Notch receptor ligand, patched receptor ligand, punt receptor ligand, Ror receptor ligand, saxophone receptor ligand, SE20 receptor ligand, sevenless receptor ligand, smooth receptor ligand, thickveins receptor ligand, Toll receptor ligand, Torso receptor ligand, death receptor ligand, scavenger receptor ligand, neuroligin, integrin ligand, hormones, pheromones, growth factors, and sulfonylurea receptor ligand.

Pharmaceutical compositions including such proteins or protein encoding sequences, antibodies directed against such proteins or polynucleotides capable of altering expression of such proteins, may be used to treat diseases involved in impaired hormone function or diseases which involve abnormal secretion of proteins which may be due to abnormal presence, absence or impaired normal response to normal levels of secreted proteins. Those secreted proteins include hormones, neurotransmitters, and various other proteins secreted by cells to the extracellular environment. Antibodies and polynucleotides such as PCR primers and molecular probes designed to identify such proteins or protein encoding sequences may be used for diagnosis of such diseases.

Examples of such diseases include, but are not limited to, analgesia inhibited by orphanin FQ/nociceptin [Shane R., et al., (2001) Brain Res., 907(1-2):109-16], stroke protected by estrogen [Alkayed N., (2001) J. Neurosci., 21(19):7543-50], atherosclerosis associated with growth hormone deficiency [Elhadd T. A., et al., (2001) J. Clin. Endocrinol. Metab., 86(9):4223-32], diabetes inhibited by α-galactosylceramide [Hong S., et al., (2001) Nat. Med., 7(9):1052-6], and Huntington's disease associated with huntingtin deficiency [Rao D. S., et al., (2001) Mol. Cell. Biol., 21(22):7796-806].

Signal Transducer:

The term “signal transducers” refers to proteins such as activin inhibitors receptor-associated proteins, α-2 macroglobulin receptors, morphogens, quorum sensing signal generators, quorum sensing response regulators, receptor signaling proteins, ligands, receptors, two-component sensor molecules, and two-component response regulators.

Pharmaceutical compositions including such proteins or protein encoding sequences, antibodies directed against such proteins or polynucleotides capable of altering expression of such proteins, may be used to treat diseases in which the signal-transduction is impaired, either as a cause, or as a result of the disease. Antibodies and polynucleotides such as PCR primers and molecular probes designed to identify such proteins or protein encoding sequences may be used for diagnosis of such diseases.

Examples of such diseases include, but are not limited to, altered sexual dimorphism associated with signal transducer activator of transcription 5[Udy G. B., et al., (1997) Proc. Natl. Acad. Sci. USA, 94(14):7239-44], multiple sclerosis associated with sgp130 deficiency [Padberg F., et al., (1999) J. Neuroimmunol., 99(2):218-23], intestinal inflammation associated with elevated signal transducer and activator of transcription 3 activity [Suzuki A., et al., (2001) J Exp Med, 193(4):471-81], carcinoid tumor inhibited by increased signal transducer and activators of transcription 1 and 2 [Zhou Y., et al., (2001) Oncology, 60(4):330-8], and esophageal cancer associated with loss of EGF-STAT1 pathway [Watanabe G., et al., (2001) Cancer J., 7(2):132-9].

RNA Polymerase II Transcription Factors:

The phrase “RNA polymerase II transcription factors” refers to proteins such as specific and non-specific RNA polymerase II transcription factors, enhancer binding, ligand-regulated transcription factor, and general RNA polymerase II transcription factors.

Pharmaceutical compositions including such proteins or protein encoding sequences, antibodies directed against such proteins or polynucleotides capable of altering expression of such proteins, may be used to treat diseases involving impaired function of RNA polymerase II transcription factors. Antibodies and polynucleotides such as PCR primers and molecular probes designed to identify such proteins or protein encoding sequences may be used for diagnosis of such diseases.

Examples of such diseases include, but are not limited to, cardiac diseases [Cell Cycle. 2003, 2(2):99-104], xeroderma pigmentosum: [Bioessays. 2001, 23(8):671-3; Biochim. Biophys. Acta. 1997, 1354(3):241-51], muscular atrophy [J. Cell Biol. 2001, 152(1):75-85], neurological diseases such as Alzheimer's disease [Front Biosci. 2000, 5:D244-57], cancerous diseases such as breast cancer [Biol. Chem. 1999, 380(2):117-28], and autoimmune disorders [Clin. Exp. Immunol. 1997, 109(3):488-94].

RNA Binding Proteins:

The phrase “RNA binding proteins” refers to RNA binding proteins involved in splicing and translation regulation such as tRNA binding proteins, RNA helicases, double-stranded RNA and single-stranded RNA binding proteins, mRNA binding proteins, snRNA binding proteins, 5S RNA and 7S RNA binding proteins, poly-pyrimidine tract binding proteins, snRNA binding proteins, and AU-specific RNA binding proteins.

Pharmaceutical compositions including such proteins or protein encoding sequences, antibodies directed against such proteins or polynucleotides capable of altering expression of such proteins, may be used to treat diseases involving transcription and translation factors such as helicases, isomerases, histones and nucleases, diseases where there is impaired transcription, splicing, post-transcriptional processing, translation or stability of the RNA. Antibodies and polynucleotides such as PCR primers and molecular probes designed to identify such proteins or protein encoding sequences may be used for diagnosis of such diseases.

Examples of such diseases include, but are not limited to, cancerous diseases such as lymphomas [Tumori. 2003, 89(3):278-84], prostate cancer [Prostate. 2003, 57(1):80-92] or lung cancer [J. Pathol. 2003, 200(5):640-6], blood diseases, such as fanconi anemia [Curr. Hematol. Rep. 2003, 2(4):335-40], cardiovascular diseases such as atherosclerosis [J. Thromb. Haemost. 2003, 1(7):1381-90] muscle diseases [Trends Cardiovasc. Med. 2003, 3(5):188-95] and brain and neuronal diseases [Trends Cardiovasc. Med. 2003, 13(5):188-95; Neurosci. Lett. 2003, 342(1-2):41-4].

Nucleic Acid Binding Proteins:

The phrase “nucleic acid binding proteins” refers to proteins involved in RNA and DNA synthesis and expression regulation such as transcription factors, RNA and DNA binding proteins, zinc fingers, helicase, isomerase, histones, nucleases, ribonucleoproteins, and transcription and translation factors.

Pharmaceutical compositions including such proteins or protein encoding sequences, antibodies directed against such proteins or polynucleotides capable of altering expression of such proteins, may be used to treat diseases involving DNA or RNA binding proteins such as helicases, isomerases, histones and nucleases, for example diseases where there is abnormal replication or transcription of DNA and RNA respectively. Antibodies and polynucleotides such as PCR primers and molecular probes designed to identify such proteins or protein encoding sequences may be used for diagnosis of such diseases.

Examples of such diseases include, but are not limited to, neurological diseases such as renitis pigmentoas [Am. J. Ophthalmol. 2003, 136(4):678-87] parkinsonism [Proc. Natl. Acad. Sci. USA. 2003, 100(18):10347-52], Alzheimer [J. Neurosci. 2003, 23(17):6914-27] and canavan diseases [Brain Res Bull. 2003, 61(4):427-35], cancerous diseases such as leukemia [Anticancer Res. 2003, 23(4):3419-26] or lung cancer [J. Pathol. 2003, 200(5):640-6], miopathy [Neuromuscul Disord. 2003, 13(7-8):559-67] and liver diseases [J. Pathol. 2003, 200(5):553-60].

Proteins Involved in Metabolism:

The phrase “proteins involved in metabolism” refers to proteins involved in the totality of the chemical reactions and physical changes that occur in living organisms, comprising anabolism and catabolism; may be qualified to mean the chemical reactions and physical processes undergone by a particular substance, or class of substances, in a living organism. This group includes proteins involved in the reactions of cell growth and maintenance such as metabolism resulting in cell growth carbohydrate metabolism, energy pathways, electron transport, nucleobase, nucleoside, nucleotide and nucleic acid metabolism, protein metabolism and modification, amino acid and derivative metabolism, protein targeting, lipid metabolism, aromatic compound metabolism, one-carbon compound metabolism, coenzymes and prosthetic group metabolism, sulfur metabolism, phosphorus metabolism, phosphate metabolism, oxygen and radical metabolism, xenobiotic metabolism, nitrogen metabolism, fat body metabolism (sensu Insecta), protein localization, catabolism, biosynthesis, toxin metabolism, methylglyoxal metabolism, cyanate metabolism, glycolate metabolism, carbon utilization and antibiotic metabolism.

Pharmaceutical compositions including such proteins or protein encoding sequences, antibodies directed against such proteins or polynucleotides capable of altering expression of such proteins, may be used to treat diseases involving cell metabolism. Antibodies and polynucleotides such as PCR primers and molecular probes designed to identify such proteins or protein encoding sequences may be used for diagnosis of such diseases.

Examples of such metabolism-related diseases include, but are not limited to, multisystem mitochondrial disorder caused by mitochondrial DNA cytochrome C oxidase II deficiency. [Campos Y., et al., (2001) Ann. Neurol. 50(3):409-13], conduction defects and ventricular dysfunction in the heart associated with heterogeneous connexin43 expression [Gutstein D. E., et al., (2001) Circulation, 104(10):1194-9], atherosclerosis associated with growth suppressor p27 deficiency [Diez-Juan A., and Andres V. (2001) FASEB J., 15(11):1989-95], colitis associated with glutathione peroxidase deficiency [Esworthy R. S., et al., (2001) Am. J. Physiol. Gastrointest. Liver Physiol., 281(3):G848-55], systemic lupus erythematosus associated with deoxyribonuclease I deficiency [Yasutomo K., et al., (2001) Nat. Genet., 28(4):313-4], alcoholic pancreatitis [Pancreas. 2003, 27(4):281-5], amyloidosis and diseases that are related to amyloid metabolism, such as FMF, atherosclerosis, diabetes, and especially diabetes long term consequences, neurological diseases such as Creutzfeldt-Jakob disease, and Parkinson or Rasmussen's encephalitis.

Cell Growth and/or Maintenance Proteins:

The phrase “Cell growth and/or maintenance proteins” refers to proteins involved in any biological process required for cell survival, growth and maintenance, including proteins involved in biological processes such as cell organization and biogenesis, cell growth, cell proliferation, metabolism, cell cycle, budding, cell shape and cell size control, sporulation (sensu Saccharomyces), transport, ion homeostasis, autophagy, cell motility, chemi-mechanical coupling, membrane fusion, cell-cell fusion, and stress response.

Pharmaceutical compositions including such proteins or protein encoding sequences, antibodies directed against such proteins or polynucleotides capable of altering expression of such proteins, may be used to treat or prevent diseases such as cancer, degenerative diseases, for example neurodegenerative diseases or conditions associated with aging or alternatively, diseases wherein apoptosis which should have taken place, does not take place. Antibodies and polynucleotides such as PCR primers and molecular probes designed to identify such proteins or protein encoding sequences may be used for diagnosis of such diseases, detection of pre-disposition to a disease, and determination of the stage of a disease.

Examples of such diseases include, but are not limited to, ataxia-telangiectasia associated with ataxia-telangiectasia mutated deficiency [Hande et al., (2001) Hum. Mol. Genet., 10(5):519-28], osteoporosis associated with osteonectin deficiency [Delany et al., (2000) J. Clin. Invest., 105(7):91.5-23], arthritis caused by membrane-bound matrix metalloproteinase deficiency [Holmbeck et al., (1999) Cell, 99(1):81-92], defective stratum corneum and early neonatal death associated with transglutaminase 1 deficiency [Matsuki et al., (11998) Proc. Natl. Acad. Sci. USA, 95(3):1044-9], and Alzheimer's disease associated with estrogen [Simpkins et al., (1997) Am. J. Med., 103(3A):19S-25S].

Chaperones

Information derived from proteins such as ribosomal chaperone, peptidylprolyl isomerase, lectin-binding chaperone, nucleosome assembly chaperone chaperonin ATPase, cochaperone, heat shock protein, HSP70/HSP90 organizing protein fimbrial chaperone, metallochaperone, tubulin folding, HSC7-interacting protein can be used to diagnose/treat diseases involving pathological conditions, which are associated with non-normal protein activity or structure. Biding of the products of the proteins of this family, or antibodies reactive therewith, can modulate a plurality of protein activities as well as change protein structure. Alternatively, diseases in which there is abnormal degradation of other proteins, which may cause non-normal accumulation of various proteinaceous products in cells, caused non-normal prolonged or shortened) activity of proteins, etc.

Example of diseases that involve chaperones are cancerous diseases, such as prostate cancer (Semin Oncol. 2003 October; 30(5):709-16.); infectious diseases, such as prion infection (EMBO J. 2003 Oct. 15; 22(20):5435-5445.); neurological syndromes (J Neuropathol Exp Neurol. 2003 July; 62(7):751-64; Antioxid Redox Signal. 2003 June; 5(3):337-48; J. Neurochem. 2003 July; 86(2):394-404.)

Variants of Proteins which Accumulate an Element/Compound

Variant proteins which their wild type version naturally binds a certain compound or element inside the cell for storage of accumulation may have terapoetic effect as secreted variants. Ferritin, accumulates iron inside the cells. A secreted variant of this protein is expected to bind plasma iron, reduce its levels and therefore have a desired therapeutic effect in the syndrome of Hemosiderosis characterized by high levels of iron in the blood.

### Diseases that May be Treated/Diagnosed Using the Biomolecular Sequences of the Present Invention

Inflammatory Diseases

Examples of inflammatory diseases include, but are not limited to, chronic inflammatory diseases and acute inflammatory diseases.

Inflammatory Diseases Associated with Hypersensitivity

Examples of hypersensitivity include, but are not limited to, Types I-IV hypersensitivity, immediate hypersensitivity, antibody mediated hypersensitivity immune complex mediated hypersensitivity, T lymphocyte mediated hypersensitivity and DTH. An example of type I or immediate hypersensitivity is asthma. Examples of type II hypersensitivity include, but are not limited to, rheumatoid diseases, rheumatoid autoimmune diseases, rheumatoid arthritis [Krenn V et al., Histol Histopathol 2000 July; 15 (3):791], spondylitis, ankylosing spondylitis [Jan Voswinkel et al., Arthritis Res 2001; 3 (3): 189], systematic diseases, systemic autoimmune diseases, systemic lupus erythematosus [Erikson J. et al., Immunol Res 1998; 17 (1-2):49], sclerosis, systemic sclerosis [Renaudineau Y. et al., Clin Diagn Lab Immunol. 1999 March; 6 (2):156; Chan O T. et al., Immunol Rev 1999 June; 169:107], glandular diseases, glandular autoimmune diseases, pancreatic autoimmune diseases, diabetes, Type I diabetes [Zimmet P. Diabetes Res Clin Pract 1996 October; 34 Suppl:S125], thyroid diseases, autoimmune thyroid diseases, Graves' disease [Orgiazzi J. Endocrinol Metab Clin North Am 2000 June; 29 (2):339], thyroiditis, spontaneous autoimmune thyroiditis [Braley-Mullen H. and Yu S, J Immunol 2000 Dec. 15; 165 (12):7262], Hashimoto's thyroiditis [Toyoda N. et al., Nippon Rinsho 1999 August; 57 (8):1810], myxedema, idiopathic myxedema [Mitsuma T. Nippon Rinsho. 1999 August; 57 (8):1759]; autoimmune reproductive diseases, ovarian diseases, ovarian autoimmunity [Garza K M. et al., J Reprod Immunol 1998 February; 37 (2):87], autoimmune anti-sperm infertility [Diekman A B. et al., Am J Reprod Immunol. 2000 March; 43 (3):134], repeated fetal loss [Tincani A. et al. Lupus 1998; 7 Suppl 2:S107-9], neurodegenerative diseases, neurological diseases, neurological autoimmune diseases, multiple sclerosis [Cross A H. et al., J Neuroimmunol 2001 Jan. 1; 112 (1-2)-1], Alzheimer's disease [Oron L. et al., J Neural Transm Suppl. 1997; 49:77], myasthenia gravis [Infante A J. and Kraig E, Int Rev Immunol 1999; 18 (1-2):83], motor neuropathies [Kornberg A J. J Clin Neurosci. 2000 May; 7 (3):191], Guillain-Barre syndrome, neuropathies and autoimmune, neuropathies [Kusunoki S. Am J Med Sci. 2000 April; 319 (4):234], myasthenic diseases, Lambert-Eaton myasthenic syndrome [Takamori M. Am J Med Sci. 2000 April; 3319 (4):204], paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies [Antoine J C. and Honnorat J. Rev Neurol (Paris) 2000 January; 156 (1):23], neuropathies, dysimmune neuropathies [Nobile-Orazio E. et al., Electroencephalogr Clin Neurophysiol Suppl 1999; 50:419], neuromyotonia, acquired neuromyotonia, arthrogryposis multiplex congenita [Vincent A. et al., Ann NY Acad Sci. 1998 May 13; 841:482], cardiovascular diseases, cardiovascular autoimmune diseases, atherosclerosis [Matsuura E. et al., Lupus. 1998; 7 Suppl-2:S135], myocardial infarction [Vaarala O. Lupus. 1998; 7 Suppl 2:S132], thrombosis [Tincani A. et al., Lupus 1998; 7 Suppl 2:S107-9], granulomatosis, Wegener's granulomatosis, arteritis, Takayasu's arteritis and Kawasaki syndrome [Praprotnik S. et al., Wien Klin Wochenschr 2000 Aug. 25; 112 (15-16):660], anti-factor VIII autoimmune disease [Lacroix-Desmazes S. et al., Semin Thromb Hemost 2000; 26 (2): 157], vasculitises, necrotizing small vessel vasculitises, microscopic polyangiitis, Churg and Strauss syndrome, glomerulonephritis, pauci-immune focal necrotizing glomerulonephritis, crescentic glomerulonephritis [Noel. L H. Ann. Med Interne (Paris). 2000 May; 151 (3):178], antiphospholipid syndrome [Flamholz R. et al., J Clin Apheresis 1999; 14 (4):171], heart failure, agonist-like β-adrenoceptor antibodies in heart failure [Wallukat G. et al., Am J Cardiol. 1999 Jun. 17; 83 (12A):75H], thrombocytopenic purpura [Moccia F. Ann Ital Med Int. 1999 April-June; 14 (2):114], hemolytic anemia, autoimmune hemolytic anemia [Efremov D G. et al.; Leuk Lymphoma 1998 January; 28 (3-4):285], gastrointestinal diseases, autoimmune diseases of the gastrointestinal tract, intestinal diseases, chronic inflammatory intestinal disease [Garcia Herola A. et al., Gastroenterol Hepatol. 2000 January; 23 (1): 16], celiac disease. [Landau Y E. and Shoenfeld Y. Harefuah 2000 Jan. 16; 138 (2):122], autoimmune diseases of the musculature, myositis, autoimmune myositis, Sjogren's syndrome [Feist E. et. al., Int Arch Allergy Immunol 2000 September; 123 (1):92], smooth muscle autoimmune disease [Zauli D. et al., Biomed Pharmacother 1999 June; 53 (5-6):234], hepatic diseases, hepatic autoimmune diseases, autoimmune hepatitis. [Manns M P. J Hepatol 2000 August; 33 (2):326] and primary biliary cirrhosis [Strassburg C P. et al., Eur J Gastroenterol Hepatol. 1999 June; 11 (6):595].

Examples of type IV or T cell mediated hypersensitivity, include, but are not limited to, rheumatoid diseases, rheumatoid arthritis [Tisch R, McDevitt H O. Proc Natl Acad Sci USA 1994 Jan. 18; 91 (2):437], systemic diseases, systemic autoimmune diseases, systemic lupus erythematosus [Datta S K., Lupus 1998; 7 (9):591], glandular diseases, glandular autoimmune diseases, pancreatic diseases, pancreatic autoimmune diseases, Type 1 diabetes [Castano L. and Eisenbarth G S. Ann. Rev. Immunol. 8:647], thyroid diseases, autoimmune thyroid diseases, Graves' disease [Sakata S. et al., Mol Cell Endocrinol 1993 March; 92 (1):77], ovarian diseases [Garza K M. et al., J Reprod Immunol 1998 February; 37(2):87], prostatitis, autoimmune prostatitis [Alexander R B. et al., Urology 1997 December; 50 (6):893], polyglandular syndrome, autoimmune polyglandular syndrome, Type I autoimmune polyglandular syndrome [Hara T. et al., Blood. 1991 Mar. 1; 77 (5):1127], neurological diseases, autoimmune neurological diseases, multiple sclerosis, neuritis, optic neuritis [Soderstrom M. et al., J Neurol Neurosurg Psychiatry 1994 May; 57 (5):544], myasthenia gravis [Oshima M. et al., Eur J Immunol 1990 December; 20 (12):2563], stiff-man syndrome [Hiemstra H S. et al., Proc Natl Acad Sci USA 2001 Mar. 27; 98 (7):3988], cardiovascular diseases, cardiac, autoimmunity in Chagas' disease [Cunha-Neto E. et al., J Clin Invest 1996 Oct. 15; 98 (8):1709], autoimmune thrombocytopenic purpura [Semple J W. et al., Blood 1996 May 15; 87 (10):4245], anti-helper T lymphocyte autoimmunity [Caporossi A P. et al., Viral Immunol 1998; 11 (1):9], hemolytic anemia [Sallah S. et al., Ann Hematol 1997 March; 74 (3):139], hepatic diseases, hepatic autoimmune diseases, hepatitis, chronic active hepatitis [Franco A. et al., Clin Immunol Immunopathol 1990 March; 54 (3):382], biliary cirrhosis, primary biliary cirrhosis [Jones D E. Clin Sci (Colch) 1996 November; 91 (5):551], nephric diseases, nephric autoimmune diseases, nephritis, interstitial nephritis [Kelly C J. J Am Soc Nephrol 1990 August; 1 (2):140], connective tissue diseases, ear diseases, autoimmune connective tissue diseases, autoimmune ear disease [Yoo T J. et al., Cell Immunol 1994 August; 157 (1):249], disease of the inner ear [Gloddek B. et al., Ann NY Acad Sci 1997 Dec. 29; 830:266], skin diseases cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Autoimmune Diseases

Examples of autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases related to reproduction, connective tissue diseases and systemic diseases.

Examples of autoimmune cardiovascular and blood diseases include, but are not limited to atherosclerosis [Matsuura E. et al., Lupus. 1998; 7. Suppl. 2:S135], myocardial infarction [Vaarala O. Lupus. 1998; 7-Suppl 2:S132], thrombosis [Tincani A. et al., Lupus. 1998; 7 Suppl 2:S107-9], Wegener's granulomatosis, Takayasu's arteritis, Kawasaki syndrome [Praprotnik S. et al., Wien Klin Wochenschr 2000 Aug. 25; 112 (15-16):660], anti-factor VIII autoimmune disease [Lacroix-Desmazes S. et al., Semin Thromb Hemost. 2000; 26 (2): 157], necrotizing small vessel vasculitis, microscopic polyangiitis, Churg and Strauss syndrome, pauci-immune focal necrotizing and crescentic glomerulonephritis [Noel L H. Ann Med Interne (Paris). 2000 May; 151 (3):178], antiphospholipid syndrome [Flamholz R. et al., J. Clin Apheresis 1999; 14 (4): 171], antibody-induced heart failure [Wallukat G et al., J Cardiol. 1999 Jun. 17; 83 (12A):75H], thrombocytopenic purpura 4[Moccia F. Ann Ital Med Int. 1999 April-June; 14 (2):114; Semple J W. et al., Blood 1996 May 15; 87 (10):4245], autoimmune hemolytic anemia [Efremov D G. et. al., Leuk Lymphoma 1998 January; 28 (3-4):285; Sallah S. et al., Ann Hematol. 1997 March; 74 (3):139], cardiac autoimmunity in Chagas' disease [Cunha-Neto E. et al., J Clin Invest 1996 Oct. 15; 98 (8):1709) and anti-helper T lymphocyte autoimmunity [Caporossi A P. et al., Viral Immunol 1998; 11 (1):9].

Examples of autoimmune rheumatoid diseases include, but are not limited to rheumatoid arthritis [Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis [Jan Voswinkel et al., Arthritis Res 2001; 3 (3): 189].

Examples of autoimmune glandular diseases include, but are not limited to, pancreatic disease, Type I diabetes, Type II diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune, prostatitis and Type I autoimmune polyglandular syndrome diseases include, but are not limited to autoimmune diseases of the pancreas, Type 1 diabetes [Castano L. and Eisenbarth G S. Ann. Rev. Immunol. 8:647; Zimmet P. Diabetes Res Clin Pract 1996 October; 34 Suppl:S125], autoimmune thyroid diseases, Graves' disease [Orgiazzi J. Endocrinol Metab Clin North Am 2000 June; 29 (2):339; Sakata S. et al., Mol Cell Endocrinol 1993 March; 92(1):77], spontaneous autoimmune thyroiditis [Braley-Mullen H. and Yu S, J Immunol 2000 Dec. 15; 165 (112):7262], Hashimoto's thyroiditis. [Toyoda N. et al., Nippon Rinsho. 1999 August; 57 (8):1810], idiopathic myxedema [Mitsuma T. Nippon Rinsho. 1999 August; 57 (8):1759], ovarian autoimmunity [Garza K M. et al., J Reprod Immunol 1998 February; 37 (2):87], autoimmune anti-sperm infertility [Diekman A B. et al., Am J Reprod Immunol. 2000 March; 43 (3):134], autoimmune prostatitis [Alexander R B. et al., Urology 1997 December; 50 (6):893) and Type I autoimmune polyglandular syndrome [Hara T. et al., Blood. 1991 Mar. 1; 77 (5):1127].

Examples of autoimmune gastrointestinal diseases include but are not limited to, chronic inflammatory intestinal diseases [Garcia Herola A. et al., Gastroenterol Hepatol. 2000 January; 23 (1):16], celiac disease [Landau Y E. and Shoenfeld Y. Harefuah 2000 Jan. 16; 138 (2):122], colitis, ileitis and Crohn's disease and ulcerative colitis.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis [Franco A. et al., Clin Immunol Immunopathol 1990 March; 54 (3):382], primary biliary cirrhosis [Jones D E. Clin Sci (Colch) 1996 November; 91 (5):551; Strassburg C P. et al., Eur J Gastroenterol Hepatol. 1999 June; 11 (6):595) and autoimmune hepatitis [Manns M P. J Hepatol 2000 August; 33 (2):326].

Examples of autoimmune neurological diseases include, but are not limited to, multiple sclerosis [Cross A H. et al., J. Neuroimmunol 2001 Jan. 1; 112 (1-2):1], Alzheimer's disease [Oron L. et al., J Neural Transm Suppl. 1997; 49:77], myasthenia gravis [Infante A J. and Kraig E, Int Rev Immunol 1999; 18 (1-2):83; Oshima M. et al., Eur J Immunol 1990 December; 20 (12):2563], neuropathies, motor neuropathies [Kornberg A J. J Clin Neurosci. 2000 May; 7 (3) 191], Guillain-Barre syndrome and autoimmune neuropathies: [Kusunoki S. Am J Med Sci. 2000 April; 319, (4):234], myasthenia, Lambert-Eaton myasthenic syndrome [Takamori M. Am J Med Sci. 2000 April; 319 (4):204], paraneoplastic neurological diseases, cerebellar atrophy, paraneoplastic cerebellar atrophy and stiff-man syndrome [Hiemstra H S. et al., Proc Natl Acad Sci units S A 2001 Mar. 27; 98 (7):3988], non-paraneoplastic stiff man syndrome, progressive cerebellar atrophies, encephalitis, Rasmussen's encephalitis amyotropic lateral sclerosis, Sydeham chorea, Gilles de la Tourette syndrome and autoimmune polyendocrinopathies [Antoine J C. and Honnorat J. Rev Neurol (Paris) 2000 January; 156 (1):23], dysimmune neuropathies [Nobile-Orazio E. et al., Electroencephalogr Clin Neurophysiol Suppl 1999; 50:419], acquired neuromyotonia, arthrogyposis multiplex congenita [Vincent A. et al., Ann NY Acad Sci. 1998 May 13; 841:482], neuritis, optic neuritis [Soderstrom M. et al., J Neurol Neurosurg Psychiatry 1994 May; 57 (5):544) multiple sclerosis and neurodegenerative diseases.

Examples of autoimmune muscular diseases include, but are not limited to, myositis, autoimmune myositis and primary Sjogren's syndrome. [Feist E. et al., Int Arch Allergy Immunol 2000 September; 123 (1):92) and smooth muscle autoimmune disease [Zauli D. et al., Biomed Pharmacother 1999 June; 53 (5-6):234].

Examples of autoimmune nephric diseases include, but are not limited to, nephritis and autoimmune interstitial nephritis [Kelly C J. J Am Soc Nephrol 1990 August; 1 (2):140], glommerular nephritis.

Examples of autoimmune diseases related to reproduction include, but are not limited to, repeated fetal loss [Tincani A. et al., Lupus 1998; 7 Suppl 2:S107-9].

Examples of autoimmune connective tissue diseases include, but are not limited to, ear diseases, autoimmune ear diseases [Yoo T J. et al., Cell Immunol 1994 August; 157 (1):249) and autoimmune diseases of the inner ear [Gloddek B. et al., Ann NY Acad Sci 1997 Dec. 29; 830:266].

Examples of autoimmune systemic diseases include, but are not limited to, systemic lupus erythematosus [Erikson J. et al., Immunol Res 1998; 17 (1-2):49) and systemic sclerosis [Renaudineau Y. et al., Clin Diagn Lab Immunol. 1999 March; 6 (2):156; Chan O T. et al., Immunol Rev 1999 June; 169:107].

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal, diseases, mycoplasma diseases, and prion diseases.

Graft Rejection Diseases

Examples of diseases associated with transplantation of a graft include, but are not limited to, graft rejection, chronic graft rejection, subacute graft rejection, hyperacute graft rejection, acute graft rejection, and graft versus host disease.

Allergic Diseases

Examples of allergic diseases include, but not limited to, asthma, hives urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils. Acute monocytic leukemia. Acute myelomonocytic leukemia with eosinophilia; malignant lymphoma, such as Birkitt's Non-Hodgkin's; Lymphoctyic leukemia, such as, acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast; skin, prostate, and ovarian.

### Example 8

### Data Files Supporting Designation of Alternative Exons

File DataOnExons.txt—contains the summary of all details according to which the exon was declared as alternative. Each line in this file begins with the name of the exon, and thereafter contains the following fields:

1. #MOUSE_EXON—the name of the orthologous matching mouse exon. File mouse_exons.fasta contains the sequences of the mouse exons that correspond to the human exons (matching to, the #MOUSE_EXON field in file DataOnExons.txt file).
#ST strand of this exon on the DNA
#EXON_LEN length of exon
#EXON_DIVIDABLE_BY_{—}3—is the exon divisable by; 3 (1=yes, 0=no)
#EXON_ALN_LEN—length of human/mouse local exon alignment
#EXON_ALN_IDN—identity level in human/mouse local exon alignment
#UPSTREAM_ALN_LEN—length of human/mouse local alignment of upstream intronic sequences
#UPSTREAM_ALN_IDN—identity level of human/mouse local alignment of upstream intronic sequences
#DOWNSTREAM_ALN_LEN—length of human/mouse local alignment of downstream intronic sequences
#DOWNSTREAM_ALN_IDN—identity level of human/mouse local alignment of downstream intronic sequences
#EXON_GLOBAL_ALN_LEN—length of human/mouse global exon alignment
#EXON_GLOBAL_ALN_IDN—identity level in human/mouse global exon alignment
#PERC_CONST—percent of constitutive exons in training set that correspond to these combination of features
#PERC_ALT—percent of alternative exons in training set that correspond to these combination of features
#SCORE—alternativeness score, calculated as described in the text

### Example 9

### Description of CD-ROM3

Enclosed CD-ROM3 contains the following files:

1. “CROG_localization_{—}1”, containing protein cellular localization information.

2. “crog_proteins_ipr_report_{—}1_dos”, containing information related to Interpro analysis of domains.

3. “CROG_expression_x”, wherein “x” may be 1 or 2, containing information related to expression of transcripts according to oligonucleotide data.

4. “oligo probs abbreviations for patent”, containing information about abbreviations of tissue names for oligonucleotide probe binding.

5. “crog_report_x_{—}1” wherein “x” may be from 1 to 45, containing comparison reports between known protein sequences and variant protein sequences according to the present invention, including identifying unique regions therein.

6. “variants_report.txt”, containing the information about the different variants of the known protein sequences (for example, due to known amino acid changes because of an SNP).

All tables are best viewed by using a text editor with the “word wrap” function disabled (to preserve line integrity) and in a fixed width font, such as Courier for example, preferably in font size 10. Table spacing is described for each table as a guide to assist in reading the tables.

With regard to protein cellular localization information, table structure is as follows: column 1 features the protein identifier as used throughout the application to identify this sequence column 2 features the name of the protein; column 3 shows localization (which may be intracellular, membranal or secreted); and column 4 gives the reason for this localization in terms of results from particular software programs that were used to determine localization. Spacing for this table is as follows: column 1: characters 1-9; column 2: characters 10-45; column 3: 46-61; and column 4: characters 62-121.

Information given in the text with regard to cellular localization was determined according to four different software programs: (i) tmhmm (from Center for Biological Sequence Analysis, Technical University of Denmark DTU, http://www.cbs.dtu.dk/services/TMHMM/TMHMM2.0b.guide.php) or (ii) tmpred (from EMBnet, maintained by the ISREC Bionformatics group and the LICR Information Technology Office, Ludwig Institute for Cancer Research, Swiss Institute of Bioinformatics, http://www.ch.embnet.org/software/TMPRED_form.html) for transmembrane region prediction; (iii) signalp_hmm or (iv) signalp_nn (both from Center for Biological Sequence Analysis, Technical University of Denmark DTU, http://www.cbs.dtu.dk/services/SignaIP/background/prediction.php) for signal peptide prediction. The terms “signalp_hmm” and “signalp_nn” refer to two modes of operations for the program SignaIP:hmm refers to Hidden Markov Model, while nn refers to neural networks. Localization was also determined through manual inspection of known protein localization and/or gene structure, and the use of heuristics by the individual inventor. In some cases for the manual inspection of cellular localization prediction inventors used the ProLoc computational platform [Einat Hazkani-Covo, Erez Levanon, Galit Rotman, Dan Graur and Amit Novik; (2004) “Evolution of multicellularity in metazoa: comparative analysis of the subcellular localization of proteins in Saccharomyces, Drosophila and Caenorhabditis.” Cell Biology International 2004; 28(3):171-8.], which predicts protein localization based on various parameters including, protein domains (e.g., prediction of trans-membranous regions and localization thereof within the protein), pI, protein length, amino acid composition, homology to pre-annotated proteins, recognition of sequence patterns which direct the protein to a certain organelle (such as, nuclear localization signal, NLS, mitochondria localization signal), signal peptide and anchor modeling and using unique domains from Pfam that are specific to a single compartment.

With regard to Interpro analysis of domains, table structure is as follows: column 1 features the protein identifier as used throughout the application to identify this sequence; column 2 features the name of the protein; column 3 features the Intepro identifier; column 4 features the analysis type; column 5 features the domain description; and column 6 features the position(s) of the amino acid residues that are relevant to this domain on the protein (amino acid sequence). Spacing for this table is as follows: column 1: characters 1-8; column 2: characters 9-48; column 3: 49-72; column 4: characters 13-96; column 5: characters 97-136; and column 6: 137-168.

Interpro provides information with regard to the analysis of amino acid sequences to identify, domains having certain functionality (see Mulder et al (2003), The InterPro Database, 2003 brings increased-coverage and new features, Nucleic Acids Res. 31, 315-318 for a reference). It features a database of protein families, domains and functional sites in which identifiable features found in known proteins can be applied to unknown protein sequences. The analysis type relates to the type of software used to determine the domain: Pfam (see Bateman A, et al (2004) The Pfam protein families database. Nucleic Acids Res. 32, 138-41), SMART (see Letunic I, et al (2004) SMART 40: towards genomic data integration. Nucleic Acids Res. 32, 142-4), TIGRFAMs (see Haft D H, et al (2003) The TIGRFAMs database of protein families. Nucleic Acids Res. 31, 371-373), PIRSF (see Wu C H et al (2003) The Protein Information Resource. Nucleic Acids Res. 31, 345-347), and SUPERFAMILY (see Gough J et al (2001) Assignment of homology to genome sequences using a library of Hidden Markov. Models that represent all proteins of known structure. Journal Molecular Biol. 313, 903-919) all use hidden Markov models (HMMs) to determine the location of domains on protein sequences.

With regard to transcript expression information, table structure is as follows: column 1 features the transcript identifier as used throughout the application to identify this sequence; column 2 features the name of the transcript; column 3 features the name of the probeset used in the chip experiment; and column 4 relates to the tissue and level of expression found. Spacing for this table is as follows: column 1: characters 1-9; column 2: characters 10-27; column 3: 28-41; and column 4: characters 42-121. Information given in the text with regard to expression was determined according to oligonucleotide binding to arrays. Information is given with regard to overexpression of a cluster in cancer based on microarrays. As a microarray reference, in the specific segment paragraphs, the unabbreviated tissue name was used as the reference to the type of chip for which expression was measured. Oligonucleotide microarray results were taken from Affymetrix data, available from Affymetrix Inc, Santa Clara, Calif., USA (see for example data regarding the Human Genome U133 (HG-U133) Set at www.affymetrix.com/products/arrays/specific/hgu133.affx; GeneChip Human Genome U133A 2.0 Array at www.affymetrix.com/products/arrays/specific/hgu133av2.affx; and Human Genome U133 Plus 2.0 Array at www.affymetrix.com/products/arrays/specific/hgu133plus.affx). The data is available from NCBI Gene Expression Omnibus (see www.ncbi.nlm.nih.gov/projects/geo/ and Edgar et al, Nucleic Acids Research, 2002, Vol. 30, No. 1 207-210). The dataset (including results) is available from www.ncbi.nlm.nih.gov/geo/query/acc.cgi?acc=GSE1133 for the Series GSE1133 database (published on March 2004); a reference to these results is as follows: Su et al (Proc Natl Acad Sci USA. 2004 Apr. 20, 101(16):6062-7. Epub 2004 Apr. 09).

With regard to comparison reports between variant protein according to the present invention and known protein, table structure is as follows: column 1 features the protein identifier as used throughout the application to identify this sequence, column 2 features the name of the protein; column 3 reports on the differences between the variant protein sequence and the known protein sequence (including the name of the known protein); and column 4 shows the alignment between the variant protein sequence and the known protein sequence. Spacing for this table is as follows: characters 1-18: column 1; characters 19-32: column 2; characters 33-92: column 3; and characters 97-170: column 4.

Information given in the text with regard to the Homology to the known proteins was determined by Smith-Waterman version 5.1.2 using special (non default) parameters as follows:
model=sw.model
GAPEXT=0
GAPOP=100.0
MATRIX=blosum100

In some cases, the known protein sequence was included with one or more known variations in order to assist in the above comparison. These sequences are given in variants_report.txt: column 1 features the name of the protein sequence as it appears in the comparison to the variant protein(s); column 2 features the altered protein sequence; column 3 features the type of variation (for example init_met refers to lack of methioinine at the beginning of the original sequence); column 4 states the location of the variation in terms of the amino acid(s) that is/are changed, column 5 shows FROM; and column 6 shows TO (FROM and TO—start and end of the described feature on the protein sequence). Spacing for this table is as follows: column 1: characters 1-24; column 2: characters 25-96; column 3: characters 97-120; column 4: characters 121-144, and column 5: characters: 145-169.

The comparison reports herein may optionally include such features as bridges, tails, heads and/or insertions (unique regions), and/or analogs, homologs and derivatives of such peptides (unique regions).

As used herein a “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding known protein, while at least a second portion of the variant comprises the tail.

As used herein a “head” refers to a peptide sequence at the beginning of an amino acid sequence that is unique to a splice variant according to the present invention. Therefore, a splice variant having such a head may optionally be considered as a chimera, in that at least a first portion of the splice variant comprises the head, while at least a second portion is typically highly homologous (often 100% identical) to a portion of the corresponding known protein.

As used herein “an edge portion” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a known protein” portion of a variant, or a join between an insertion and a “known protein” portion of a variant.

Optionally and preferably, a bridge between a tail or a head or a unique insertion, and a “known protein” portion of a variant, comprises at least about 10 amino acids, more preferably at least about 20 amino acids, most preferably at least about 30 amino acids, and even more preferably at least about 40 amino acids, in which at least one amino acid is from the tail/head/insertion and at least one amino acid is from the “known protein” portion of a variant. Also optionally, the bridge may comprise any number of amino acids from about 10 to about 40 amino acids (for example, 10, 11, 12, 13 . . . 37, 38, 39, 40 amino acids in length, or any number in between).

It should be noted that bridge cannot be extended beyond the length of the sequence in either direction, and it should be assumed that every bridge description is to be read in such manner that the bridge length does not extend beyond the sequence itself.

Furthermore, bridges are described with regard to a sliding window in certain contexts below. For example, certain descriptions of the bridges feature the following format: a bridge between two edges (in which a portion of the known protein is not present in the variant) may optionally be described as follows: a bridge portion of CONTIG-NAME_P1 (representing the name of the protein), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise XX (2 amino acids in the center of the bridge, one from each end of the edge), having a structure a follows (numbering according to the sequence of, CONTIG-NAME_P1): a sequence starting from any of amino acid numbers 49-x to 49 (for example); and ending at any of amino acid numbers 50+((n−2)−x) (for example), in which x varies from 0 to n−2. In this example, it should also be read as including bridges in which n is any number of amino acids between 10-50 amino acids in length. Furthermore, the bridge polypeptide cannot extend beyond the sequencer so it should be read such that 49-x (for example) is not less than 1 nor 50+((n−1)−x) (for example) greater than the total sequence length.

In another embodiment, this invention provides antibodies specifically recognizing the splice variants and polypeptide fragments thereof of this invention. Preferably such antibodies differentially recognize splice variants of the present invention but do not recognize a corresponding known protein, optionally and more preferably through recognition of a unique region as described herein.

All nucleic acid sequences and/or amino acid sequences shown herein as embodiments of the present invention relate to their isolated form, as isolated polynucleotides (including for all transcripts), oligonucleotides (including for all segments, amplicons and primers), peptides (including for all tails, bridges, insertions or heads, optionally including other antibody epitopes as describe herein) and/or polypeptides (including for all proteins). It should be noted that oligonucleotide and polynucleotide, or peptide and polypeptide, may optionally be used interchangeably.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of identifying alternatively spliced exons, the method comprising, scoring each of a plurality of exon sequences derived from genes of a species according to at least one sequence parameter, wherein exon sequences of said plurality of exon sequences scoring above a predetermined threshold represent alternatively spliced exons, thereby identifying the alternatively spliced exons.

2. The method of claim 1, wherein said at least one sequence parameter is selected from the group consisting of:
(i) exon length;
(ii) division by 3;
(iii) conservation level between said plurality of exon sequences of genes of a species and corresponding exon sequences of genes of an ortholohgous species;
(iv) length of conserved intron sequences upstream of each of said plurality of exon sequences;
(v) length of conserved intron sequences downstream of each of said plurality of exon sequences;
(vi) conservation level of said intron sequences upstream of each of said plurality of exon sequences; and
(vii) conservation level of said intron sequences downstream of each of said plurality of exon sequences;

3. The method of claim 2, wherein said exon length does not exceed 1000 bp.

4. The method of claim 2, wherein said conservation level is at least 95%.

5. The method of claim 2, wherein said length of conserved intron sequences upstream of each of said plurality of exon sequences is at least 12.

6. The method of claim 2, wherein said length of conserved intron sequences downstream of each of said plurality of exon sequences is at least 15.

7. The method of claim 2, wherein said conservation level of said intron sequences upstream of each of said plurality of exon sequences is at least 85%.

8. The method of claim 2, wherein said conservation level of said intron sequences downstream of each of said plurality of exon sequences is at least 60%.

9. A system for generating a database of alternatively spliced exons, the system comprising a processing unit, said processing unit executing a software application configured for:
(a) scoring each of a plurality of exon sequences derived from genes of a species according to at least one sequence parameter, wherein exon sequences of said plurality of exon sequences scoring above a predetermined threshold represent alternatively spliced exons, to thereby identify the alternatively spliced exons; and
(b) storing said identified alternatively spliced exons to thereby generate the database of alternatively spliced exons.

10. The system of claim 9, wherein said at least one sequence parameter is selected from the group consisting of:
(i) exon length;
(i) division by 3;
(iii) conservation level between said plurality of exon sequences of genes of a species and corresponding exon sequences of genes of an ortholohgous species;
(iv) length of conserved intron sequences upstream of each of said plurality of exon sequences;
(v) length of conserved intron sequences downstream of each of said plurality of exon sequences;
(vi) conservation level of said intron sequences upstream of each of said plurality of exon sequences; and
(vii) conservation level of said intron sequences downstream of each of said plurality of exon sequences;

11. The system of claim 10, wherein said exon length does not exceed 1000 bp.

12. The system of claim 10, wherein said conservation level is at least 95%.

13. The system of claim 10, wherein said length of conserved intron sequences upstream of each of said plurality of exon sequences is at least 12.

14. The system of claim 10, wherein said length of conserved intron sequences downstream of each of said plurality of exon sequences is at least 15.

15. The system of claim 10, wherein said conservation level of said intron sequences upstream of each of said plurality of exon sequences is at least 85%.

16. The system of claim 10, wherein said conservation level of said intron sequences downstream of each of said plurality of exon sequences is at least 60%.

17. A computer readable storage medium comprising data stored in a retrievable manner, said data including sequence information as set forth in the files “transcripts. fasta” and “proteins.fasta” of enclosed CD-ROM1 and the files forth in the file “AnnotationForPatent.txt” of enclosed CD-ROM1.

18. A method of predicting expression products of a gene of interest, the method comprising:
(a) scoring exon sequences of the gene of interest according to at least one sequence parameter and identifying exon sequences scoring above a predetermined threshold as alternatively spliced exons of the gene of interest; and
(b) analyzing chromosomal location of each of said alternatively spliced exons with respect to coding, sequence of the gene of interest to thereby predict expression products of the gene of interest.

19. The method of claim 18, wherein said at least one sequence parameter is selected from the group consisting of:
(i) exon length;
(ii) division by 3;
(iii) conservation level between said plurality of exon sequences of genes of a species and corresponding exon sequences of genes of an
(iv) orthologous species;
(iv) length of conserved intron sequences upstream of each of said plurality of exon sequences;
(v) length of conserved intron sequences downstream of each of said plurality of exon sequences;
(vi) conservation level of said intron sequences upstream of each of said plurality of exon sequences; and
(vii) conservation level of said intron sequences downstream of each of said plurality of exon sequences;

20. The method of claim 19, wherein said exon length does not exceed 1000 bp.

21. The method of claim 19, wherein said conservation level is at least 95%.

22. The method of claim 19, wherein said length of conserved intron sequences upstream of each of said plurality of exon sequences is at least 12.

23. The method of claim 19, wherein said length of conserved intron sequences downstream of each of said plurality of exon sequences is at least 15.

24. The method of claim 19, wherein said conservation level of said intron sequences upstream of each of said plurality of exon sequences is at least 85%.

25. The method of claim 19, wherein said conservation level of said intron sequences downstream of each of said plurality of exon sequences is at least 60%.

26. A method of predicting expression products of a gene of interest in a given species, the method comprising:
(a) providing a contig of exon sequences of the gene of interest of a first species;
(b) identifying exon sequences of an orthologue of the gene of interest of said first species which align to a genome of said first species;
(c) assembling said exon sequences of said orthologue of the gene of interest in said contig, thereby generating a hybrid contig;
(d) identifying in said hybrid contig, exon sequences of said orthologue of the gene of interest, which do not align with said exon sequences of the gene of interest of said first species, thereby uncovering non-overlapping exon sequences of the gene of interest; and
(e) analyzing chromosomal location of non-overlapping exon sequences of the gene of interest with respect to the chromosomal location of the gene of interest to thereby predict expression products of the gene of interest in a given species.

27. The method of claim 26, wherein at least a portion of said exon sequences are alternatively spliced sequences.

28. The method of claim 27, wherein said alternatively spliced sequences are identified by scoring exon sequences of the gene of interest according to at least one sequence parameter, wherein exon sequences scoring above a predetermined threshold represent said alternatively spliced exons of the gene of interest.

29. The method of claim 28, wherein said at least one sequence parameter is selected from the group consisting of:
(i) exon length;
(ii) division by 3;
(iii) conservation level between said plurality of exon sequences of genes of a species and corresponding exon sequences of genes of an orthologous species;
(iv) length of conserved intron sequences upstream of each of said plurality of exon sequences;
(v) length of conserved intron sequences downstream of each of said
plurality of exon sequences;
(vi) conservation level of said intron sequences upstream of each of said plurality of exon sequences; and
(vii) conservation level of said intron sequences downstream of each of said plurality of exon sequences;

30. The method of claim 29, wherein said exon length does not exceed 1000 bp.

31. The method of claim 29, wherein said conservation level is at least 95%.

32. The method of claim 29, wherein said length of conserved intron sequences upstream of each of said plurality of exon sequences is at least 12.

33. A The method of claim 29, wherein said length of conserved intron sequences downstream of each of said plurality of exon sequences is at least 15.

34. The method of claim 29, wherein said conservation level of said intron sequences upstream of each of said plurality of exon sequences is at least 85%.

35. The method of claim 29, wherein said conservation level of said intron sequences downstream of each of said plurality of exon sequences is at least 60%.

36. An isolated polynucleotide comprising a nucleic acid sequence being at least 70% identical to a nucleic acid sequence of the sequences set forth in file “transcripts.fasta” of CD-ROM1 or in the file “transcripts” of CD-ROM2.

37. The isolated polynucleotide of claim 36, wherein said nucleic acid sequence is set forth in the file “transcripts.fasta” of enclosed CD-ROM1 or in the file “transcripts” of enclosed CD-ROM 2.

38. An isolated polynucleotide comprising a nucleic acid sequence encoding a polypeptide having an amino acid sequence at least 70% homologous to a sequence set forth in the file “proteins.fasta” of enclosed CD-ROM or in the file “proteins” of enclosed CD-ROM2.

39. An isolated polypeptide having an amino acid sequence at least 80% homologous to a sequence set forth in the file proteins.fasta” of enclosed CD-ROM1 or in the file “proteins” of enclosed CD-ROM2.

40. Use of a polynucleotide or polypeptide set forth in the file “transcripts.fasta” of CD-ROM1 or in the file “transcripts” of CD-ROM2 or in the file “proteins.fasta” of enclosed CD-ROM1 or in the file “proteins” of enclosed CD-ROM2 for the diagnosis and/or treatment of the diseases listed in Example 8.
